(19) United States
(12) Patent Application Publication
(10) Pub. No.: US 2024/0190824 A1
(43) Pub. Date: Jun. 13, 2024

(54) INHIBITORS OF TREK (TWIK RELATED K+ CHANNELS) CHANNEL FUNCTION

(71) Applicants: ONO PHARMACEUTICAL CO., LTD., Osaka-shi, Osaka (JP); VANDERBILT UNIVERSITY, Nashville, TN (US)

(72) Inventors: Craig W LINDSLEY, Brentwood, TN (US); Darren W ENGERS, Brentwood, TN (US); Elizabeth S CHILDRESS, Franklin, TN (US); Sean R BOLLINGER, Chapel Hill, TN (US); Joza SCHMITT, Nashville, TN (US); Trevor C CHOPKO, Akron, OH (US); Jerod S DENTON, Nashville, TN (US); Motoyuki TANAKA, Mishima-gun (JP); Haruto KURATA, Mishima-gun (JP)

(73) Assignees: ONO PHARMACEUTICAL CO., LTD., Osaka-shi, Osaka (JP); VANDERBILT UNIVERSITY, Nashville, TN (US)

(21) Appl. No.: 17/769,300

(22) PCT Filed: Oct. 23, 2020
(86) PCT No.: PCT/JP2020/039994

§ 371 Date: Apr. 14, 2022

Related U.S. Application Data
(60) Provisional application No. 62/925,475, filed on Oct. 24, 2019.

(51) Int. Cl. C07D 213/75; C07D 241/20; C07D 401/04; C07D 401/12; C07D 405/12; C07D 405/14; C07D 413/04; C07D 413/14; C07D 417/12

(52) U.S. Cl. CPC C07D 213/75; C07D 241/20; C07D 401/04; C07D 401/12; C07D 405/12; C07D 405/14; C07D 413/04; C07D 413/14; C07D 417/12

(57) ABSTRACT

Disclosed is a compound of formula (I): in which all symbols are defined in the description. Also disclosed are pharmaceutical compositions including the compounds, methods of making the compounds, kits comprising the compounds and methods of using the compounds to treat disorders associated with dysregulation of TREK-1, TREK-2 or both TREK-1 and TREK-2 in a mammal.

### TECHNICAL FIELD

The present disclosure relates to compounds, compositions, and methods for treating disorders associated with K2P K⁺ channels, specifically TREK (TWIK RElated K⁺ Channel) dysfunction for which inhibitors of TREK-1, TREK-2 or both TREK-1 and TREK-2 would provide therapeutic benefit.

### BACKGROUND

Potassium (K⁺) channels are membrane proteins that are expressed in virtually every cell of the organism. K⁺ channel subunits (˜80 genes) can be divided into three main structural classes comprising shaker type voltage-gated (Kv), inward rectifier (Kir) and K⁺ channels with two-pore domains (K2P) (Kubo et al., Pharmacol Rev. 2005, 57, 509, Gutman, et al. Pharmacol Rev. 2005, 57, 473, Goldstein et al. Pharmacol Rev. 2005, 57, 527). The third family of K⁺ channels was discovered 20 years ago (Leasge et al. EMBO J. 1996, 15, 1004). The 15 human K2P K⁺ channels have been identified so far and classified into 6 structural subgroups: TWIK, TREK (TWIK RElated K⁺ channels), TASK (TWIK related Acid-Sensitive K⁺ channels), TALK (TWIK related ALkaline pH-activated K⁺ channels), THIK (Tandem pore domain Halothane Inhibited K⁺ channels) and TRESK (TWIK RElated Spinal cord K⁺ channel) (Enyedi et al. Physiol. Rev. 2010, 90, 559). K2P K⁺ channels are responsible for background or ‘leak’ K⁺ currents. These channels are regulated by various physical and chemical stimuli, including membrane stretch, temperature, acidosis, lipids and inhalational anaesthetics. Furthermore, channel activity is tightly controlled by membrane receptor stimulation and second messenger phosphorylation pathways. Several members of this novel family of K⁺ channels are highly expressed in the central and peripheral nervous systems in which they are proposed to play an important physiological role (TRENDs in Neurosci. 2001).

TREK-1, TREK-2, which belong to TREK subgroup, are thermo- and mechano-gated K⁺ channel that is activated by lysophospholipids and PUFAs including arachidonic acid. They are regulated by G-protein-coupled receptors through PKA and PKC phosphorylation (Channels (Austin). 2011 September-October; 5(5):402-9). TREK-1 gene is widely expressed in the CNS with limited distribution in the periphery. In the CNS, TREK-1 expression is highest in the striatal tissues, the caudate and the putamen, as well as in spinal cord, foetal brain, amygdala and thalamus. In the periphery, TREK-1 expression is observed in heart, stomach and small intestine. TREK-2 gene has quite a similar expression profile compared to TREK-1 with high expression in particularly caudate, putamen and foetal brain. However, in contrast to TREK-1, TREK-2 is also highly expressed in cerebellum and corpus callosum well as in several peripheral tissues, particularly kidney (Mol. Brain Res. 2001, 86, 101).

TREK-1 deficient mice display an increased efficacy of serotonin (5-HT) neurotransmission, and a depression-resistant phenotype (Nature Neurosci. 2006, 9, 1134). Spadin, a naturally occurring peptide, blocks TREK-1 and results in a rapid onset of antidepressant efficacy (Br. J. Pharmacol. 2014, 172, 771). Moreover, antidepressants such as fluoxetine and paroxetine directly inhibit TREK channels (Nat. Neurosci. 2006, 9, 1134; Br. J. Pharmacol. 2005, 144, 821). Thus, inhibition of TREK-1 with a small molecule holds promise for the treatment of depression, as well as other mood disorders (Front. Pharmacol. 2018, 9, 863).

Inhibition of TREK-1 protects mice from cognitive impairment induced by anesthesia and, coupled with a high density in the hippocampus, TREK-1 is a potential therapeutic target against memory impairment induced by volatile anesthetics and in other CNS disorders with cognitive deficits (Neurobiology of Learning and Memory, 2017, 145, 199). TREK-1 gene expression is increased in hippocampus of patients with schizophrenia compared to healthy control (Neuropsychopharmacology 2010, 35, 239-57.). Intrathecal injection of microRNA targeting to TREK-1 ameliorates neuropathic pain induced by chronic constriction sciatic nerve injury (Neurochem Res. 2018, 43, 1143), suggesting that inhibition of TREK-1 may be efficacious in cognitive disorders and neuropathic pain. Knockdown of TREK-1 significantly inhibits prostatic cancer cell proliferation in vitro and in vivo, and induces a G1/S cell cycle arrest (Cancer Res. 2008, 68, 1197-203., Oncotarget. 2015, 6, 18460-8.). TREK-1 is also overexpressed in human ovarian cancer tissues, and the treatment of TREK-1 inhibitors (curcumin and L-methionine) suppress ovarian cancer cell proliferation and increase late apoptosis (Clin. Transl. Oncol. 2013, 15, 910-8.). Thus, TREK-1 inhibitors can be useful for the treatment of prostatic and ovarian cancer.

Neurotensin (NT) suppresses TREK-2 current through NT receptor 1-mediated activation of PLC/PKC pathway in entorhinal cortex layer II stellate neurons, leading to depolarization of membrane potential and enhancement of neuronal excitability. Furthermore, NT-induced enhancement of spatial learning is diminished in TREK-2 KO mice, suggesting that TREK-2 inhibitors may be useful for the treatment of cognitive impairment, such as Alzheimer's disease (J. Neurosci. 2014, 34, 7027-42.). TREK-2 is expressed in human bladder carcinoma cell in which TREK-2 contributes to the regulation of resting membrane potential. TREK-2 KD decreases the cell proliferation (Korean J. Physiol. Pharmacol. 2013, 17, 511-6.). Thus, TREK-2 inhibitors also may be efficacious in the treatment of bladder carcinoma.

Selective inhibition of TREK-1, by a small inhibitor, has potential therapeutic benefit for: depression, schizophrenia, cognitive disorders including dementia, neuropathic pain, stroke, prostatic and ovarian cancer (Nat. Neurosci. 2006, 9, 1134, Neuropsychopharmacology 2010, 35, 239, Neurobiol. Learn Mem. 2017, 145, 199, Neurochem Res. 2018, 43, 1143, Neurosci Lett. 2018, 671, 93, Cancer Res. 2008, 68, 1197, Clin. Transl. Oncol. 2013, 15, 910).

Selective inhibition of TREK-2, by a small molecule inhibitor, has potential therapeutic benefit for: cognitive disorders including dementia, stroke and bladder carcinoma (J. Neurosci. 2014, 34, 7027, Biochem. Biophys. Res. Commun. 2005, 327, 1163, Korean J. Physiol. Pharmacol. 2013, 17, 511). The expression level of TREK-2 is increased in cortex and hippocampus of acute rat cerebral ischemia model (Biochem. Biophys. Res. Commun. 2005, 327, 1163-9.) Thus, TREK-2 inhibitors may be useful for the treatment of stroke.

All of the above mentioned disorders may also be effectively treated by a dual TREK1/TREK2 inhibitor with varying degrees of TREK1 and TREK2 preference.

Despite advances in K2P channel research and TREK1/TREK2 pharmacology channel research, there is still a scarcity of compounds that are potent, efficacious, and selective inhibitors of the either TREK1, TREK2 or both TREK1 and TREK2 and also effective in the treatment of neurological, inflammatory, respiratory, renal and cardiovascular disorders associated with K2P K⁺ channels, specifically TREK (TWIK RElated K⁺ channels) dysfunction for which inhibitors of TREK1, TREK2 or both TREK1 and TREK2 would offer therapeutic benefit.

### SUMMARY

In one aspect, disclosed are compounds of formula (I),

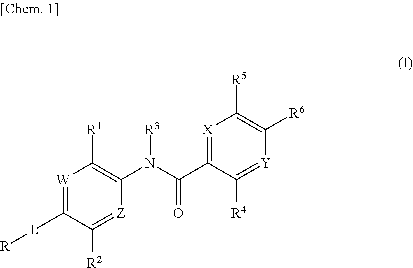

or a pharmaceutically acceptable salt thereof;
wherein: all symbols are defined as below.

Also disclosed are pharmaceutical compositions comprising the compounds, methods of making the compounds, kits comprising the compounds, and methods of using the compounds, compositions and kits for prevention and/or treatment of disorders, such as neurological and/or psychiatric disorders, associated with TREK1, TREK2 or both TREK1/TREK2 dysfunction which inhibitors of TREK1, TREK2 or both TREK1 and TREK2 would offer therapeutic benefit in a mammal.

### DESCRIPTION OF EMBODIMENTS

Disclosed herein are inhibitors of TREK1, TREK2 or both TREK1/TREK2.

That is, the invention relates to;

[1] A compound of formula (I):

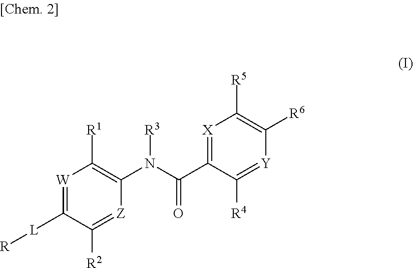

or a pharmaceutically acceptable salt thereof;
wherein:
L is selected from (1) bond, (2) C2-C4-alkynylene, (3) —(C1-C10-alkylene)-O—, (4) —O—(C1-C10-alkylene)-, (5)-(6 to 15 membered aryl)-, (6)-(5 to 15 membered heteroaryl)-, (7)-(3 to 15 membered heterocycle)- and (8) —(C3-C10-cycloalkane)-;
W is selected from (1) CH, (2) CR⁷ and (3) N;
Z is selected from (1) CH, (2) CR⁸ and (3) N;
R⁷ and R⁸ are each independently selected from (1) cyano, (2) halogen, (3) pentahalosulfanyl, (4) C1-C10-thioalkyl, (5) C1-C10-alkoxy, (6) C1-C10-alkyl, (7) C2-C10-alkenyl, (8) C2-C10-alkynyl, (9) —OR⁹, (10) 6 to 15 membered aryl, (11) 5 to 15 membered heteroaryl, (12) C3-C10-cycloalkyl, (13) C2-C10-heteroalkyl, (14) 3 to 15 membered heterocycle, and (15) —(CR¹⁰R¹¹)ₙ-Q;
wherein each of (4)-(8) in R⁷ or R⁸ may be optionally substituted with 1 to 10 halogen, and each of (10)-(14) in R⁷ or R⁸ may be optionally substituted with 1 to 10 substituents selected from (1) halogen, (2) C1-C10-alkyl, and (3) C1-C10-haloalkyl;
R⁹ is selected from (1) hydrogen, (2) C1-C10-alkyl, and (3) C1-C10-haloalkyl;
R¹⁰ is selected from (1) hydrogen, (2) halogen, (3) C1-C10-alkyl and (4) C1-C10-haloalkyl;
R¹¹ is selected from (1) hydrogen, (2) halogen, (3) C1-C10-alkyl and (4) C1-C10-haloalkyl;
or when R¹⁰ and R¹¹ is C1-C10-alkyl, R¹⁰ and R¹¹ may optionally form a C3-C10-cycloalkyl together with the carbon atom to which they are attached;
n is 0, 1, 2, 3 or 4;
Q is selected from (1) halogen, (2) cyano, (3) —OR¹⁰¹, (4) —SR¹⁰², (5) —C(═O)R¹⁰³, (6) —C(═O)OR¹⁰⁴, (7) —S(═O)R¹⁰⁵, (8) —SO₂R¹⁰⁶, (9) —NR¹⁰⁷R¹⁰⁸, (10) —C(═O)NR¹⁰⁹R¹¹⁰, (11) —SO₂NR¹¹¹R¹¹², (12) 6 to 15 membered aryl, (13) 5 to 15 membered heteroaryl, (14) C3-C10-cycloalkyl, and (15) 3 to 15 membered heterocycle, wherein each of (12)-(15) in Q may be optionally substituted with 1 to 10 substituents selected from (1) halogen, (2) C1-C10-alkyl and (3) C1-C10-haloalkyl;
R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹ and R¹² are each independently selected from (1) hydrogen, (2) C1-C10-alkyl, and (3) C1-C10-haloalkyl;
R⁶ is selected from (1) hydrogen and (2) —NH₂;
X is selected from (1) CH, (2) CR¹² and (3) N;
R¹² is selected from (1) halogen, (2) C1-C10-alkyl and (3) C1-C10-haloalkyl;
Y is selected from (1) CH, (2) CR¹³ and (3) N;
R¹³ is selected from (1) halogen, (2) C1-C10-alkyl and (3) C1-C10-haloalkyl;
R is selected from (1) 6 to 15 membered aryl and (2) 5 to 15 membered heteroaryl, each of which may be optionally substituted with 1 to 5 R¹⁴;
wherein multiple R¹⁴ may be the same as or different from each other;
R¹⁴ is selected from (1) halogen, (2) cyano, (3) pentahalosulfanyl, (4) C1-C10-alkyl, (5) C1-C10-thioalkyl, (6) C1-C10-alkoxy, (7) C2-C10-alkenyl, (8) C2-C10-alkynyl, (9) 6 to 15 membered aryl and (10) —OR⁹, wherein each of (4)-(9) in R¹⁴ may be optionally substituted with 1 to 10 halogen;
R¹ is selected from (1) C1-C10-alkyl, (2) halogen, (3) C1-C10-alkoxy, (4) C1-C10-haloalkyl, (5) C1-C10-haloalkoxy and (6) cyano;
R² is selected from (1) hydrogen, (2) C1-C10-alkyl, (3) halogen, (4) C1-C10-alkoxy, (5) C1-C10-haloalkyl, (6) C1-C10-haloalkoxy and (7) cyano;
R³ is selected from (1) hydrogen and (2) C1-C10-alkyl;
R⁴ is selected from (1) hydrogen, (2) halogen, (3) C1-C10-alkyl, (4) C1-C10-haloalkyl, (5) cyano and (6) C3-C10-cycloalkyl which may be optionally substituted with 1 to 10 substituents selected from (1) halogen (2) C1-C10-alkyl and (3) C1-C10-haloalkyl;
or R³ and R⁴ may be optionally taken together to form (1) —CR⁴¹R⁴²—, (2) —CR⁴³R⁴⁴—CR⁴⁵R⁴⁶—, (3) —CR⁴⁷═CR⁴⁸—;
R⁴¹, R⁴², R⁴³, R⁴⁴, R⁴⁵, R⁴⁶, R⁴⁷ and R⁴⁸ are each independently selected from (1) hydrogen and (2) C1-C10-alkyl;
R⁵ is selected from (1) hydrogen, (2) cyano, (3) —NH₂, (4) C1-C10-alkyl, (5) C1-C10-alkoxy, (6) —NH—C(═O)—R¹⁵, (7) —NH—C(═O)—O—R¹⁶, (8) —O—R¹⁷, (9) —O—(C1-C10-alkylene)-R¹⁸, (10) —C(═O)—R¹⁹, (11) —C(═O)—NH—R²⁰, (12) —(C1-C10-alkylene)-(CR²¹R²²)ₚ—R²³, (13) 6 to 15 membered aryl, (14) 5 to 15 membered heteroaryl, (15) C3-C10-cycloalkyl and (16) 3 to 15 membered heterocycle; wherein each of (4), (5), (9) and (12) in R⁵ may be optionally substituted with 1 to 10 substituents selected from (1) halogen (2) cyano and (3) —OH, and each of (13)-(16) in R⁵ may be optionally substituted with 1 to 5 R²⁴;
provided that when R⁵ is hydrogen, R⁶ is —NH₂;
R¹⁵, R¹⁶, R¹⁷, R¹⁸, R¹⁹ and R²⁰ are each independently selected from (1) C1-C10-alkyl, (2) C1-C10-alkoxy, (3) 6 to 15 membered aryl, (4) 5 to 15 membered heteroaryl, (5) C3-C10-cycloalkyl and (6) 3 to 15 membered heterocycle, wherein each of (1) and (2) in R¹⁵, R¹⁶, R¹⁷, R¹⁸, R¹⁹ or R²⁰ may be optionally substituted with 1 to 10 substituents selected from (1) halogen (2) cyano and (3) —OH, and each of (3)-(6) in R¹⁵, R¹⁶, R¹⁷, R¹⁸, R¹⁹ or R²⁰ may be optionally substituted with 1 to 5 R²⁵;
R²¹ and R²² are each independently selected from (1) hydrogen, (2) halogen, (3) C1-C10-alkyl and (4) C1-C10-haloalkyl;
or when R²¹ and R²² is C1-C10-alkyl, R²¹ and R²² may optionally form a C3-C10-cycloalkyl together with the carbon atom to which they are attached;
p is 0, 1, 2, 3 or 4;
R²³ is selected from (1) halogen, (2) cyano, (3) —OR²⁰¹, (4) —SR²⁰², (5) —C(═O)R²⁰³, (6) —C(═O)OR²⁰⁴, (7) —S(═O)R²⁰⁵, (8) —SO 2R²⁰⁶, (9) —NR²⁰⁷R²⁰⁸, (10) —C(═O)NR²⁰⁹R²¹⁰, (11) —SO₂NR²¹¹R²¹², (12) 6 to 15 membered aryl, (13) 5 to 15 membered heteroaryl, (14) C3-C10-cycloalkyl, and (15) 3 to 15 membered heterocycle, wherein each of (12) —(15) in R²³ may be optionally substituted with 1 to 10 substituents selected from (1) halogen, (2) C1-C10-alkyl and (3) C1-C10-haloalkyl;
R²⁰¹, R²⁰², R²⁰³, R²⁰⁴, R²⁰⁵, R²⁰⁶, R²⁰⁷, R²⁰⁸, R²⁰⁹, R²¹⁰, R²¹¹ and R²¹² are each independently selected from (1) hydrogen, (2) C1-C10-alkyl, and (3) C1-C10-haloalkyl;
R²⁴ and R²⁵ are each independently selected from (1) halogen, (2) cyano, (3) —OH, (4) oxo, (5) C1-C10-alkyl, (6) C1-C10-alkoxy, (7) —(C1-C10-alkylene)-NR²⁶R²⁷, (8) C3-C10-cycloalkyl, (9) 3 to 15 membered heterocycle and (10) —(C1-C10-alkylene)-(3 to 15 membered heterocycle), wherein each of (5)-(7) in R²⁴ or R²⁵ may be optionally substituted with 1 to 5 substituents selected with (1) halogen, (2) —OH and (3) cyano, and each of (8)-(10) in R²⁴ or R²⁵ may be optionally substituted with 1 to 5 substituents selected with (1) halogen, (2) —OH, (3) cyano, (4) C1-C10-alkyl and (5) C1-C10-haloalkyl;
R²⁶ and R²⁷ are each independently selected from (1) hydrogen and (2) C1-C10-alkyl; multiple R²⁴ or R²⁵ may be the same as or different from each other.

[1-1] The compound or a pharmaceutically acceptable salt thereof according to [1],
wherein
L is selected from selected from (1) ethynylene, (2) —CH₂—O— and (3) —5 membered heteroaryl (preferably thienyl, pyrazolyl, isoxazolyl or oxadiazolyl (for example, 1,2,4-oxadiazole), more preferably pyrazolyl, isoxazolyl or oxadiazolyl (for example, 1,2,4-oxadiazole)).

[2] The compound or a pharmaceutically acceptable salt thereof according to [1], which is a compound of formula (Ia):

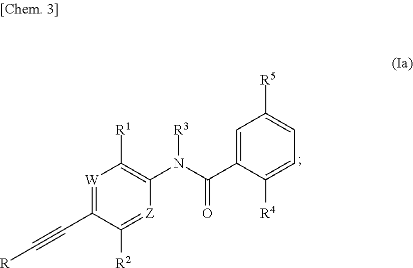

wherein all symbols are as defined in [1].

[3-1] The compound or a pharmaceutically acceptable salt thereof according to [2], wherein
W is selected from (1) CH, (2) CR⁷ and (3) N;
Z is selected from (1) CH, (2) CR′ and (3) N;
R⁷ and R⁸ are each independently selected from (1) cyano, (2) halogen or (3) C1-C4-alkyl which may be optionally substituted with 1 to 5 halogen (preferably (1) halogen or (2) C1-C4-alkyl which may be optionally substituted with 1 to 5 halogen, more preferably (1) halogen or (2) methyl);
R is selected from (1) 6 to 10 membered aryl which may be optionally substituted with 1 to 3 R¹⁴ or (2) 5 to 10 membered heteroaryl which may be optionally substituted with 1 to 3 R¹⁴ (preferably 6 to 10 membered aryl which may be optionally substituted with 1 to 3 R¹⁴, more preferably phenyl or pyridine, each of which may be optionally substituted with 1 to 3 R¹⁴, most preferably phenyl which may be optionally substituted with 1 to 3 R¹⁴);
wherein multiple R¹⁴ may be the same as or different from each other;
R¹⁴ is selected from (1) halogen, (2) cyano, (3) C1-C4-alkyl which may be optionally substituted with 1 to 5 halogen or (4) C1-C4-alkoxy which may be optionally substituted with 1 to 5 halogen (more preferably (1) halogen, (3) C1-C4-alkyl which may be optionally substituted with 1 to 5 halogen or (4) C1-C4-alkoxy which may be optionally substituted with 1 to 5 halogen, most preferably (1) halogen or (3) methyl which may be optionally substituted with 1 to 3 halogen);
R¹ is selected from (1) halogen, (2) C1-C4-alkyl, (3) C1-C4-alkoxy, (4) C1-C4-haloalkyl or (5) C1-C4-haloalkoxy (preferably (1) halogen, (2) C1-C4-alkyl or (3) C1-C4-alkoxy, more preferably (1) halogen (2) methyl or (3) methoxy);
R² is selected from (1) hydrogen, (2) halogen, (3) C1-C4-alkyl, (4) C1-C4-alkoxy, (5) C1-C4-haloalkyl or (6) C1-C4-haloalkoxy (preferably hydrogen);
R³ is selected from (1) hydrogen and (2) C1-C4-alkyl (preferably hydrogen);
R⁴ is halogen;
R⁵ is selected from (1) C1-C6-alkyl, (2) C1-C6-alkoxy, (3) —NH₂, (4) —NH—C(═O)—R¹⁵, (5) —NH—C(═O)—O—R¹⁶, (6) —O—R¹⁷, (7) —O—(C1-C10-alkylene)-R¹⁸, (8) —C(═O)—R¹⁹, (9) —C(═O)—NH—R²⁰, (10) 6 to 15 membered aryl, (11) 5 to 15 membered heteroaryl, (12) C3-C10-cycloalkyl or (13) 3 to 15 membered heterocycle (preferably (4) —NH—C(═O)—R¹⁵, (5) —NH—C(═O)—O—R¹⁶, (6) —O—R¹⁷, (7) —O—(C1-C4-alkylene)-R¹⁸, (8) —C(═O)—R¹⁹, (9) —C(═O)—NH—R²⁰ or (11) 5 to 10 membered heteroaryl),
wherein each of (1), (2) and (7) in R⁵ may be optionally substituted with 1 to 5 substituents selected from (1) halogen (2) cyano and (3) —OH, and each of (10)-(13) in R⁵ may be optionally substituted with 1 to 5 R²⁴;
R²⁴ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (4) C1-C4-alkyl, (7) 3 to 10 membered heterocycle or (8) —(C1-C4-alkylene)-(3 to 10 membered heterocycle), more preferably (7) 3 to 10 membered heterocycle), wherein each of (4)-(5) in R²⁴ may be optionally substituted with 1 to 3 substituents selected with (1) halogen, (2) —OH and (3) cyano and each of (6)-(8) in R²⁴ may be optionally substituted with 1 to 3 substituents selected with (1) halogen, (2) —OH, (3) cyano, (4) C1-C4-alkyl and (5) C1-C4-haloalkyl;
R¹⁵, R¹⁶, R¹⁷, R¹⁸, R¹⁹ and R²⁰ are each independently selected from (1) C1-C4-alkyl, (2) C1-C4-alkoxy, (3) 6 to 10 membered aryl, (4) 5 to 10 membered heteroaryl, (5) C3-C6-cycloalkyl or (6) 3 to 10 membered heterocycle, wherein each of (1) and (2) in R¹⁵, R¹⁶, R¹⁷, R¹⁸, R¹⁹ or R²⁰ may be optionally substituted with 1 to 5 substituents selected from (1) halogen (2) cyano and (3) —OH, and each of (3)-(6) in R¹⁵, R¹⁶, R¹⁷, R¹⁸, R¹⁹ or R²⁰ may be optionally substituted with 1 to 5 R²⁵;
R²⁵ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (1) halogen, (2) —OH, (4) C1-C4-alkyl or (5) C1-C4-alkoxy, more preferably (1) halogen, (2) —OH, (4) methyl which may be optionally substituted with 1 to 3 substituents selected with halogen, —OH and cyano, or (5) methoxy which may be optionally substituted with 1 to 3 substituents selected with halogen, —OH and cyano), wherein each of (4)-(5) in R²⁵ may be optionally substituted with 1 to 3 substituents selected with (1) halogen, (2) —OH and (3) cyano and each of (6)-(8) in R²⁵ may be optionally substituted with 1 to 3 substituents selected with (1) halogen, (2) —OH, (3) cyano, (4) C1-C4-alkyl and (5) C1-C4-haloalkyl.

[3-2] The compound according to [2], wherein
R⁵ is selected from (1) cyano, (2) —NH₂, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) 6 to 15 membered aryl, (10) 5 to 15 membered heteroaryl, (11) C3-C10-cycloalkyl and (12) 3 to 15 membered heterocycle;
wherein (6) in R⁵ may be optionally substituted with 1 to 10 substituents selected from (1) halogen (2) cyano and (3) —OH, and each of (9)-(12) in R⁵ may be optionally substituted with 1 to 5 R²⁴;
and the other symbols are as defined in [1].

[4] The compound or a pharmaceutically acceptable salt thereof according to [3-2], which is a compound of formula (Ia-1-1):

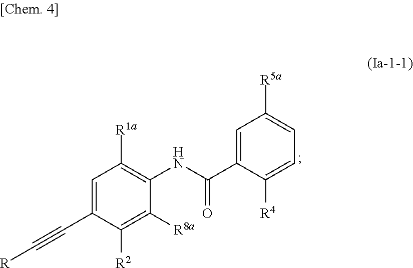

wherein
R^{1a} is selected from (1) halogen, (2) C1-C10-alkyl and (3) C1-C10-haloalkyl;
R^{8a} is selected from (1) hydrogen, (2) halogen, (3) C1-C10-alkyl and (4) C1-C10-haloalkyl;
R^{5a} is selected from (1) cyano, (2) —NH₂, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) 6 to 15 membered aryl, (10) 5 to 15 membered heteroaryl, (11) C3-C10-cycloalkyl and (12) 3 to 15 membered heterocycle;
wherein (6) in R⁵ may be optionally substituted with 1 to 10 substituents selected from (1) halogen (2) cyano and (3) —OH, and each of (9)-(12) in R⁵ may be optionally substituted with 1 to 5 R²⁴;
and the other symbols are as defined in [1].

[5] The compound according to [4], wherein R^{5a} is selected from (1) —NH—C(═O)—(C3-C10-cycloalkyl), (2) —NH—C(═O)-(3 to 15 membered heterocycle), (3) —NH—C(═O)—O—(C1-C10-alkyl) which may be optionally substituted with 1 to 5 substituents selected from halogen, cyano and —OH, (4) —O—(C1-C10-alkyl) which may be optionally substituted with 1 to 5 substituents selected from halogen, cyano and —OH, (5) —O—(C1-C10-alkylene)-(3 to 15 membered heterocycle), (6) —C(═O)—(C3-C10-cycloalkyl), (7) —C(═O)-(3 to 15 membered heterocycle), (8) —C(═O)—NH—(C1-C10-alkyl) and (9) 5 to 15 membered heteroaryl;
wherein each of (1), (2) and (5)-(9) in R^{5a} may be optionally substituted with 1 to 5 R⁵¹;
multiple R⁵¹ may be the same as or different from each other;
R⁵¹ is independently selected from (1) halogen, (2) cyano, (3) —OH, (4) C1-C10-alkyl, (5) C1-C10-alkoxy, (6) —(C1-C10-alkylene)-(3 to 15 membered heterocycle), (7) C3-C10-cycloalkyl and (8) 3 to 15 membered heterocycle, wherein each of (4)-(8) in R⁵¹ may be optionally substituted with 1 to 10 substituents selected from (1) halogen, (2) —OH, (3) cyano, (4) C1-C10-alkyl and (5) C1-C10-haloalkyl.

[6] The compound or a pharmaceutically acceptable salt thereof according to [3], which is a compound of formula (Ia-1-2):

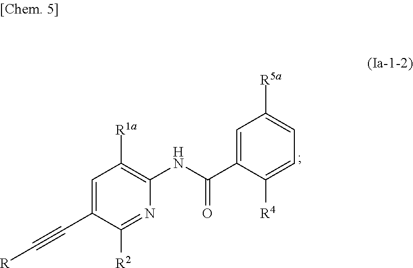

wherein all symbols are as defined in [1] or [4].

[7] The compound or a pharmaceutically acceptable salt thereof according to [6], wherein R^{5a} is selected from (1) —NH—C(═O)—(C3-C10-cycloalkyl), (2) —NH—C(═O)-(3 to 15 membered heterocycle), (3) —NH—C(═O)—O—(C1-C10-alkyl) which may be optionally substituted with 1 to 5 substituents selected from halogen, cyano and —OH, (4) —O—(C1-C10-alkyl) which may be optionally substituted with 1 to 5 substituents selected from halogen, cyano and —OH, (5) —O—(C1-C10-alkylene)-(3 to 15 membered heterocycle), (6) —C(═O)—(C3-C10-cycloalkyl), (7) —C(═O)-(3 to 15 membered heterocycle), (8) —C(═O)—NH—(C1-C10-alkyl) and (9) 5 to 15 membered heteroaryl; wherein each of (1), (2) and (5)-(9) in R⁵¹ may be optionally substituted with 1 to 5 R⁵¹.
multiple R⁵¹ may be the same as or different from each other;
R⁵¹ is independently selected from (1) halogen, (2) cyano, (3) —OH, (4) C1-C10-alkyl, (5) C1-C10-alkoxy, (6) —(C1-C10-alkylene)-(3 to 15 membered heterocycle), (7) C3-C10-cycloalkyl and (8) 3 to 15 membered heterocycle, wherein each of (4)-(8) in R⁵¹ may be optionally substituted with 1 to 10 substituents selected from (1) halogen, (2) —OH, (3) cyano, (4) C1-C10-alkyl and (5) C1-C10-haloalkyl.

[8] The compound or a pharmaceutically acceptable salt thereof according to any one of [2] to [7], wherein R is phenyl which may be optionally substituted with 1 to 5 R¹⁴.

[9] The compound or a pharmaceutically acceptable salt thereof according to [1], which is a compound of formula (Ib):

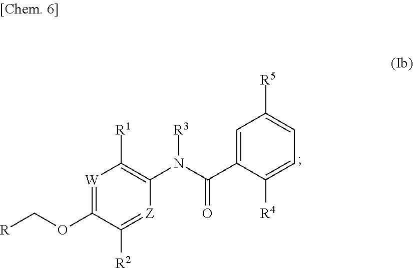

wherein all symbols are as defined in [1].

[10] The compound or a pharmaceutically acceptable salt thereof according to [9],
wherein
W is selected from (1) CH, (2) CR⁷ and (3) N;
Z is selected from (1) CH, (2) CR⁸ and (3) N;
R⁷ and R⁸ are each independently selected from (1) cyano, (2) halogen or (3) C1-C4-alkyl which may be optionally substituted with 1 to 5 halogen (preferably (1) halogen or (2) C1-C4-alkyl which may be optionally substituted with 1 to 5 halogen, more preferably (1) halogen or (2) methyl);
R is selected from (1) 6 to 10 membered aryl which may be optionally substituted with 1 to 3 R¹⁴ or (2) 5 to 10 membered heteroaryl which may be optionally substituted with 1 to 3 R¹⁴ (preferably 6 to 10 membered aryl which may be optionally substituted with 1 to 3 R¹⁴, more preferably phenyl or pyridine, each of which may be optionally substituted with 1 to 3 R¹⁴, most preferably phenyl which may be optionally substituted with 1 to 3 R¹⁴);
wherein multiple R¹⁴ may be the same as or different from each other;
R¹⁴ is selected from (1) halogen, (2) cyano, (3) C1-C4-alkyl which may be optionally substituted with 1 to 5 halogen or (4) C1-C4-alkoxy which may be optionally substituted with 1 to 5 halogen (more preferably (1) halogen, (3) C1-C4-alkyl which may be optionally substituted with 1 to 5 halogen or (4) C1-C4-alkoxy which may be optionally substituted with 1 to 5 halogen, most preferably (1) halogen or (3) methyl which may be optionally substituted with 1 to 3 halogen);
R¹ is selected from (1) halogen, (2) C1-C4-alkyl, (3) C1-C4-alkoxy, (4) C1-C4-haloalkyl or (5) C1-C4-haloalkoxy (preferably (1) halogen, (2) C1-C4-alkyl or (3) C1-C4-alkoxy, more preferably (1) halogen (2) methyl or (3) methoxy);
R² is selected from (1) hydrogen, (2) halogen, (3) C1-C4-alkyl, (4) C1-C4-alkoxy, (5) C1-C4-haloalkyl or (6) C1-C4-haloalkoxy (preferably hydrogen);
R³ is selected from (1) hydrogen and (2) C1-C4-alkyl (preferably hydrogen);
R⁴ is halogen;
R⁵ is selected from (1) C1-C6-alkyl, (2) C1-C6-alkoxy, (3) —NH₂, (4) —NH—C(═O)—R¹⁵, (5) —NH—C(═O)—O—R¹⁶, (6) —O—R¹⁷, (7) —O—(C1-C10-alkylene)-R¹⁸, (8) —C(═O)—R¹⁹, (9) —C(═O)—NH—R²⁰, (10) 6 to 15 membered aryl, (11) 5 to 15 membered heteroaryl, (12) C3-C10-cycloalkyl or (13) 3 to 15 membered heterocycle (preferably (4) —NH—C(═O)—R¹⁵, (5) —NH—C(═O)—O—R¹⁶, (6) —O—R¹⁷, (7) —O—(C1-C4-alkylene)-R¹⁸, (8) —C(═O)—R¹⁹, (9) —C(═O)—NH—R²⁰ or (11) 5 to 10 membered heteroaryl), wherein each of (1), (2) and (7) in R⁵ may be optionally substituted with 1 to 5 substituents selected from (1) halogen (2) cyano and (3) —OH, and each of (10)-(13) in R⁵ may be optionally substituted with 1 to 5 R²⁴;
R²⁴ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (4) C1-C4-alkyl, (7) 3 to 10 membered heterocycle or (8) —(C1-C4-alkylene)-(3 to 10 membered heterocycle), more preferably (7) 3 to 10 membered heterocycle), wherein each of (4)-(5) in R²⁴ may be optionally substituted with 1 to 3 substituents selected with (1) halogen, (2) —OH and (3) cyano and each of (6)-(8) in R²⁴ may be optionally substituted with 1 to 3 substituents selected with (1) halogen, (2) —OH, (3) cyano, (4) C1-C4-alkyl and (5) C1-C4-haloalkyl;
R¹⁵, R¹⁶, R¹⁷, R¹⁸, R¹⁹ and R²⁰ are each independently selected from (1) C1-C4-alkyl, (2) C1-C4-alkoxy, (3) 6 to 10 membered aryl, (4) 5 to 10 membered heteroaryl, (5) C3-C6-cycloalkyl or (6) 3 to 10 membered heterocycle, wherein each of (1) and (2) in R¹⁵, R¹⁶, R¹⁷, R¹⁸, R¹⁹ or R²⁰ may be optionally substituted with 1 to 5 substituents selected from (1) halogen (2) cyano and (3) —OH, and each of (3)-(6) in R¹⁵, R¹⁶, R¹⁷, R¹⁸, R¹⁹ or R²⁰ may be optionally substituted with 1 to 5 R²⁵;
R²⁵ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (1) halogen, (2) —OH, (4) C1-C4-alkyl or (5) C1-C4-alkoxy, more preferably (1) halogen, (2) —OH, (4) methyl which may be optionally substituted with 1 to 3 substituents selected with halogen, —OH and cyano, or (5) methoxy which may be optionally substituted with 1 to 3 substituents selected with halogen, —OH and cyano), wherein each of (4)-(5) in R²⁵ may be optionally substituted with 1 to 3 substituents selected with (1) halogen, (2) —OH and (3) cyano and each of (6)-(8) in R²⁵ may be optionally substituted with 1 to 3 substituents selected with (1) halogen, (2) —OH, (3) cyano, (4) C1-C4-alkyl and (5) C1-C4-haloalkyl.

[11] The compound or a pharmaceutically acceptable salt thereof according to [10],
wherein
R is phenyl which may be optionally substituted with 1 to 5 R¹⁴;
R⁴ is halogen,
R⁵ is selected from (1) C1-C10-alkyl, (2) C1-C10-alkoxy, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) —(C1-C10-alkylene)-(CR²¹R²²)ₚ—R²³, (10) 6 to 15 membered aryl, (11) 5 to 15 membered heteroaryl, (12) C3-C10-cycloalkyl and (13) 3 to 15 membered heterocycle;
wherein each of (1), (2), (6) and (9) in R⁵ may be optionally substituted with 1 to 10 substituents selected from (1) halogen (2) cyano and (3) —OH, and each of (10)-(13) in R⁵ may be optionally substituted with 1 to 5 R²⁴;
and the other symbols are as defined in [1].

[11-1] The compound according to [11], wherein
R⁵ is selected from (1) —NH—C(═O)—R¹⁵ and (2) —NH—C(═O)—O—R¹⁶;
and the other symbols are as defined in [1].

[12] The compound or a pharmaceutically acceptable salt thereof according to [1], which is a compound of formula (Ic):

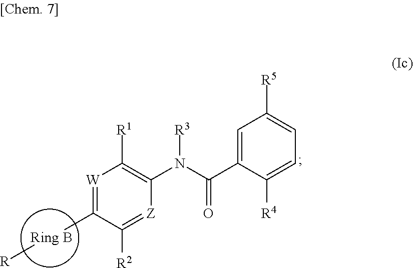

wherein Ring B is selected from (1) 6 to 15 membered aryl and (2) 5 to 15 membered heteroaryl;
and the other symbols are as defined in [1].

[13] The compound or a pharmaceutically acceptable salt thereof according to [12], wherein
W is selected from (1) CH, (2) CR⁷ and (3) N;
Z is selected from (1) CH, (2) CR′ and (3) N;
R⁷ and R⁸ are each independently selected from (1) cyano, (2) halogen or (3) C1-C4-alkyl which may be optionally substituted with 1 to 5 halogen (preferably (1) halogen or (2) C1-C4-alkyl which may be optionally substituted with 1 to 5 halogen, more preferably (1) halogen or (2) methyl);
R is selected from (1) 6 to 10 membered aryl which may be optionally substituted with 1 to 3 R¹⁴ or (2) 5 to 10 membered heteroaryl which may be optionally substituted with 1 to 3 R¹⁴ (preferably 6 to 10 membered aryl which may be optionally substituted with 1 to 3 R¹⁴, more preferably phenyl or pyridine, each of which may be optionally substituted with 1 to 3 R¹⁴, most preferably phenyl which may be optionally substituted with 1 to 3 R¹⁴);
wherein multiple R¹⁴ may be the same as or different from each other;
R¹⁴ is selected from (1) halogen, (2) cyano, (3) C1-C4-alkyl which may be optionally substituted with 1 to 5 halogen or (4) C1-C4-alkoxy which may be optionally substituted with 1 to 5 halogen (more preferably (1) halogen, (3) C1-C4-alkyl which may be optionally substituted with 1 to 5 halogen or (4) C1-C4-alkoxy which may be optionally substituted with 1 to 5 halogen, most preferably (1) halogen or (3) methyl which may be optionally substituted with 1 to 3 halogen);
R¹ is selected from (1) halogen, (2) C1-C4-alkyl, (3) C1-C4-alkoxy, (4) C1-C4-haloalkyl or (5) C1-C4-haloalkoxy (preferably (1) halogen, (2) C1-C4-alkyl or (3) C1-C4-alkoxy, more preferably (1) halogen (2) methyl or (3) methoxy);
R² is selected from (1) hydrogen, (2) halogen, (3) C1-C4-alkyl, (4) C1-C4-alkoxy, (5) C1-C4-haloalkyl or (6) C1-C4-haloalkoxy (preferably hydrogen);
R³ is selected from (1) hydrogen and (2) C1-C4-alkyl (preferably hydrogen);
R⁴ is halogen;
R⁵ is selected from (1) C1-C6-alkyl, (2) C1-C6-alkoxy, (3) —NH₂, (4) —NH—C(═O)—R¹⁵, (5) —NH—C(═O)—O—R¹⁶, (6) —O—R¹⁷, (7) —O—(C1-C10-alkylene)-R¹⁸, (8) —C(═O)—R¹⁹, (9) —C(═O)—NH—R²⁰, (10) 6 to 15 membered aryl, (11) 5 to 15 membered heteroaryl, (12) C3-C10-cycloalkyl or (13) 3 to 15 membered heterocycle (preferably (4) —NH—C(═O)—R¹⁵, (5) —NH—C(═O)—O—R¹⁶, (6) —O—R¹⁷, (7) —O—(C1-C4-alkylene)-R¹⁸, (8) —C(═O)—R¹⁹, (9) —C(═O)—NH—R²⁰ or (11) 5 to 10 membered heteroaryl),
wherein each of (1), (2) and (7) in R⁵ may be optionally substituted with 1 to 5 substituents selected from (1) halogen (2) cyano and (3) —OH, and each of (10)-(13) in R⁵ may be optionally substituted with 1 to 5 R²⁴;
R²⁴ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (4) C1-C4-alkyl, (7) 3 to 10 membered heterocycle or (8) —(C1-C4-alkylene)-(3 to 10 membered heterocycle), more preferably (7) 3 to 10 membered heterocycle), wherein each of (4)-(5) in R²⁴ may be optionally substituted with 1 to 3 substituents selected with (1) halogen, (2) —OH and (3) cyano and each of (6)-(8) in R²⁴ may be optionally substituted with 1 to 3 substituents selected with (1) halogen, (2) —OH, (3) cyano, (4) C1-C4-alkyl and (5) C1-C4-haloalkyl;
R¹⁵, R¹⁶, R¹⁷, R¹⁸, R¹⁹ and R²⁰ are each independently selected from (1) C1-C4-alkyl, (2) C1-C4-alkoxy, (3) 6 to 10 membered aryl, (4) 5 to 10 membered heteroaryl, (5) C3-C6-cycloalkyl or (6) 3 to 10 membered heterocycle, wherein each of (1) and (2) in R¹⁵, R¹⁶, R¹⁷, R¹⁸, R¹⁹ or R²⁰ may be optionally substituted with 1 to 5 substituents selected from (1) halogen (2) cyano and (3) —OH, and each of (3)-(6) in R¹⁵, R¹⁶, R¹⁷, R¹⁸, R¹⁹ or R²⁰ may be optionally substituted with 1 to 5 R²⁵;
R²⁵ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (1) halogen, (2) —OH, (4) C1-C4-alkyl or (5) C1-C4-alkoxy, more preferably (1) halogen, (2) —OH, (4) methyl which may be optionally substituted with 1 to 3 substituents selected with halogen, —OH and cyano, or (5) methoxy which may be optionally substituted with 1 to 3 substituents selected with halogen, —OH and cyano), wherein each of (4)-(5) in R²⁵ may be optionally substituted with 1 to 3 substituents selected with (1) halogen, (2) —OH and (3) cyano and each of (6)-(8) in R²⁵ may be optionally substituted with 1 to 3 C1-C4-haloalkyl.

[14] The compound or a pharmaceutically acceptable salt thereof according to [13], wherein
Ring B is selected from

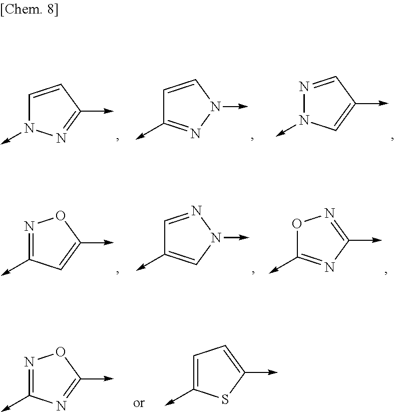

wherein right arrow is connecting position with 6 membered ring and left arrow is connecting position with R;
R is phenyl which may be optionally substituted with 1 to 5 R¹⁴;
R⁴ is halogen,
R⁵ is selected from (1) C1-C10-alkyl, (2) C1-C10-alkoxy, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) —(C1-C10-alkylene)-(CR²¹R²²)ₚ—R²³, (10) 6 to 15 membered aryl, (11) 5 to 15 membered heteroaryl, (12) C3-C10-cycloalkyl and (13) 3 to 15 membered heterocycle;
wherein each of (1), (2), (6) and (9) in R⁵ may be optionally substituted with 1 to 10 substituents selected from (1) halogen (2) cyano and (3) —OH, and each of (10)-(13) in R⁵ may be optionally substituted with 1 to 5 R²⁴;
and the other symbols are as defined in [1].

[15] The compound or a pharmaceutically acceptable salt thereof according to [14], wherein
Ring B is selected from

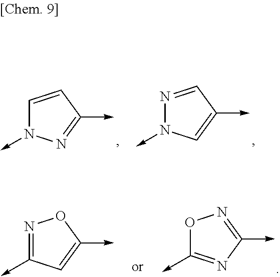

R⁵ is selected from (1) —NH—C(═O)—R¹⁵ and (2) —NH—C(═O)—O—R¹⁶; and the other symbols are as defined in [1].

[16] The compound or a pharmaceutically acceptable salt thereof according to [1], wherein the compound is
(1) tert-butyl N-[4-chloro-3-[[3-fluoro-5-(2-phenylethynyl)-2-pyridyl]carbamoyl]phenyl]carbamate,
(2) 2-chloro-N-[3-fluoro-5-(2-phenylethynyl)-2-pyridyl]-5-[[(1S,2S)-2-methylcyclopropanecarbonyl]amino]benzamide,
(3) 2-chloro-N-[3-fluoro-5-(2-phenylethynyl)-2-pyridyl]-5-[(2-methylcyclopropanecarbonyl)amino]benzamide,
(4) 2-chloro-N-[3-fluoro-5-[2-(3-fluorophenyl)ethynyl]-2-pyridyl]-5-[[(1S,2S)-2-methylcyclopropanecarbonyl]amino]benzamide,
(5) tert-butyl N-[4-chloro-3-[[5-[2-(3-fluorophenyl)ethynyl]-3-methyl-2-pyridyl]carbamoyl]phenyl]carbamate,
(6) 2-chloro-5-[(1-fluorocyclopropanecarbonyl) amino]-N-[3-fluoro-5-(2-phenylethynyl)-2-pyridyl]benzamide,
(7) 2-chloro-5-(cyclopropanecarbonylamino)-N-[3-fluoro-5-[2-(4-fluorophenyl)ethynyl]-2-pyridyl]benzamide,
(8) tert-butyl N-[4-chloro-3-[[5-[2-(4-fluorophenyl)ethynyl]-3-methyl-2-pyridyl]carbamoyl]phenyl]carbamate,
(9) 2-chloro-5-(cyclopropanecarbonylamino)-N-[3-methyl-5-(2-phenylethynyl)-2-pyridyl]benzamide,
(10) N-[4-chloro-3-[[3-fluoro-5-(2-phenylethynyl)-2-pyridyl]carbamoyl]phenyl]-1,4-dioxane-2-carboxamide,
(11) tert-butyl N-[4-chloro-3-[[6-[2-(3-fluorophenyl)ethynyl]-2,4-dimethyl-3-pyridyl]carbamoyl]phenyl]carbamate,
(12) N-[4-chloro-3-[[5-[2-(4-fluorophenyl)ethynyl]-3-methyl-2-pyridyl]carbamoyl]phenyl]oxetane-2-carboxamide,
(13) N-[4-chloro-3-[[5-[2-(3-fluorophenyl)ethynyl]-3-methyl-2-pyridyl]carbamoyl]phenyl]-1,4-dioxane-2-carboxamide,
(14) N-[4-chloro-3-[[5-[2-(4-fluorophenyl)ethynyl]-3-methyl-2-pyridyl]carbamoyl]phenyl]-1,4-dioxane-2-carboxamide,
(15) tert-butyl N-[4-chloro-3-[[3-methyl-5-[2-(2-pyridyl)ethynyl]-2-pyridyl]carbamoyl]phenyl]carbamate,
(16) 2-chloro-5-(3,3-difluoro azetidine-1-carbonyl)-N-[3-methyl-5-(2-phenylethynyl)-2-pyridyl]benzamide,
(17) 4-chloro-N1-(2-hydroxy-1,1-dimethyl-ethyl)-N3-[3-methyl-5-(2-phenylethynyl)-2-pyridyl]benzene-1,3-dicarboxamide,
(18) 2-chloro-N-[5-[2-(3-fluorophenyl)ethynyl]-3-methyl-2-pyridyl]-5-(3-hydroxy-3-methy 1-azetidine-1-carbonyl)benzamide,
(19) 2-chloro-N-[3-methyl-5-(2-phenylethynyl)-2-pyridyl]-5-(5-tetrahydrofuran-3-yl-1,2,4-oxadiazol-3-yl)benzamide,
(20) 2-chloro-N-[3-methyl-5-(2-phenylethynyl)-2-pyridyl]-5-(tetrahydropyran-4-ylmethoxy) benzamide,
(21) 2-chloro-5-[[(2R)-1,4-dioxan-2-yl]methoxy]-N-[3-methyl-5-(2-phenylethynyl)-2-pyridyl]benzamide,
(22) 2-chloro-5-(1,4-dioxan-2-ylmethoxy)-N-[3-methyl-5-(2-phenylethynyl)-2-pyridyl]benzamide,
(23) 2-chloro-5-[[(2S)-1,4-dioxan-2-yl]methoxy]-N-[3-methyl-5-(2-phenylethynyl)-2-pyrid yl]benzamide,
(24) 2-chloro-5-(2-methoxyethoxy)-N-[3-methyl-5-(2-phenylethynyl)-2-pyridyl]benzamide,
(25) tert-butyl (3-((4-(benzyloxy)-2-methylphenyl)carbamoyl)-4-chlorophenyl)carbamate,
(26) tert-butyl (4-chloro-3-((2-methyl-4-(phenoxymethyl)phenyl)carbamoyl)phenyl)carbamate, or a pharmaceutically acceptable salt thereof.

[17] A pharmaceutical composition comprising a compound of formula (I), or a pharmaceutically acceptable salt thereof, and a pharmaceutically acceptable carrier.

[18] The pharmaceutical composition according to [17], which is a TREK1, TREK2 or both TREK1/TREK2 inhibitor.

[19] The pharmaceutical composition according to [18], which is a preventive and/or therapeutic agent for a disorder associated with TREK1, TREK2 or dual TREK1/TREK2 dysfunction in which inhibitors of TREK1, TREK2 or both TREK1 and TREK2 would offer therapeutic benefit.

[20] The pharmaceutical composition according to [19], wherein the disorder associated with TREK1, TREK2 or dual TREK1/TREK2 dysfunction in which inhibitors of TREK1, TREK2 or both TREK1 and TREK2 would offer therapeutic benefit is a neurological and/or psychiatric disorder.

[21] The pharmaceutical composition according to [20], wherein the neurological and/or psychiatric disorder is selected from depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain or cerebral infarction.

[22] A medicament comprising the compound of formula (I), or a pharmaceutically acceptable salt thereof with at least one selected from typical antipsychotics and atypical antipsychotics.

[23] A method for treating and/or preventing a disorder associated with TREK1, TREK2 or dual TREK1/TREK2 dysfunction in which inhibitors of TREK1, TREK2 or both TREK1/TREK2 would offer therapeutic benefit in a mammal, comprising a step of administering to the mammal in need thereof a therapeutically effective amount a compound of any one of [1] to [10], or pharmaceutically acceptable salt thereof.

[24] The method according to [23], wherein a disorder associated with TREK1, TREK2 or dual TREK1/TREK2 dysfunction in which inhibitors of TREK1, TREK2 or both TREK1/TREK2 would offer therapeutic benefit in a mammal is a neurological and/or psychiatric disorder associated with TREK1, TREK2 or dual TREK1/TREK2 dysfunction in which inhibitors of TREK1, TREK2 or both TREK1/TREK2 would offer therapeutic benefit in a mammal.

[25] The method according to [24], wherein the neurological and/or psychiatric disorder associated with TREK1, TREK2 or dual TREK1/TREK2 dysfunction in which inhibitors of TREK1, TREK2 or both TREK1/TREK2 would offer therapeutic benefit in a mammal is selected from depression, schizophrenia, anxiety disorders, bipolar depression, Alzheimer's disease, Parkinson's disease, Huntington's disease, Amyotrophic lateral sclerosis, neuropathic pain or cerebral infarction.

[26] A compound of formula (I), or a pharmaceutically acceptable salt thereof for use in preventing and/or treating of a neurological and/or psychiatric disorder associated with TREK1, TREK2 or dual TREK1/TREK2 dysfunction in which inhibitors of TREK1, TREK2 or both TREK1 and TREK2 would offer therapeutic benefit.

[27] Use of the compound of formula (I), or a pharmaceutically acceptable salt thereof for production of a preventive and/or therapeutic agent against a neurological and/or psychiatric disorder associated with TREK1, TREK2 or dual TREK1/TREK2 dysfunction in which inhibitors of TREK1, TREK2 or both TREK1 and TREK2 would offer therapeutic benefit.

[28] A kit comprising a compound or a pharmaceutically acceptable salt thereof of any one of [1] to [16], and one or more of: (a) at least one agent known to decrease TREK1 channel activity; (b) at least one agent known to decrease TREK2 channel activity; (c) at least one agent known to prevent and/or treat a disorder associated with TREK channel dysfunction in which inhibitors of TREK1, TREK2 or both TREK1/TREK2 would offer therapeutic benefit in a mammal; (d) instructions for preventing and/or treating a disorder associated with TREK dysfunction in which inhibitors of TREK1, TREK2 or both TREK1/TREK2 would offer therapeutic benefit in a mammal; and (e) instructions for administering the compound in connection with cognitive behavioral therapy.

Disclosed herein are inhibitors of the TREK (TWIK RElated K⁺ channels)—subtypes 1 and 2 (TREK1 and TREK2), methods of making same, pharmaceutical compositions comprising same, and methods of preventing and/or treating neurological, psychiatric, inflammatory, respiratory, renal and cardiovascular disorders associated with TREK channel dysfunction using same. The compounds include, but not limited to,
(1) tert-butyl N-[4-chloro-3-[[3-fluoro-5-(2-phenylethynyl)-2-pyridyl]carbamoyl]phenyl]carbamate,
(2) 2-chloro-N-[3-fluoro-5-(2-phenylethynyl)-2-pyridyl]-5-[[(1S,2S)-2-methylcyclopropanecarbonyl]amino]benzamide,
(3) 2-chloro-N-[3-fluoro-5-(2-phenylethynyl)-2-pyridyl]-5-[(2-methylcyclopropanecarbonyl)amino]benzamide,
(4) 2-chloro-N-[3-fluoro-5-[2-(3-fluorophenyl)ethynyl]-2-pyridyl]-5-[[(1S,2S)-2-methylcyclopropanecarbonyl]amino]benzamide,
(5) tert-butyl N-[4-chloro-3-[[5-[2-(3-fluorophenyl)ethynyl]-3-methyl-2-pyridyl]carbamoyl]phenyl]carbamate,
(6) 2-chloro-5-[(1-fluorocyclopropanecarbonyl)amino]-N-[3-fluoro-5-(2-phenylethynyl)-2-pyridyl]benzamide,
(7) 2-chloro-5-(cyclopropanecarbonylamino)-N-[3-fluoro-5-[2-(4-fluorophenyl)ethynyl]-2-pyridyl]benzamide,
(8) tert-butyl N-[4-chloro-3-[[5-[2-(4-fluorophenyl)ethynyl]-3-methyl-2-pyridyl]carbamoyl]phenyl]carbamate,
(9) 2-chloro-5-(cyclopropanecarbonylamino)-N-[3-methyl-5-(2-phenylethynyl)-2-pyridyl]benzamide,
(10) N-[4-chloro-3-[[3-fluoro-5-(2-phenylethynyl)-2-pyridyl]carbamoyl]phenyl]-1,4-dioxane-2-carboxamide,
(11) tert-butyl N-[4-chloro-3-[[6-[2-(3-fluorophenyl)ethynyl]-2,4-dimethyl-3-pyridyl]carbamoyl]phenyl]carbamate,
(12) N-[4-chloro-3-[[5-[2-(4-fluorophenyl)ethynyl]-3-methyl-2-pyridyl]carbamoyl]phenyl]oxetane-2-carboxamide,
(13) N-[4-chloro-3-[[5-[2-(3-fluorophenyl)ethynyl]-3-methyl-2-pyridyl]carbamoyl]phenyl]-1,4-dioxane-2-carboxamide,
(14) N-[4-chloro-3-[[5-[2-(4-fluorophenyl)ethynyl]-3-methyl-2-pyridyl]carbamoyl]phenyl]-1,4-dioxane-2-carboxamide,
(15) tert-butyl N-[4-chloro-3-[[3-methyl-5-[2-(2-pyridyl)ethynyl]-2-pyridyl]carbamoyl]phenyl]carbamate,
(16) 2-chloro-5-(3,3-difluoroazetidine-1-carbonyl)-N-[3-methyl-5-(2-phenylethynyl)-2-pyridyl]benzamide,
(17) 4-chloro-N1-(2-hydroxy-1,1-dimethyl-ethyl)-N3-[3-methyl-5-(2-phenylethynyl)-2-pyridyl]benzene-1,3-dicarboxamide,
(18) 2-chloro-N-[5-[2-(3-fluorophenyl)ethynyl]-3-methyl-2-pyridyl]-5-(3-hydroxy-3-methyl-azetidine-1-carbonyl)benzamide,
(19) 2-chloro-N-[3-methyl-5-(2-phenylethynyl)-2-pyridyl]-5-(5-tetrahydrofuran-3-yl-1,2,4-oxadiazol-3-yl)benzamide,
(20) 2-chloro-N-[3-methyl-5-(2-phenylethynyl)-2-pyridyl]-5-(tetrahydropyran-4-ylmethoxy)benzamide,
(21) 2-chloro-5-[[(2R)-1,4-dioxan-2-yl]methoxy]-N-[3-methyl-5-(2-phenylethynyl)-2-pyrid yl]benzamide,
(22) 2-chloro-5-(1,4-dioxan-2-ylmethoxy)-N-[3-methyl-5-(2-phenylethynyl)-2-pyridyl]benz amide,
(23) 2-chloro-5-[[(2S)-1,4-dioxan-2-yl]methoxy]-N-[3-methyl-5-(2-phenylethynyl)-2-pyridyl]benzamide,
(24) 2-chloro-5-(2-methoxyethoxy)-N-[3-methyl-5-(2-phenylethynyl)-2-pyridyl]benzamide,
(25) tert-butyl (3-((4-(benzyloxy)-2-methylphenyl)carbamoyl)-4-chlorophenyl)carbamate,
(26) tert-butyl (4-chloro-3-((2-methyl-4-(phenoxymethyl)phenyl)carbamoyl)phenyl)carbamate, or a pharmaceutically acceptable salt thereof.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

In some instances, the number of carbon atoms in a hydrocarbyl substituent (e.g., alkyl, alkoxy, alkenyl, alkenylene, alkynyl, alkylene, alkynylene, cycloalkyl, cycloalkane, haloalkyl, haloalkoxy, heteroalkyl or thioalkyl) is indicated by the prefix “Cx-Cy-”, wherein x is the minimum and y is the maximum number of carbon atoms in the substituent. Thus, for example, “C1-C3-alkyl” refers to an alkyl substituent containing from 1 to 3 carbon atoms, “C1-C10-alkoxy” refers to an alkoxy substituent containing from 1 to 10 carbon atoms, “C2-C10-alkenyl” refers to an alkenyl substituent containing from 2 to 10 carbon atoms, “C2-C4-alkenylene” refers to an alkenylene substituent containing from 2 to 4 carbon atoms, “C2-C10-alkynyl” refers to an alkynyl substituent containing from 2 to 10 carbon atoms, “C2-C10-alkylene” refers to an alkylene substituent containing from 2 to 10 carbon atoms, “C2-C4-alkynylene” refers to an alkynylene substituent containing from 2 to 4 carbon atoms, “C3-C10-cycloalkyl” refers to a cycloalkyl substituent containing from 3 to 10 carbon atoms, “C3-C10-cycloalkane” refers to a cycloalkane containing from 3 to 10 carbon atoms, “C1-C10-haloalkyl” refers to a haloalkyl substituent containing from 1 to 10 carbon atoms, “C1-C10-haloalkoxy” refers to a haloalkoxy substituent containing from 1 to 10 carbon atoms, “C2-C10-heteroalkyl” refers to a heteroalkyl substituent containing from 2 to 10 carbon atoms or “C1-C10-thioalkyl” refers to a thioalkyl substituent containing from 2 to 10 carbon atoms.

The term “alkyl,” as used herein, means a straight or branched, saturated hydrocarbon chain containing from 1 to 10 carbon atoms. The term “lower alkyl” or “C1-C6-alkyl” means a straight or branched chain hydrocarbon containing from 1 to 6 carbon atoms. The term “C1-C4-alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

As C1-C10 alkyl of R⁷, C1-C4-alkyl is preferred. More preferable C1-C10-alkyl of R⁷ is methyl.

As C1-C10-alkyl of substituents in R⁷, C1-C4-alkyl is preferred.

As C1-C10 alkyl of R⁸, C1-C4-alkyl is preferred. More preferable C1-C10-alkyl of R 8 is methyl.

As C1-C10-alkyl of substituents in R⁸, C1-C4-alkyl is preferred.

As C1-C10 alkyl of R⁹, C1-C4-alkyl is preferred.

As C1-C10 alkyl of R¹⁰, C1-C4-alkyl is preferred.

As C1-C10 alkyl of R¹¹, C1-C4-alkyl is preferred.

As C1-C10-alkyl of substituents in Q, C1-C4-alkyl is preferred.

As C1-C10-alkyl of R¹⁰¹, C1-C4-alkyl is preferred.

As C1-C10-alkyl of R¹⁰², C1-C4-alkyl is preferred.

As C1-C10-alkyl of R¹⁰³, C1-C4-alkyl is preferred.

As C1-C10-alkyl of R¹⁰⁴, C1-C4-alkyl is preferred.

As C1-C10-alkyl of R¹⁰⁵, C1-C4-alkyl is preferred.

As C1-C10-alkyl of R¹⁰⁶, C1-C4-alkyl is preferred.

As C1-C10-alkyl of R¹⁰⁷, C1-C4-alkyl is preferred.

As C1-C10-alkyl of R¹⁰⁸, C1-C4-alkyl is preferred.

As C1-C10-alkyl of R¹⁰⁹, C1-C4-alkyl is preferred.

As C1-C10-alkyl of R¹¹⁰, C1-C4-alkyl is preferred.

As C1-C10-alkyl of R¹¹¹, C1-C4-alkyl is preferred.

As C1-C10-alkyl of R¹¹², C1-C4-alkyl is preferred.

As C1-C10-alkyl of R¹², C1-C4-alkyl is preferred.

As C1-C10-alkyl of R¹³, C1-C4-alkyl is preferred.

As C1-C10-alkyl of R¹⁴, C1-C4-alkyl is preferred.

As C1-C10-alkyl of R¹, C1-C4-alkyl is preferred. More preferable C1-C10-alkyl of R 1 is methyl.

As C1-C10-alkyl of R², C1-C4-alkyl is preferred.

As C1-C10-alkyl of R³, C1-C4-alkyl is preferred. C1-C10-alkyl of R⁴, C1-C4-alkyl is preferred. More preferable C1-C10-alkyl of R⁴ is methyl.

As C1-C10-alkyl of substituents in C3-C10-cycloalkyl of R⁴, C1-C4-alkyl is preferred.

As C1-C10-alkyl of R⁴¹, C1-C4-alkyl is preferred.

As C1-C10-alkyl of R⁴², C1-C4-alkyl is preferred.

As C1-C10-alkyl of R⁴³, C1-C4-alkyl is preferred.

As C1-C10-alkyl of R⁴⁴, C1-C4-alkyl is preferred.

As C1-C10-alkyl of R⁴⁵, C1-C4-alkyl is preferred.

As C1-C10-alkyl of R⁴⁶, C1-C4-alkyl is preferred.

As C1-C10-alkyl of R⁴⁷, C1-C4-alkyl is preferred.

As C1-C10-alkyl of R⁴⁸, C1-C4-alkyl is preferred.

As C1-C10-alkyl of R⁵, C1-C6-alkyl is preferred. More preferable C1-C10-alkyl of R⁵ is C1-C4-alkyl.

As C1-C10-alkyl of R¹⁵, C1-C4-alkyl is preferred. More preferable C1-C10-alkyl of R¹⁵ is tert-butyl.

As C1-C10-alkyl of R¹⁶, C1-C4-alkyl is preferred. More preferable C1-C10-alkyl of R¹⁶ is tert-butyl.

As C1-C10-alkyl of R¹⁷, C1-C4-alkyl is preferred.

As C1-C10-alkyl of R¹⁸, C1-C4-alkyl is preferred.

As C1-C10-alkyl of R¹⁹, C1-C4-alkyl is preferred.

As C1-C10-alkyl of R²⁰, C1-C4-alkyl is preferred.

As C1-C10-alkyl of R²¹, C1-C4-alkyl is preferred.

As C1-C10-alkyl of R²², C1-C4-alkyl is preferred.

As C1-C10-alkyl of substituents in R²³, C1-C4-alkyl is preferred.

As C1-C10-alkyl of R²⁰¹, C1-C4-alkyl is preferred.

As C1-C10-alkyl of R²⁰², C1-C4-alkyl is preferred.

As C1-C10-alkyl of R²⁰³, C1-C4-alkyl is preferred.

As C1-C10-alkyl of R²⁰⁴, C1-C4-alkyl is preferred.

As C1-C10-alkyl of R²⁰⁵, C1-C4-alkyl is preferred.

As C1-C10-alkyl of R²⁰⁶, C1-C4-alkyl is preferred.

As C1-C10-alkyl of R²⁰⁷, C1-C4-alkyl is preferred.

As C1-C10-alkyl of R²⁰⁸, C1-C4-alkyl is preferred.

As C1-C10-alkyl of R²⁰⁹, C1-C4-alkyl is preferred.

As C1-C10-alkyl of R²¹⁰, C1-C4-alkyl is preferred.

As C1-C10-alkyl of R²¹¹, C1-C4-alkyl is preferred.

As C1-C10-alkyl of R²¹², C1-C4-alkyl is preferred.

As C1-C10-alkyl of R²⁴, C1-C4-alkyl is preferred.

As C1-C10-alkyl of substituents in R²⁴, C1-C4-alkyl is preferred.

As C1-C10-alkyl of R²⁵, C1-C4-alkyl is preferred.

As C1-C10-alkyl of substituents in R²⁵, C1-C4-alkyl is preferred. Most preferable C1-C10-alkyl of R²⁵ is methyl.

As C1-C10-alkyl of R²⁶, C1-C4-alkyl is preferred.

As C1-C10-alkyl of R²⁷, C1-C4-alkyl is preferred.

As C1-C10-alkyl of R^{1a}, C1-C4-alkyl is preferred. More preferable C1-C10-alkyl of R^{1a} is methyl.

As C1-C10-alkyl of R^{8a}, C1-C4-alkyl is preferred. More preferable C1-C10-alkyl of R^{8a} is methyl.

As C1-C10-alkyl of substituents in R^{5a}, C1-C4-alkyl is preferred.

As C1-C10-alkyl of —NH—C(═O)—O—(C1-C10-alkyl) in R^{5a}, C1-C4-alkyl is preferred.

As C1-C10-alkyl of —O—(C1-C10-alkyl) in R^{5a}, C1-C4-alkyl is preferred.

As C1-C10-alkyl of —C(═O)—NH—(C1-C10-alkyl) in R^{5a}, C1-C4-alkyl is preferred.

As C1-C10-alkyl of R⁵¹, C1-C4-alkyl is preferred.

As C1-C10-alkyl of substituents in R⁵¹, C1-C4-alkyl is preferred.

As C1-C10-alkyl of R^{1a}, C1-C4-alkyl is preferred. More preferable C1-C10-alkyl of R^{1a} is methyl.

The term “alkoxy,” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

As C1-C10-alkoxy of R⁷, C1-C4-alkoxy is preferred.

As C1-C10-alkoxy of R⁸, C1-C4-alkoxy is preferred.

As C1-C10-alkoxy of R¹⁴, C1-C6-alkoxy is preferred. More preferable C1-C10-alkoxy of R¹⁴ is C1-C4-alkoxy.

As C1-C10-alkoxy of R¹, C1-C4-alkoxy is preferred. More preferable C1-C10-alkoxy of R¹ is methoxy.

As C1-C10-alkoxy of R², C1-C4-alkoxy is preferred.

As C1-C10-alkoxy of R¹⁵, C1-C4-alkoxy is preferred.

As C1-C10-alkoxy of R¹⁶, C1-C4-alkoxy is preferred.

As C1-C10-alkoxy of R¹⁷, C1-C4-alkoxy is preferred.

As C1-C10-alkoxy of R¹⁸, C1-C4-alkoxy is preferred.

As C1-C10-alkoxy of R¹⁹, C1-C4-alkoxy is preferred.

As C1-C10-alkoxy of R²⁰, C1-C4-alkoxy is preferred.

As C1-C10-alkoxy of R²⁴, C1-C4-alkoxy is preferred.

As C1-C10-alkoxy of R²⁵, C1-C4-alkoxy is preferred. Most preferable C1-C10-alkoxy of R²⁵ is methoxy.

As C1-C10-alkoxy of R⁵¹, C1-C4-alkoxy is preferred.

As C1-C10-alkoxy of R^{1a}, C1-C4-alkoxy is preferred. More preferable C1-C10-alkoxy of R^{1a} is methoxy.

The term “alkenyl,” as used herein, means a straight or branched, hydrocarbon chain containing at least one carbon-carbon double bond and from 2 to 10 carbon atoms.

As C2-C10-alkenyl of R⁷, C2-C4-alkenyl is preferred.

As C2-C10-alkenyl of R⁸, C2-C4-alkenyl is preferred.

As C2-C10-alkenyl of R¹⁴, C2-C4-alkenyl is preferred.

The term “alkenylene” as used herein, refers to a divalent group derived from a straight or branched hydrocarbon chain containing at least one carbon-carbon double bond and from 2 to 10 carbon atoms.

As C2-C4-alkenylene of L, ethenylene is preferred.

The term “alkynyl,” as used herein, means a straight or branched, hydrocarbon chain containing at least one carbon-carbon triple bond and from 2 to 10 carbon atoms.

As C2-C10-alkynyl of R⁷, C2-C4-alkynyl is preferred.

As C2-C10-alkynyl of R⁸, C2-C4-alkynyl is preferred.

As C2-C10-alkynyl of R¹⁴, C2-C4-alkynyl is preferred.

The term “alkoxyalkyl,” as used herein, refers to an alkoxy group, as defined herein, appended to the parent molecular moiety through an alkyl group, as defined herein.

The term “alkoxyfluoroalkyl,” as used herein, refers to an alkoxy group, as defined herein, appended to the parent molecular moiety through a fluoroalkyl group, as defined herein.

The term “alkylene,” as used herein, refers to a divalent group derived from a straight or branched chain hydrocarbon of 1 to 10 carbon atoms, for example, of 2 to 5 carbon atoms. Representative examples of alkylene include, but are not limited to, —CH₂—, —CH₂CH₂—, —CH₂CH₂CH₂—, —CH₂CH₂CH₂CH₂—, —C(CH₃)₂, —CH₂—, —CH₂CH₂CH₂CH₂CH₂—, —CH₂—C(CH₃)₂—, —CH(CH₃)—CH₂—, —CH₂—CH(CH₃)— and —CH(CH₃)—.

As C1-C10-alkylene of (C1-C10-alkylene)-O— in L, —C1-C4-alkylene is preferred. More preferable C1-C10-alkylene of (C1-C10-alkylene)-O— in L is —CH₂—.

As C1-C10-alkylene of —O—(C1-C10-alkylene)- in L, —C1-C4-alkylene is preferred. More preferable C1-C10-alkylene of —O—(C1-C10-alkylene)- in L is —CH₂—.

As C1-C10-alkylene of —O—(C1-C10-alkylene)-R¹⁸ in R⁵, C1-C4-alkylene is preferred.

As C1-C10-alkylene of —(C1-C10-alkylene)-(CR²¹R²²)ₚ—R²³ in R⁵, C1-C4-alkylene is preferred.

As C1-C10-alkylene of —(C1-C10-alkylene)-NR²⁶R²⁷ in R²⁴, C1-C4-alkylene is preferred.

As C1-C10-alkylene of —(C1-C10-alkylene)-NR²⁶R²⁷ in R²⁵, C1-C4-alkylene is preferred.

As C1-C10-alkylene of —(C1-C10-alkylene)-(3 to 15 membered heterocycle) in R²⁴, C1-C4-alkylene is preferred.

As C1-C10-alkylene of —(C1-C10-alkylene)-(3 to 15 membered heterocycle) in R²⁵, C1-C4-alkylene is preferred.

As C1-C10-alkylene of —O—(C1-C10-alkylene)-R¹¹ in R^{5a}, C1-C4-alkylene is preferred.

As C1-C10-alkylene of —O—(C1-C10-alkylene)-(3 to 15 membered heterocycle) in R^{5a}, C1-C4-alkylene is preferred.

As C1-C10-alkylene of —(C1-C10-alkylene)-(3 to 15 membered heterocycle) in R⁵¹, C1-C4-alkylene is preferred.

The term “alkynylene” as used herein, refers to a divalent group derived from a straight or branched hydrocarbon chain containing at least one carbon-carbon triple bond and from 2 to 10 carbon atoms.

As C2-C4-alkynylene of L, ethynylene is preferred.

The term “alkylamino,” as used herein, means at least one alkyl group, as defined herein, is appended to the parent molecular moiety through an amino group, as defined herein.

The term “amide,” as used herein, means —C(O)NR^{E}— or —NR^{E}C(O)—, wherein RE may be hydrogen, alkyl, cycloalkyl, aryl, heteroaryl, heterocycle, alkenyl, or heteroalkyl.

The term “aminoalkyl,” as used herein, means at least one amino group, as defined herein, is appended to the parent molecular moiety through an alkylene group, as defined herein.

The term “amino,” as used herein, means —NR^{F}R^{G}, wherein R^{F} and R^{G} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NR^{H}—, wherein R^{H} may be hydrogen, alkyl, cycloalkyl, aryl, heteroaryl, heterocycle, alkenyl, or heteroalkyl.

The term “aryl,” as used herein, refers to a phenyl group, or a bicyclic fused ring system. Bicyclic fused ring systems are exemplified by a phenyl group appended to the parent molecular moiety and fused to a cycloalkyl group, as defined herein, a phenyl group, a heteroaryl group, as defined herein, or a heterocycle, as defined herein. Such a bicyclic fused ring system is comprised of no more than fifteen atoms. The term “6 to 15 membered aryl” means the ring system comprising of 6 to 15 atoms. The term “6 to 10 membered aryl” means the ring system comprising of 6 to 10 atoms. Representative examples of aryl include, but are not limited to, indolyl, naphthyl, phenyl, tetrahydroquinolinyl, 2,3-dihydrobenzo[1,4]dioxine-6-yl, indazole-5-yl, and benzo[1,3]dioxole-5-yl, benzofuran-5-yl.

As -(6 to 15 membered aryl)- of L, 6 to 10 membered aryl is preferred.

As 6 to 15 membered aryl of R⁷, 6 to 10 membered aryl is preferred.

As 6 to 15 membered aryl of R⁸, 6 to 10 membered aryl is preferred.

As 6 to 15 membered aryl of Q, 6 to 10 membered aryl is preferred.

As 6 to 15 membered aryl of R, 6 to 10 membered aryl is preferred. More preferable 6 to 15 membered aryl of R is phenyl.

As 6 to 15 membered aryl of R¹⁴, 6 to 10 membered aryl is preferred.

As 6 to 15 membered aryl of R⁵, 6 to 10 membered aryl is preferred.

As 6 to 15 membered aryl of R¹⁵, 6 to 10 membered aryl is preferred.

As 6 to 15 membered aryl of R¹⁶, 6 to 10 membered aryl is preferred.

As 6 to 15 membered aryl of R¹⁷, 6 to 10 membered aryl is preferred.

As 6 to 15 membered aryl of R¹⁸, 6 to 10 membered aryl is preferred.

As 6 to 15 membered aryl of R¹⁹, 6 to 10 membered aryl is preferred.

As 6 to 15 membered aryl of R²⁰, 6 to 10 membered aryl is preferred.

As 6 to 15 membered aryl of R²³, 6 to 10 membered aryl is preferred.

As 6 to 15 membered aryl of R^{5a}, 6 to 10 membered aryl is preferred.

As 6 to 15 membered aryl of Ring B, 6 to 10 membered aryl is preferred.

The term “cyanoalkyl,” as used herein, means at least one —CN group, is appended to the parent molecular moiety through an alkylene group, as defined herein.

The term “cyanofluoroalkyl,” as used herein, means at least one —CN group, is appended to the parent molecular moiety through a fluoroalkyl group, as defined herein.

The term “cycloalkoxy,” as used herein, refers to a cycloalkyl group, as defined herein, appended to the parent molecular moiety through an oxygen atom.

The term “cycloalkyl,” as used herein, refers to a monocarbocyclic ring system or a bicarbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein (e.g., a phenyl group), a heteroaryl group as defined herein, or a heterocycle as defined herein. Representative examples of cycloalkyl also include, but are not limited to, 4,5,6,7-tetrahydro-1H-indazolyl. The bicarbocyclic ring system is a monocarobocyclic ring system fused to a monocarbocyclic ring system, a spiro cycloalkyl group or a bridged monocarbocyclic ring system in which two non-adjacent atoms of the ring are linked by an alkylene bridge of 1, 2, 3, 4 carbon atoms. Representative example bicarbocyclic ring systems include, but are not limited, spiro[2.2]pentanyl, spiro[2.4]heptanyl, spiro[3.5]nonanyl, bicyclo[2.2.1]heptanyl or bicyclo[2.2.2]octanyl.

As C3-C10-cycloalkyl of R⁷, C3-C6-cycloalkyl is preferred.

As C3-C10-cycloalkyl of R⁸, C3-C6-cycloalkyl is preferred.

As C3-C10-cycloalkyl which is formed R¹⁰ and R¹¹ together with the carbon atom to which they are attached, C3-C7-cycloalkyl is preferred.

As C3-C10-cycloalkyl of Q, C3-C8-cycloalkyl is preferred.

As C3-C10-cycloalkyl of R⁴, C3-C6-cycloalkyl is preferred.

As C3-C10-cycloalkyl of R⁵, C3-C6-cycloalkyl is preferred.

As C3-C10-cycloalkyl of R¹⁵, C3-C6-cycloalkyl is preferred.

As C3-C10-cycloalkyl of R¹⁶, C3-C6-cycloalkyl is preferred.

As C3-C10-cycloalkyl of R¹⁷, C3-C6-cycloalkyl is preferred.

As C3-C10-cycloalkyl of R¹⁸, C3-C6-cycloalkyl is preferred.

As C3-C10-cycloalkyl of R¹⁹, C3-C6-cycloalkyl is preferred.

As C3-C10-cycloalkyl of R²⁰, C3-C6-cycloalkyl is preferred.

As C3-C10-cycloalkyl which is formed R²¹ and R²² together with the carbon atom to which they are attached, C3-C7-cycloalkyl is preferred.

As C3-C10-cycloalkyl of R²³, C3-C8-cycloalkyl is preferred.

As C3-C10-cycloalkyl of R²⁴, C3-C6-cycloalkyl is preferred.

As C3-C10-cycloalkyl of R²⁵, C3-C6-cycloalkyl is preferred.

As C3-C10-cycloalkyl of R^{5a}, C3-C7-cycloalkyl is preferred.

As C3-C10-cycloalkyl of —C(═O)—(C3-C10-cycloalkyl) in R^{5a}, C3-C7-cycloalkyl is preferred.

As C3-C10-cycloalkyl of R⁵¹, C3-C7-cycloalkyl is preferred.

The term “cycloalkane,” as used herein, refers to a monocarbocyclic ring system or a bicarbocyclic ring system containing three to ten carbon atoms, zero heteroatoms and zero double bonds. Representative examples of cycloalkane include, but are not limited to, cyclopropane, cyclobutane, cyclopentane, cyclohexane, cycloheptane, cyclooctane, cyclononane and cyclodecane. “Cycloalkane” also includes carbocyclic ring systems in which a cycloalkane group is appended to the parent molecular moiety and is fused to an aryl group as defined herein (e.g., a phenyl group), a heteroaryl group as defined herein, or a heterocycle as defined herein. Representative examples of cycloalkane also include, but are not limited to, 4,5,6,7-tetrahydro-1H-indazole. The bicarbocyclic ring system is a monocarobocyclic ring system fused to a monocarbocyclic ring system, a spiro cycloalkane group or a bridged monocarbocyclic ring system in which two nonadjacent atoms of the ring are linked by an alkylene bridge of 1, 2, 3, 4 carbon atoms. Representative example bicarbocyclic ring systems include, but are not limited, spiro[2.2]pentane, spiro[2.4]heptane, spiro[3.5]nonane, bicyclo[2.2.1]heptane or bicyclo[2.2.2]octane.

As C3-C10-cycloalkane of —(C3-C10-cycloalkane)- in L, C3-C6-cycloalkane is preferred.

The term “cycloalkenyl,” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “fluoroalkyl,” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “fluoroalkoxy,” as used herein, means at least one fluoroalkyl group, as defined herein, is appended to the parent molecular moiety through an oxygen atom. Representative examples of fluoroalkoxy include, but are not limited to, difluoromethoxy, trifluoromethoxy and 2,2,2-trifluoroethoxy.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

As C1-C10-haloalkyl of substituents in R⁷, C1-C4-haloalkyl is preferred.

As C1-C10-haloalkyl of substituents in R⁸, C1-C4-haloalkyl is preferred.

As C1-C10-haloalkyl of R⁹, C1-C4-haloalkyl is preferred.

As C1-C10-haloalkyl of R¹⁰, C1-C4-haloalkyl is preferred.

As C1-C10-haloalkyl of R¹¹, C1-C4-haloalkyl is preferred.

As C1-C10-haloalkyl of substituents in Q, C1-C4-haloalkyl is preferred.

As C1-C10-haloalkyl of R¹⁰¹, C1-C4-haloalkyl is preferred.

As C1-C10-haloalkyl of R¹⁰², C1-C4-haloalkyl is preferred.

As C1-C10-haloalkyl of R¹⁰³, C1-C4-haloalkyl is preferred.

As C1-C10-haloalkyl of R¹⁰⁴, C1-C4-haloalkyl is preferred.

As C1-C10-haloalkyl of R¹⁰⁵, C1-C4-haloalkyl is preferred.

As C1-C10-haloalkyl of R¹⁰⁶, C1-C4-haloalkyl is preferred.

As C1-C10-haloalkyl of R¹⁰⁷, C1-C4-haloalkyl is preferred.

As C1-C10-haloalkyl of R¹⁰⁸, C1-C4-haloalkyl is preferred.

As C1-C10-haloalkyl of R¹⁰⁹, C1-C4-haloalkyl is preferred.

As C1-C10-haloalkyl of R¹¹⁰, C1-C4-haloalkyl is preferred.

As C1-C10-haloalkyl of R¹¹¹, C1-C4-haloalkyl is preferred.

As C1-C10-haloalkyl of R¹¹², C1-C4-haloalkyl is preferred.

As C1-C10-haloalkyl of R¹², C1-C4-haloalkyl is preferred.

As C1-C10-haloalkyl of R¹³, C1-C4-haloalkyl is preferred.

As C1-C10-haloalkyl of R¹, C1-C4-haloalkyl is preferred.

As C1-C10-haloalkyl of R², C1-C4-haloalkyl is preferred.

As C1-C10-haloalkyl of R⁴, C1-C4-haloalkyl is preferred.

As C1-C10-haloalkyl of substituents in C3-C10-cycloalkyl of R⁴, C1-C4-haloalkyl is preferred.

As C1-C10-haloalkyl of R²¹, C1-C4-haloalkyl is preferred.

As C1-C10-haloalkyl of R²², C1-C4-haloalkyl is preferred.

As C1-C10-haloalkyl of substituents in R²³, C1-C4-haloalkyl is preferred.

As C1-C10-haloalkyl of R²⁰¹, C1-C4-haloalkyl is preferred.

As C1-C10-haloalkyl of R²⁰², C1-C4-haloalkyl is preferred.

As C1-C10-haloalkyl of R²⁰³, C1-C4-haloalkyl is preferred.

As C1-C10-haloalkyl of R²⁰⁴, C1-C4-haloalkyl is preferred.

As C1-C10-haloalkyl of R²⁰⁵, C1-C4-haloalkyl is preferred.

As C1-C10-haloalkyl of R²⁰⁶, C1-C4-haloalkyl is preferred.

As C1-C10-haloalkyl of R²⁰⁷, C1-C4-haloalkyl is preferred.

As C1-C10-haloalkyl of R²⁰⁸, C1-C4-haloalkyl is preferred.

As C1-C10-haloalkyl of R²⁰⁹, C1-C4-haloalkyl is preferred.

As C1-C10-haloalkyl of R²¹⁰, C1-C4-haloalkyl is preferred.

As C1-C10-haloalkyl of R²¹¹, C1-C4-haloalkyl is preferred.

As C1-C10-haloalkyl of R²¹², C1-C4-haloalkyl is preferred.

As C1-C10-haloalkyl of substituents in R²⁴, C1-C4-haloalkyl is preferred.

As C1-C10-haloalkyl of substituents in R²⁵, C1-C4-haloalkyl is preferred.

As C1-C10-haloalkyl of substituents in R⁵¹, C1-C4-haloalkyl is preferred.

The term “haloalkoxy,” as used herein, means at least one haloalkyl group, as defined herein, is appended to the parent molecular moiety through an oxygen atom.

As C1-C10-haloalkoxy of R¹, C1-C4-haloalkyl is preferred.

As C1-C10-haloalkoxy of R², C1-C4-haloalkyl is preferred.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

As C2-C10-heteroalkyl of R⁷, C2-C4-heteroalkyl is preferred.

As C2-C10-heteroalkyl of R⁸, C2-C4-heteroalkyl is preferred.

The term “heteroaryl,” as used herein, refers to an aromatic monocyclic ring or an aromatic bicyclic ring system. The term “5 to 15 membered heteroaryl” means the ring system comprising of 5 to 15 atoms. The term “5 to 10 membered heteroaryl” means the ring system comprising of 5 to 10 atoms. The term “5 membered heteroaryl” means the ring system comprising of 5 atoms. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered six membered aromatic monocyclic rings have three double bonds. The bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. Such an aromatic bicyclic ring system is comprised of no more than fifteen atoms. Representative examples of heteroaryl include, but are not limited to, indolyl, pyridinyl (including pyridin-2-yl, pyridin-3-yl, pyridin-4-yl), pyrimidinyl, pyrimidine-5-yl, pyrazinyl, pyridazinyl, pyrazolyl, pyrazole-1,3-yl, pyrazole-1,4-yl, pyrrolyl, benzopyrazolyl, 1,2,3-triazolyl, thiophene-2-yl, 1,3,4-thiadiazolyl, 1,2,4-thiadiazolyl, isoxazol-3,5-yl, 1,3,4-oxadiazolyl, 1,2,4-oxadiazolyl, imidazolyl, thiazolyl, isothiazolyl, triazolyl, thiazole-5-yl, tetrazolyl, thienyl, benzimidazolyl, benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl, quinazolinyl, 1,2,4-triazinyl, 1,3,5-triazinyl, isoquinolinyl, quinolinyl, quinoline-3-yl, 6,7-dihydro-1,3-benzothiazolyl, imidazo[1,2-a]pyridinyl, naphthyridinyl, pyridoimidazolyl, thiazolo[5,4-b]pyridin-2-yl, thiazolo[5,4-d]pyrimidin-2-yl, [1,2,4]triazolo[1,5-a]pyridine, [1,2,4]triazolo[4,3-a]pyridine, [1,2,4]triazolo[1,5-a]pyridine and 4,5,6,7-tetrahydro-1H-indazolyl.

As -(5 to 15 membered heteroaryl)- of L, 5 to 10 membered heteroaryl is preferred.

More preferable (5 to 15 membered heteroaryl)- of L is 5-membered heteroaryl.

As 5 to 15 membered heteroaryl of R⁷, 5 to 10 membered heteroaryl is preferred.

As 5 to 15 membered heteroaryl of R⁸, 5 to 10 membered heteroaryl is preferred.

As 5 to 15 membered heteroaryl of Q, 5 to 10 membered heteroaryl is preferred.

As 5 to 15 membered heteroaryl of R, 5 to 10 membered heteroaryl is preferred.

As 5 to 15 membered heteroaryl of R⁵, 5 to 10 membered heteroaryl is preferred.

As 5 to 15 membered heteroaryl of R¹⁵, 5 to 10 membered heteroaryl is preferred.

As 5 to 15 membered heteroaryl of R¹⁶, 5 to 10 membered heteroaryl is preferred.

As 5 to 15 membered heteroaryl of R¹⁷, 5 to 10 membered heteroaryl is preferred.

As 5 to 15 membered heteroaryl of R¹⁸, 5 to 10 membered heteroaryl is preferred.

As 5 to 15 membered heteroaryl of R¹⁹, 5 to 10 membered heteroaryl is preferred.

As 5 to 15 membered heteroaryl of R²⁰, 5 to 10 membered heteroaryl is preferred.

As 5 to 15 membered heteroaryl of R²³, 5 to 10 membered heteroaryl is preferred.

As 5 to 15 membered heteroaryl of R^{5a}, 5 to 10 membered heteroaryl is preferred.

As 5 to 15 membered heteroaryl of Ring B, 5 to 10 membered heteroaryl is preferred.

More preferable 5 to 15 membered heteroaryl of Ring B is 5-membered heteroaryl.

More preferable 5 to 15 membered heteroaryl of Ring B is also thienyl, pyrazolyl, isoxazolyl or oxadiazolyl. Furthermore preferable 5 to 15 membered heteroaryl of Ring B is

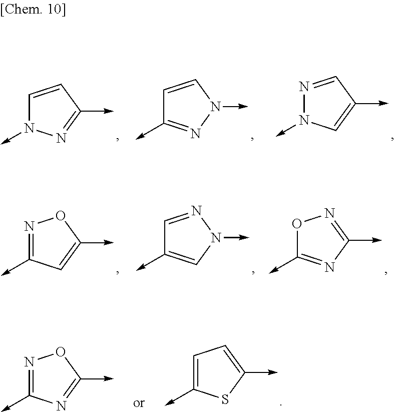

Most preferable 5 to 15 membered heteroaryl of Ring B is

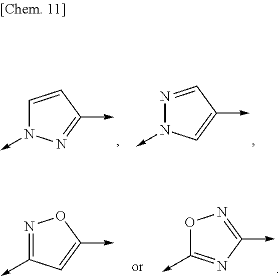

The term “heterocycle” or “heterocyclic,” as used herein, means a monocyclic heterocycle, a bicyclic heterocycle, or a tricyclic heterocycle that is comprised of three to fifteen atoms. The term “3 to 15 membered heterocycle” means the ring system comprising of 3 to 15 atoms. The term “3 to 10 membered heterocycle” means the ring system comprising of 3 to 10 atoms. The term “4 to 6 membered heterocycle” means the ring system comprising of 4 to 6 atoms. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocycles include, but are not limited to, azetidinyl, azetidine-3-yl, azepanyl, azepane-4-yl, aziridinyl, diazepanyl, 1,3-dioxanyl, 1,4-dioxane-2-yl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, morpholine-2-yl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperazine-1-yl, piperidinyl, piperidine-3-yl, piperidine-4-yl, pyranyl, pyrazolinyl, pyrazolidinyl, pyrrolinyl, pyrrolidine-3-yl, pyrrolidinyl, tetrahydrofuranyl, tetrahydrofuran-2-yl, tetrahydrofuran-3-yl, tetrahydropyranyl, tetrahydropyran-2-yl, tetrahydropyran-3-yl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, trithianyl, 3-azabicyclo[3.1.1]heptan, and 7-oxabicyclo[2.2.1]heptan. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two nonadjacent atoms of the ring are linked by an alkylene bridge of 1, 2, 3, or 4 carbon atoms, or an alkenylene bridge of 2, 3, or 4 carbon atoms, an alkoxy.bridge of 1, 2, 3, or 4 carbon atoms and 1, 2 oxygen atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, 2-azaspiro[3.3]heptane-6-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indolyl, 6-azaspiro[3.4]octane-2-yl, 7-azaspiro[3.5]nonane-2-yl, isoindolinyl, octahydrocyclopenta[c]pyrrolyl, octahydrocyclopenta[c]pyrrole-5-yl, octahydropyrrolopyridinyl, tetrahydroisoquinolinyl, [1,2,4]triazolo[1,5-a]pyridine, [1,2,4]triazolo[4,3-a]pyridine, [1,2,4]triazolo[1,5-a]pyridine and oxabicyclo[2.2.1]heptane-2-yl, 3-azabicyclo[3.1.1]heptane-6-yl.

Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocycles are connected to the parent molecular moiety through any carbon atom or any nitrogen atom contained within the rings, and can be unsubstituted or substituted.

As 3 to 15 membered heterocycle of -(3-15 membered heterocycle)- in L, 3-10 membered heterocycle is preferred. More preferable 3 to 15 membered heterocycle of -(3 to 15 membered heterocycle)- in L is 4 to 6 membered heterocycle.

As 3 to 15 membered heterocycle of R⁷, 4 to 6 membered heterocycle is preferred.

As 3 to 15 membered heterocycle of R⁸, 4 to 6 membered heterocycle is preferred.

As 3 to 15 membered heterocycle of Q, 4 to 6 membered heterocycle is preferred.

As 3 to 15 membered heterocycle of R⁵, 3 to 10 membered heterocycle is preferred.

As 3 to 15 membered heterocycle of R¹⁵, 3 to 10 membered heterocycle is preferred.

As 3 to 15 membered heterocycle of R¹⁶, 3 to 10 membered heterocycle is preferred.

As 3 to 15 membered heterocycle of R¹⁷, 3 to 10 membered heterocycle is preferred.

As 3 to 15 membered heterocycle of R¹⁸, 3 to 10 membered heterocycle is preferred.

As 3 to 15 membered heterocycle of R¹⁹, 3 to 10 membered heterocycle is preferred.

As 3 to 15 membered heterocycle of R²⁰, 3 to 10 membered heterocycle is preferred.

As 3 to 15 membered heterocycle of R²³, 3 to 10 membered heterocycle is preferred.

As 3 to 15 membered heterocycle of R²⁴, 3 to 10 membered heterocycle is preferred.

As 3 to 15 membered heterocycle of R²⁵, 3 to 10 membered heterocycle is preferred.

As 3 to 15 membered heterocycle of —(C1-C10-alkylene)-(3 to 15 membered heterocycle) in R²⁴, 3 to 10 membered heterocycle is preferred.

As 3 to 15 membered heterocycle of —(C1-C10-alkylene)-(3 to 15 membered heterocycle) in R²⁵, 3 to 10 membered heterocycle is preferred.

As 3 to 15 membered heterocycle of R^{5a}, 3 to 10 membered heterocycle is preferred.

As 3 to 15 membered heterocycle of —NH—C(═O)-(3 to 15 membered heterocycle) in R^{5a}, 3 to 10 membered heterocycle is preferred.

As 3 to 15 membered heterocycle of —O—(C1-C10-alkylene)-(3 to 15 membered heterocycle) in R^{5a}, 3 to 10 membered heterocycle is preferred.

As 3 to 15 membered heterocycle of —C(═O)-(3 to 15 membered heterocycle) in R^{5a}, 3 to 10 membered heterocycle is preferred.

As 3 to 15 membered heterocycle of —(C1-C10-alkylene)-(3 to 15 membered heterocycle) in R⁵¹, 3 to 10 membered heterocycle is preferred.

As 3 to 15 membered heterocycle of R⁵¹, 3 to 10 membered heterocycle is preferred.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

The term “hydroxyfluoroalkyl,” as used herein, means at least one —OH group, is appended to the parent molecular moiety through a fluoroalkyl group, as defined herein.

The term “Pentahalosulfanyl”, as used herein, includes, but is not limited, SF₅.

The term “thioalkyl”, as used herein, means a alkyl group, as defined herein, is appended to the parent moiety through a sulfer atom.

As C1-C10-thioalkyl of R⁷, C1-C4-thioalkyl is preferred.

As C1-C10-thioalkyl of R⁸, C1-C4-thioalkyl is preferred.

As C1-C10-thioalkyl of R¹⁴, C1-C4-thioalkyl is preferred.

The term “sulfonamide,” as used herein, means —S(O)₂NR^{K}— or —NR^{K}S(O)—, wherein R^{K} may be hydrogen, alkyl, cycloalkyl, aryl, heteroaryl, heterocycle, alkenyl, or heteroalkyl.

The term

“ [symbol] ” [Chem.12]
designates a single bond

(—) [Chem.13]
or a double bond

(═). [Chem.14]

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

The term “inhibitor” as used herein refers to a molecular entity (e.g., but not limited to, a disclosed compound) that decreases or disappears the activity of the target receptor protein.

The term “ligand” as used herein refers to a natural or synthetic molecular entity that is capable of associating or binding to a receptor to form a complex and mediate, prevent or modify a biological effect. Thus, the term “ligand” encompasses allosteric modulators, inhibitors, activators, agonists, antagonists, natural substrates and analogs of natural substrates.

The terms “natural ligand” and “endogenous ligand” as used herein are used interchangeably, and refer to a naturally occurring ligand, found in nature, which binds to a receptor.

The term “thallium flux assay” herein refers to a fluorescence-based assay used to monitor the activity of TREK channels. Thallium is a congener of potassium that readily fluxes through the pore of TREK channels. Thallium flux is measured using a commercially available, thallium-sensitive fluorescent dye called Thallos. The detail method is described below.

The term “patch clamp technique” herein refers to the “gold standard” technique for evaluating TREK channel pharmacology. The detail method is described below.

The term “MK-801-induced novel object recognition test” herein refers to the experiment to evaluate in vivo efficacy in the schizophrenic cognitive impairment animal model. The detail method is described below. MK-801 is also known as dizocilpine.

The term “TREK inhibitor” as used herein refers to any exogenously administered compound or agent that directly or indirectly inhibits the channel in an animal, in particular a mammal, for example a human.

The term “dysfunction” as used herein refers to any abnormal functions that induce activation or inhibition of the channel in an animal, in particular a mammal, for example a human.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Compounds

In one aspect, disclosed is a compound of formula (I):

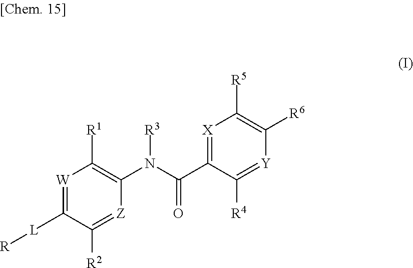

or a pharmaceutically acceptable salt thereof, wherein: all symbols are defined as below.

In some embodiments, the formula (I) is preferably the formula (Ia):

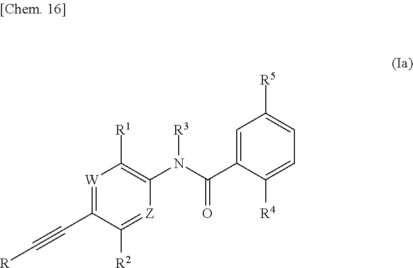

wherein all symbols are defined as below.

In some embodiments, the formula (I) is preferably the formula (Ia-1):

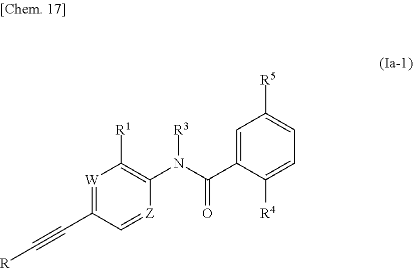

wherein all symbols are defined as below.

In some embodiments, the formula (I) is preferably the formula (Ia-1-1):

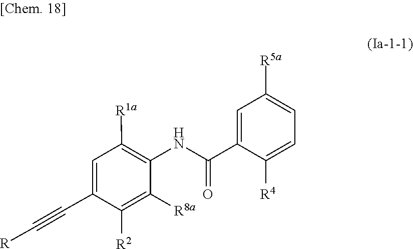

wherein all symbols are defined as below.

In some embodiments, the formula (I) is preferably the formula (Ia-1-1a):

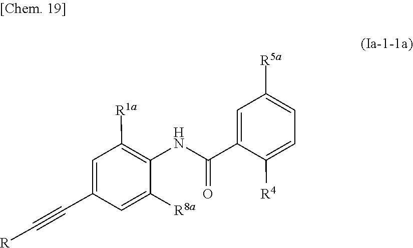

wherein all symbols are defined as below.

In some embodiments, the formula (I) is preferably the formula (Ia-1-2):

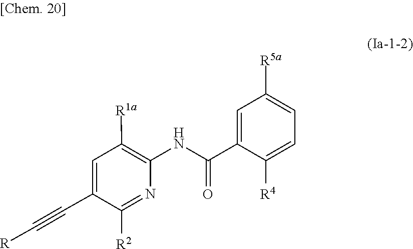

wherein all symbols are defined as below.

In some embodiments, the formula (I) is preferably the formula (Ia-1-2a):

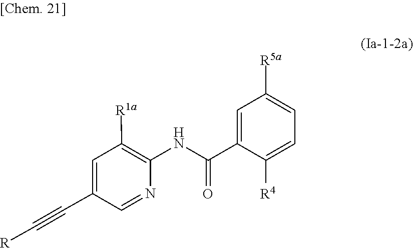

wherein all symbols are defined as below.

In some embodiments, the formula (I) is preferably the formula (II)

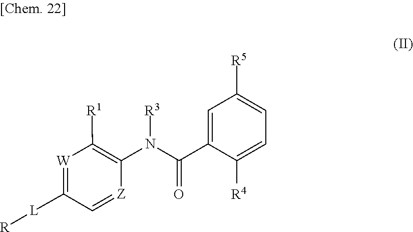

or a pharmaceutically acceptable salt thereof; wherein
L is selected from (1) ethynylene, (2) —CH₂—O— and (3) —5 membered heteroaryl (preferably thienyl, pyrazolyl, isoxazolyl or oxadiazolyl (for example, 1,2,4-oxadiazole), more preferably pyrazolyl, isoxazolyl or oxadiazolyl (for example, 1,2,4-oxadiazole));
W is selected from (1) CH, (2) CR⁷ and (3) N (preferably CH);
Z is selected from (1) CH, (2) CR⁸ and (3) N (preferably CR^{8a} or N);
R⁷ and R⁸ are each independently is cyano, halogen or C1-C4-alkyl which may be optionally substituted with 1 to 5 halogen (preferably halogen or C1-C4-alkyl which may be optionally substituted with 1 to 5 halogen, more preferably halogen or methyl);
R^{8a} is hydrogen, halogen or C1-C4-alkyl which may be optionally substituted with 1 to 5 halogen (preferably halogen or methyl);
R is selected from (1) 6 to 10 membered aryl which may be optionally substituted with 1 to 3 R¹⁴ or (2) 5 to 10 membered heteroaryl which may be optionally substituted with 1 to 3 R¹⁴ (preferably 6 to 10 membered aryl which may be optionally substituted with 1 to 3 R¹⁴, more preferably phenyl or pyridine, each of which may be optionally substituted with 1 to 3 R¹⁴, most preferably phenyl which may be optionally substituted with 1 to 3 R¹⁴);
wherein multiple R¹⁴ may be the same as or different from each other;
R¹⁴ is selected from (1) halogen, (2) cyano, (3) C1-C4-alkyl which may be optionally substituted with 1 to 5 halogen or (4) C1-C4-alkoxy which may be optionally substituted with 1 to 5 halogen (more preferably (1) halogen, (3) C1-C4-alkyl which may be optionally substituted with 1 to 5 halogen or (4) C1-C4-alkoxy which may be optionally substituted with 1 to 5 halogen, most preferably (1) halogen or (3) methyl which may be optionally substituted with 1 to 3 halogen);
R¹ is selected from (1) halogen, (2) C1-C4-alkyl, (3) C1-C4-alkoxy, (4) C1-C4-haloalkyl or (5) C1-C4-haloalkoxy (preferably (1) halogen, (2) C1-C4-alkyl or (3) C1-C4-alkoxy, more preferably (1) halogen (2) methyl or (3) methoxy);
R² is selected from (1) hydrogen, (2) halogen, (3) C1-C4-alkyl, (4) C1-C4-alkoxy, (5) C1-C4-haloalkyl or (6) C1-C4-haloalkoxy (preferably hydrogen);
R³ is selected from (1) hydrogen or (2) C1-C4-alkyl (preferably hydrogen);
R⁴ is halogen;
R⁵ is selected from (1) C1-C6-alkyl, (2) C1-C6-alkoxy, (3) —NH₂, (4) —NH—C(═O)—R¹⁵, (5) —NH—C(═O)—O—R¹⁶, (6) —O—R¹⁷, (7) —O—(C1-C10-alkylene)-R¹⁸, (8) —C(═O)—R¹⁹, (9) —C(═O)—NH—R²⁰, (10) 6 to 15 membered aryl, (11) 5 to 15 membered heteroaryl, (12) C3-C10-cycloalkyl or (13) 3 to 15 membered heterocycle (preferably (4) —NH—C(═O)—R¹⁵, (5) —NH—C(═O)—O—R¹⁶, (6) —O—R¹⁷, (7) —O—(C1-C4-alkylene)-R¹⁸, (8) —C(═O)—R¹⁹, (9) —C(═O)—NH—R²⁰ or (11) 5 to 10 membered heteroaryl, wherein each of (1), (2) and (7) in R⁵ may be optionally substituted with 1 to 5 substituents selected from (1) halogen (2) cyano and (3) —OH, and each of (10)-(13) in R⁵ may be optionally substituted with 1 to 5 R²⁴ (more preferably (1) —NH—C(═O)—(C3-C10-cycloalkyl), (2) —NH—C(═O)— (3 to 15 membered heterocycle), (3) —NH—C(═O)—O—(C1-C10-alkyl) which may be optionally substituted with 1 to 5 substituents selected from halogen, cyano and —OH, (4) —O—(C1-C4-alkyl) which may be optionally substituted with 1 to 5 substituents selected from halogen, cyano and —OH, (5) —O—(C1-C6-alkylene)-(3 to 15 membered heterocycle), (6) —C(═O)—(C3-C10-cycloalkyl), (7) —C(═O)-(3 to 15 membered heterocycle), (8) —C(═O)—NH—(C1-C10-alkyl) and (9) 5 to 10 membered heteroaryl, furthermore preferably (1) —NH—C(═O)—(C3-C6-cycloalkyl), (2) —NH—C(═O)— (3 to 10 membered heterocycle), β) -NH—C(═O)—O—(C1-C4-alkyl) which may be optionally substituted with 1 to 5 substituents selected from halogen, cyano and —OH, (4) —O—(C1-C4-alkyl) which may be optionally substituted with 1 to 5 substituents selected from halogen, cyano and —OH, (5) —O—(C1-C4-alkylene)-(3 to 10 membered heterocycle), (6) —C(═O)—(C3-C6-cycloalkyl), (7) —C(═O)-(3 to 10 membered heterocycle), (8) —C(═O)—NH—(C1-C4-alkyl) and (9) 5 to 10 membered heteroaryl);
wherein each of (1), (2) and (5)-(9) in R⁵ may be optionally substituted with 1 to 5 R⁵¹;
multiple R⁵¹ may be the same as or different from each other,
R²⁴ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (4) C1-C4-alkyl, (7) 3 to 10 membered heterocycle or (8) —(C1-C4-alkylene)-(3 to 10 membered heterocycle), more preferably (7) 3 to 10 membered heterocycle), wherein each of (4)-(5) in R²⁴ may be optionally substituted with 1 to 3 substituents selected with (1) halogen, (2) —OH and (3) cyano and each of (6)-(8) in R²⁴ may be optionally substituted with 1 to 3 C1-C4-haloalkyl;
R⁵¹ is independently selected from (1) halogen, (2) cyano, (3) —OH, (4) C1-C10-alkyl, (5) C1-C10-alkoxy, (6)-(C1-C10-alkylene)-(3 to 15 membered heterocycle), (7) C3-C10-cycloalkyl and (8) 3 to 15 membered heterocycle, wherein each of (4)-(8) in R⁵¹ may be optionally substituted with 1 to 10 substituents selected from (1) halogen, (2) —OH, (3) cyano, (4) C1-C10-alkyl and (5) C1-C10-haloalkyl (preferably (1) halogen, (2) C1-C4-alkyl, (3) C1-4-haloalkyl, (4) C1-C4-alkoxy and (5) C1-4-haloalkoxy);
R¹⁵, R¹⁶, R¹⁷, R¹⁸, R¹⁹ and R²⁰ are each independently selected from (1) C1-C4-alkyl, (2) C1-C4-alkoxy, (3) 6 to 10 membered aryl, (4) 5 to 10 membered heteroaryl, (5) C3-C6-cycloalkyl or (6) 3 to 10 membered heterocycle, wherein each of (1) and (2) in R¹⁵, R¹⁶, R¹⁷, R¹⁸, R¹⁹ or R²⁰ may be optionally substituted with 1 to 5 substituents selected from (1) halogen (2) cyano and (3) —OH, and each of (3)-(6) in R¹⁵, R¹⁶, R¹⁷, R¹⁸, R¹⁹ or R²⁰ may be optionally substituted with 1 to 5 R²⁵;
R²⁵ is selected from (1) halogen, (2) —OH, (3) cyano, (4) C1-C4-alkyl, (5) C1-C4-alkoxy, (6) C3-C10-cycloalkyl, (7) 3 to 10 membered heterocycle, or (8) —(C1-C4-alkylene)-(3 to 10 membered heterocycle) (preferably (1) halogen, (2) —OH, (4) C1-C4-alkyl or (5) C1-C4-alkoxy, more preferably (1) halogen, (2) —OH, (4) methyl which may be optionally substituted with 1 to 3 substituents selected with halogen, —OH and cyano, or (5) methoxy which may be optionally substituted with 1 to 3 substituents selected with halogen, —OH and cyano), wherein each of (4)-(5) in R²⁵ may be optionally substituted with 1 to 3 substituents selected with (1) halogen, (2) —OH and (3) cyano and each of (6)-(8) in R²⁵ may be optionally substituted with 1 to 3 substituents selected with (1) halogen, (2) —OH, (3) cyano, (4) C1-C4-alkyl and (5) C1-C4-haloalkyl.

In some embodiments, the formula (I) is preferably the formula (Ia-1), (Ib) or (Ic):

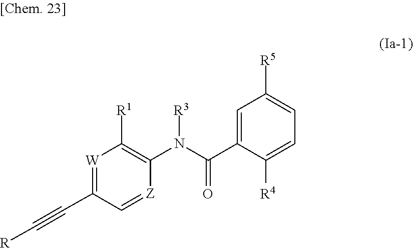

or a pharmaceutically acceptable salt thereof; wherein
R⁵ is selected from (1) cyano, (2) —NH₂, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) 6 to 15 membered aryl, (10) 5 to 15 membered heteroaryl, (11) C3-C10-cycloalkyl and (12) 3 to 15 membered heterocycle;
wherein (6) in R⁵ may be optionally substituted with 1 to 10 substituents selected from (1) halogen (2) cyano and (3) —OH, and each of (9)-(12) in R⁵ may be optionally substituted with 1 to 5 R²⁴;
the formula (Ib):

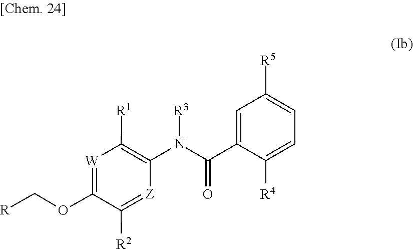

or a pharmaceutically acceptable salt thereof; wherein
R is phenyl which may be optionally substituted with 1 to 5 R¹⁴;
R⁴ is halogen,
R⁵ is selected from (1) C1-C10-alkyl, (2) C1-C10-alkoxy, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) —(C1-C10-alkylene)-(CR²¹R²²)ₚ—R²³, (10) 6 to 15 membered aryl, (11) 5 to 15 membered heteroaryl, (12) C3-C10-cycloalkyl and (13) 3 to 15 membered heterocycle;
wherein each of (1), (2), (6) and (9) in R⁵ may be optionally substituted with 1 to 10 substituents selected from (1) halogen (2) cyano and (3) —OH, and each of (10)-(13) in R⁵ may be optionally substituted with 1 to 5 R²⁴; or
the formula (Ic):

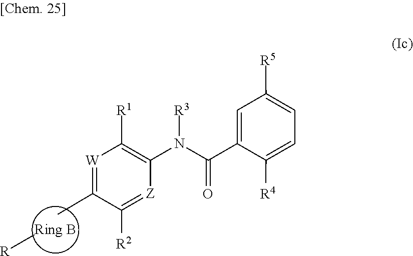

or a pharmaceutically acceptable salt thereof;
wherein Ring B is selected from

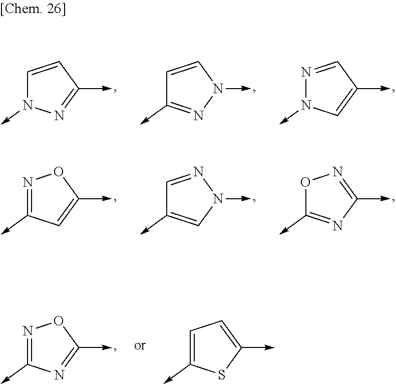

wherein right arrow is connecting position with 6 membered ring and left arrow is connecting position with R;
R is phenyl which may be optionally substituted with 1 to 5 R¹⁴;
R⁴ is halogen,
R⁵ is selected from (1) C1-C10-alkyl, (2) C1-C10-alkoxy, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) —(C1-C10-alkylene)-(CR²¹R²²)ₚ—R²³, (10) 6 to 15 membered aryl, (11) 5 to 15 membered heteroaryl, (12) C3-C10-cycloalkyl and (13) 3 to 15 membered heterocycle;
wherein each of (1), (2), (6) and (9) in R⁵ may be optionally substituted with 1 to 10 substituents selected from (1) halogen (2) cyano and (3) —OH, and each of (10)-(13) in R⁵ may be optionally substituted with 1 to 5 R²⁴;
and the other symbols are as defined as below.

In some embodiments, the formula (I) is preferably the formula (Ib):

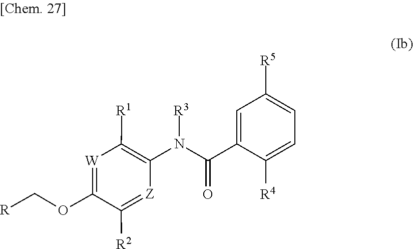

wherein all symbols are defined as below.

In some embodiments, the formula (I) is preferably the formula (Ib-1):

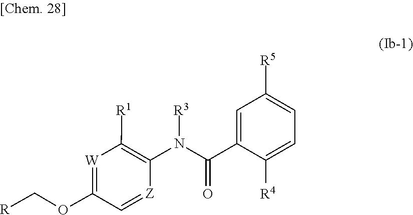

wherein all symbols are defined as below.

In some embodiments, the formula (I) is preferably the formula (Ic):

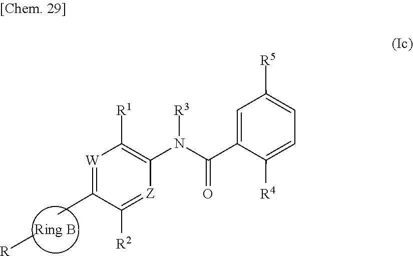

wherein all symbols are defined as below.

In some embodiments, the formula (I) is preferably the formula (Ic):

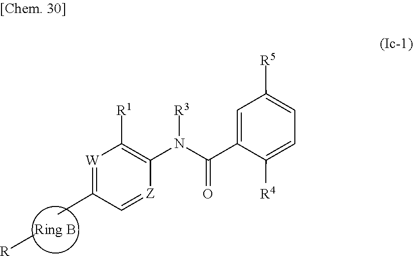

wherein all symbols are defined as below.
L is preferably bond, C2-C4-alkynylene (preferably, ethynylene), C2-C4-alkenylene(preferably, ethenylene), —(C1-C4-alkylene)-O— (preferably, —CH₂—O—), —O—(C1-C4-alkylene)- (preferably, —O—CH₂—), -(6 to 10 membered aryl)- or -(5 to 10 membered heteroaryl)-. More preferable L is C2-C4-alkynylene (preferably, ethynylene), —(C1-C4-alkylene)-O— (preferably, —CH₂—O—), —O—(C1-C4-alkylene)(preferably, —O—CH₂—), or -(5 to 10 membered heteroaryl)- (preferably, -5 membered heteroaryl). Most preferable L is C2-C4-alkynylene(preferably, ethynylene). Most preferable L is also -(5 to 6 membered heteroaryl)- (preferably, -5 membered heteroaryl). Most preferable L is also —(C1-C4-alkylene)-O— (preferably, —CH₂—O—).
W is preferably CH, or CR⁷. Most preferable W is CH.
Z is preferably CR^{8a} or N. Most preferable Z is N.
R⁷ is preferably cyano, halogen or C1-C4-alkyl which may be optionally substituted with 1 to 5 halogen. More preferable R⁷ is halogen or C1-C4-alkyl which may be optionally substituted with 1 to 5 halogen. Most preferable R⁷ is halogen or methyl.
R⁸ is preferably cyano, halogen or C1-C4-alkyl which may be optionally substituted with 1 to 5 halogen. More preferable R⁸ is halogen or C1-C4-alkyl which may be optionally substituted with 1 to 5 halogen. Most preferable R⁸ is halogen or methyl.
R^{8a} is preferably hydrogen, halogen or C1-C4-alkyl which may be optionally substituted with 1 to 5 halogen. More preferable R^{8a} is halogen or methyl.
R⁹ is preferably hydrogen, C1-C4-alkyl or C1-C4-haloalkyl. More preferable R⁹ is hydrogen or C1-C4-alkyl.
R¹⁰ is preferably hydrogen, C1-C4-alkyl or C1-C4-haloalkyl. More preferable R¹⁰ is C1-C4-alkyl.
R¹¹ is preferably hydrogen, C1-C4-alkyl or C1-C4-haloalkyl. More preferable R¹¹ is C1-C4-alkyl.
R¹⁰ and R¹¹ are preferably attached with the carbon atom together and formed a

C3-C10-cycloalkyl. More preferable R¹⁰ and R¹¹ are attached with the carbon atom together and formed a C3-C7-cycloalkyl.
n is preferably 1, 2, 3 or 4.
Q is preferably halogen, cyano, 6 to 10 membered aryl which may be may be optionally substituted with 1 to 5 substituents selected from (1)halogen, (2) C1-C4-alkyl and (3) C1-C4-haloalkyl, 5 to 10 membered heteroaryl which may be may be optionally substituted with 1 to 5 substituents selected from (1)halogen, (2) C1-C4-alkyl and (3) C1-C4-haloalkyl, C3-C8-cycloalkyl which may be may be optionally substituted with 1 to 5 substituents selected from (1)halogen, (2) C1-C4-alkyl and (3) C1-C4-haloalkyl, or 4 to 6 membered heterocycle which may be may be optionally substituted with 1 to 5 substituents selected from (1)halogen, (2) C1-C4-alkyl and (3) C1-C4-haloalkyl.

More preferable Q is halogen, cyano, C3-C8-cycloalkyl which may be may be optionally substituted with 1 to 5 substituents selected from (1)halogen, (2) C1-C4-alkyl and (3) C1-C4-haloalkyl, or 4 to 6 membered heterocycle which may be may be optionally substituted with 1 to 5 substituents selected from (1)halogen, (2) C1-C4-alkyl and (3) C1-C4-haloalkyl.
R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹ and R¹¹² are each preferably hydrogen, C1-C4-alkyl or C1-C4-haloalkyl.
R⁶ is preferably hydrogen.
X is preferably CH.
Y is preferably CH.
R¹² is preferably halogen, C1-C4-alkyl or C1-C4-haloalkyl. More preferable R¹² is halogen.
R¹³ is preferably halogen, C1-C4-alkyl or C1-C4-haloalkyl. More preferable R¹³ is halogen.
R is preferably 6 to 10 membered aryl which may be optionally substituted with 1 to 3 R¹⁴ or 5 to 10 membered heteroaryl which may be optionally substituted with 1 to 3 R¹⁴. More preferable R is 6 to 10 membered aryl which may be optionally substituted with 1 to 3 R¹⁴. More preferable R is phenyl or pyridine, each of which may be optionally substituted with 1 to 3 R¹⁴. Most preferable R is phenyl which may be optionally substituted with 1 to 3 R¹⁴.
R¹⁴ is preferably halogen, cyano, C1-C4-alkyl which may be optionally substituted with 1 to 5 halogen or C1-C4-alkoxy which may be optionally substituted with 1 to 5 halogen. More preferable R¹⁴ is halogen, C1-C4-alkyl which may be optionally substituted with 1 to 5 halogen, C1-C4-alkoxy which may be optionally substituted with 1 to 5 halogen. Most preferable R¹⁴ is halogen. Most preferable R¹⁴ is also methyl which may be optionally substituted with 1 to 3 halogen.
R¹ is preferably C1-C4-alkyl, halogen, C1-C4-alkoxy, C1-C4-haloalkyl or C1-C4-haloalkoxy. More preferable R¹ is C1-C4-alkyl or halogen. Most preferable R¹ is methyl, methoxy or halogen (more preferably methyl, halogen).

R^{1a} is preferably halogen, C1-C4-alkyl or C1-C4-haloalkyl. More preferable R^{1a} is methyl, methoxy or halogen. Most preferable R^{1a} is methyl or halogen.
R² is preferably hydrogen, C1-C4-alkyl, halogen, C1-C4-alkoxy, C1-C4-haloalkyl or C1-C4-haloalkoxy. More preferable R² is hydrogen. Especially, when R⁸ or R^{8a} is not hydrogen, preferable R² is hydrogen.
R³ is preferably hydrogen or C1-C4-alkyl. More preferable R³ is hydrogen.
R⁴ is preferably halogen, C1-C4-alkyl or C1-C4-haloalkyl. More preferable R⁴ is C1-C4-alkyl or halogen. Most preferable R⁴ is halogen.

As one preferable embodiment, R³ and R⁴ is taken together to form —CR⁴¹R⁴²— (preferably —CH₂—).
R⁴¹, R⁴², R⁴³, R⁴⁴, R⁴⁵, R⁴⁶, R⁴⁷ and R⁴⁸ are each preferably hydrogen or C1-C4-alkyl, More preferable are each hydrogen.
R⁵ is preferably C1-C6-alkyl which may be optionally substituted with 1 to 5 substituents selected from (1) halogen (2) cyano and (3) —OH,
C1-C6-alkoxy which may be optionally substituted with 1 to 5 substituents selected from (1) halogen (2) cyano and (3) —OH, cyano, —NH₂, —NH—C(═O)—R¹⁵, —NH—C(═O)—O—R¹⁶, —O—R¹⁷,
—O—(C1-C10-alkylene)-R¹¹ which may be optionally substituted with 1 to 5 substituents selected from (1) halogen (2) cyano and (3) —OH, —C(═O)—R¹⁹, —C(═O)—NH—R²⁰,
6 to 15 membered aryl which may be optionally substituted with 1 to 5 R²⁴,
5 to 15 membered heteroaryl which may be optionally substituted with 1 to 5 R²⁴,
C3-C10-cycloalkyl which may be optionally substituted with 1 to 5 R²⁴ or 3 to 15 membered heterocycle which may be optionally substituted with 1 to 5 R²⁴.

More preferable R⁵ is-NH—C(═O)—R¹⁵, —NH—C(═O)—O—R¹⁶, —O—R¹⁷, —O—(C1-C4-alkylene)-R¹¹ which may be optionally substituted with 1 to 5 substituents selected from (1) halogen (2) cyano and (3) —OH, —C(═O)—R¹⁹, —C(═O)—NH—R²⁰, or
5 to 10 membered heteroaryl which may be optionally substituted with 1 to 5 R²⁴.
R⁵ is also preferably R^{5a}.

More preferable R⁵ and R^{5a} are each NH—C(═O)—R¹⁵, —NH—C(═O)—O—R¹⁶, —O—R¹⁷, —O—(C1-C4-alkylene)-R¹¹ which may be optionally substituted with 1 to 5 substituents selected from (1) halogen (2) cyano and (3) —OH, —C(═O)—R¹⁹, —C(═O)—NH—R²⁰, or
5 to 15 membered heteroaryl which may be optionally substituted with 1 to 5 R²⁴.
R^{5a} is preferably —NH—C(═O)—(C1-C4-alkyl) which may be optionally substituted with 1 to 5 substituents selected from (1) halogen (2) cyano and (3) —OH, —NH—C(═O)—(C3-C10-cycloalkyl) which may be optionally substituted with 1 to 5 R²⁵, —NH—C(═O)-(3 to 10 membered heterocycle) which may be optionally substituted with 1 to 5 R²⁵,
—NH—C(═O)—O—(C1-C4-alkyl) which may be optionally substituted with 1 to 5 substituents selected from (1) halogen (2) cyano and (3) —OH,
—O—(C1-C4-alkylene)-(C1-C4-alkoxy) which may be optionally substituted with 1 to 5 substituents selected from (1) halogen (2) cyano and (3) —OH,
—O—(C1-C4-alkylene)-(5 to 10 membered heteroaryl) which may be optionally substituted with 1 to 5 R²⁵,
—O—(C1-C4-alkylene)-(3 to 10 membered heterocycle) which may be optionally substituted with 1 to 5 R²⁵,
—C(═O)— (5 to 10 membered heteroaryl) which may be optionally substituted with 1 to 5 R²⁵,
—C(═O)— (3 to 10 membered heterocycle) which may be optionally substituted with 1 to 5 R²⁵,
—C(═O)—NH—(C1-C4-alkyl) which may be optionally substituted with 1 to 5 substituents selected from (1) halogen (2) cyano and (3) —OH, or 5 to 10 membered heteroaryl may be optionally substituted with 1 to 5 R²⁵.

More preferable R^{5a} is
—NH—C(═O)—(C3-C10-cycloalkyl) which may be optionally substituted with 1 to 5 R²⁵,
—NH—C(═O)-(3 to 10 membered heterocycle) which may be optionally substituted with 1 to 5 R²⁵,
—NH—C(═O)—O—(C1-C4-alkyl) which may be optionally substituted with 1 to 5 substituents selected from (1) halogen (2) cyano and (3) —OH,
—O—(C1-C4-alkylene)-(3 to 10 membered heterocycle) which may be optionally substituted with 1 to 5 R²⁵,
—C(═O)— (3 to 10 membered heterocycle) which may be optionally substituted with 1 to 5 R²⁵, or
5 to 10 membered heteroaryl may be optionally substituted with 1 to 5 R²⁵. More preferable R⁵ and R^{5a} are each (1) —NH—C(═O)—(C3-C10-cycloalkyl), (2) —NH—C(═O)— (3 to 15 membered heterocycle), (3) —NH—C(═O)—O—(C1-C10-alkyl), which may be optionally substituted with 1 to 5 substituents selected from halogen, —OH and cyano, (4) —O—(C1-C10-alkyl), which may be optionally substituted with 1 to 5 substituents selected from halogen, —OH and cyano, (5) —O—(C1-C10-alkylene)-(3 to 15 membered heterocycle), (6) —C(═O)—(C3-C10-cycloalkyl), (7) —C(═O)-(3 to 15 membered heterocycle), (8) —C(═O)—NH—(C1-C10-alkyl) and (9) 5 to 15 membered heteroaryl; wherein each of (1), (2) and (5)-(9) in R^{5a} may be optionally substituted with 1 to 5 R⁵¹;
multiple R⁵¹ may be the same as or different from each other;
R⁵¹ is independently selected from (1) halogen, (2) cyano, (3) —OH, (4) C1-C10-alkyl, (5) C1-C10-alkoxy, (6) —(C1-C10-alkylene)-(3 to 15 membered heterocycle), (7) C3-C10-cycloalkyl and (8) 3 to 15 membered heterocycle,
wherein each of (4)-(8) in R⁵¹ may be optionally substituted with 1 to 10 substituents selected from (1) halogen, (2) —OH, (3) cyano, (4) C1-C10-alkyl and (5) C1-C10-haloalkyl.

Furthermore preferable R⁵ and R^{5a} are each (1) —NH—C(═O)—(C3-C6-cycloalkyl), (2) —NH—C(═O)-(3 to 10 membered heterocycle), (3) —NH—C(═O)—O—(C1-C4-alkyl) which may be optionally substituted with 1 to 5 substituents selected from halogen, —OH and cyano, (4) —O—(C1-C4-alkyl) which may be optionally substituted with 1 to 5 substituents selected from halogen, —OH and cyano, (5) —O—(C1-C4-alkylene)-(3 to 10 membered heterocycle), (6) —C(═O)—(C3-C6-cycloalkyl), (7) —C(═O)-(3 to 10 membered heterocycle), (8) —C(═O)—NH—(C1-C4-alkyl) and (9) 5 to 10 membered heteroaryl;
wherein each of (1), (2) and (5)-(9) in R^{5a} may be optionally substituted with 1 to 5 R^{51a}.
multiple R^{51a} may be the same as or different from each other;
R^{51a} is independently selected from (1) halogen, (2) cyano, (3) —OH, (4) C1-C6-alkyl, (5) C1-C6-alkoxy, (6) —(C1-C6-alkylene)-(3 to 10 membered heterocycle), (7) C3-C6-cycloalkyl and (8) 3 to 10 membered heterocycle.
R⁵¹ and R^{51a} are each preferably (1) halogen, C1-C4-alkyl, C1-4 haloalkyl, C1-C4-alkoxy or C1-4 haloalkoxy.
R¹⁵, R¹⁶, R¹⁷, R¹⁸, R¹⁹ and R²⁰ are each preferably
C1-C4-alkyl which may be optionally substituted with 1 to 5 substituents selected from (1) halogen (2) cyano and (3) —OH,
C1-C4-alkoxy which may be optionally substituted with 1 to 5 substituents selected from (1) halogen (2) cyano and (3) —OH,
6 to 10 membered aryl which may be optionally substituted with 1 to 5 R²⁵,
5 to 10 membered heteroaryl which may be optionally substituted with 1 to 5 R²⁵,
C3-C6-cycloalkyl which may be optionally substituted with 1 to 5 R²⁵, or
3 to 10 membered heterocycle which may be optionally substituted with 1 to 5 R²⁵.

More preferable R¹⁵ is
C1-C4-alkyl which may be optionally substituted with 1 to 5 substituents selected from (1) halogen (2) cyano and (3) —OH, C3-C6-cycloalkyl which may be optionally substituted with 1 to 5 R²⁵, or 3 to 10 membered heterocycle which may be optionally substituted with 1 to 5 R²⁵.

Most preferable R¹⁵ is
tert-butyl which may be optionally substituted with 1 to 3 substituents selected from (1) halogen (2) cyano and (3) —OH,
C3-C6-cycloalkyl which may be optionally substituted with 1 to 5 R²⁵, or
3 to 10 membered heterocycle which may be optionally substituted with 1 to 5 R²⁵.

More preferable R¹⁶ is
C1-C4-alkyl which may be optionally substituted with 1 to 5 substituents selected from (1) halogen (2) cyano and (3) —OH.

Most preferable R¹⁶ is
tert-butyl which may be optionally substituted with 1 to 5 substituents selected from (1) halogen (2) cyano and (3) —OH.

More preferable R¹⁷ is
C1-C4-alkoxy which may be optionally substituted with 1 to 5 substituents selected from (1) halogen (2) cyano and (3) —OH, or
3 to 10 membered heterocycle which may be optionally substituted with 1 to 5 R²⁵.

Most preferable R¹⁷ is
3 to 10 membered heterocycle which may be optionally substituted with 1 to 5 R²⁵.

More preferable R¹⁸ is
C1-C4-alkoxy which may be optionally substituted with 1 to 5 substituents selected from (1) halogen (2) cyano and (3) —OH, or
3 to 10 membered heterocycle which may be optionally substituted with 1 to 5 R²⁵.

Most preferable R¹⁸ is
3 to 10 membered heterocycle which may be optionally substituted with 1 to 5 R²⁵.

More preferable R¹⁹ is
6 to 10 membered aryl which may be optionally substituted with 1 to 5 R²⁵,
5 to 10 membered heteroaryl which may be optionally substituted with 1 to 5 R²⁵,
C3-C6-cycloalkyl which may be optionally substituted with 1 to 5 R²⁵, or
3 to 10 membered heterocycle which may be optionally substituted with 1 to 5 R²⁵.

Most preferable R¹⁹ is
C3-C6-cycloalkyl which may be optionally substituted with 1 to 5 R²⁵
5 to 10 membered heteroaryl which may be optionally substituted with 1 to 5 R²⁵, or
3 to 10 membered heterocycle which may be optionally substituted with 1 to 5 R²⁵.

More preferable R²⁰ is
C1-C4-alkyl which may be optionally substituted with 1 to 5 substituents selected from (1) halogen (2) cyano and (3) —OH, or
C1-C4-alkoxy which may be optionally substituted with 1 to 5 substituents selected from (1) halogen (2) cyano and (3) —OH.

Most preferable R²⁰ is
C1-C4-alkyl which may be optionally substituted with 1 to 5 substituents selected from (1) halogen (2) cyano and (3) —OH.
R²¹ and R²² are each preferably hydrogen, C1-C4-alkyl or C1-C4-haloalkyl. More preferable R²¹ and R²² are each C1-C4-alkyl.
R²¹ and R²² are preferably attached with the carbon atom together and formed a C3-C10-cycloalkyl.
p is preferably 1, 2, 3 or 4. More preferable p is 1.
R²³ is preferably halogen, cyano,
C3-C10-cycloalkyl which may be optionally substituted with 1-10 substituents selected from (1)halogen, (2) C1-C4-alkyl and (3) C1-C4-haloalkyl, or
3 to 10 membered heterocycle which may be optionally substituted with 1-10 substituents selected from (1)halogen, (2) C1-C4-alkyl and (3) C1-C4-haloalkyl.

More preferable R²³ is halogen or cyano.
R²⁰¹, R²⁰², R²⁰³, R²⁰⁴, R²⁰⁵, R²⁰⁶, R²⁰⁷, R²⁰⁸, R²⁰⁹, R²¹⁰, R²¹¹ and R²¹² are each preferably C1-C4-alkyl, or C1-C4-haloalkyl.
R²⁴ and R²⁵ are each preferably —OH, halogen, cyano,
C1-C4-alkyl which may be optionally substituted with 1 to 3 substituents selected with (1) halogen, (2) —OH and (3) cyano,
C1-C4-alkoxy which may be optionally substituted with 1 to 3 substituents selected with (1) halogen, (2) —OH and (3) cyano,
C3-C10-cycloalkyl which may be optionally substituted with 1 to 3 substituents selected with (1) halogen, (2) —OH, (3) cyano, (4) C1-C4-alkyl and (5) C1-C4-haloalkyl,
3 to 10 membered heterocycle which may be optionally substituted with 1 to 3 substituents selected with (1) halogen, (2) —OH, (3) cyano, (4) C1-C4-alkyl and (5) C1-C4-haloalkyl, or
—(C1-C4-alkylene)-(3 to 10 membered heterocycle) which may be optionally substituted with 1 to 3 substituents selected with (1) halogen, (2) —OH, (3) cyano, (4) C1-C4-alkyl and (5) C1-C4-haloalkyl.

More preferable R²⁴ is
C1-C4-alkyl which may be optionally substituted with 1 to 3 substituents selected with (1) halogen, (2) —OH and (3) cyano,
3 to 10 membered heterocycle which may be optionally substituted with 1 to 3 substituents selected with (1) halogen, (2) —OH, (3) cyano, (4) C1-C4-alkyl and (5) C1-C4-haloalkyl or
—(C1-C4-alkylene)-(3 to 10 membered heterocycle) which may be optionally substituted with 1 to 3 substituents selected with (1) halogen, (2) —OH, (3) cyano, (4) C1-C4-alkyl and (5) C1-C4-haloalkyl.

Most preferable R²⁴ is 3 to 10 membered heterocycle which may be optionally substituted with 1 to 3 substituents selected with (1) halogen, (2) —OH, (3) cyano, (4) C1-C4-alkyl and (5) C1-C4-haloalkyl.

More preferable R²⁵ is halogen, —OH,
C1-C4-alkyl which may be optionally substituted with 1 to 3 substituents selected with (1) halogen, (2) —OH and (3) cyano, or
C1-C4-alkoxy which may be optionally substituted with 1 to 3 substituents selected with (1) halogen, (2) —OH and (3) cyano.

Most preferable R²⁵ is halogen, —OH,
methyl which may be optionally substituted with 1 to 3 substituents selected with (1) halogen, (2) —OH and (3) cyano, or
methoxy which may be optionally substituted with 1 to 3 substituents selected with (1) halogen, (2) —OH and (3) cyano.
R²⁶ and R²⁷ are each preferably hydrogen or C1-C4-alkyl.

More preferable R²⁶ is hydrogen.

More preferable R²⁷ is hydrogen.

Ring B is preferably 6 to 10 membered aryl or 5 to 10 membered heteroaryl. More preferable Ring B is 5 to 10 membered heteroaryl. More preferable Ring B is also 5 membered heteroaryl (for example, thienyl, pyrazolyl, isoxazolyl or oxadiazolyl (for example, 1,2,4-oxadiazole), more preferable Ring B is pyrazolyl, isoxazolyl or oxadiazolyl (for example, 1,2,4-oxadiazole).

Furthermore preferable Ring B is

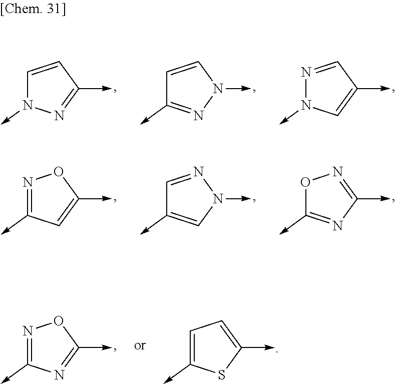

Most preferable Ring B is

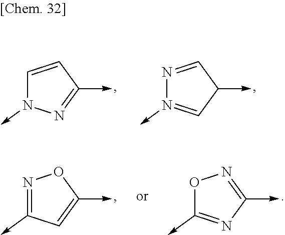

In some embodiments, the formula (I) is also preferably the formula (Ia).

In some embodiments, the formula (I) is also preferably the formula (Ia-1).

In some embodiments, the formula (I) is also preferably the formula (Ia-1-1).

In some embodiments, the formula (I) is also preferably the formula (Ia-1-2).

In some embodiments, the formula (I) is also preferably the formula (Ia-1-2a).

In some embodiments, the formula (I) is also preferably the formula (Ib).

In some embodiments, the formula (I) is also preferably the formula (Ib-1).

In some embodiments, the formula (I) is also preferably the formula (Ic).

In some embodiments, the formula (I) is also preferably the formula (Ic-1).

In some embodiments, the compound is preferably, but is not limited to:
(1) tert-butyl N-[4-chloro-3-[[3-fluoro-5-(2-phenylethynyl)-2-pyridyl]carbamoyl]phenyl]carbamate,
(2) 2-chloro-N-[3-fluoro-5-(2-phenylethynyl)-2-pyridyl]-5-[[(1S,2S)-2-methylcyclopropanecarbonyl]amino]benzamide,
(3) 2-chloro-N-[3-fluoro-5-(2-phenylethynyl)-2-pyridyl]-5-[(2-methylcyclopropanecarbonyl)amino]benzamide,
(4) 2-chloro-N-[3-fluoro-5-[2-(3-fluorophenyl)ethynyl]-2-pyridyl]-5-[[(1S,2S)-2-methylcyclopropanecarbonyl]amino]benzamide,
(5) tert-butyl N-[4-chloro-3-[[5-[2-(3-fluorophenyl)ethynyl]-3-methyl-2-pyridyl]carbamoyl]phenyl]carbamate,
(6) 2-chloro-5-[(1-fluorocyclopropanecarbonyl)amino]-N-[3-fluoro-5-(2-phenylethynyl)-2-pyridyl]benzamide,
(7) 2-chloro-5-(cyclopropanecarbonylamino)-N-[3-fluoro-5-[2-(4-fluorophenyl)ethynyl]-2-pyridyl]benzamide,
(8) tert-butyl N-[4-chloro-3-[[5-[2-(4-fluorophenyl)ethynyl]-3-methyl-2-pyridyl]carbamoyl]phenyl]carbamate,
(9) 2-chloro-5-(cyclopropanecarbonylamino)-N-[3-methyl-5-(2-phenylethynyl)-2-pyridyl]benzamide,
(10) N-[4-chloro-3-[[3-fluoro-5-(2-phenylethynyl)-2-pyridyl]carbamoyl]phenyl]-1,4-dioxane-2-carboxamide,
(11) tert-butyl N-[4-chloro-3-[[6-[2-(3-fluorophenyl)ethynyl]-2,4-dimethyl-3-pyridyl]carbamoyl]phenyl]carbamate,
(12) N-[4-chloro-3-[[5-[2-(4-fluorophenyl)ethynyl]-3-methyl-2-pyridyl]carbamoyl]phenyl]oxetane-2-carboxamide,
(13) N-[4-chloro-3-[[5-[2-(3-fluorophenyl)ethynyl]-3-methyl-2-pyridyl]carbamoyl]phenyl]-1,4-dioxane-2-carboxamide,
(14) N-[4-chloro-3-[[5-[2-(4-fluorophenyl)ethynyl]-3-methyl-2-pyridyl]carbamoyl]phenyl]-1,4-dioxane-2-carboxamide,
(15) tert-butyl N-[4-chloro-3-[[3-methyl-5-[2-(2-pyridyl)ethynyl]-2-pyridyl]carbamoyl]phenyl]carbamate,
(16) 2-chloro-5-(3,3-difluoroazetidine-1-carbonyl)-N-[3-methyl-5-(2-phenylethynyl)-2-pyridyl]benzamide,
(17) 4-chloro-N1-(2-hydroxy-1,1-dimethyl-ethyl)-N3-[3-methyl-5-(2-phenylethynyl)-2-pyridyl]benzene-1,3-dicarboxamide,
(18) 2-chloro-N-[5-[2-(3-fluorophenyl)ethynyl]-3-methyl-2-pyridyl]-5-(3-hydroxy-3-methy 1-azetidine-1-carbonyl)benzamide,
(19) 2-chloro-N-[3-methyl-5-(2-phenylethynyl)-2-pyridyl]-5-(5-tetrahydrofuran-3-yl-1,2,4-oxadiazol-3-yl)benzamide,
(20) 2-chloro-N-[3-methyl-5-(2-phenylethynyl)-2-pyridyl]-5-(tetrahydropyran-4-ylmethoxy) benzamide,
(21) 2-chloro-5-[[(2R)-1,4-dioxan-2-yl]methoxy]-N-[3-methyl-5-(2-phenylethynyl)-2-pyrid yl]benzamide,
(22) 2-chloro-5-(1,4-dioxan-2-ylmethoxy)-N-[3-methyl-5-(2-phenylethynyl)-2-pyridyl]benzamide,
(23) 2-chloro-5-[[(2S)-1,4-dioxan-2-yl]methoxy]-N-[3-methyl-5-(2-phenylethynyl)-2-pyrid yl]benzamide,
(24) 2-chloro-5-(2-methoxyethoxy)-N-[3-methyl-5-(2-phenylethynyl)-2-pyridyl]benzamide,
(25) tert-butyl (3-((4-(benzyloxy)-2-methylphenyl)carbamoyl)-4-chlorophenyl)carbamate,
(26) tert-butyl (4-chloro-3-((2-methyl-4-(phenoxymethyl)phenyl)carbamoyl)phenyl)carbamate,

or a pharmaceutically acceptable salt thereof.

In some embodiments, more preferable compound is
tert-butyl N-[4-chloro-3-[[3-fluoro-5-(2-phenylethynyl)-2-pyridyl]carbamoyl]phenyl]carbamate, or a pharmaceutically acceptable salt thereof.

In some embodiments, more preferable compound is
2-chloro-N-[3-fluoro-5-(2-phenylethynyl)-2-pyridyl]-5-[[(1S,2S)-2-methylcyclopropanecarbonyl]amino]benzamide,

or a pharmaceutically acceptable salt thereof.

In some embodiments, more preferable compound is
2-chloro-N-[3-fluoro-5-(2-phenylethynyl)-2-pyridyl]-5-[(2-methylcyclopropanecarbonyl)amino]benzamide,

or a pharmaceutically acceptable salt thereof.

In some embodiments, more preferable compound is
2-chloro-N-[3-fluoro-5-[2-(3-fluorophenyl)ethynyl]-2-pyridyl]-5-[[(1S,2S)-2-methyl cyclopropanecarbonyl]amino]benzamide,

or a pharmaceutically acceptable salt thereof.

In some embodiments, more preferable compound is
tert-butyl N-[4-chloro-3-[[5-[2-(3-fluorophenyl)ethynyl]-3-methyl-2-pyridyl]carbamoyl]phenyl]carbamate,

or a pharmaceutically acceptable salt thereof.

In some embodiments, more preferable compound is
2-chloro-5-[(1-fluorocyclopropanecarbonyl)amino]-N-[3-fluoro-5-(2-phenylethynyl)2-pyridyl]benzamide,

or a pharmaceutically acceptable salt thereof.

In some embodiments, more preferable compound is
2-chloro-5-(cyclopropanecarbonylamino)-N-[3-fluoro-5-[2-(4-fluorophenyl)ethynyl]-2-pyridyl]benzamide,

or a pharmaceutically acceptable salt thereof.

In some embodiments, more preferable compound is
tert-butyl N-[4-chloro-3-[[5-[2-(4-fluorophenyl)ethynyl]-3-methyl-2-pyridyl]carbamoyl]phenyl]carbamate,

or a pharmaceutically acceptable salt thereof.

In some embodiments, more preferable compound is
2-chloro-5-(cyclopropanecarbonylamino)-N-[3-methyl-5-(2-phenylethynyl)-2-pyridyl]benzamide,

or a pharmaceutically acceptable salt thereof.

In some embodiments, more preferable compound is
N-[4-chloro-3-[[3-fluoro-5-(2-phenylethynyl)-2-pyridyl]carbamoyl]phenyl]-1,4-dioxane-2-carboxamide,

or a pharmaceutically acceptable salt thereof.

In some embodiments, more preferable compound is
tert-butyl N-[4-chloro-3-[[6-[2-(3-fluorophenyl)ethynyl]-2,4-dimethyl-3-pyridyl]carbamoyl]phenyl]carbamate,

or a pharmaceutically acceptable salt thereof.

In some embodiments, more preferable compound is N-[4-chloro-3-[[5-[2-(4-fluorophenyl)ethynyl]-3-methyl-2-pyridyl]carbamoyl]phenyl]oxetane-2-carboxamide,

or a pharmaceutically acceptable salt thereof.

In some embodiments, more preferable compound is
N-[4-chloro-3-[[5-[2-(3-fluorophenyl)ethynyl]-3-methyl-2-pyridyl]carbamoyl]phenyl]-1,4-dioxane-2-carboxamide,

or a pharmaceutically acceptable salt thereof.

In some embodiments, more preferable compound is
N-[4-chloro-3-[[5-[2-(4-fluorophenyl)ethynyl]-3-methyl-2-pyridyl]carbamoyl]phenyl]-1,4-dioxane-2-carboxamide,

or a pharmaceutically acceptable salt thereof.

In some embodiments, more preferable compound is
tert-butyl N-[4-chloro-3-[[3-methyl-5-[2-(2-pyridyl)ethynyl]-2-pyridyl]carbamoyl]phenyl]carbamate, or a pharmaceutically acceptable salt thereof.

In some embodiments, more preferable compound is
2-chloro-5-(3,3-difluoroazetidine-1-carbonyl)-N-[3-methyl-5-(2-phenylethynyl)-2-pyridyl]benzamide,

or a pharmaceutically acceptable salt thereof.

In some embodiments, more preferable compound is
4-chloro-N1-(2-hydroxy-1,1-dimethyl-ethyl)-N3-[3-methyl-5-(2-phenylethynyl)-2-pyridyl]benzene-1,3-dicarboxamide,

or a pharmaceutically acceptable salt thereof.

In some embodiments, more preferable compound is
2-chloro-N-[5-[2-(3-fluorophenyl)ethynyl]-3-methyl-2-pyridyl]-5-(3-hydroxy-3-methyl-azetidine-1-carbonyl)benzamide,

or a pharmaceutically acceptable salt thereof.

In some embodiments, more preferable compound is
2-chloro-N-[3-methyl-5-(2-phenylethynyl)-2-pyridyl]-5-(5-tetrahydrofuran-3-yl-1,2,4-oxadiazol-3-yl)benzamide,

or a pharmaceutically acceptable salt thereof.

In some embodiments, more preferable compound is
2-chloro-N-[3-methyl-5-(2-phenylethynyl)-2-pyridyl]-5-(tetrahydropyran-4-ylmethoxy)benzamide,

or a pharmaceutically acceptable salt thereof.

In some embodiments, more preferable compound is
2-chloro-5-[[(2R)-1,4-dioxan-2-yl]methoxy]-N-[3-methyl-5-(2-phenylethynyl)-2-pyridyl]benzamide,

or a pharmaceutically acceptable salt thereof.

In some embodiments, more preferable compound is
2-chloro-5-(1,4-dioxan-2-ylmethoxy)-N-[3-methyl-5-(2-phenylethynyl)-2-pyridyl]benzamide,

or a pharmaceutically acceptable salt thereof.

In some embodiments, more preferable compound is
2-chloro-5-[[(2S)-1,4-dioxan-2-yl]methoxy]-N-[3-methyl-5-(2-phenylethynyl)-2-pyridyl]benzamide,

or a pharmaceutically acceptable salt thereof.

In some embodiments, more preferable compound is
2-chloro-5-(2-methoxyethoxy)-N-[3-methyl-5-(2-phenylethynyl)-2-pyridyl]benzamide,

or a pharmaceutically acceptable salt thereof.

In some embodiments, more preferable compound is
tert-butyl (3-((4-(benzyloxy)-2-methylphenyl)carbamoyl)-4-chlorophenyl)carbamate,

or a pharmaceutically acceptable salt thereof.

In some embodiments, more preferable compound is
tert-butyl (4-chloro-3-((2-methyl-4-(phenoxymethyl)phenyl)carbamoyl)phenyl)carbamate, or a pharmaceutically acceptable salt thereof.

The compound of the formula (I) is preferably such a compound that some or all of the above-mentioned preferred examples for R, L, W, Z, X, Y, R¹, R², R³, R⁴, R⁵ and R⁶ are combined.

The compound of the formula (Ia) is preferably such a compound that some or all of the above-mentioned preferred examples for R, W, Z, R¹, R², R³, R⁴ and R⁵ are combined.

The compound of the formula (Ia-1) is preferably such a compound that some or all

of the above-mentioned preferred examples for R, W, Z, R¹, R³, R⁴ and R⁵ are combined.

The compound of the formula (Ia-1-1) is preferably such a compound that some or all of the above-mentioned preferred examples for R, R^{1a}, R⁴, R^{5a} and R^{8a} are combined.

The compound of the formula (Ia-1-2) is preferably such a compound that some or all of the above-mentioned preferred examples for R, R^{1a}, R², R⁴ and R^{5a} are combined.

The compound of the formula (Ia-1-2a) is preferably such a compound that some or all of the above-mentioned preferred examples for R, R^{1a}, R⁴ and R^{5a} are combined.

The compound of the formula (Ib) is preferably such a compound that some or all of the above-mentioned preferred examples for R, W, Z, R¹, R², R³, R⁴ and R⁵ are combined.

The compound of the formula (Ib-1) is preferably such a compound that some or all of the above-mentioned preferred examples for R, W, Z, R¹, R³, R⁴ and R⁵ are combined.

The compound of the formula (Ic) is preferably such a compound that some or all of the above-mentioned preferred examples for R, Ring B, W, Z, R¹, R², R³, R⁴ and R⁵ are combined.

The compound of the formula (Ic-1) is preferably such a compound that some or all of the above-mentioned preferred examples for R, Ring B, W, Z, R¹, R³, R⁴ and R⁵ are combined.

The compound of the formula (II) is preferably such a compound that some or all of the above-mentioned preferred examples for R, L, W, Z, R¹, R³, R⁴ and R⁵ are combined.

A disorder associated with TREK1, TREK2 or dual TREK1/TREK2 dysfunction in which inhibitors of TREK1, TREK2 or both TREK1 and TREK2 would offer therapeutic benefit is preferably neurological and/or psychiatric disorder.

More preferable disorder associated with TREK1, TREK2 or dual TREK1/TREK2 dysfunction in which inhibitors of TREK1, TREK2 or both TREK1 and TREK2 would offer therapeutic benefit is depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain or cerebral infarction.

Furthermore preferable disorder associated with TREK1, TREK2 or dual

TREK1/TREK2 dysfunction in which inhibitors of TREK1, TREK2 or both TREK1 and TREK2 would offer therapeutic benefit is depression, schizophrenia, anxiety disorders, bipolar disorder. Most preferable disorder associated with TREK1, TREK2 or dual TREK1/TREK2 dysfunction in which inhibitors of TREK1, TREK2 or both TREK1 and TREK2 would offer therapeutic benefit is depression or schizophrenia.

Compound names are assigned by using the Struct=Name naming algorithm as part of CHEMDRAW(Registered Trademark) ULTRA v. 15.0.

In the present invention, unless otherwise specified,

the symbol:

[symbol]

[Chem.33]

represents that a substituent binds to the back side on the paper surface (in other words, α-configuration), the symbol:

[symbol]

[Chem.34]

represents that a substituent binds to the front side on the paper surface (in other words, β-configuration), the symbol:

[symbol]

[Chem.35]

represents mixture of α-configuration and β-configuration.

The compound may exist as a stereoisomer wherein asymmetric or chiral centers are

present. The stereoisomer is “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The disclosure contemplates various stereoisomers and mixtures thereof and these are specifically included within the scope of this invention. Stereoisomers include enantiomers and diastereomers, and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry,” 5th edition (1989), Longman Scientific & Technical, Essex CM20 2JE, England, or (2) direct separation of the mixture of optical enantiomers on chiral chromatographic columns, or (3) fractional recrystallization methods.

It should be understood that the compound may possess tautomeric forms, as well as geometric isomers, and that these also constitute embodiments of the disclosure.

The present disclosure also includes an isotopically-labeled compound, which is identical to those recited in formula (I), but for the fact that one or more atoms are replaced by an atom having an atomic mass or mass number different from the atomic mass or mass number usually found in nature. Examples of isotopes suitable for inclusion in the compounds of the invention are hydrogen, carbon, nitrogen, oxygen, phosphorus, sulfur, fluorine, and chlorine, such as, but not limited to ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes can afford certain therapeutic advantages resulting from greater metabolic stability, for example increased in vivo half-life or reduced dosage requirements and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated in compounds of formula (I) are ¹¹C, ¹³N, ¹⁵O, and ¹⁸F. Isotopically-labeled compounds of formula (I) can generally be prepared by conventional techniques known to those skilled in the art or by processes analogous to those described in the accompanying Examples using appropriate isotopically-labeled reagent in place of non-isotopically-labeled reagent.

Pharmaceutically Acceptable Salts

The disclosed compounds may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

In the present invention, all the mentions of the compound of the present invention include a compound represented by formula (I), or a salt, a solvate, or a cocrystal thereof.

The compound represented by formula (I) and a salt thereof may be present in a not-solvation form, or in a solvation form with pharmaceutically acceptable solvent such as water or ethanol. Preferable solvates include hydrate. The compound represented by formula (I) and a salt thereof can be converted into a solvate by a well-known method.

The compound represented by formula (I) can form a cocrystal with an appropriate cocrystal former. As the cocrystal, pharmaceutically acceptable cocrystal that is formed with a pharmaceutically acceptable cocrystal former is preferable. The cocrystal is typically defined as a crystal that is formed of two or more different molecules by intermolecular interaction that is different from ionic bond. Furthermore, the cocrystal may be a composite of a neutral molecule and a salt. The cocrystal can be prepared by recrystallization from a solvent by a well-known method, for example, melting crystallization, or physically pulverizing the components together. Appropriate cocrystal formers include ones described in WO2006/007448.

The compound represented by the formula (I) can be administered as a prodrug. The prodrug of the compound represented by the formula (I) refers to a compound which is converted in vivo to the compound represented by the formula (I) by the reaction with enzymes, gastric acid and the like. Examples of the prodrug of the compound represented by the formula (I) include, when the compound represented by the formula (I) has an amino group, compounds in which the amino group is acylated, alkylated or phosphorylated (e.g. compounds represented by the formula (I) in which the amino group thereof is converted to eicosanoyl, aranyl, pentylaminocarbonyl, (5-methyl-2-oxo-1,3-dioxolen-4-yl)methoxycarbonyl, tetrahydrofuranyl, pyrrolidylmethyl, pivaloyloxymethyl, acetoxymethyl, tert-butyl or the like); when the compound represented by the formula (I) has a hydroxy group, compounds in which the hydroxy group is acylated, alkylated, phosphorylated or converted to borate (e.g. compounds represented by the formula (I) in which the hydroxy group thereof is converted to acetyl, palmitoyl, propanoyl, pivaloyl, succinyl, fumaryl, alanyl, dimethylaminomethylcarbonyl or the like); when the compound represented by the formula (I) has a carboxy group, compounds in which the carboxy group is esterified or amidated (e.g. compounds represented by the formula (I) in which the carboxy group thereof is converted to methyl ester, ethyl ester, isopropyl ester, phenyl ester, carboxymethyl ester, dimethylaminomethyl ester, pivaloyloxymethyl ester, phthalidyl ester, 1-{(ethoxycarbonyl)oxy}ethyl ester, (5-methyl-2-oxo-1,3-dioxolen-4-yl)methyl ester, 1-{[(cyclohexyloxy)carbonyl]oxy}ethyl ester, methylamide or the like) and the like. The prodrug of the compound represented by the formula (I) may be the one which is converted to the compound represented by the formula (I) under the physiological condition such as those disclosed in “lyakuhin no Kaihatsu”, vol. 7 “Bunshi Sekkei”, p. 163-198, 1990, Hirokawa Shoten Co.

General Synthesis

Compounds of formula (I) may be prepared by synthetic processes or by metabolic processes. Preparation of the compounds by metabolic processes includes those occurring in the human or animal body (in vivo) or processes occurring in vitro. Abbreviations which have been used in the descriptions of the Schemes that follow

are: IPA is isopropyl alcohol; AcOH is acetic acid; TFA is trifuloroacetic acid; DCE is 1,2-Dichloroethane; DCM is dichloromethane; DIEA is N,N-diisopropylethylamine; DMF is N,N-dimethylformamide; HATU for 1-[bis(dimethylamino)methylene]-1H-1,2,3-triazolo[4,5-b]pyridinium 3-oxid hexafluorophosphate; MW is microwave (referring to a microwave reactor); PyClU is 1-(chloro-1-pyrrolidinylmethylene)pyrrolidinium hexafluorophosphate; rt is room temperature and Xantphos is 4,5-Bis(diphenylphosphino)-9,9-dimethylxanthene; Boc is tert-butyloxycarbonyl; dppf is 1,1′-bis(diphenylphosphino)ferrocene; dba is dibenzylideneacetone.

Compounds of formula (I), specifically compounds of formula (Ia), formula (Ib) and formula (Ic) can be synthesized as shown in Scheme I to MV.

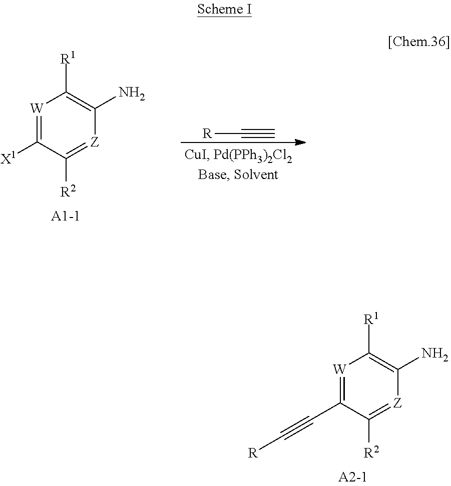

wherein X¹ is selected from (1)halogen, (2)tosylate, (3)mesylate and (4)triflate and the other symbols are defined as below.

As shown in Scheme I, compounds A1-1 may be coupled with a variety of alkynes under Sonogashira coupling conditions, generally known in the art, to provide intermediates A2-1. For example, the reaction may be conducted with a palladium catalyst such as Pd(PPh₃)₂Cl₂ in the presence of a base (e.g, DIEA) and a copper source (e.g., Copper (I) Iodide) in a solvent such as dimethylformamide with heating up to around 60° C.

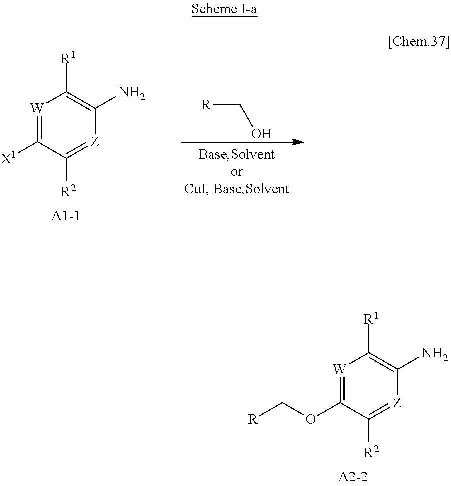

wherein all symbols are defined as below.

As shown in Scheme I-a, compounds A1-1 may be coupled with a variety of alcohols under transition metal-catalyzed coupling conditions or basic conditions, generally known in the art, to provide intermediates A2-2. For example, the reaction may be conducted in the presence of a transition metal catalyst (e.g, CuI) and a base (e.g, Cs₂CO₃) in a solvent such as toluene with heating up to around 80° C. Or the reaction may be conducted in the presence of a base (e.g, NaH) with heating up to around 120° C.

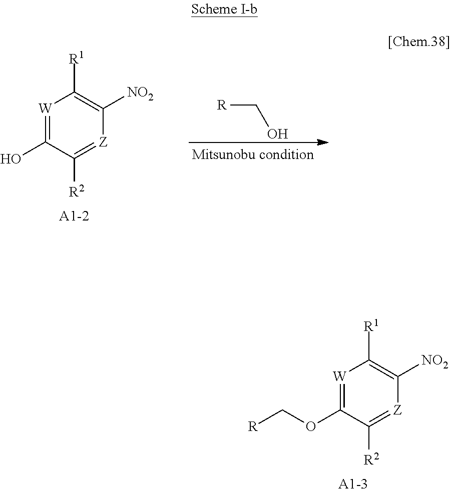

wherein all symbols are defined as below.

As shown in Scheme I-b, compounds A1-2 may be coupled with a variety of alcohols under Mitsunobu conditions, generally known in the art, to provide intermediates A1-3. For example, the reaction may be conducted with a diazo-derivatives such as diisopropyl azodicarboxylate in the presence of a phosphine (e.g, PPh₃) in a solvent such as THF.

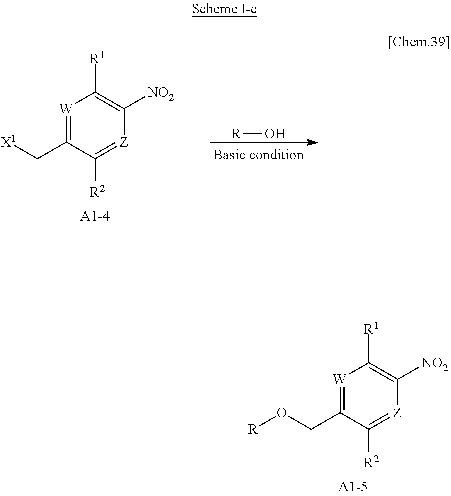

wherein all symbols are defined as below.

As shown in Scheme I-c, compounds A1-4 may be coupled with a variety of alcohols under basic conditions, generally known in the art, to provide intermediates A1-5. For example, the reaction may be conducted with an Ar—OH such as phenol in the presence of a base (e.g, Cs₂CO₃) in a solvent such as DMF. Ar means aryl as below.

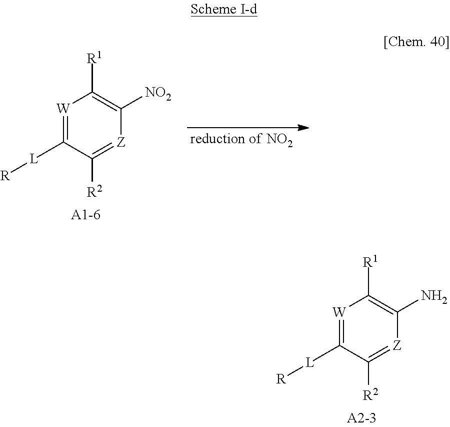

wherein all symbols are defined as below.

As shown in Scheme I-d, compounds A1-6 may be converted to aniline intermediates A2-3 under reductive conditions, generally known in the art. For example, the reaction may be conducted with hydrogen source such as an iron powder in the presence of an acid (e.g, ammonium chloride) in a solvent such as THF—H₂O with heating up to around 75° C. or with hydrogen source such as hydrogen in the presence of palladium catalyst (e.g, 5% palladium on carbon) in a solvent such as ethyl acetate.

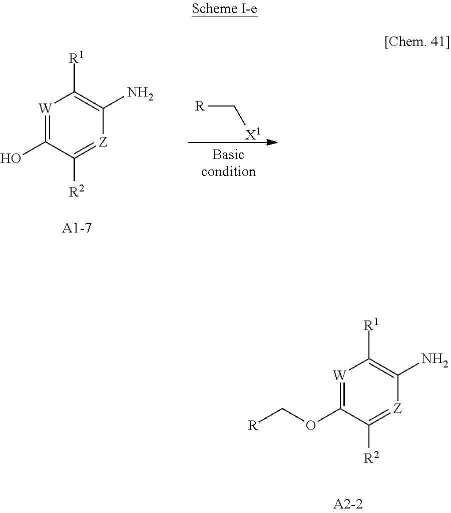

wherein all symbols are defined as below.

As shown in Scheme I-e, compounds A1-7 may be coupled with a variety of alkyl halides under basic conditions, generally known in the art, to provide intermediates A2-2. For example, the reaction may be conducted with an alkyl halide such as benzyl bromide in the presence of a base (e.g, Cs₂CO₃) in a solvent such as DMF.

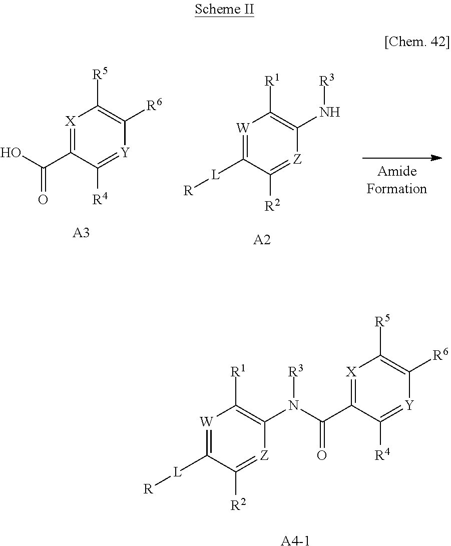

wherein all symbols are defined as below.

Scheme II illustrates a general route to compounds of formula (A4-1). Compounds A4-1 may be formed by standard amide coupling conditions (e.g. PyClU, pyridine, DCE) heating to around 60° C.

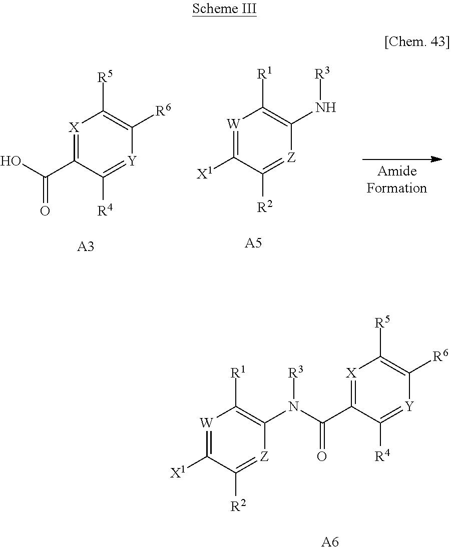

wherein all symbols are defined as below.

Scheme III illustrates a general route to compounds of formula (A6). Compounds A6 may be formed by standard amide coupling conditions (e.g. PyClU, pyridine, DCE) heating to around 60° C.

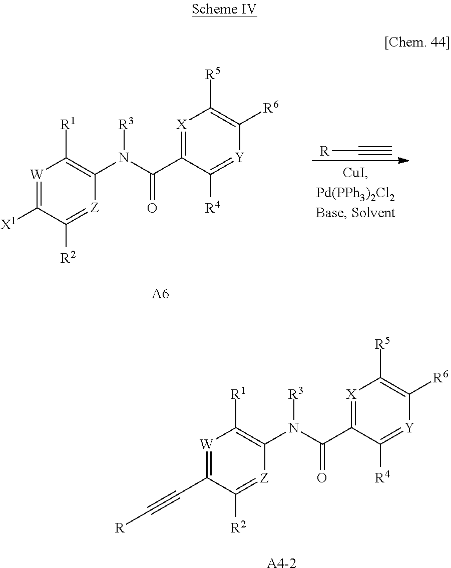

wherein all symbols are defined as below.

As shown in Scheme IV, compounds A6 may be coupled with a variety of alkynes under Sonogashira coupling conditions, generally known in the art, to provide A4-2. For example the reaction may be conducted with a palladium catalyst such as Pd(PPh₃) 2012 in the presence of a base (e.g, DIEA) and a copper source (e.g., Copper (I) Iodide) in a solvent such as dimethylformamide with heating up to around 60° C.

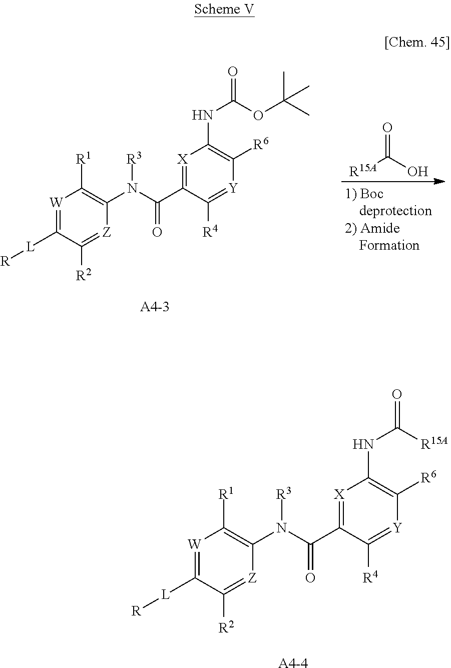

wherein R^{15A} is selected from 5 to 15 membered heteroaryl or 3 to 15 membered heterocycle; and the other symbols are defined as below.

As shown in Scheme V, A4-3 under acidic conditions (e.g. TFA/DCM) may provide the aniline that subsequently may be coupled under standard amide coupling conditions (e.g. HATU, DIEA, carboxylic acid or R¹⁵COCl, DIEA, DCM) to form amides A4-4.

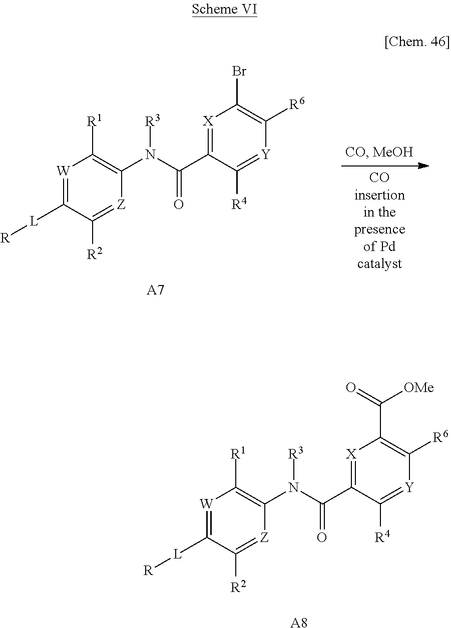

wherein all symbols are defined as below.

Scheme VI illustrates a general route to compounds of formula (A8). Compounds A8 may be formed by standard CO insertion conditions in the presence of Pd catalyst (e.g. CO(g), K₂CO₃, Pd(OAc)₂, MeOH, DMF) heating to around 60° C.

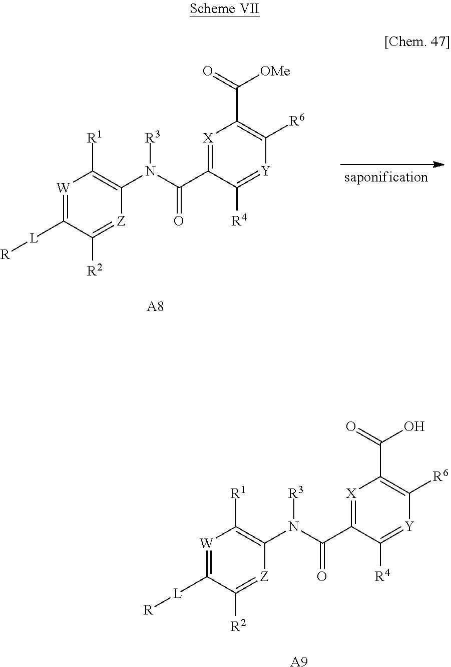

wherein all symbols are defined as below.

Scheme VII illustrates a general route to compounds of formula (A9). Compounds A9 may be formed by standard saponification conditions (e.g. NaOH (s), H₂O, MeOH, rt).

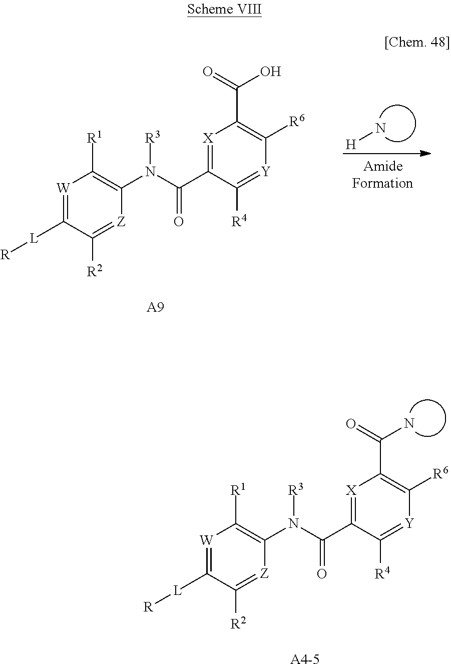

wherein

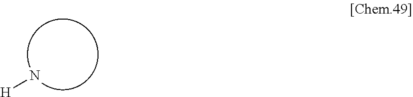

is selected from 5 to 15 membered heteroaryl or 3 to 15 membered heterocycle with nitrogen amenable to amide formation; and the other symbols are defined as below.

As shown in Scheme VIII, the intermediate A9 may be coupled under standard amide coupling conditions (e.g. HATU, DIEA, amine

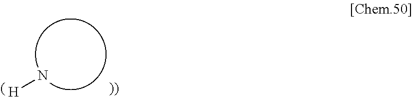

to form amides A4-5.

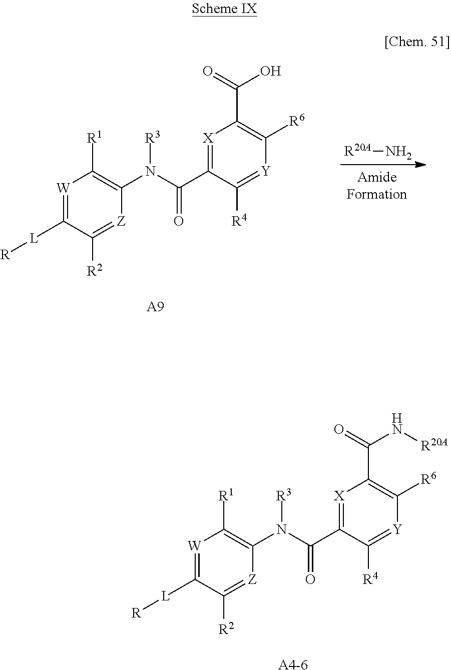

wherein R^{20A} is selected from C1-C10-alkyl which may be optionally substituted with substituents selected from (1) halogen (2) cyano and (3) —OH; and the other symbols are defined as below.

As shown in Scheme IX, the intermediate A9 may be coupled under standard amide coupling conditions (e.g. HATU, DIEA, amine(R^{20A}—NH₂)) to form amides A4-6.

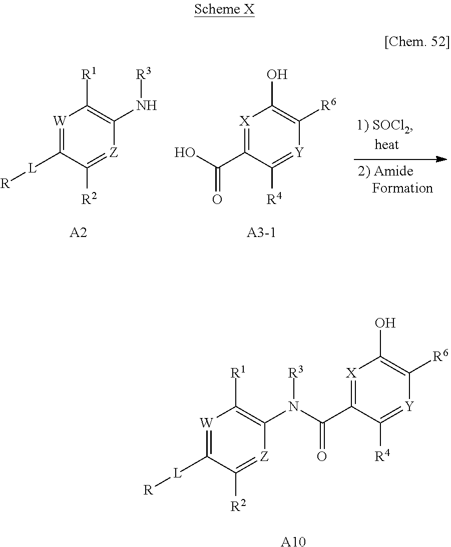

wherein all symbols are defined as below.

As shown in Scheme X, intermediate A3-1 may form an acid chloride (e.g. SOCl₂) and subsequently undergo an amide formation in the presence of base (e.g. DIEA), solvent (e.g. DCM) and amine A2 to provide amide A10.

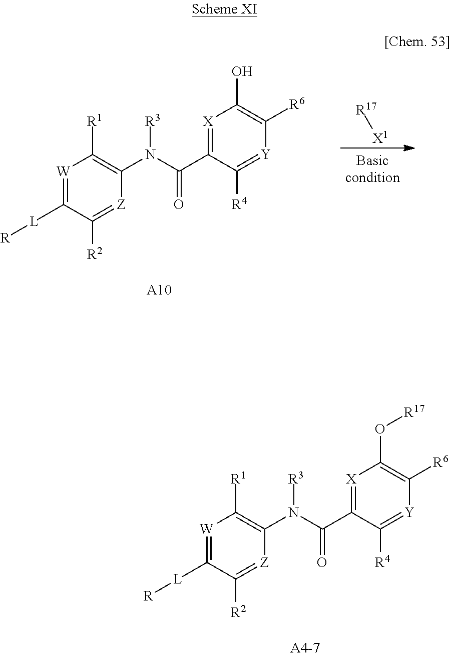

wherein all symbols are defined as below.

As shown in Scheme XI, compounds A10 may be coupled with a variety of alkyl halides under basic conditions to provide ethers A4-7. For example, the reaction may be conducted with an alkyl halide such as 2-(bromomethyl)dioxane in the presence of a base (e.g, Cs₂CO₃) in a solvent such as DMF.

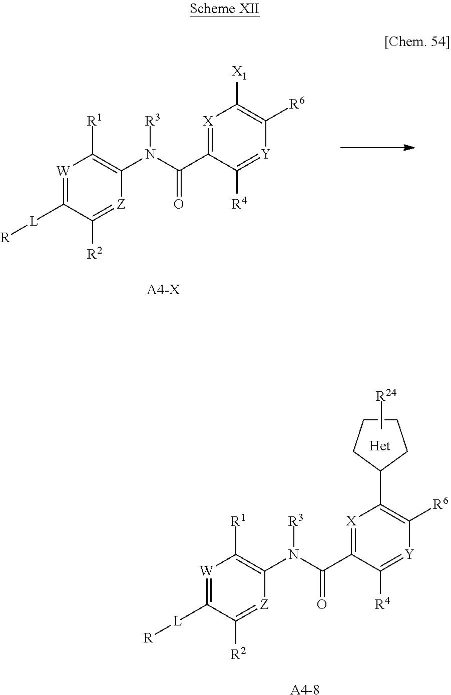

wherein

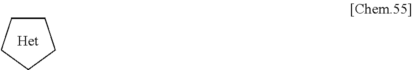

is 5 membered heteroaryl, and the other symbols are defined as below.

As shown in Scheme XII, compound A4-8 may be prepared by Suzuki cross-coupling conditions, generally known in the art. For example the reaction may be conducted with a boron reagent, including boronic acid like

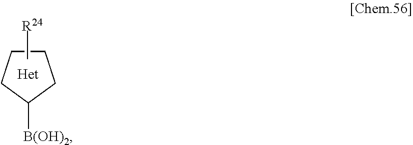

boronate ester like

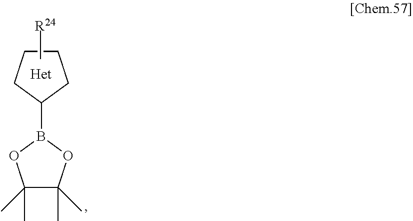

as well as a palladium catalyst (e.g. Pd(dppf)Cl₂) in the presence of a base (e.g. K₂CO₃) in a suitable solvent (e.g. 1,4-dioxane and water) with heating up to 85° C.

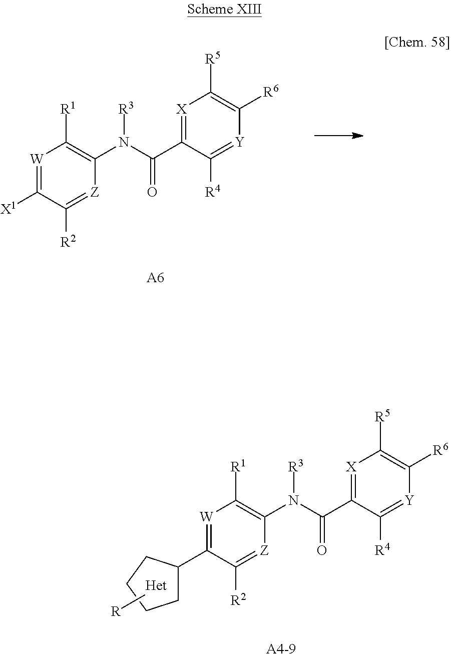

wherein

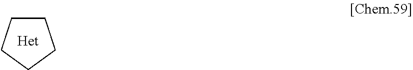

is 5 membered heteroaryl, and the other symbols are defined as below.

As shown in Scheme XIII, compound A4-9 may be prepared by Suzuki cross-coupling conditions, generally known in the art. For example the reaction may be conducted with a boron reagent, including boronic acid like

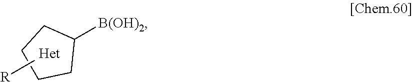

boronate ester like

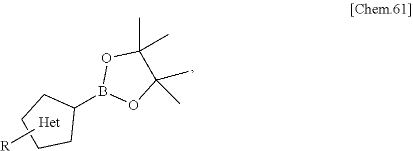

as well as a palladium catalyst (e.g. Pd(dppf)Cl₂) in the presence of a base (e.g. K₂CO₃) in a suitable solvent (e.g. 1,4-dioxane and water) with heating up to 85° C.

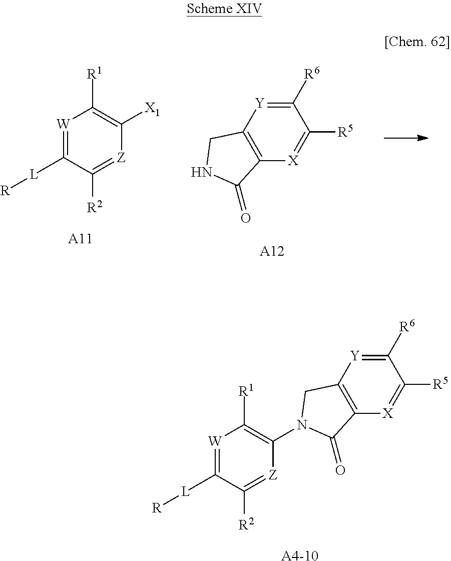

wherein all symbols are defined as below.

Scheme XIV illustrates the cross coupling between intermediates All and amides A12. For example, the reaction may be conducted with intermediates All and A12, a palladium catalyst (e.g. Pd₂(dba)₃), a suitable ligand (e.g. Xantphos), base (e.g. Cs₂CO₃) and solvent (e.g. DMF) with heating up to 100° C.

The compounds used as the starting material in each of the reactions is known or can be easily prepared by known method.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in P G M Wuts and T W Greene, in Greene's book titled Protective Groups in Organic Synthesis (4th ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

TREK1 and TREK2 Inhibitor Activity

Method 1 (Thallium flux assay); CHO-K1 cells stably expressing human TREK-1 or HEK293 cells stably expressing human TREK-2 are plated in 384-well plates, cultured overnight, loaded with Thallos dye the following day, treated with test compounds or control compound (tert-butyl (3-((4-(benzyloxy)-2-methylphenyl)carbamoyl)-4-chlorophenyl)carbamate) or 0.3% DMSO (vehicle control) for 10 min, and then treated with thallium stimulus buffer to initiate thallium flux. To measure the efficacy and potency of test compounds, the change in fluorescence intensity (ΔRatio) and % inhibition were calculated using the following equations:

ΔRatio=(fluorescence intensity at 25 seconds after thallium addition)/(average of fluorescent intensity before thallium addition)

% inhibition={1−(ΔRatio of test compound−ΔRatio of 10 μM control compound)/(ΔRatio of 0.3% DMSO−ΔRatio of 10 μM control compound)}×100

Method 2 (Patch Clamp Technique):

CHO-K1 cells stably expressing human TREK-1 or HEK293 cells stably expressing human TREK-2 are plated on glass coverslips, and voltage clamped in the whole-cell configuration of the patch clamp technique. Cells were voltage clamped at a holding potential of −80 mV and the stepped to 0 mV for 500 msec. The voltage was subsequently ramped from −120 mV to +80 mV over a 500 msec duration. This step-ramp protocol was repeated every 10 sec. The bathing solution contained the following: 135 mM NaCl, 5 mM KCl, 2 mM CaCl₂, 1 mM MgCl₂, 5 mM D-Glucose, 10 mM HEPES, 10 mM sucrose (adjusted to pH 7.4 with NaOH, 300 mosmol/kg H₂O). The pipette solution contained the following: 135 mM KCl, 2 mM MgCl₂, 1 mM EGTA, 10 mM HEPES, 2 mM Na 2 ATP (adjusted to pH 7.35 with KOH, 285 mosmol/kg H₂O). Test compounds were dissolved into the bathing solution. Experiments on TREK-2 were terminated with the addition of the control compound (tert-butyl (3-((4-(benzyloxy)-2-methylphenyl)carbamoyl)-4-chlorophenyl)carbamate) so that maximal inhibition could be determined. The effects of test compound on the currents were calculated at 0 mV using the following equations:

For CHO cells expressing TREK-1 channel: % inhibition=(1-post current/pre current)×100

For HEK293 cells expressing TREK-2 channel: % inhibition={1−(post current−current in the presence of 10 μM control compound)/(pre current−current in the presence of 10 μM control compound)}×100

In some experiments, the disclosed compounds inhibit TREK1 channel response as a decrease in thallium fluorescence in thallium flux assay or a decrease in current measured at 0 mV in patch clamp electrophysiology assays.

The disclosed compounds may inhibit TREK-1 and/or TREK-2 via an inhibit mechanism or through an allosteric modulation mechanism.

Method 3 (MK-801-Induced Novel Object Recognition Test):

Testing was performed using 6-week-old male Sprague Dawley rats. On the testing day, rats were habituated at the empty box for 10 minutes individually. The disclosed compound was administered orally 1 h prior, followed by administration of MK-801 (0.2 mg/kg, i.p. or s.c.) 30 min prior to the acquisition trial for 10 min. The retention trial for 10 min was performed with the intertrial interval for 80 min. The rat was allowed to explore the same objects in the acquisition trial. In the retention trial, one of the objects used in the acquisition trial was replaced with a novel one. The exploration time of licking, sniffing or touching was measured in the retention trial. Efficacy of each compound at the dose of 0.3, 1, 3 or 10 mg/kg p.o. (n=12-15) was determined by the recognition index (exploration time to the novel object/total exploration time). The scores of MK-801/vehicle and MK-801/compound-treated groups were statistically analyzed by Dunnett's test.

The disclosed compounds may inhibit TREK1 selectively. The disclosed compounds may inhibit TREK2 selectively. The disclosed compounds may inhibit both TREK1 and TREK2 to varying degrees. The disclosed compounds may inhibit TREK1 and/or TREK2 via a competitive antagonist mechanism or through an allosteric, noncompetitive mechanism.

The disclosed compounds may inhibit TREK1 and/or TREK2 response in TREK1 or TREK2-transfected CHO-K1 cells with an IC 50 less than, or equivalent to the IC 50 for TREK1 or TREK2. That is, a disclosed compound can have selectivity for the TREK1 vis-a-vis TREK2, a disclosed compound can have selectivity for the TREK2 vis-a-vis TREK1, or no selectivity. For example, in some embodiments, a disclosed compound can inhibit TREK1 response with an IC 50 of about 5-fold less, about 10-fold less, about 20-fold less, about 30-fold less, about 50-fold less, about 100-fold less, about 200-fold less, about 300-fold less, about 400-fold less, or greater than about 500-fold less than that for TREK2. In some embodiments, a disclosed compound can inhibit TREK2 response with an IC 50 of about 5-fold less, about 10-fold less, about 20-fold less, about 30-fold less, about 50-fold less, about 100-fold less, about 200-fold less, about 300-fold less, about 400-fold less, or greater than about 500-fold less than that for TREK1. In some embodiments, a disclosed compound can inhibit TREK1 and TREK2 responses with comparable IC 50 values.

Pharmaceutical Compositions and Formulations

The disclosed compounds may be incorporated into pharmaceutical compositions suitable for administration to a subject (such as a patient, which may be a human or non-human). The disclosed compounds may also be provided as formulations, such as spray-dried dispersion formulations.

The pharmaceutical compositions and formulations may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the invention (e.g., a compound of formula (I)) are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of a compound of formula (I), may be about 0.01 mg to about 1000 mg at a time, about 0.05 mg to about 500 mg at a time, about 0.1 mg to about 500 mg at a time, about 0.5 mg to about 300 mg at a time, about 1 mg to about 250 mg at a time, about 5 mg to about 200 mg at a time and about 10 mg to about 150 mg at a time, by oral administration to a patient for once to several times per day, or about 0.01 mg to about 1000 mg at a time, about 0.05 mg to about 500 mg at a time, about 0.1 mg to about 500 mg at a time, about 0.5 mg to about 300 mg at a time, about 1 mg to about 250 mg at a time, about 5 mg to about 200 mg at a time and about 10 mg to about 150 mg at a time, by parenteral administration to a patient, or continuous administration to a patient for 30 minutes to 24 hours per day intravenously. It may be administrated to patients once to several times per day.

Needless to say, as mentioned above, the effective amount to be used vary dependent upon various conditions. Therefore, effective amount lower than the ranges specified above may be sufficient in some cases, and effective amount higher than the ranges specified above are needed in some cases.

The pharmaceutical compositions and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Thus, the compounds and their physiologically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

The route by which the disclosed compounds are administered and the form of the composition will dictate the type of carrier to be used. The composition may be in a variety of forms, suitable, for example, for systemic administration (e.g., oral, rectal, nasal, sublingual, buccal, implants, or parenteral) or topical administration (e.g., dermal, pulmonary, nasal, aural, ocular, liposome delivery systems, or iontophoresis).

Carriers for systemic administration typically include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in

a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, PA) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of an active compound (e.g., a compound of formula (I)) and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound (e.g., a compound of formula (I)), and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT(Registered Trademark) coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed compounds can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions include: a disclosed compound (e.g., a compound of formula (I)), and a carrier. The carrier of the topical composition preferably aids penetration of the compounds into the skin. The carrier may further include one or more optional components.

The amount of the carrier employed in conjunction with a disclosed compound is sufficient to provide a practical quantity of composition for administration per unit dose of the compound. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, din-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

The amount of fragrance in a topical composition is typically about 0% to about 0.5%, particularly, about 0.001% to about 0.1%.

Suitable pH adjusting additives include HCl or NaOH in amounts sufficient to adjust the pH of a topical pharmaceutical composition.

In some embodiments, the following components are mixed with each other in a usual method and punched out to obtain 10,000 tablets each containing 5 mg of the active ingredient.
tert-butyl N-[4-chloro-3-[[5-[2-(4-fluorophenyl)ethynyl]-3-methyl-2-pyridyl]carbamoyl]phenyl]carbamate (50 g);
   carboxymethylcellulose calcium (disintegrating agent) (20 g);
   magnesium stearate (lubricant) (10 g);
   microcrystalline cellulose (920 g).

Spray-Dried Dispersion Formulations

The disclosed compounds may be formulated as a spray-dried dispersion (SDD). An SDD is a single-phase, amorphous molecular dispersion of a drug in a polymer matrix. It is a solid solution with the compound molecularly “dissolved” in a solid matrix. SDDs are obtained by dissolving drug and a polymer in an organic solvent and then spray-drying the solution. The use of spray drying for pharmaceutical applications can result in amorphous dispersions with increased solubility of Biopharmaceutics Classification System (BCS) class II (high permeability, low solubility) and class IV (low permeability, low solubility) drugs. Formulation and process conditions are selected so that the solvent quickly evaporates from the droplets, thus allowing insufficient time for phase separation or crystallization. SDDs have demonstrated long-term stability and manufacturability. For example, shelf lives of more than 2 years have been demonstrated with SDDs. Advantages of SDDs include, but are not limited to, enhanced oral bioavailability of poorly water-soluble compounds, delivery using traditional solid dosage forms (e.g., tablets and capsules), a reproducible, controllable and scalable manufacturing process and broad applicability to structurally diverse insoluble compounds with a wide range of physical properties.

This in one embodiment, the disclosure may provide a spray-dried dispersion formulation comprising a compound of formula (I).

Methods of Use

The disclosed compounds, pharmaceutical compositions and formulations may be used in methods for treatment of disorders, such as neurological, pshychiatric, inflammatory, respiratory, renal and cardiovascular disorders associated with K₂P K⁺ channels, specifically TREK (TWIK RElated K⁺ channels) dysfunction for which inhibitors of TREK1, TREK2 or both TREK1 and TREK2 would offer therapeutic benefit.

Treating Disorders

The disclosed compounds, pharmaceutical compositions and formulations may be used in methods for prevention and/or treatment of disorders, such as neurological and/or psychiatric disorders, associated with TREK channel dysfunction in which inhibitors of TREK1, TREK2 or both TREK1 and TREK2 would offer therapeutic benefit. The methods of prevention and/or treatment may comprise administering to a subject in need of such prevention and/or treatment a therapeutically effective amount of the compound of formula (I), or a pharmaceutical composition comprising a therapeutically effective amount of a compound of formula (I).

In some embodiments, the disclosure provides to a method for enhancing cognition and/or treating, preventing, ameliorating, controlling or reducing the risk of psychiatric symptoms such as schizophrenia and depression in a mammal comprising the step of administering to the mammal a therapeutically effective amount of the compound of formula (I), or a pharmaceutical composition comprising a therapeutically effective amount of a compound of formula (I).

The compounds and compositions disclosed herein may be useful for treating, preventing, ameliorating, controlling or reducing the risk of a variety of disorders associated with selective TREK channel inhibition. Thus, provided is a method of treating or preventing a disorder in a subject comprising the step of administering to the subject at least one disclosed compound or at least one disclosed pharmaceutical composition, in an amount effective to treat the disorder in the subject.

Also provided is a method for the prevention and/or treatment of one or more disorders associated with TREK channel activity in a subject comprising the step of administering to the subject a therapeutically effective amount of the compound of formula (I), or a pharmaceutical composition comprising a therapeutically effective amount of a compound of formula (I).

In some embodiments, the disclosure provides a method for the prevention and/or treatment of a disorder associated with TREK channel dysfunction in which inhibitors of TREK1, TREK2 or both TREK1 and TREK2 would offer therapeutic benefit in a mammal, comprising the step of administering to the mammal an effective amount of at least one disclosed compound or a pharmaceutically acceptable salt thereof, or a pharmaceutical composition comprising at least one disclosed compound or pharmaceutically acceptable salt thereof.

In some embodiments, the disclosed compounds and compositions have utility in preventing and/or treating a variety of neurological, psychiatric and cognitive disorders or cancer associated with the TREK-1 and/or 2 inhibition in which inhibitors of TREK1, TREK2 or both TREK1 and TREK2 would offer therapeutic benefit, including one or more of the following conditions or diseases: schizophrenia, psychotic disorder NOS, brief psychotic disorder, schizophreniform disorder, schizoaffective disorder, delusional disorder, shared psychotic disorder, catastrophic schizophrenia, postpartum psychosis, psychotic depression, psychotic break, tardive psychosis, myxedematous psychosis, occupational psychosis, menstrual psychosis, secondary psychotic disorder, bipolar I disorder with psychotic features, substance-induced psychotic disorder, neuropathic pain, prostatic and ovarian cancer. In some embodiments, the psychotic disorder is a psychosis associated with an illness selected from major depressive disorder, postpartum depression, treatment-resistant depression, affective disorder, bipolar disorder, electrolyte disorder, Alzheimer's disease, neurological disorder, hypoglycemia, AIDS, lupus, and post-traumatic stress disorder and 22q11.2 deletion disorder.

In some embodiments, the neurological disorder is selected from brain tumor, dementia with Lewy bodies, cerebrovascular dementia, multiple sclerosis, sarcoidosis, Lyme disease, syphilis, Alzheimer's disease, Parkinson's disease, and anti-NMDA receptor encephalitis.

In some embodiments, the psychotic disorder is selected from schizophrenia, brief psychotic disorder, schizophreniform disorder, schizoaffective disorder, delusional disorder, and shared psychotic disorder. In some embodiments, the schizophrenia is selected from catastrophic schizophrenia, catatonic schizophrenia, paranoid schizophrenia, residual schizophrenia, disorganized schizophrenia, and undifferentiated schizophrenia. In some embodiments, the disorder is selected from schizoid personality disorder, schizotypal personality disorder, and paranoid personality disorder. In some embodiments, the psychotic disorder is due to a general medical condition and is substance-induced or drug-induced (phencyclidine, ketamine and other dissociative anesthetics, amphetamine and other psychostimulants, and cocaine).

In some embodiments, schizophrenia, brief psychotic disorder, schizophreniform disorder, schizoaffective disorder, delusional disorder, and shared psychotic disorder are preferred for the neurological disorder.

The term “schizophrenia” used herein includes negative symptoms of schizophrenia and cognitive impairment associated with schizophrenia (CIAS).

In some embodiments, the present disclosure provides a method for preventing and/or treating a cognitive disorder, comprising administering to a patient in need thereof an effective amount of a compound or a composition of the present disclosure. In some embodiments, cognitive disorders include dementia (associated with Alzheimer's disease, ischemia, multi-infarct dementia, trauma, vascular problems or stroke, HIV disease, Parkinson's disease, Huntington's disease, Pick's disease, Creutzfeldt-Jacob disease, perinatal hypoxia, other general medical conditions or substance abuse), delirium, amnestic disorder, substance-induced persisting delirium, dementia due to HIV disease, dementia due to Huntington's disease, dementia due to Parkinson's disease, Parkinsonian-ALS demential complex, dementia of the Alzheimer's type, agerelated cognitive decline, and mild cognitive impairment.

The text revision of the fourth edition of the Diagnostic and Statistical Manual of Mental Disorders (DSM-IV-TR) (2000, American Psychiatric Association, Washington DC) provides a diagnostic tool that includes cognitive disorders including dementia, delirium, amnestic disorders and age-related cognitive decline. The fifth edition of the Diagnostic and Statistical Manual of Mental Disorders (DSM-5) (2013, American Psychiatric Association, Washington DC) provides a diagnostic tool for neurocognitive disorders (NCDs) that include delirium, followed by the syndromes of major NCD, mild NCD, and their etiological subtypes. The major or mild NCD subtypes include NCD due to Alzheimer's disease, vascular NCD, NCD with Lewy bodies, NCD due to Parkinson's disease, frontotemporal NCD, NCD due to traumatic brain injury, NCD due to HIV infection, substance/medication-induced NCD, NCD due to Huntington's disease, NCD due to prion disease, NCD due to another medical condition, NCD due to multiple etiologies, and unspecified NCD. The NCD category in DSM-5 encompasses the group of disorders in which the primary clinical deficit is in cognitive function, and that are acquired rather than developmental. As used herein, the term “cognitive disorders” includes prevention and/or treatment of those cognitive disorders and neurocognitive disorders as described in DSM-IV-TR or DSM-5. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In some embodiments, the present disclosure provides a method for preventing and/or treating schizophrenia or psychosis, comprising administering to a patient in need thereof an effective amount of a compound or composition of the present disclosure. Particular schizophrenia or psychosis pathologies are paranoid, disorganized, catatonic or undifferentiated schizophrenia and substance-induced psychotic disorder. DSM-IV-TR provides a diagnostic tool that includes paranoid, disorganized, catatonic, undifferentiated or residual schizophrenia, and substance-induced psychotic disorder. DSM-5 eliminated the subtypes of schizophrenia, and instead includes a dimensional approach to rating severity for the core symptoms of schizophrenia, to capture the heterogeneity in symptom type and severity expressed across individuals with psychotic disorders. As used herein, the term “schizophrenia or psychosis” includes prevention and/or treatment of those mental disorders as described in DSM-IV-TR or DSM-5. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

The compounds and compositions may be further useful in a method for the prevention, treatment, control, amelioration, or reduction of risk of the diseases, disorders and conditions noted herein. The compounds and compositions may be further useful in a method for the prevention, treatment, control, amelioration, or reduction of risk of the aforementioned diseases, disorders and conditions, in combination with other agents.

In the prevention and/or treatment of conditions which require inhibition of a TREK channel (TREK1, TREK2 or dual TREK1/2), an appropriate dosage level may be about 0.01 to 500 mg per day, which can be administered to a patient in single or multiple doses. The dosage level may be about 1 to about 300 mg per day, or about 5 to about 200 mg per day, which can be administered to a patient in single or multiple doses. A suitable dosage level can be about 1 to 250 mg per day, about 5 to 200 mg per day, or about 10 to 150 mg per day, which can be administered to a patient in single or multiple doses. Within this range the dosage can be 0.05 to 0.5, 0.5 to 5 or 5 to 50 mg per day, which can be administered to a patient in single or multiple doses. For oral administration, the compositions may be provided in the form of tablets containing 1.0 to 500 milligrams of the active ingredient, particularly 1.0, 5.0, 10, 15, 20, 25, 50, 75, 100, 150, 200, 250, 300, 400 or 500 milligrams of the active ingredient for the symptomatic adjustment of the dosage to the patient to be treated. The compounds can be administered on a regimen of 1 to 4 times per day, preferably once or twice per day. This dosage regimen can be adjusted to provide the optimal therapeutic response. It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In some embodiments, the disorder in which inhibitors of TREK1, TREK2 or both TREK1 and TREK2 would offer therapeutic benefit can be selected from psychosis, schizophrenia, conduct disorder, disruptive behavior disorder, bipolar disorder, psychotic episodes of anxiety, anxiety associated with psychosis, psychotic mood disorders such as severe major depressive disorder; mood disorders associated with psychotic disorders, acute mania, depression associated with bipolar disorder, mood disorders associated with schizophrenia, behavioral manifestations of mental retardation, autistic disorder, movement disorders, Tourette's syndrome, akinetic-rigid syndrome, movement disorders associated with Parkinson's disease, tardive dyskinesia, drug induced and neurodegeneration based dyskinesias, attention deficit hyperactivity disorder, cognitive disorders, dementias, and memory disorders.

In some embodiments, the disorder in which inhibitors of TREK1, TREK2 or both TREK1 and TREK2 would offer therapeutic benefit is Alzheimer's disease.

Co-Therapeutic Methods

The present invention is further directed to administration of a selective TREK channel inhibitor for improving treatment outcomes in the context of cognitive or behavioral therapy. That is, in some embodiments, the invention relates to a co-therapeutic method comprising a step of administering to a mammal an effective amount and dosage of at least one disclosed compound, or a pharmaceutically acceptable salt thereof.

In some embodiments, administration improves treatment outcomes in the context of cognitive or behavioral therapy. Administration in connection with cognitive or behavioral therapy can be continuous or intermittent. Administration need not be simultaneous with therapy and can be before, during, and/or after therapy. For example, cognitive or behavioral therapy can be provided within 1, 2, 3, 4, 5, 6, 7 days before or after administration of the compound. As a further example, cognitive or behavioral therapy can be provided within 1, 2, 3, or 4 weeks before or after administration of the compound. As a still further example, cognitive or behavioral therapy can be provided before or after administration within a period of time of 1, 2, 3, 4, 5, 6, 7, 8, 9, or 10 half-lives of the administered compound.

It is understood that the disclosed cotherapeutic methods can be used in connection with the disclosed compounds, compositions, kits, and uses.

Combination Therapies

In the methods of use described herein, additional therapeutic agent(s) may be administered simultaneously or sequentially with the disclosed compounds and compositions. Sequential administration includes administration before or after the disclosed compounds and compositions. In some embodiments, the additional therapeutic agent or agents may be administered in the same composition as the disclosed compounds. In other embodiments, there may be an interval of time between administration of the additional therapeutic agent and the disclosed compounds. In some embodiments, administration of an additional therapeutic agent with a disclosed compound may allow lower doses of the other therapeutic agents and/or administration at less frequent intervals. When used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula (I). The above combinations include combinations of a compound of the present invention not only with one other active compound, but also with two or more other active compounds.

The disclosed compounds can be used as single agents or in combination with one or more other drugs in the treatment, prevention, control, amelioration or reduction of risk of the aforementioned diseases, disorders and conditions for which the compound or the other drugs have utility, where the combination of drugs together are safer or more effective than either drug alone. The other drug(s) can be administered by a route and in an amount commonly used therefor, contemporaneously or sequentially with a disclosed compound. When a disclosed compound is used contemporaneously with one or more other drugs, a pharmaceutical composition in unit dosage form containing such drugs and the disclosed compound may be used. However, the combination therapy can also be administered on overlapping schedules. It is also envisioned that the combination of one or more active ingredients and a disclosed compound can be more efficacious than either as a single agent. Thus, when used in combination with one or more other active ingredients, the disclosed compounds and the other active ingredients can be used in lower doses than when each is used singly.

The pharmaceutical compositions and methods of the present invention can further comprise other therapeutically active compounds as noted herein which are usually applied in the prevention and/or treatment of the above mentioned pathological conditions.

The above combinations include combinations of a disclosed compound not only with one other active compound, but also with two or more other active compounds. Likewise, disclosed compounds can be used in combination with other drugs that are used in the prevention, treatment, control, amelioration, or reduction of risk of the diseases or conditions for which disclosed compounds are useful. Such other drugs can be administered, by a route and in an amount commonly used therefor, contemporaneously or sequentially with a compound of the present invention. When a compound of the present invention is used contemporaneously with one or more other drugs, a pharmaceutical composition containing such other drugs in addition to a disclosed compound is preferred. Accordingly, the pharmaceutical compositions include those that also contain one or more other active ingredients, in addition to a compound of the present invention.

The weight ratio of a disclosed compound to the second active ingredient can be varied and will depend upon the effective dose of each ingredient. Generally, an effective dose of each will be used. Thus, for example, when a compound of the present invention is combined with another agent, the weight ratio of a disclosed compound to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations a disclosed compound and other active agents can be administered separately or in conjunction. In addition, the administration of one element can be prior to, concurrent to, or subsequent to the administration of other agent(s).

Accordingly, the disclosed compounds can be used alone or in combination with other agents which are known to be beneficial in the subject indications or other drugs that affect receptors or enzymes that either increase the efficacy, safety, convenience, or reduce unwanted side effects or toxicity of the disclosed compounds. The subject compound and the other agent can be coadministered, either in concomitant therapy or in a fixed combination.

In some embodiments, the compound can be employed in combination with anti-Alzheimer's agents, beta-secretase inhibitors, cholinergic agents, gamma-secretase inhibitors, HMG-CoA reductase inhibitors, M₁ allosteric agonists, M₁ positive allosteric modulators, NSAIDs including ibuprofen, vitamin E, and anti-amyloid antibodies. In another embodiment, the subject compound can be employed in combination with sedatives, hypnotics, anxiolytics, antipsychotics (typical and atypical), antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound can be administered in conjunction with the use of physical methods such as with light therapy or electrical stimulation.

In some embodiments, the compound can be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist can be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In some embodiments, the compound can be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound can be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound can be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In some embodiments, the compound can be employed in combination with an antidepressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT1A agonists or antagonists, especially 5-HT1A partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

In some embodiments, the compounds can be coadministered with orthosteric muscarinic agonists, muscarinic potentiators, or cholinesterase inhibitors. In some embodiments, the compounds can be coadministered with GlyT1 inhibitors and the like such as, but not limited to: risperidone, clozapine, haloperidol, fluoxetine, prazepam, xanomeline, lithium, phenobarbitol, and salts thereof and combinations thereof.

In some embodiments, the other drugs for the prevention and/or treatment of schizophrenia is at least one drug selected from typical antipsychotics, atypical antipsychotics, and the like.

The typical antipsychotics may include, for example, chlorpromazine, fluphenazine, haloperidol, sulpiride and the like.

The atypical antipsychotics may include, for example, serotonin-dopamine antagonist, multi-acting receptor targeted antipsychotics, dopamine partial agonist and the like.

The serotonin-dopamine antagonist may include, for example, risperidone, perospirone, ziprasidone, blonanserin and the like.

The multi-acting receptor targeted antipsychotics may include, for example, olanzapine, quetiapine, clozapine, lurasidone and the like.

The dopamine partial agonist may include, for example, aripiprazole, cariprazine and the like.

In some embodiments, the other drugs for the prevention and/or treatment of depression is at least one drug selected from benzodiazepine antianxiety drug, thienodiazepine antianxiety drug, non-benzodiazepine antianxiety drug, neurokinin-1 (NK1) antagonist, tricyclic antidepressant, tetracyclic antidepressant, monoamine oxidase (MAO) inhibitor, triazolopyridine antidepressant, serotonin and noradrenaline reuptake inhibitor (SNRI), selective serotonin reuptake inhibitor (SSRI), serotonin reuptake inhibitor, noradrenergic and specific serotonergic antidepressant (NaSSA), noradrenaline and dopamine disinhibition drug (NDDI), selective serotonin reuptake enhancer (SSRE), and the like.

The benzodiazepine antianxiety drug may include, for example, alprazolam, oxazepam, oxazolam, cloxazolam, clorazepate dipotassium, chlordiazepoxide, diazepam, tofisopam, triazolam, prazepam, fludiazepam, flutazolam, flutoprazepam, bromazepam, mexazolam, medazepam, ethyl loflazepate lorazepam and the like.

The thienodiazepine antianxiety drug may include, for example, etizolam, clotiazepam and the like.

The non-benzodiazepine antianxiety drug may include, for example, citric acid tandospirone, hydroxyzine hydrochloride and the like.

The neurokinin-1 (NK1) antagonist may include, for example, aprepitant, fosaprepitant meglumine and the like.

The tricyclic antidepressant may include, for example, amitriptyline hydrochloride, imipramine hydrochloride, clomipramine hydrochloride, dosulepin hydrochloride, nortriptyline hydrochloride, lofepramine hydrochloride, trimipramine maleate, amoxapine and the like.

The tetracyclic antidepressant may include, for example, maprotiline hydrochloride, mianserin hydrochloride, setiptiline maleate and the like.

The monoamine oxidase (MAO) inhibitor may include, for example, safrazine hydrochloride and the like.

The triazolopyridine antidepressant may include, for example, Trazodone Hydrochloride and the like.

The serotonin and noradrenaline reuptake inhibitor (SNRI) may include, for example, milnacipran hydrochloride, venlafaxine hydrochloride, duloxetine hydrochloride and the like.

The selective serotonin reuptake inhibitor (SSRI) may include, for example, fluvoxamine maleate, paroxetine hydrochloride, fluoxetine hydrochloride, citalopram hydrochloride, sertraline hydrochloride, escitalopram oxalate and the like.

The serotonin reuptake inhibitor may include, for example, trazodone hydrochloride and the like.

The noradrenergic and specific serotonergic antidepressant (NaSSA) may include, for example, mirtazapine and the like.

The noradrenaline and dopamine disinhibition drug (NDDI) may include, for example, agomelatine and the like.

The selective serotonin reuptake enhancer (SSRE) may include, for example, tianeptine and the like.

Modes of Administration

Methods of prevention and/or treatment may include any number of modes of administering a disclosed composition. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. Gelucire™). In the pharmaceutical composition, the agent may also be dispersed in a microparticle, e.g. a nanoparticulate composition.

For parenteral administration, the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration, the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

The present compound has low toxicity and thus can be safely used as a medicament.

Kits

In one aspect, the disclosure provides kits comprising at least one disclosed compound or a pharmaceutically acceptable salt thereof, and one or more of:
(a) at least one agent known to decrease TREK1 activity;
(b) at least one agent known to decrease TREK2 activity;
(c) at least one agent known to prevent and/or treat a disorder associated with TREK dysfunction in which inhibitors of TREK1, TREK2 or both TREK1/TREK2 would offer therapeutic benefit in a mammal;
(d) instructions for preventing and/or treating a disorder associated with TREK dysfunction in which inhibitors of TREK1, TREK2 or both TREK1/TREK2 would offer therapeutic benefit in a mammal; or
(e) instructions for administering the compound in connection with cognitive or behavioral therapy.

In some embodiments, the at least one disclosed compound and the at least one agent are co-formulated. In some embodiments, the at least one disclosed compound and the at least one agent are co-packaged. The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

That the disclosed kits can be employed in connection with disclosed methods of use.

The kits may include information, instructions, or both that use of the kit will provide prevention and/or treatment for medical conditions in mammals (particularly humans). The information and instructions may be in the form of words, pictures, or both, and the like. In addition or in the alternative, the kit may include the compound, a composition, or both; and information, instructions, or both, regarding methods of application of compound, or of composition, preferably with the benefit of preventing and/or treating medical conditions in mammals (e.g., humans).

The compounds and processes of the invention will be better understood by reference to the following examples, which are intended as an illustration of and not a limitation upon the scope of the invention.

### EXAMPLES

All NMR spectra were recorded on a 400 MHz AMX Bruker NMR spectrometer, a 400 MHz AVANCE III HD Bruker NMR spectrometer or a 500 MHz AVANCE III HD Bruker NMR spectrometer. ¹H chemical shifts are reported in δ values in ppm downfield with the deuterated solvent as the internal standard. Data are reported as follows: chemical shift, multiplicity (s=singlet, bs=broad singlet, d=doublet, t=triplet, q=quartet, dd=doublet of doublets, m=multiplet, ABq=AB quartet), coupling constant, integration.

LCMS analysis conditions are as below. The ES-MS data and retention time in Examples were measured by Reversed-phase LCMS method-1 unless otherwise specified.

Reversed-phase LCMS analysis was performed using:

Reversed-phase LCMS method-1: an Agilent 1200 system comprised of a binary pump with degas ser, high-performance autosampler, thermostatted column compartment, C18 column, diode-array detector (DAD) and an Agilent 6150 MSD with the following parameters. The gradient conditions were 5% to 95% acetonitrile with the aqueous phase 0.1% TFA in water over 1.4 minutes. Samples were separated on a Waters Acquity UPLC BEH C18 column (1.7 μm, 1.0×50 mm) at 0.5 mL/min, with column and solvent temperatures maintained at 55° C. The DAD was set to scan from 190 to 300 nm, and the signals used were 220 nm and 254 nm (both with a band width of 4 nm). The MS detector was configured with an electrospray ionization source, and the low-resolution mass spectra were acquired by scanning from 140 to 700 AMU with a step size of 0.2 AMU at 0.13 cycles/second, and peak width of 0.008 minutes. The drying gas flow was set to 13 liters per minute at 300° C. and the nebulizer pressure was set to 30 psi. The capillary needle voltage was set at 3000 V, and the fragmentor voltage was set at 100V. Data acquisition was performed with Agilent Chemstation and Analytical Studio Reviewer software.

Reversed-phase LCMS method-2: Reversed-phase LCMS analysis was performed using a Waters Acquity UPLC I-Class System comprised of a binary pump, high-performance autosampler, thermostatted column compartment, C18 column, diode-array detector (DAD), ELSD and SQD2 with the following parameters. Samples were separated on a YMC Triart C18 column (1.9 μm, 2.0×30 mm) at 1.0 mL/min, with column and solvent temperatures maintained at 30° C. The gradient conditions were mobile phase: A) 0.1% TFA in water, B) 0.1% TFA in acetonitrile, gradient condition (Acetonitrile %); 0.00-0.10 min. 5%, 0.10-1.20 min. 5-95%, 1.20-1.40 min. 95%. The DAD was set to scan from 210 to 400 nm, and the signals used were 220 nm and 254 nm (both with a band width of 1.2 nm). The MS detector was configured with an electrospray ionization source, and the low-resolution mass spectra were acquired by scanning from 140 to 700 AMU at 2 cycles/second. The drying gas flow was set to 650 liters per hour at 350° C. and the cone gas flow was set to 50 liters per hour. The capillary needle voltage was set at 2200 V, and the cone voltage was set at 30V. Data acquisition was performed with Waters MassLynx and OpenLynx software.

Reversed-phase LCMS method-3: Reversed-phase LCMS analysis was performed using a Shimadzu NexeraX2 System comprised of a binary pump, high-performance autosampler, thermostatted column compartment, C18 column, diode-array detector (DAD), ELSD and LCMS-2020 with the following parameters. Samples were separated on a YMC Triart C18 column (1.9 μm, 2.0×30 mm) at 1.0 mL/min, with column and solvent temperatures maintained at 30° C. The gradient conditions were mobile phase: A) 0.1% TFA in water, B) 0.1% TFA in acetonitrile, gradient condition (Acetonitrile %); 0.00-0.10 min. 5%, 0.10-1.20 min. 5-95%, 1.20-1.50 min. 95%. The DAD was set to scan from 210 to 400 nm, and the signals used were 220 nm and 254 nm (both with a band width of 1.2 nm). The MS detector was configured with an electrospray ionization source, and the low-resolution mass spectra were acquired by scanning from 160 m/z to 1000 m/z at 5,000 u/second. The drying gas flow was set to 15 liters per minute at 400° C. and the nebulizer gas flow was set to 1.5 liters per minute. The probe voltage was set at 4.5 kV. Data acquisition was performed with Shimadzu Labsolution software.

Abbreviations which have been used in the descriptions of following examples are: AcOH is acetic acid; BOP is (benzotriazol-1-yloxy)tris(dimethylamino)phosphonium hexafluorophosphate; DBU is 1,8-diazabicyclo(5.4.0)undec-7-ene; DCM is dichloromethane; DIEA is N,N-diisopropylethylamine; DMF is N,N-dimethylformamide; DMSO is dimethyl sulfoxide; EtOAc is ethyl acetate; IPA is isopropyl alcohol; NMP is N-methyl-2-pyrrolidone; MW is microwave (referring to a microwave reactor); PyClU is chlorodipyrrolidinocarbenium hexafluorophosphate; HCTU is O-(6-Chlorobenzotriazol-1-yl)-N,N,N′,N′-tetramethyluronium hexafluorophosphate; RBF is round-bottomed flask; RT or rt is room temperature; and T₃P is propylphosphonic anhydride.

Intermediates:

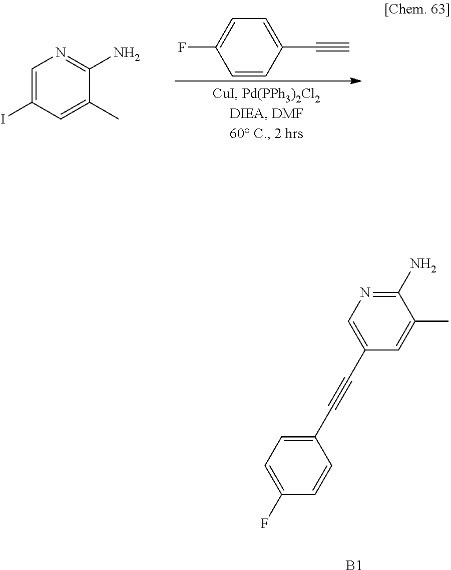

5-((4-fluorophenyl)ethynyl)-3-methylpyridin-2-amine (B1): To a microwave vial was added 5-iodo-2-amino-3-picoline (2000 mg, 8.55 mmol), N,N-diisopropylethylamine (10.42 mL, 59.82 mmol), trans-dichlorobis(triphenylphosphine)palladium(II) (602 mg, 0.85 mmol), copper(I) iodide (163 mg, 0.85 mmol), and 4-fluorophenylacetylene (2.94 mL, 25.64 mmol). The vial was capped, evacuated and purged with N₂ (3×) and then diluted with DMF (2 mL). The mixture was heated to 60° C. for 2 hours in the microwave. After LCMS of the reaction confirmed product, the sample was filtered through a pad of celite and rinsed with (3:1) CHCl₃:IPA. The sample was concentrated and then purified by normal phase column chromatography (gradient: 0-80% EtOAc in Hexanes) to yield (B1) (1564 mg, 81% yield). ES-MS [M+1]⁺: 227.2, ¹H NMR (400 MHz, CDCl₃) δ 8.15 (d, J=1.3 Hz, 1H), 7.49-7.42 (m, 2H), 7.41 (d, J=0.7 Hz, 1H), 7.03 (m, 2H), 4.58 (s, 2H), 2.13 (s, 3H).

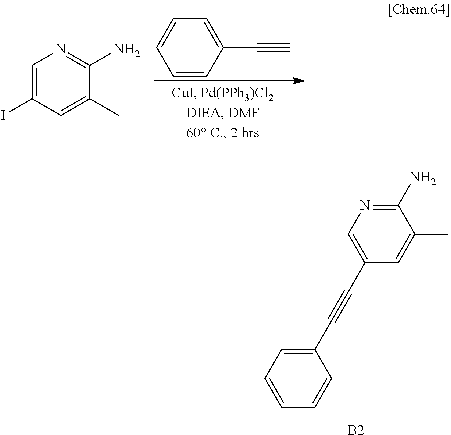

3-methyl-5-(phenylethynyl)pyridin-2-amine (B2): Compound B2 is prepared in a similar manner as Compound B1 to yield the title compound B2 (1807 mg, 68% yield). ES-MS [M+1]⁺: 209.4, ¹H NMR (400 MHz, CDCl₃) δ 8.15 (s, 1H), 7.51-7.48 (m, 2H), 7.45 (s, 1H), 7.03 (m, 3H), 4.76 (s, 2H), 2.14 (s, 3H).

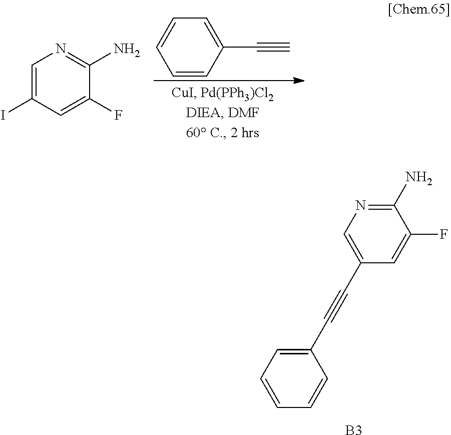

3-fluoro-5-(phenylethynyl)pyridin-2-amine (B3): Compound B3 is prepared in a similar manner as Compound B1 to yield the title compound B3 (26.0 mg, 97% yield). ES-MS [M+1]⁺: 213.2, ¹H NMR (400 MHz, CDCl₃) δ 8.07 (s, 1H), 7.51-7.49 (m, 2H), 7.36-7.33 (m, 4H), 4.76 (s, 2H).

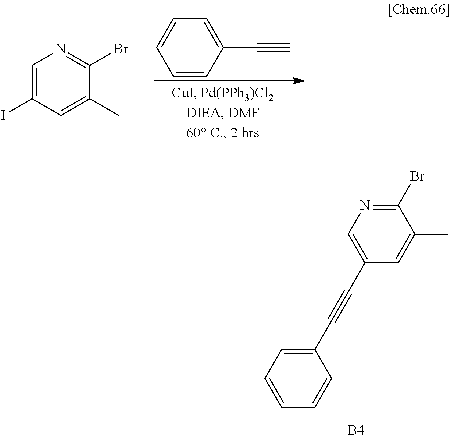

2-bromo-3-methyl-5-(phenylethynyl)pyridine (B4): Compound B4 is prepared in a similar manner as Compound B1 to yield the title compound B4 (585 mg, 64% yield). ES-MS [M+1]⁺: 272.2, ¹H NMR (400 MHz, D4-MeOD) δ 8.30 (d, J=1.9 Hz, 1H), 7.84-7.83 (m, 1H), 7.57-7.52 (m, 2H), 7.42-7.38 (m, 3H), 2.41 (s, 3H).

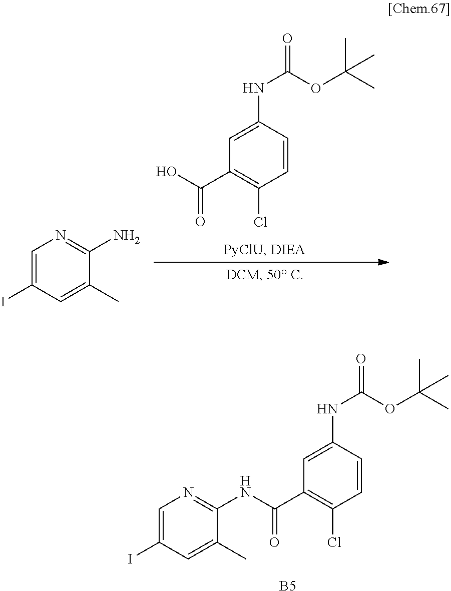

tert-butyl (4-chloro-3-((5-iodo-3-methylpyridin-2-yl)carbamoyl)phenyl)carbamate (B5): To a colorless solution of 5-(tert-butoxycarbonylamino)-2-chloro-benzoic acid (5000 mg, 18.4 mmol), 5-iodo-3-methyl-pyridin-2-amine (4738 mg, 20.2 mmol), DIEA (9.55 mL, 55.2 mmol) in DCM (50 mL) was added PyClU (9183 mg, 27.6 mmol). The reaction mixture was heated to 50° C. After 1.5h, LCMS indicated product formation. The mixture was poured into NaHCO₃-aq and THF was added to dissolve desired product. The mixture was extracted with ethyl acetate twice. The combined organic layer was washed with water, subsequently NH₄Cl-aq, water, brine and dried over MgSO₄. The filtrate was concentrated under reduced pressure to give a crude product, which was triturated with mixture of DCM (55.5 mL) and hexane (55.5 mL) to yield (B5) as a white powder (7.11 g, 79% yield). ES-MS [M+1]⁺: 488, ¹H NMR (400 MHz, DMSO-d₆) δ 10.61 (br, 1H), 9.67 (br, 1H), 8.50 (br, 1H), 8.14 (d, J=1.5 Hz, 1H), 7.73 (d, J=2.5 Hz, 1H), 7.52 (dd, J=9.0, 2.5 Hz, 1H), 7.41 (d, J=9.0 Hz, 1H), 2.26 (s, 3H), 1.49 (s, 9H).

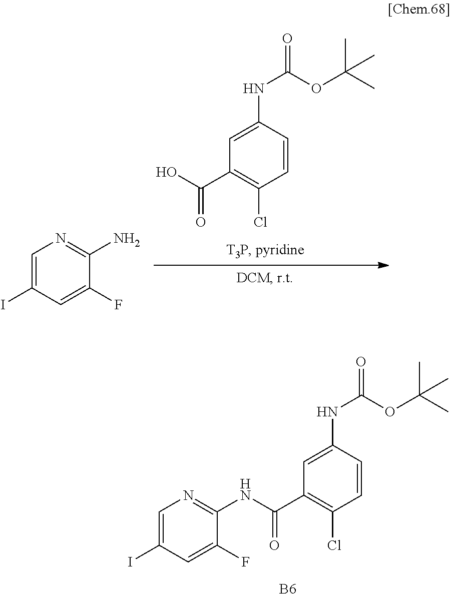

tert-butyl (4-chloro-3-((3-fluoro-5-iodopyridin-2-yl)carbamoyl)phenyl)carbamate (B6): To a beige suspension of 5-(tert-butoxycarbonylamino)-2-chloro-benzoic acid (10000 mg, 36.8 mmol), 3-fluoro-5-iodopyridin-2-amine (9636 mg, 40.5 mmol), pyridine (8.93 mL, 110.4 mmol) in DCM (80 mL) was added a solution of propylphosphonic anhydride (T₃P) in ethyl acetate (50%, 32.5 mL, 55.2 mmol). The reaction mixture was stirred at ambient temperature for 19h. The mixture was poured into NaHCO₃-aq and THF was added to dissolve desired product. The mixture was extracted with a mixture of THF and ethyl acetate twice and then ethyl acetate. The combined organic layer was washed with NaHCO₃-aq, water, subsequently NH₄Cl-aq (×2), water, brine and dried over MgSO₄. The filtrate was concentrated under reduced pressure to give a crude product (20.2 g), which was triturated with mixture of ethyl acetate (60 mL) and hexane (120 mL) at 70° C. for 30 min and then at ambient temperature for 2h. the powder was collected through filtration to yield (B6) as a pale yellow powder (13.74 g, 76% yield). ES-MS [M+1]⁺: 492, ¹H NMR (400 MHz, DMSO-d₆) δ 10.92 (br, 1H), 9.69 (br, 1H), 8.53 (br, 1H), 8.32 (dd, J=9.0, 1.5 Hz, 1H), 7.75 (d, J=2.5 Hz, 1H), 7.52 (dd, J=9.0, 2.5 Hz, 1H), 7.43 (d, J=9.0 Hz, 1H), 1.49 (s, 9H).

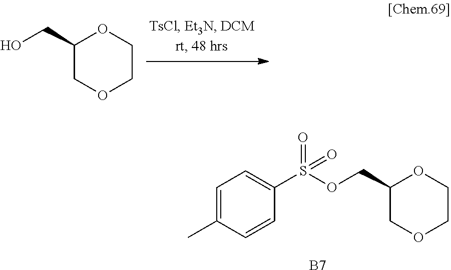

((2S)-1,4-dioxan-2-yl]methyl 4-methylbenzenesulfonate (B7): To a solution of (R)-2-(hydroxymethyl)dioxane (2000 mg, 16.9 mmol) in DCM (20 mL) was added p-toluenesulfonyl chloride (0.74 mL, 20.3 mmol) and triethylamine (4.72 mL, 33.9 mmol). After 48 hours at room temperature, the reaction was partially concentrated and diluted with water (500 ml). The dilution was extracted with EtOAc (2×500 mL). The combined organic layers were concentrated. The crude residue was purified by normal-phase flash chromatography (gradient: 10-80% EtOAc in hexanes). The fractions were combined and concentrated to yield B7 (4610 mg, 95% yield) ES-MS [M+1]⁺: 273.2, ¹H NMR (400 MHz, CDCl₃) δ 7.79 (d, J=8.0 Hz, 2H), 7.35 (d, J=7.9 Hz, 2H), 4.04-3.94 (m, 2H), 3.81-3.77 (m, 1H), 3.76-3.72 (m, 2H), 3.70-3.64 (m, 2H), 3.59-3.52 (m, 1H), 3.35 (dd, J=10.9, 10.2 Hz, 1H), 2.45 (s, 3H).

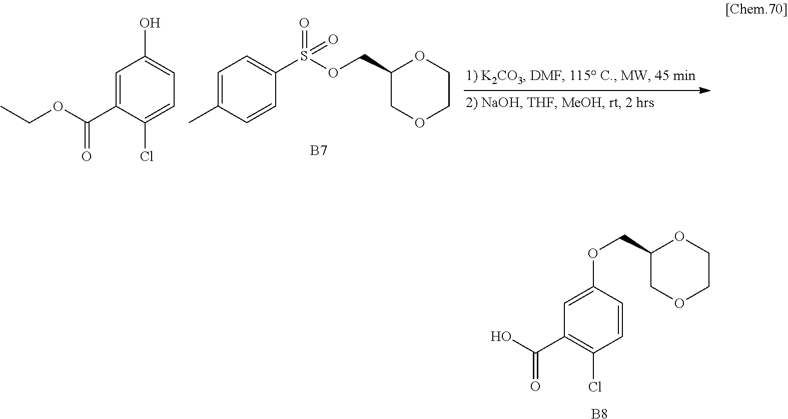

2-chloro-5-[[(2S)-1,4-dioxan-2-yl]methoxy]benzoic acid B8): A solution of ethyl 2-chloro-5-hydroxybenzoate (2578 mg, 12.9 mmol), intermediate B7 (3500 mg, 12.9 mmol), and potassium carbonate (7209 mg, 51.4 mmol) in DMF (103 mL) was heated to 115° C. in a microwave synthesizer for 45 min. The solution was diluted with water and extracted with EtOAc (2×500 mL). The combined organic layers were washed with water (2X) and concentrated in vacuo. The residue was dissolved in THF (200 mL), Methanol (80 mL), and aqueous sodium hydroxide solution (103 mL, 2N). After 2 hours at room temperature, the solution was diluted with water and EtOAc and treated with 2N aqueous HCL solution to lower the pH to <5 pH. The reaction mixture was extracted with EtOAc (2×500 mL). The organic layers were washed with water (3×1 L). Each water wash was extracted with EtOAc (3×250 mL). The combined organic layers were concentrated in vacuo. The residue was dissolved in DCM and filtered through a hydrophobic filter to yield intermediate B8. The product was used for subsequent reactions without further purification. ES-MS [M+1]⁺: 273, ¹H NMR (400 MHz, CDCl₃) δ 7.56 (d, J=3.0 Hz, 1H), 7.37 (d, J=8.8 Hz, 1H), 7.04 (dd. J=8.8, 3.0 Hz, 1H), 4.05-3.94 (m, 3H), 3.93-3.87 (m, 2H), 3.82 (ddd, J=10.6, 2.6, 2.6 Hz, 1H), 3.77 (d, J=11.5 Hz, 1H), 3.68 (ddd, J=11.0, 11.0, 3.1 Hz, 1H), 3.56 (m, 1H).

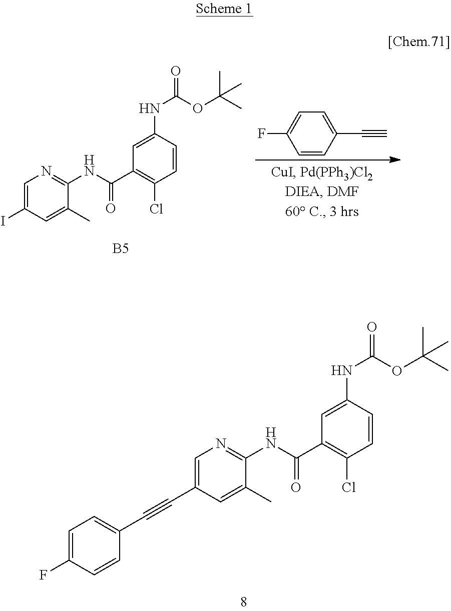

tert-butyl N-[4-chloro-3-[[5-[2-(4-fluorophenyl)ethynyl]-3-methyl-2-pyridyl]carbamoyl]phenyl]carbamate (8): To a solution of B5 (500 mg, 1.03 mmol), 1-ethynyl-4-fluorobenzene (0.59 mL, 5.1 mmol), DIEA (1.77 mL, 10.3 mmol) in DMF (4 mL) was added CuI (39.1 mg, 0.21 mmol) and Pd(PPh₃)₂Cl₂. The reaction mixture was heated to 60° C. After 3h, LCMS indicated product formation. The reaction mixture was diluted with ethyl acetate and water was added. The mixture was extracted with ethyl acetate twice. The combined organic layer was diluted with hexane and washed with water (×2), brine and dried over MgSO₄. The filtrate was concentrated under reduced pressure to give a crude product, which was purified by column chromatography on silica gel and the concentrated residue was triturated with a mixture of ethyl acetate, tert-butyl methyl ether and hexane to yield (8) as an off-white powder (103 mg, 22% yield). ES-MS [M+1]⁺: 480.5, LCMS Retention time: 1.14 min (LCMS method-2), ¹H NMR (400 MHz, DMSO-d₆) δ 10.71 (br, 1H), 9.68 (br, 1H), 8.46 (br, 1H), 7.94 (d, J=1.5 Hz, 1H), 7.75 (d, J=2.5 Hz, 1H), 7.68-7.65 (m, 2H), 7.53 (dd, J=9.0, 2.5 Hz, 1H), 7.42 (d, J=9.0 Hz, 1H), 7.34-7.29 (m, 2H), 2.31 (s, 3H), 1.49 (s, 9H).

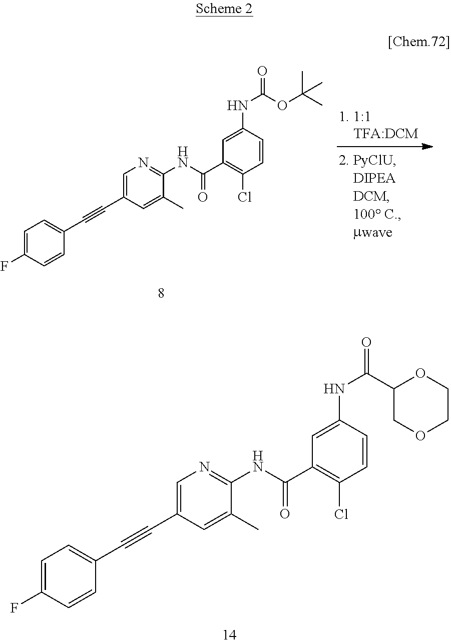 [SMILES: Cc1cc(C#Cc2ccc(F)cc2)cnc1NC(=O)c1cc(NC(=O)OC(C)(C)C)ccc1Cl ; Cc1cc(C#Cc2ccc(F)cc2)cnc1NC(=O)c1cc(NC(=O)C2COCCO2)ccc1Cl]

N-(4-chloro-3-((5-((4-fluorophenyl)ethynyl)-3-methylpyridin-2-yl)carbamoyl)phenyl)1,4-dioxane-2-carboxamide (14): A mixture of compound 8 (27 mg, 0.060 mmol) in DCM: trifluoroacetic acid (1:1; 1 mL) was stirred at rt for 1 h. The mixture was concentrated in vacuo and washed with diethyl ether to give a tan solid. ES-MS [M+1]+: 380.0. The crude residue (5 mg, 0.013 mmol), PyClU (9 mg, 0.026 mmol), DIEA (0.012 mL, 0.066 mmol), and dioxin-2-carboxylic acid (0.005 mL, 0.039 mmol) in DCM (1.0 mL) was heated to 100° C. in a microwave synthesizer for 45 min. The sample was cooled to rt, filtered, concentrated in vacuo, and purified by reverse phase HPLC (gradient: 55-90% MeCN in water (w/0.05% NH 4 OH)) to give an off-white solid (2 mg, 31% yield). ES-MS [M+1]⁺: 494.0, LCMS Retention time: 0.92 min, ¹H NMR (400 MHz, DMSO-d₆) δ 10.74 (s, 1H), 10.08 (s, 1H), 8.45 (s, 1H), 8.01 (d, J=2.6 Hz, 1H), 7.94 (d, J=1.7 Hz, 1H), 7.83 (dd, J=8.8, 2.6 Hz, 1H), 7.71-7.60 (m, 2H), 7.48 (d, J=8.8 Hz, 1H), 7.34-7.25 (m, 2H), 4.26 (dd, J=9.2, 3.1 Hz, 1H), 3.99-3.85 (m, 2H), 3.78-3.68 (m, 2H), 3.61-3.52 (m, 2H), 2.31 (s, 3H).

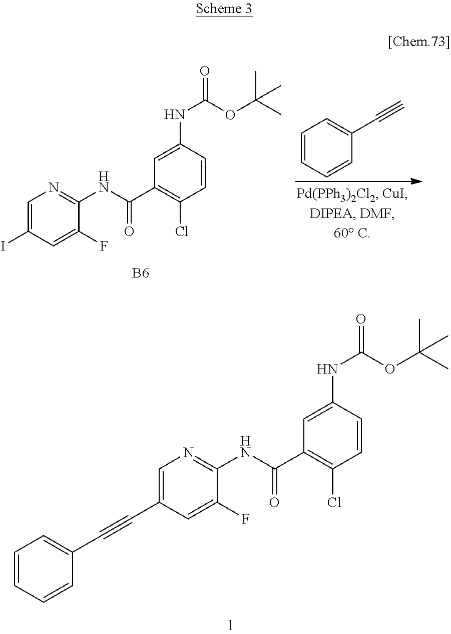

tert-butyl (4-chloro-3-((3-fluoro-5-(phenylethynyl)pyridin-2-yl)carbamoyl)phenyl)carbamate (1): Compound 1 was synthesized using a similar condition for the synthesis of compound 8. Compound 1 was obtained as an off-white solid (45 mg, 48% yield). ES-MS [M+1]+: 466.3, LCMS Retention time: 1.04 min, ¹H NMR (400 MHz, DMSO-d₆) δ 11.03 (s, 1H), 9.69 (s, 1H), 8.48 (dd, J=1.8, 0.8 Hz, 1H), 8.10 (dd, J=10.5, 1.8 Hz, 1H), 7.76 (d, J=2.6 Hz, 1H), 7.66-7.57 (m, 2H), 7.52 (dd, J=8.8, 2.6 Hz, 1H), 7.49-7.45 (m, 3H), 7.43 (d, J=8.8 Hz, 1H), 1.48 (s, 9H).

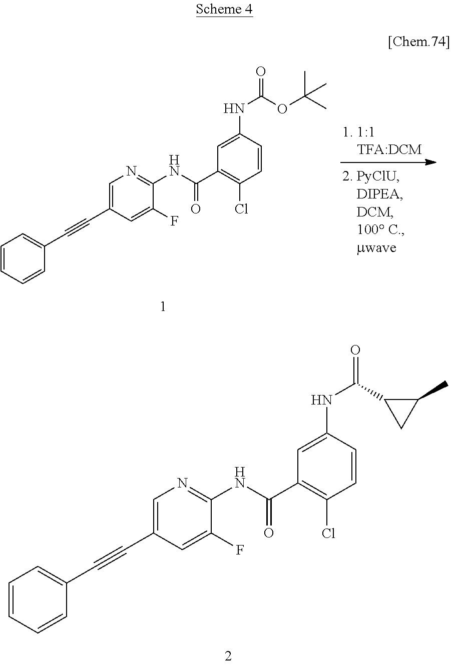

2-chloro-N-(3-fluoro-5-(phenylethynyl)pyridin-2-yl)-5-((1S,2S)-2-methylcyclopropa ne-1-carboxamido)benzamide (2): A mixture of compound 1 (45.4 mg, 0.097 mmol), DCM (1.0 mL) and trifluoroacetic acid (1.0 mL, 13.06 mmol) was stirred at rt for 1 h. The mixture was concentrated in vacuo and washed with diethyl ether to give a tan solid. ES-MS [M+1]⁺: 366.0. The resulting amine (18 mg, 0.05 mmol), PyClU (32 mg, 0.100 mmol), DIEA (0.04 mL, 0.240 mmol), and (1S,2S)-2-methylcyclopropane-1-carboxylic acid (0.010 mL, 0.150 mmol) in DCM (2.0 mL) was heated to 100° C. in a microwave synthesizer for 45 min. The sample was cooled to rt, filtered, concentrated in vacuo, and purified by reverse phase HPLC (gradient: 60-90% MeCN in water (w/0.05% NH 4 OH)) to give 2 an off-white solid (14 mg, 63% yield). ES-MS [M+1]⁺: 448.0, LCMS Retention time: 0.98 min, ¹H NMR (400 MHz, DMSO-d₆) δ 11.05 (s, 1H), 10.42 (s, 1H), 8.47 (s, 1H), 8.09 (d, J=10.5 Hz, 1H), 7.89 (d, J=2.5 Hz, 1H), 7.68 (dd, J=8.8, 2.6 Hz, 1H), 7.64-7.58 (m, 2H), 7.51-7.43 (m, 4H), 1.55-1.47 (m, 1H), 1.29-1.19 (m, 1H), 1.10 (d, J=6.0 Hz, 3H), 1.07-0.98 (m, 1H), 0.73-0.64 (m, 1H).

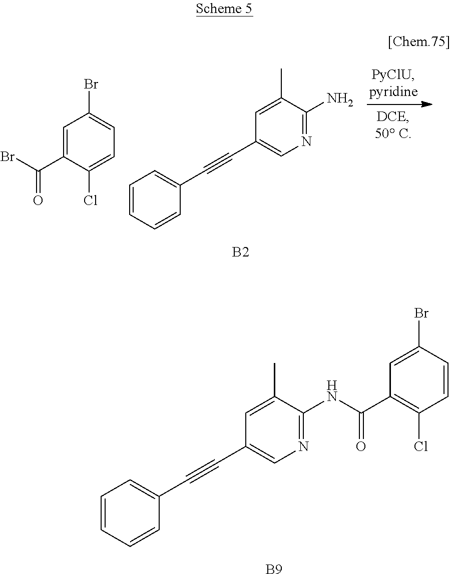

5-bromo-2-chloro-N-(3-methyl-5-(phenylethynyl)pyridin-2-yl)benzamide (B9): Compound B9 was synthesized using a similar condition described for the synthesis of B5. Compound B9 was obtained as an off-white solid (567 mg, 28% yield). ES-MS [M+H]⁺: 427.0, ¹H NMR (400 MHz, DMSO-d₆) δ 10.84 (s, 1H), 8.45 (s, 1H), 7.94 (d, J=1.3 Hz, 1H), 7.81 (d, J=2.4 Hz, 1H), 7.59-7.58 (m, OH), 7.52 (d, J=8.6 Hz, 1H), 7.49-7.42 (m, 3H), 2.32 (s, 3H).

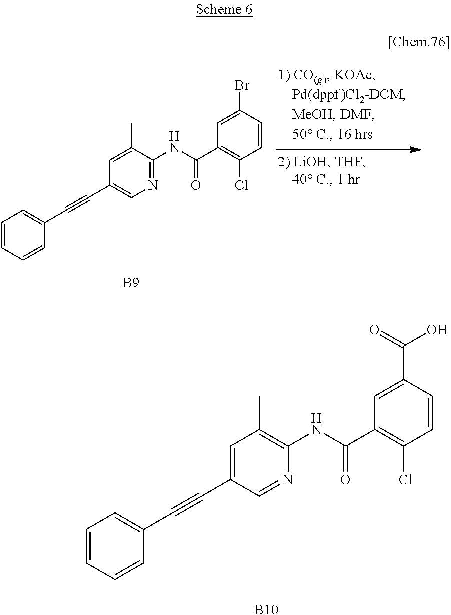

4-chloro-3-[[3-methyl-5-(2-phenylethynyl)-2-pyridyl]carbamoyl]benzoic acid (B10): To a vessel was added potassium acetate (104 mg, 1.1 mmol), intermediate B9 (150 mg, 0.35 mmol), and dichloro[1,1′-bis(diphenylphosphino)ferrocene]palladium(II) dichloromethane adduct (58 mg, 0.07 mmol). The vessel was sealed and purged with N₂. To the mixture was added Methanol (1.8 mL) and DMF (1.8 mL). An atmosphere of CO (g) was applied to the reaction vessel via a balloon. After 16 hours at 50° C., the reaction mixture was purified by reverse phase HPLC (gradient: 10-50% MeCN in water (0.1% TFA in water)). The fractions that contained desired product were combined and diluted with water and NaHCO₃ solution (aq). The mixture was extracted with EtOAc (3×10 mL) and the combined organic layers were washed with water and concentrated. To the residue was added THF (1.0 mL) and 1M LiOH (1 mL, 1 mmol). After 1 hour at 40° C., 2N HCL was added dropwise to the solution until the pH was ˜7. The mixture was concentrated and the resulting solids were washed with water and collected via vacuum filtration to yield B10 (67 mg, 49% yield over two steps), ES-MS [M+1]⁺: 391.2, ¹H NMR (400 MHz, D4-MeOD) δ 8.44 (s, 1H), 8.25 (d, J=1.2 Hz, 1H), 8.05 (dd, J=8.7, 1.5 Hz, 1H), 7.92 (s, 1H), 7.61-7.56 (m, 2H), 7.54 (d, J=8.3 Hz, 1H), 7.45-7.39 (m, 3H), 2.44 (s, 3H).

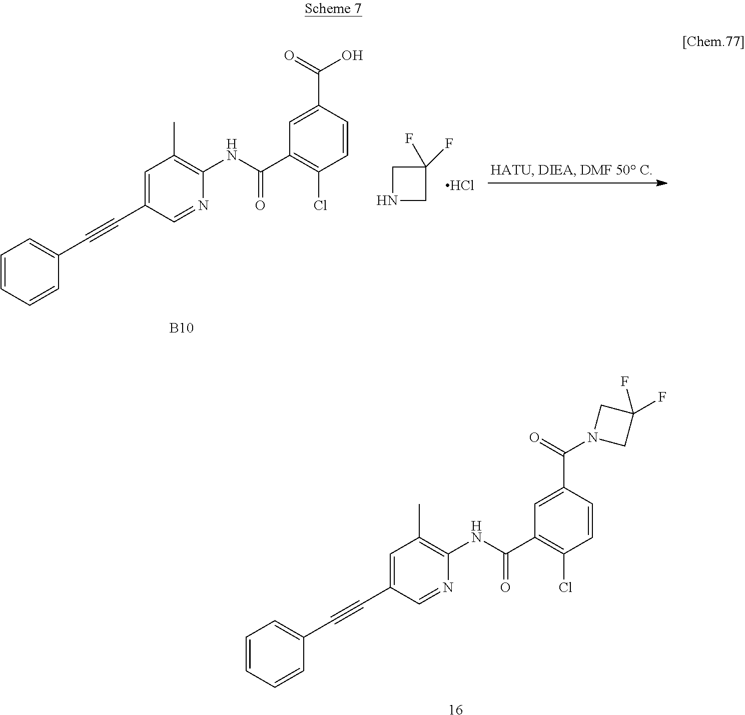

2-chloro-5-(3,3-difluoroazetidine-1-carbonyl)-N-[3-methyl-5-(2-phenylethynyl)-2-pyridyl]benzamide (16): A solution of HATU (49 mg, 0.13 mmol), 3,3-difluoroazetidine hydrochloride 8 mg, 0.06 mmol), B10 (25 mg, 0.06 mmol), and N,N-diisopropylethylamine (0.03 mL, 0.19 mmol) in DMF (1.9 mL) was heated to 50° C. The reaction mixture was purified by reverse phase HPLC (gradient: 20-60% MeCN in water (0.1% TFA in water)). Fractions that contained the desired product were combined and diluted with water and NaHCO₃ solution. The reaction mixture was extracted with EtOAc (2×10 mL). The combined organic layers were concentrated to yield compound 16 (10.2 mg, 34% yield), ES-MS [M+1]⁺: 466.0, LCMS Retention time: 0.96 min, ¹H NMR (400 MHz, CDCl₃) δ 8.94 (br s, 1H), 8.32 (s, 1H), 8.02 (d, J=1.9 Hz, 1H), 7.79 (d, J=1.3 Hz, 1H), 7.73 (dd, J=8.3, 2.1 Hz, 1H), 7.56-7.53 (m, 3H), 7.40-7.36 (m, 3H), 4.56 (t, J=11.7 Hz, 4H), 2.41 (s, 3H).

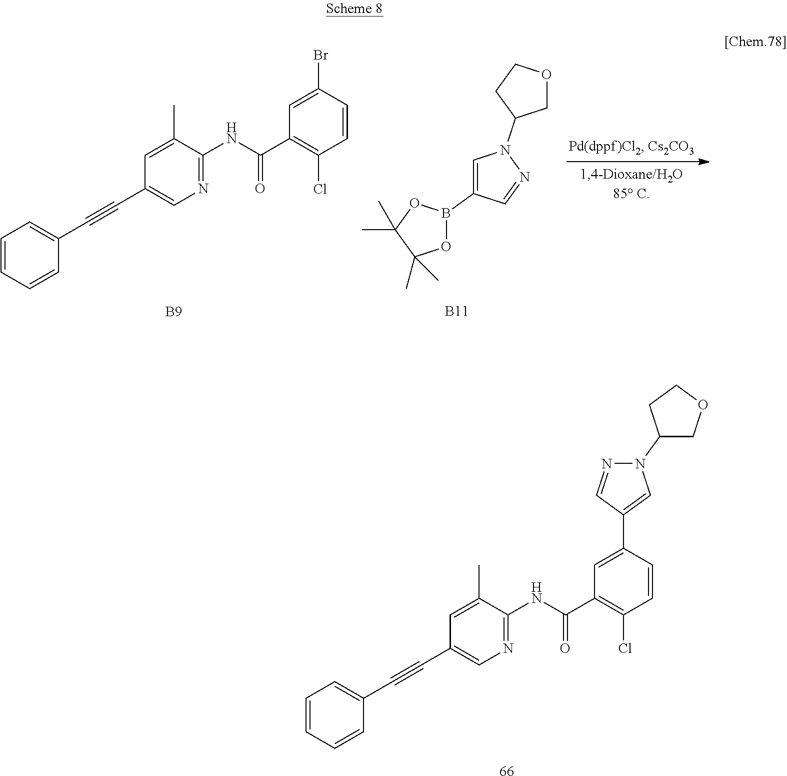

2-chloro-N-(3-methyl-5-(phenylethynyl)pyridin-2-yl)-5-(1-(tetrahydrofuran-3-yl)-1H-pyrazol-4-yl)benzamide (66): A mixture of 5-bromo-2-chloro-N-[3-methyl-5-(2-phenylethynyl)-2-pyridyl]benzamide (10 mg, 0.020 mmol), 1-tetrahydrofuran-3-yl-4-(4,4,5,5-tetramethyl-1,3,2-dioxaborolan-2-yl)pyrazole (12 mg, 0.050 mmol), Pd(dppf)Cl₂ (2 mg, 0.002 mmol), and cesium carbonate (17 mg, 0.050 mmol) in 1,4-dioxane (1 mL) and H₂O (0.05 mL) was heated to 85° C. for 2 h. The reaction mixture was cooled to rt, filtered, concentrated in vacuo, and purified by reverse phase HPLC (gradient: 55-85% MeCN in water (w/0.05% NH 4 OH)) to give 66 as a light yellow solid (6.4 mg, 56% yield). ES-MS [M+H]⁺: 483.2, LCMS Retention time: 1.11 min, ¹H NMR (400 MHz, DMSO-d₆) δ 10.77 (s, 1H), 8.47 (s, 1H), 8.42 (d, J=0.8 Hz, 1H), 8.04 (d, J=0.8 Hz, 1H), 7.95 (dd, J=2.3, 0.9 Hz, 1H), 7.83 (d, J=2.2 Hz, 1H), 7.73 (dd, J=8.4, 2.2 Hz, 1H), 7.63-7.56 (m, 2H), 7.51 (d, J=8.4 Hz, 1H), 7.48-7.43 (m, 3H), 5.08-4.98 (m, 1H), 4.09 (q, J=5.3 Hz, 2H), 4.04-3.97 (m, 2H), 3.94 (dd, J=9.4, 3.7 Hz, 1H), 3.89-3.79 (m, 1H), 2.48-2.36 (m, 1H), 2.36-2.28 (m, 4H).

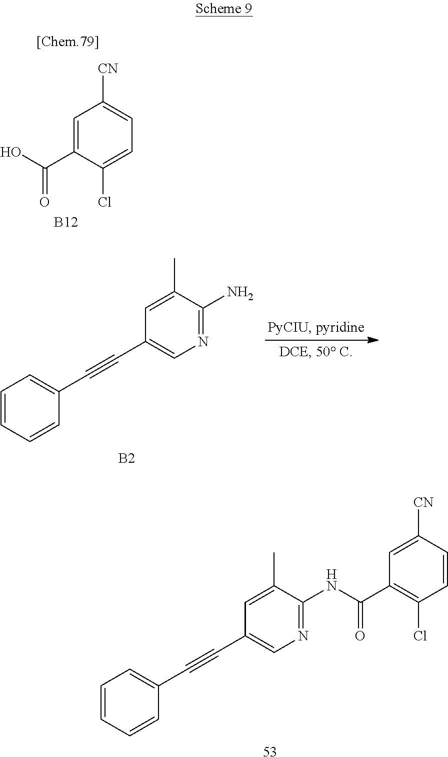

2-chloro-5-cyano-N-(3-methyl-5-(phenylethynyl)pyridin-2-yl)benzamide (53): Compound 53 was synthesized using a similar condition described for the synthesis of B5. Compound 53 was obtained as an off-white solid (15 mg, 11% yield). ES-MS [M+H]⁺: 372.2, LCMS Retention time: 1.06 min, ¹H NMR (400 MHz, DMSO-d₆) δ 10.91 (s, 1H), 8.44 (s, 1H), 8.19 (d, J=2.0 Hz, 1H), 7.99 (dd, J=8.4, 2.1 Hz, 1H), 7.97-7.93 (m, 1H), 7.80 (d, J=8.4 Hz, 1H), 7.62-7.55 (m, 2H), 7.51-7.42 (m, 3H), 2.33 (s, 3H).

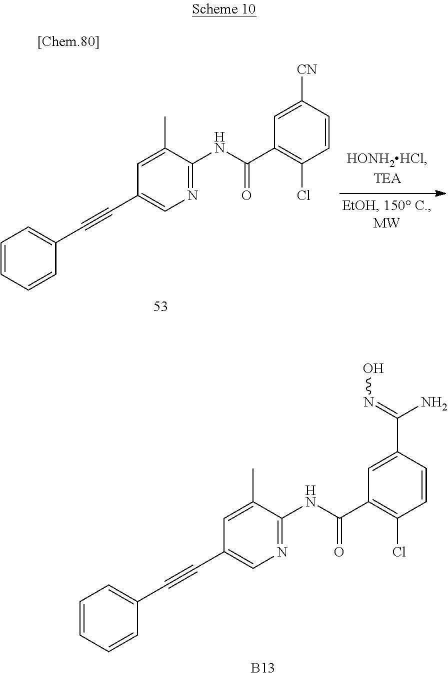

2-chloro-5-(N′-hydroxycarbamimidoyl)-N-(3-methyl-5-(phenylethynyl)pyridin-2-yl) benzamide (B13): A mixture of 2-chloro-5-cyano-N-[3-methyl-5-(2-phenylethynyl)-2-pyridyl]benzamide (20 mg, 0.05 mmol), hydroxylamine hydrochloride (19 mg, 0.27 mmol), and trimethylamine (0.04 mL, 0.27 mmol) in Ethanol (0.5 mL) was heated to 150° C. in a microwave synthesizer for 5 min. The sample was cooled to rt, filtered, concentrated in vacuo, and purified by reverse phase HPLC (gradient: 25-55% MeCN in water (w/0.1% TFA)) to give B13 as an off-white solid that was carried forward as a TFA salt (6 mg, 26% yield). ES-MS [M+H]⁺: 405.3, ¹H NMR (400 MHz, DMSO-d₆) δ 10.98 (s, 1H), 10.87 (s, 1H), 8.46 (s, 1H), 8.82-7.98 (m, 2H), 7.96 (dd, J=3.5, 2.1 Hz, 2H), 7.83 (dd, J=8.5, 2.3 Hz, 1H), 7.75 (d, J=8.5 Hz, 1H), 7.62-7.55 (m, 2H), 7.52-7.41 (m, 3H), 2.34 (s, 3H).

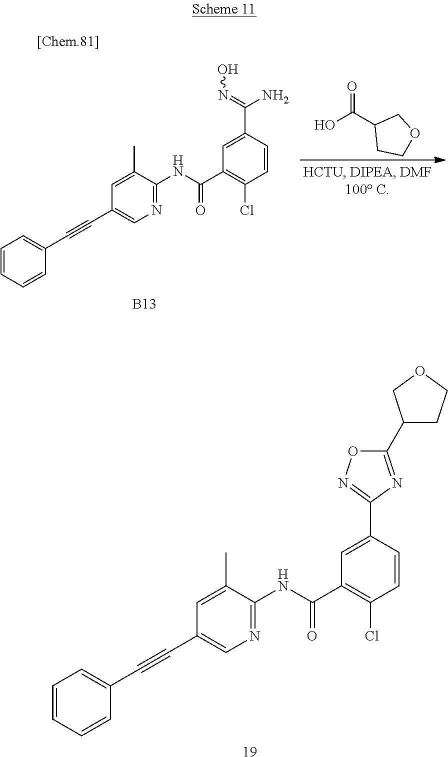

2-chloro-N-(3-methyl-5-(phenylethynyl)pyridin-2-yl)-5-(5-(tetrahydrofuran-3-yl)-1,2,4-oxadiazol-3-yl)benzamide (19): A mixture of Intermediate B13 (5 mg, 0.01 mmol), tetrahydro-3-furoic acid (0.002 mL, 0.022 mmol), HCTU (9 mg, 0.022 mmol), and DIEA (0.004 mL, 0.004 mmol) in DMF (0.5 mL) was heated at 100° C. After 17 h, the reaction mixture was filtered, concentrated in vacuo, and purified by reverse phase HPLC (gradient: 50-80% MeCN in water (w/0.05% NH 4 OH)) to give 19 as an off-white solid (3 mg, 50% yield). ES-MS [M+H]⁺: 485.2, LCMS Retention time: 1.14 min, ¹H NMR (400 MHz, DMSO-d₆) δ 10.95 (s, 1H), 8.45 (s, 1H), 8.15-8.07 (m, 2H), 7.96 (s, 1H), 7.77 (d, J=8.3 Hz, 1H), 7.63-7.55 (m, 2H), 7.48-7.43 (m, 3H), 5.33 (dd, J=8.0, 5.1 Hz, 1H), 4.10-3.80 (m, 2H), 2.45-2.36 (m, 1H), 2.35-2.33 (m, 3H), 2.33-2.22 (m, 1H), 2.11-1.96 (m, 2H).

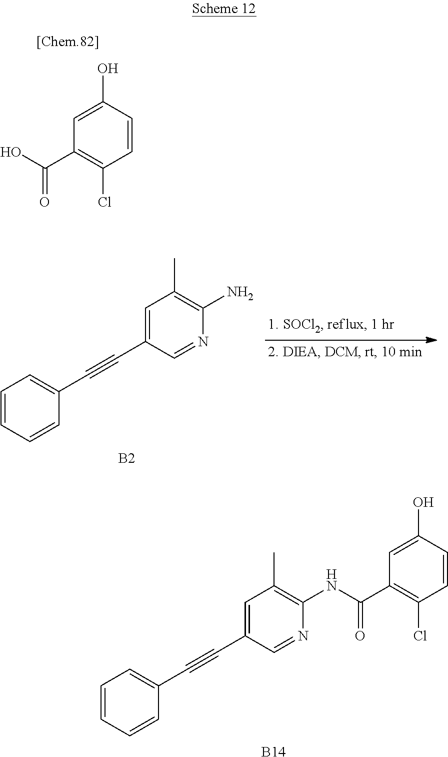

2-chloro-5-hydroxy-N-[3-methyl-5-(2-phenylethynyl)-2-pyridyl]benzamide (B14): A solution of 2-chloro-5-hydroxybenzoic acid (99 mg, 0.58 mmol) in thionyl chloride (3 mL, 41 mmol) was heated to reflux. After 1 hr, the solution was concentrated in vacuo and diluted in DCM (4 mL). A separate solution of intermediate B2 (60 mg, 0.29 mmol) and DIEA (0.05 mL, 0.29 mmol) was added drop-wise. After 10 minutes at room temperatures, to the solution was added a mixture of DIEA (0.15 mL, 0.87 mmol) in DCM dropwise. After 30 min at room temperature, to the solution was added 2N NaOH solution (2 mL, 4 mmol). After 16 hours at room temperature, the solution was diluted with DCM and water. The pH of the solution was adjusted to pH-6 by the dropwise addition of 2N HCl solution. The solution was extracted with DCM (3×10 mL). The combined organic layers were concentrated in vacuo. The residue was purified by reverse phase HPLC (gradient: 25-75% MeCN in water (0.1% TFA in water)). The fractions that contain product were combined and diluted with water and NaHCO₃ solution. The mixture was extracted with EtOAc (2×). The organic layers were washed with water (3×20 mL) and concentrated in vacuo to yield B14 (50 mg, 47% yield), ES-MS [M+1]⁺: 363.2, ¹H NMR (400 MHz, CD 3 OD) δ 8.43 (s, 1H), 7.90 (s, 1H), 7.58-7.54 (m, 2H), 7.42-7.39 (m, 3H), 7.31 (dd, J=8.7, 1.9 Hz, 1H), 7.06 (dd, J=2.5, 2.5 Hz, 1H), 6.90 (ddd, J=8.7, 2.6, 2.6 Hz, 1H), 2.41 (s, 3H).

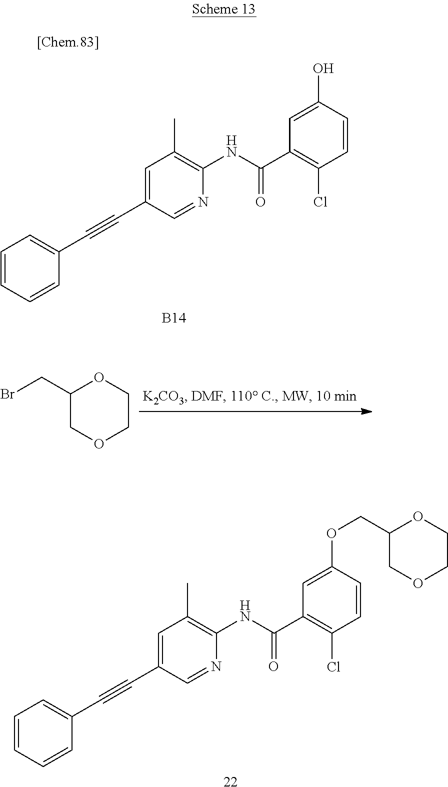

2-chloro-5-(1,4-dioxan-2-ylmethoxy)-N-[3-methyl-5-(2-phenylethynyl)-2-pyridyl]benzamide (22): A solution of 2-chloro-5-hydroxy-N-[3-methyl-5-(2-phenylethynyl)-2-pyridyl]benzamide (intermediate B14) (50 mg, 0.14 mmol), 2-(bromomethyl)dioxane (30 mg, 0.16 mmol), and K₂CO₃ (48 mg, 0.34 mmol) in DMF (2.5 mL) was heated to 110° C. in the microwave synthesizer for 10 minutes. The reaction mixture was purified by reverse phase HPLC (gradient: 30-100% MeCN in water (0.5 mL NH₄OH/1 L water)). Fractions that contain product were combined and concentrated to yield 22 (14.6 mg, 23% yield), ES-MS [M+1]⁺: 463.0. LCMS Retention time: 0.96 min, ¹H NMR (400 MHz, CD 3 OD) δ 8.43 (s, 1H), 7.91 (d, J=1.4 Hz, 1H), 7.58-7.54 (m, 2H), 7.43-7.39 (m, 4H), 7.25 (d, J=3.0 Hz, 1H), 7.08 (dd, J=8.9, 3.0 Hz, 1H), 4.09-4.01 (m, 2H), 3.99-3.94 (m, 1H), 3.89 (dd, J=11.5, 2.6 Hz, 1H), 3.85-3.81 (m, 1H), 3.79-3.71 (m, 2H), 3.63 (ddd, J=14.0, 11.0, 3.3 Hz, 1H), 3.54 (dd, J=11.5, 9.9 Hz, 1H), 2.41 (s, 3H).

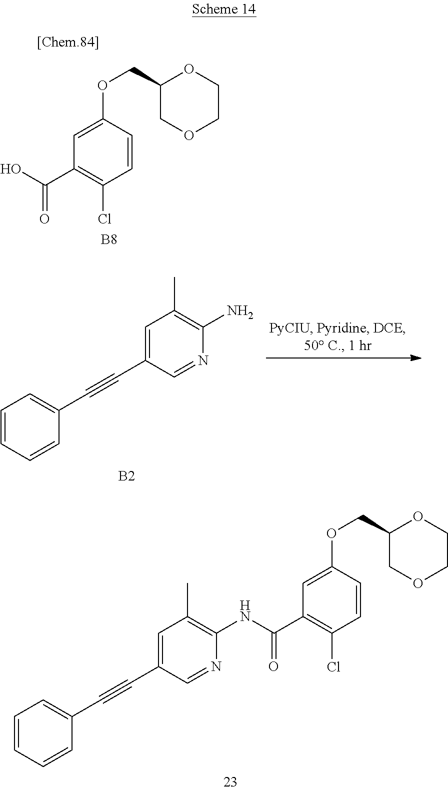

2-chloro-5-[[(2S)-1,4-dioxan-2-yl]methoxy]-N-[3-methyl-5-(2-phenylethynyl)-2-pyridyl]benzamide (23): Equally divided into 7 vials, was added pyridine (5.19 mL, 64.2 mmol), 2-chloro-5-[[(2S)-1,4-dioxan-2-yl]methoxy]benzoic acid (B8) (3500 mg, 12.8 mmol), 3-methyl-5-(2-phenylethynyl)pyridin-2-amine (B2) (4009 mg, 19.3 mmol), and DCE (67 mL). Ater 1 hour at 50° C., the solution was diluted with water (500 mL), and extracted with DCM 3×300 mL. The DCM extractions were washed with water 2×300 mL, combined, and concentrated in vacuo. The residue was purified by reverse phase HPLC (gradient: 30-80% MeCN in water (0.1% TFA in water)). The fractions were partially concentrated in vacuo and diluted with NaHCO₃ solution (0.5 mL), water, and EtOAc. The sample was extracted with EtOAc (2×500 mL). The combined organic layers were washed with water (3×300 mL) and concentrated. The resulting solids were sonicated in MeOH and the resulting slurry was filtered to afford compound 23 (1196 mg, 20% yield) ES-MS [M+1]⁺: 463.2, LCMS Retention time: 1.05 min, ¹H NMR (400 MHz, CDCl₃) δ 9.13 (br s, 1H), 8.40 (s, 1H), 7.91 (s, 1H), 7.57-7.54 (m, 2H), 7.42-7.34 (m, 5H), 7.01 (dd, J=8.9, 3.0 Hz, 1H), 4.06-4.0 (m, 3H), 3.91-3.84 (m, 2H), 3.81 (ddd, J=10.4, 2.7, 2.7 Hz, 1H), 3.75 (d, J=11.3 Hz, 1H), 3.67 (ddd, J=11.5, 10.5, 3.3 Hz, 1H), 3.55 (dd, J=11.3, 9.3 Hz, 1H), 2.44 (s, 3H).

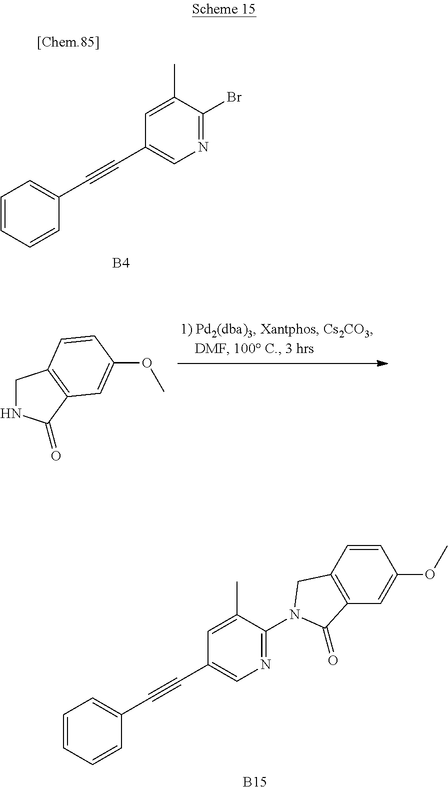

6-methoxy-2-[3-methyl-5-(2-phenylethynyl)-2-pyridyl]isoindolin-1-one (B15): A solution of 6-methoxy-1-isindolinon (50 mg, 0.31 mmol), 2-bromo-3-methyl-5-(2-phenylethynyl)pyridine (B4) (83 mg, 0.31 mmol), Xantphos (11 mg, 0.020 mmol), tris(dibenzylideneacetone)dipalladium(0), (17 mg, 0.02 mmol), and cesium carbonate, (201 mg, 0.61 mmol) in DMF (6.98 mL) was heated to 100° C. for 3 hours. The solution was filtered and the product was purified by reverse phase HPLC (gradient: 30-95% MeCN in water (0.5 mL NH₄OH/1L water)). Fractions that contain product were combined and concentrated to yield B15 (49 mg, 45% yield), ESMS [M+1]⁺: 355.4, LCMS Retention time: 1.10 min, ¹H NMR (400 MHz, CDCl₃) δ 8.49 (d, J=1.9 Hz, 1H), 7.79 (d, J=1.5 Hz, 1H), 7.57-7.55 (m, 2H), 7.42 (d, J=8.5 Hz, 1H), 7.41 (d, J=2.4 Hz, 1H), 7.39-7.36 (m, 3H), 7.19 (dd, 8.3, 2.5 Hz, 1H), 5.01 (s, 2H), 3.90 (s, 3H), 2.38 (s, 3H).

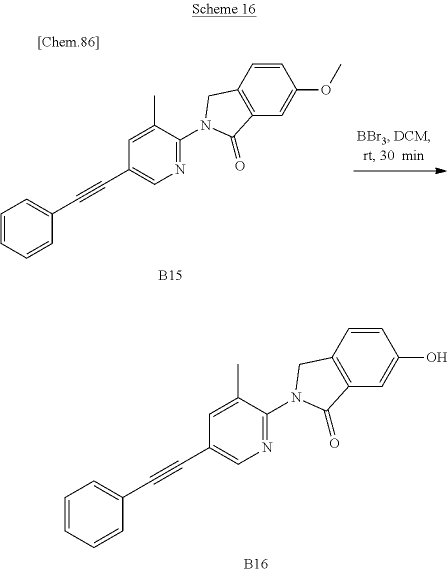

6-hydroxy-2-[3-methyl-5-(2-phenylethynyl)-2-pyridyl]isoindolin-1-one (B16): To a solution of 6-methoxy-2-[3-methyl-5-(2-phenylethynyl)-2-pyridyl]isoindolin-1-one (B15) (49 mg, 0.14 mmol) in DCM (3.97 mL) at room temperature was added dropwise boron tribromide (0.05 mL, 0.53 mmol). After 45 min at room temperature, the reaction mixture was cooled in an ice bath and water (1 mL) was added dropwise. The solution was removed from the ice bath and diluted with NaHCO₃ solution (3 mL). The mixture was extracted with DCM (2×3 mL). The combined organic layers were concentrated, and the residue was purified by reverse phase HPLC (gradient: 30-90% MeCN in water (0.5 mL NH 4 OH/1 L of water)). Fractions that contain product were combined and concentrated to yield B16 (12.6 mg, 27% yield), ES-MS [M+1]⁺: 341.4.

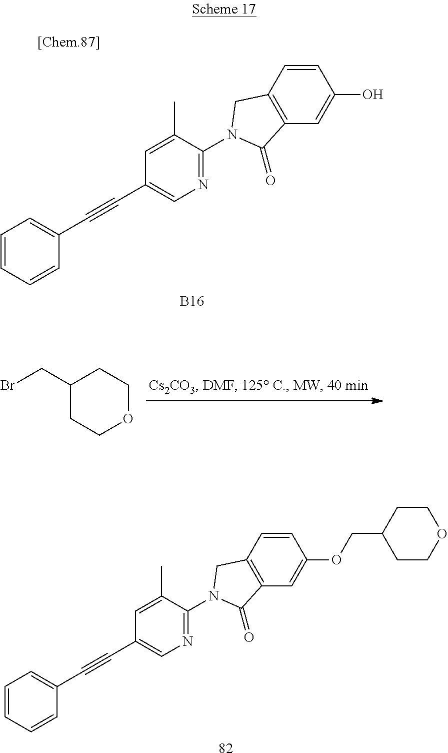

2-[3-methyl-5-(2-phenylethynyl)-2-pyridyl]-6-(tetrahydropyran-4-ylmethoxy)isoindolin-1-one (82): A solution of 6-hydroxy-2-[3-methyl-5-(2-phenylethynyl)-2-pyridyl]isoindolin-1-one (B16) (12.6 mg, 0.04 mmol), 4-bromomethyltetrahydropyran (20 mg, 0.11 mmol), and cesium carbonate (36 mg, 0.11 mmol) in DMF (0.63 mL) in a 2 mL MW vial was heated to 125° C. in a microwave synthesizer for 40 min. The solution was purified by reverse phase HPLC (gradient: 40-95% MeCN in water (0.5 mL NH 4 OH/1 L water)). Fractions that contain isolated product were concentrated to yield 82 (7.6 mg, 47% yield), ESMS [M+1]⁺: 439.4, LCMS Retention time: 1.15 min, ¹H NMR (400 MHz, D4-MeOD) δ 8.51 (d, J=1.8 Hz, 1H), 7.96 (d, J=1.5 Hz, 1H), 7.59-7.56 (m, 3H), 7.42-7.41 (m, 3H), 7.37 (d, J=2.3 Hz, 1H), 7.29 (dd, J=8.4, 2.4 Hz, 1H), 5.01 (s, 2H), 4.00 (dd, J=11.2, 3.2 Hz, 2H), 3.95 (d, J=6.3 Hz, 2H), 3.49 (ddd, J=11.9, 11.9, 2.0 Hz, 2H), 2.34 (s, 3H), 2.18-2.08 (m, 1H), 1.81 (dd, J=13.0, 1.9 Hz, 2H), 1.50 (dddd, J=12.1, 12.1, 12.1, 4.5 Hz, 2H).

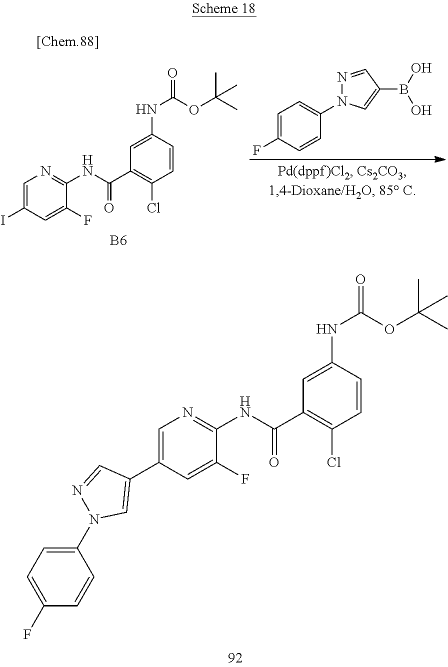

tert-butyl (4-chloro-3-((3-fluoro-5-(1-(4-fluorophenyl)-1H-pyrazol-4-yl)pyridin-2-yl)carbamoyl) phenyl)carbamate (92): To a mixture of B6 (50 mg, 0.102 mmol), 1-(4-fluorophenyl)pyrazole-4-boronic acid (42 mg, 0.203 mmol), Pd(dppf)Cl₂ (8 mg, 0.010 mmol), and Cs₂CO₃ (73 mg, 0.224 mmol) in 1,4-dioxane (1 mL) and H₂O (0.220 mL) was heated to 85° C. After 1 h, the reaction mixture was cooled to rt, filtered, concentrated in vacuo, and purified by reverse phase HPLC (gradient: 50-90% MeCN in water (w/0.05% NH 4 OH)). The desired fractions were concentrated to yield compound 92 (25 mg, 47% yield). ES-MS [M+1]⁺: 526.2, LCMS Retention time: 1.13 min, ¹H NMR (400 MHz, CDCl₃) δ 10.82 (s, 1H), 9.68 (s, 1H), 9.16 (s, 1H), 8.68 (s, 1H), 8.38 (s, 1H), 8.18 (d, J=11.1 Hz, 1H), 7.95-7.87 (m, 2H), 7.77 (s, 1H), 7.51 (dd, J=8.8, 2.6 Hz, 1H), 7.46-7.34 (m, 3H), 1.49 (s, 9H).

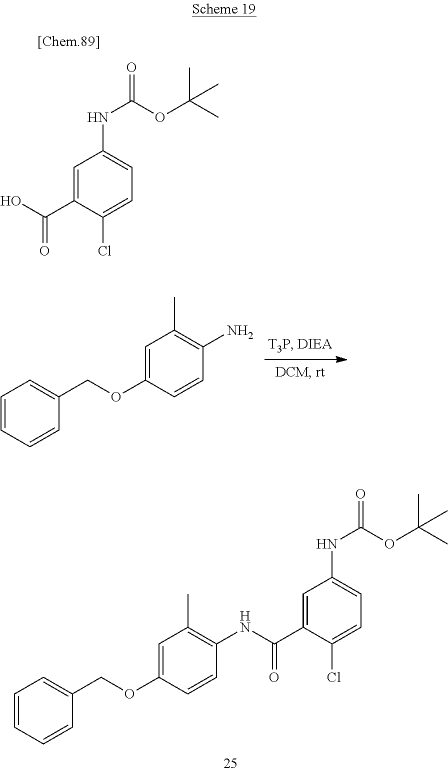 [SMILES: CC(C)(C)OC(=O)Nc1ccc(Cl)c(C(=O)O)c1 ; Cc1cc(OCc2ccccc2)ccc1N ; Cc1cc(OCc2ccccc2)ccc1NC(=O)c1cc(NC(=O)OC(C)(C)C)ccc1Cl]

tert-butyl (3-((4-(benzyloxy)-2-methylphenyl)carbamoyl)-4-chlorophenyl)carbamate (25): To a solution of 5-(tert-butoxycarbonylamino)-2-chloro-benzoic acid (500 mg, 1.84 mmol), 4-(benzyloxy)-2-methylaniline (393 mg, 1.84 mmol), DIEA (0.96 mL, 5.52 mmol) in DCM (30 mL) was added a solution of T₃P in ethyl acetate (50%, 1.6 mL, 2.7 mmol). After the reaction mixture was stirred for 32h at room temperature, the reaction mixture was poured into NH₄Cl-aq and extracted with DCM twice. The combined organic layer was washed with brine and dried over MgSO₄. The filtrate was concentrated under reduced pressure to give a crude product, which was purified by column chromatography on silica gel and the concentrated residue was triturated with methanol to yield (25) as a white powder (660 mg, 77% yield). ES-MS [M+1]⁺: 467.3, LCMS Retention time: 1.15 min (LCMS method-2), ¹H NMR (400 MHz, DMSO-d₆) δ 9.80 (br, 1H), 9.66 (br, 1H), 7.72 (d, J=2.5 Hz, 1H), 7.51 (dd, J=9.0, 2.5 Hz, 1H), 7.47-7.39 (m, 5H), 7.36-7.32 (m, 1H), 7.24 (d, J=9.0 Hz, 1H), 6.94 (d, J=2.5 Hz, 1H), 6.87 (dd, J=9.0, 2.5 Hz, 1H), 5.11 (s, 2H), 2.25 (s, 3H), 1.49 (s, 9H).

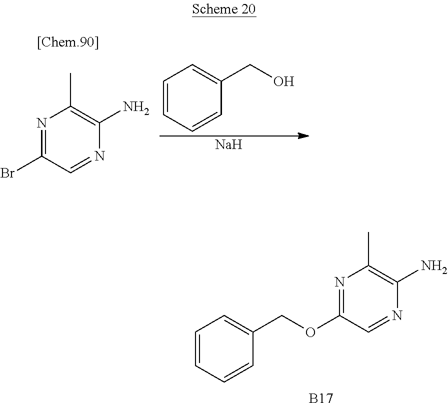

5-(benzyloxy)-3-methylpyrazin-2-amine (B17): A dry 10 mL microwave vial, equipped with a magnetic stir bar and nitrogen atmosphere, was charged with 5-bromo-3-methylpyrazin-2-amine (135 mg, 0.718 mmol) in benzyl alcohol (0.80 mL, 7.7 mmol) and the reaction mixture cooled to 0° C. 60% Sodium hydride in mineral oil (31.6 mg, 0.790 mmol) was added in two portions; one at 0° C. and the other at ambient temperature. The reaction vial was sealed and the reaction mixture heated at 120° C. for 5 h then stirred at ambient temperature overnight. After this time, the reaction mixture was diluted with water (40 mL) and extracted with dichloromethane (3×25 mL). The combined organic layers were washed with brine (20 mL) and concentrated under reduced pressure. The resulting residue was purified by chromatography (silica, heptane to 40% ethyl acetate/heptane, gradient), followed by reverse phase chromatography (C18, water to acetonitrile, gradient). The combined column fractions were diluted with water (20 mL) and extracted with ethyl acetate (3×20 mL). The combined organic layers were washed with brine (20 mL), dried over sodium sulfate, filtered, and the filtrate concentrated under reduced pressure to afford 5-(benzyloxy)-3-methylpyrazin-2-amine (B17) (52 mg, 34%) as an off-white solid: ¹H NMR (500 MHz, CDCl₃) δ 7.66-7.65 (m, 1H), 7.46-7.43 (m, 2H), 7.38-7.35 (m, 2H), 7.33-7.29 (m, 1H), 5.29 (s, 2H), 4.06 (br s, 2H), 2.35 (d, J=1.0 Hz, 3H).

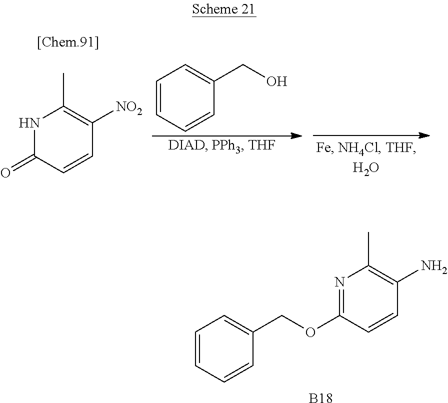

6-(benzyloxy)-2-methylpyridin-3-amine (B18): A dry 50 mL round bottom flask, equipped with a magnetic stir bar and nitrogen atmosphere, was charged with 6-methyl-5-nitropyridin-2(1H)-one (260 mg, 1.69 mmol), benzyl alcohol (177 mg, 1.64 mmol) and triphenylphosphine (878 mg, 3.35 mmol) in tetrahydrofuran (3 mL) and the reaction mixture cooled to 0° C. Diisopropyl azodicarboxylate (0.80 mL, 4.1 mmol) was added and the reaction mixture slowly warmed to ambient temperature while stirring overnight. After this time, the reaction mixture was concentrated under reduced pressure and the resulting residue purified by chromatography (silica, heptane to 30% ethyl acetate/heptane, gradient), followed by chromatography (silica, heptane to 25% ethyl acetate/heptane, gradient) to afford 6-(benzyloxy)-2-methyl-3-nitropyridine (95.5 mg, 24%) as an off-white solid: ¹H NMR (500 MHz, DMSO) δ 8.39 (d, J=9.0 Hz, 1H), 7.50-7.47 (m, 2H), 7.42-7.38 (m, 2H), 7.36-7.33 (m, 1H), 6.94 (d, J=9.0 Hz, 1H), 5.46 (s, 2H), 2.74 (s, 3H).

A dry 10 mL microwave vial, equipped with a magnetic stir bar and nitrogen atmosphere, was charged with 6-(benzyloxy)-2-methyl-3-nitropyridine (42.8 mg, 0.175 mmol) in tetrahydrofuran (3 mL). Ammonium chloride (40 mg, 0.75 mmol) in water (2 mL) was added and the reaction mixture stirred at 75° C. for 30 minutes. The reaction mixture was cooled, iron powder (39 mg, 0.70 mmol) was added and the reaction mixture stirred at 75° C. for 5 h. After this time, the reaction mixture was cooled to room temperature and filtered through a pad of diatomaceous earth. The filtrate was neutralized with saturated aqueous sodium bicarbonate (40 mL) and extracted with ethyl acetate (3×10 mL). The combined organic layers were washed with brine (20 mL), dried over sodium sulfate, filtered and the filtrate concentrated under reduced pressure to afford 6-(benzyloxy)-2-methylpyridin-3-amine (B18, 32.5 mg, 87%) as a yellow oil: ¹H NMR (500 MHz, DMSO) δ 7.42-7.40 (m, 2H), 7.37-7.34 (m, 2H), 7.30-7.27 (m, 1H), 6.99 (d, J=8.5 Hz, 1H), 6.46 (d, J=8.5 Hz, 1H), 5.19 (s, 2H), 4.51 (br s, 2H), 2.20 (s, 3H).

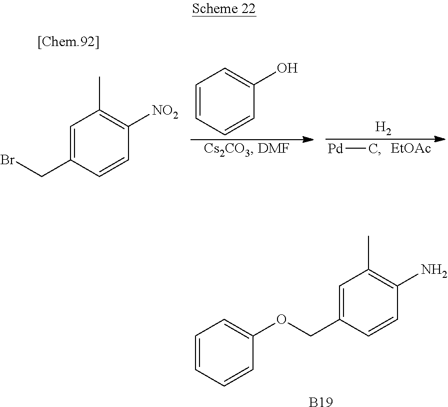

2-methyl-4-(phenoxymethyl)aniline (B19): A dry 50 mL round bottom flask, equipped with a magnetic stir bar and nitrogen atmosphere, was charged with 4-(bromomethyl)-2-methyl-1-nitrobenzene (230 mg, 1.00 mmol) and phenol (147 mg, 1.56 mmol) in N,N-dimethylformamide (2 mL). Cesium carbonate (455 mg, 1.40 mmol) was added and the reaction mixture stirred at ambient temperature overnight. After this time, the reaction mixture was diluted with water (40 mL) and extracted with ethyl acetate (3×30 mL). The combined organic layers were washed with brine (20 mL), dried over sodium sulfate, filtered and the filtrate concentrated under reduced pressure. The resulting residue was purified by chromatography (silica, heptane to 20% ethyl acetate/heptane, gradient) to afford 2-methyl-1-nitro-4-(phenoxymethyl)benzene (136 mg, 56%) as a clear oil: ¹H NMR (500 MHz, CDCl₃) 8.01 (d, J=8.0 Hz, 1H), 7.41-7.39 (m, 2H), 7.33-7.29 (m, 2H), 7.00 (tt, J=8.0, 1.0 Hz, 1H), 6.98-6.95 (m, 2H), 5.11 (s, 2H), 2.63 (s, 3H).

A dry 100 mL round bottom flask, equipped with a magnetic stir bar and nitrogen atmosphere was charged with 2-methyl-1-nitro-4-(phenoxymethyl)benzene (135 mg, 0.555 mmol) in ethyl acetate (20 mL). 5% Palladium on carbon (50% wet, 118 mg) was added, the solution sparged with hydrogen and the reaction mixture stirred under balloon pressure of hydrogen at ambient temperature for 1 h. After this time, the reaction mixture was degassed with nitrogen, filtered through diatomaceous earth, and the filter cake washed with ethyl acetate (3×15 mL). The filtrate was concentrated under reduced pressure to afford 2-methyl-4-(phenoxymethyl)aniline (B19, 93 mg, 79%) as a clear oil: ¹H NMR (500 MHz, CDCl₃) 7.29-7.26 (m, 2H), 7.14 (d, J=1.5 Hz, 1H), 7.10 (dd, J=8.0, 1.5 Hz, 1H), 6.98-6.93 (m, 3H), 6.78 (d, J=8.0 Hz, 1H), 4.91 (s, 2H), 3.60 (br s, 2H), 2.18 (s, 3H).

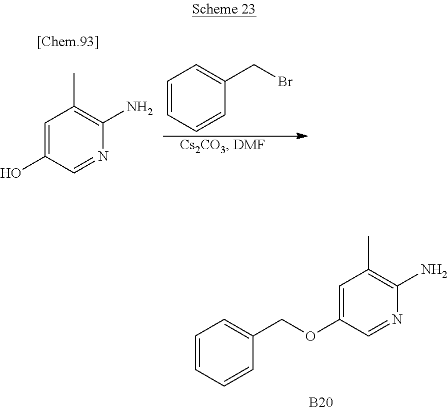

5-(benzyloxy)-3-methylpyridin-2-amine (B20): A dry 50 mL round bottom flask, equipped with a magnetic stir bar and nitrogen atmosphere, was charged with 6-amino-5-methylpyridin-3-ol (90 mg, 0.73 mmol) and cesium carbonate (360 mg, 1.11 mmol) in N,N-dimethylformamide (6 ml). Benzyl bromide (0.086 ml, 0.73 mmol) was added and the reaction mixture stirred at ambient temperature for 22 h. After this time, the reaction mixture was diluted with water (40 mL) and extracted with ethyl acetate (3×30 mL). The combined organic layers were washed with brine (20 mL) and concentrated under reduced pressure. The resulting residue was purified by chromatography (silica, heptane to ethyl acetate, gradient) to afford 5-(benzyloxy)-3-methylpyridin-2-amine (B20, 18 mg, 12%) as an off-white solid: ¹H NMR (500 MHz, CDCl₃) δ 7.72 (d, J=2.5 Hz, 1H), 7.42-7.36 (m, 4H), 7.33-7.30 (m, 1H), 7.03 (d, J=2.5 Hz, 1H), 5.00 (s, 2H), 4.12 (br s, 2H), 2.12 (s, 3H).

The compounds shown in Table 1 were prepared in an analogous manner with the appropriate starting materials, and evaluated in our TREK-1 Thallium (T1⁺) flux assay.

TABLE 1

| | | | ES- | Tl⁺ |
|---|---|---|---|---|
| | | | MS | Assay |
| Cpd. | | | [M + | IC₅₀ |
| No. | Name | Structure | 1]⁺ | (μM) |
| 1 | tert-butyl N-[4-chloro-3-[[3-fluoro-5- (2-phenylethynyl)-2- pyridyl]carbamoyl]phenyl]carbamate | 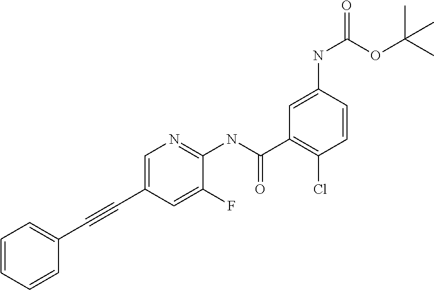 [SMILES: CC(C)(C)OC(=O)Nc1ccc(Cl)c(C(=O)Nc2ncc(C#Cc3ccccc3)cc2F)c1] | 466 | 0.11 |
| 2 | 2-chloro-N-[3-fluoro-5-(2- phenylethynyl)-2-pyridyl]-5- [[(1S,2S)-2- methylcyclopropanecarbonyl]amino] benzamide | 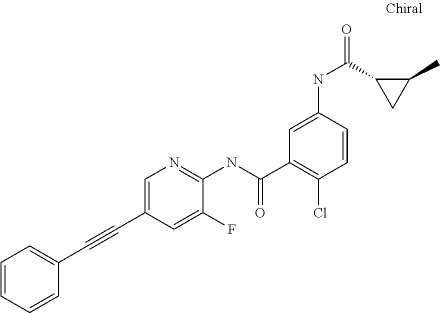 [SMILES: C[C@H]1C[C@@H]1C(=O)Nc1ccc(Cl)c(C(=O)Nc2ncc(C#Cc3ccccc3)cc2F)c1] | 448 | 0.09 |
| 3 | 2-chloro-N-[3-fluoro-5-(2- phenylethynyl)-2-pyridyl]-5-[(2- methylcyclopropanecarbonyl)amino] benzamide | 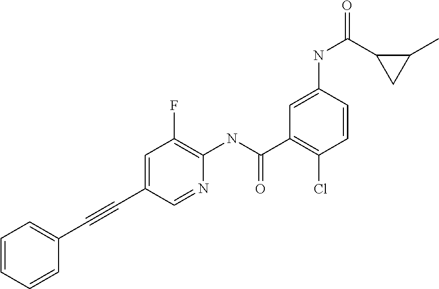 [SMILES: CC1CC1C(=O)Nc1ccc(Cl)c(C(=O)Nc2ncc(C#Cc3ccccc3)cc2F)c1] | 448.0 | 0.07 |
| 4 | 2-chloro-N-[3-fluoro-5-[2-(3- fluorophenyl)ethynyl]-2-pyridyl]-5- [[(1S,2S)-2- methylcyclopropanecarbonyl]amino] benzamide | 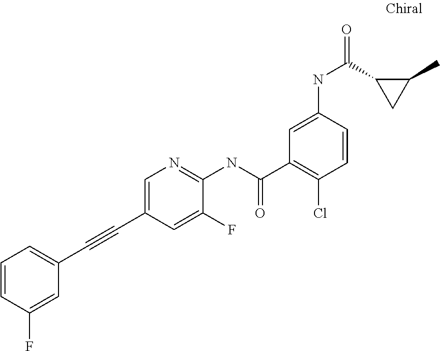 [SMILES: C[C@H]1C[C@@H]1C(=O)Nc1ccc(Cl)c(C(=O)Nc2ncc(C#Cc3cccc(F)c3)cc2F)c1] | 466 | 0.13 |
| 5 | tert-butyl N-[4-chloro-3-[[5-[2-(3- fluorophenyl)ethynyl]-3-methyl-2- pyridyl]carbamoyl]phenyl]carbamate | 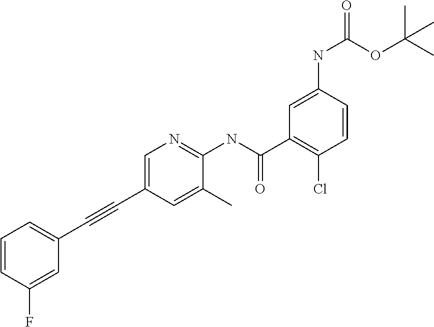 [SMILES: Cc1cc(C#Cc2cccc(F)c2)cnc1NC(=O)c1cc(NC(=O)OC(C)(C)C)ccc1Cl] | 480.3 | 0.21 |
| 6 | 2-chloro-5-[(1- fluorocyclopropanecarbonyl)amino]- N-[3-fluoro-5-(2-phenylethynyl)-2- pyridyl]benzamide | 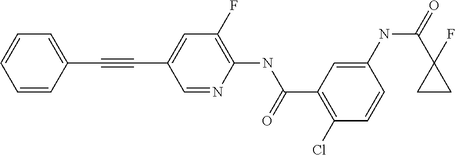 [SMILES: O=C(Nc1ncc(C#Cc2ccccc2)cc1F)c1cc(NC(=O)C2(F)CC2)ccc1Cl] | 452 | 0.17 |
| 7 | 2-chloro-5- (cyclopropanecarbonylamino)-N-[3- fluoro-5-[2-(4-fluorophenyl)ethynyl]- 2-pyridyl]benzamide | 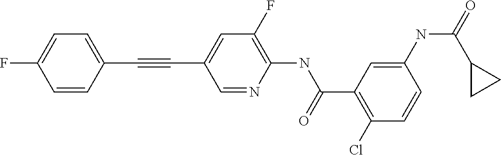 [SMILES: O=C(Nc1ncc(C#Cc2ccc(F)cc2)cc1F)c1cc(NC(=O)C2CC2)ccc1Cl] | 451.9 | 0.15 |
| 8 | tert-butyl N-[4-chloro-3-[[5-[2-(4- fluorophenyl)ethynyl]-3-methyl-2- pyridyl]carbamoyl]phenyl]carbamate | 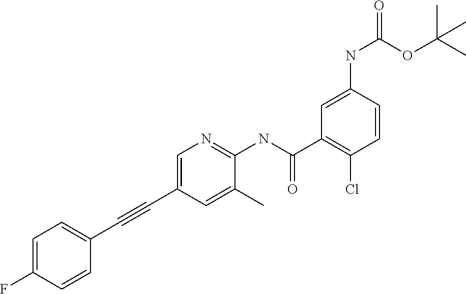 [SMILES: Cc1cc(C#Cc2ccc(F)cc2)cnc1NC(=O)c1cc(NC(=O)OC(C)(C)C)ccc1Cl] | 480.5 (meth- od-2) | 0.26 |
| 9 | 2-chloro-5- (cyclopropanecarbonylamino)-N-[3- methyl-5-(2-phenylethynyl)-2- pyridyl]benzamide | 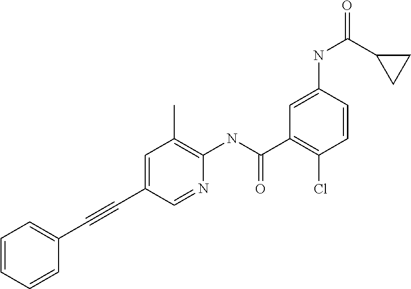 [SMILES: Cc1cc(C#Cc2ccccc2)cnc1NC(=O)c1cc(NC(=O)C2CC2)ccc1Cl] | 430.2 | 0.18 |
| 10 | N-[4-chloro-3-[[3-flluoro-5-(2- phenylethynyl)-2- pyridyl]carbamooyl]phenyl]-1,4- dioxane-2-carboxamide | 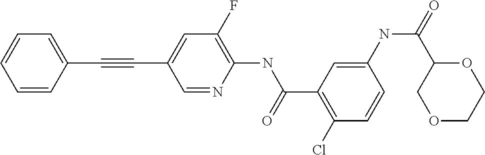 [SMILES: O=C(Nc1ncc(C#Cc2ccccc2)cc1F)c1cc(NC(=O)C2COCCO2)ccc1Cl] | 480 | 0.20 |
| 11 | tert-butyl N-[4-chloro-3-[[6-[2-(3- fluorophenyl)ethynyl]-2,4-dimethyl- 3- pyridyl]carbamoyl]phenyl]carbamate | 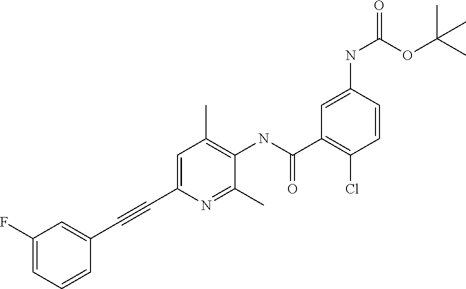 [SMILES: Cc1cc(C#Cc2cccc(F)c2)nc(C)c1NC(=O)c1cc(NC(=O)OC(C)(C)C)ccc1Cl] | 476.2 | 0.41 |
| 12 | N-[4-chloro-3-[[5-[2-(4- fluorophenyl)ethynyl]-3-methyl-2- pyridyl]carbamoyl]phenyl]oxetane-2- carboxamide | 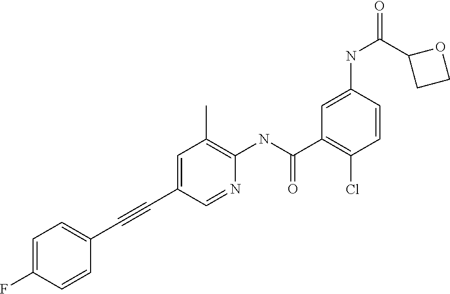 [SMILES: Cc1cc(C#Cc2ccc(F)cc2)cnc1NC(=O)c1cc(NC(=O)C2CCO2)ccc1Cl] | 464 | 0.31 |
| 13 | N-[4-chloro-3-[[5-[2-(3- fluorophenyl)ethynyl]-3-methyl-2- pyridyl]carbamoyl]phenyl]-1,4- dioxane-2-carboxamide | 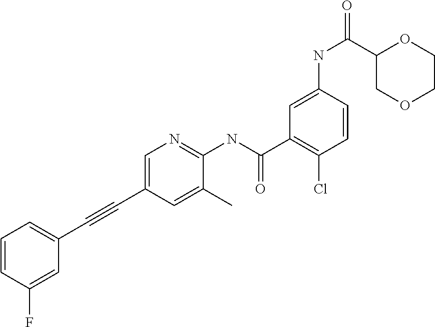 [SMILES: Cc1cc(C#Cc2cccc(F)c2)cnc1NC(=O)c1cc(NC(=O)C2COCCO2)ccc1Cl] | 494 | 0.28 |
| 14 | N-[4-chloro-3-[[5-[2-(4- fluorophenyl)ethynyl]-3-methyl-2- pyridyl]carbamoyl]phenyl]-1,4- dioxane-2-carboxamide | 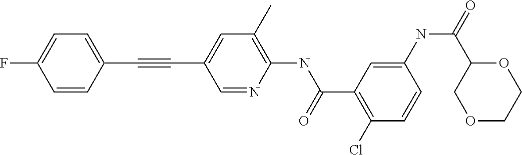 [SMILES: Cc1cc(C#Cc2ccc(F)cc2)cnc1NC(=O)c1cc(NC(=O)C2COCCO2)ccc1Cl] | 494 | 0.23 |
| 15 | tert-butyl N-[4-chloro-3-[[3-methyl- 5-[2-(2-pyridyl)ethynyl]-2- pyridyl]carbamoyl]phenyl]carbamate | 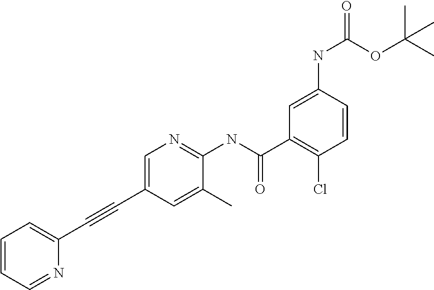 [SMILES: Cc1cc(C#Cc2ccccn2)cnc1NC(=O)c1cc(NC(=O)OC(C)(C)C)ccc1Cl] | 463.4 | 0.37 |
| 16 | 2-chloro-5-(3,3-difluoroazetidine-1- carbonyl)-N-[3-methyl-5-(2- phenylethynyl)-2-pyridyl]benzamide | 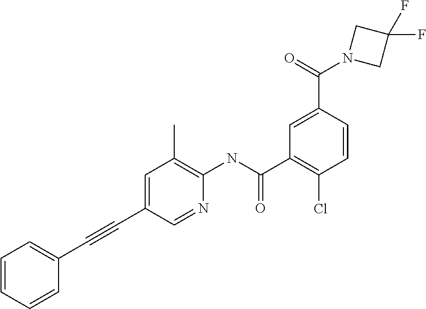 [SMILES: Cc1cc(C#Cc2ccccc2)cnc1NC(=O)c1cc(C(=O)N2CC(F)(F)C2)ccc1Cl] | 466 | 0.32 |
| 17 | 4-chloro-N1-(2-hydroxy-1,1- dimethyl-ethyl)-N3-[3-methyl-5-(2- phenylethynyl)-2-pyridyl]benzene- 1,3-dicarboxamide | 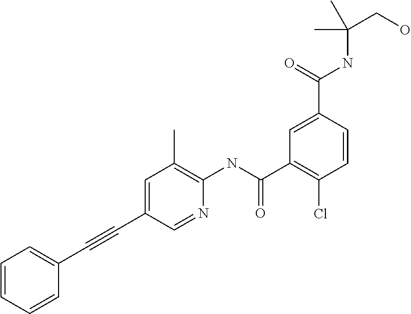 [SMILES: Cc1cc(C#Cc2ccccc2)cnc1NC(=O)c1cc(C(=O)NC(C)(C)CO)ccc1Cl] | 462.4 | 0.18 |
| 18 | 2-chloro-N-[5-[2-(3- fluorophenyl)ethynyl]-3-methyl-2- pyridyl]-5-(3-hydroxy-3-methyl- azetidine-1-carbonyl)benzamide | 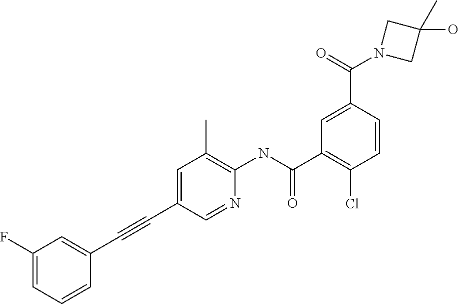 [SMILES: Cc1cc(C#Cc2cccc(F)c2)cnc1NC(=O)c1cc(C(=O)N2CC(C)(O)C2)ccc1Cl] | 478 | 0.24 |
| 19 | 2-chloro-N-[3-methyl-5-(2- phenylethynyl)-2-pyridyl]-5-(5- tetrahydrofuran-3-yl-1,2,4-oxadiazol- 3-yl)benzamide | 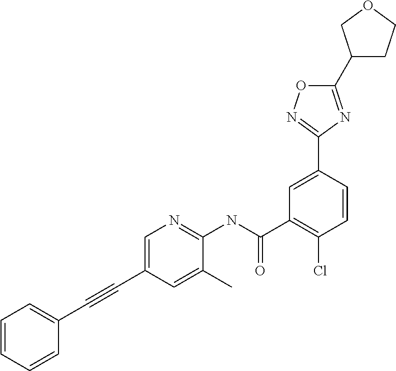 [SMILES: Cc1cc(C#Cc2ccccc2)cnc1NC(=O)c1cc(-c2noc(C3CCOC3)n2)ccc1Cl] | 485.2 | 0.05 |
| 20 | 2-chloro-N-[3-methyl-5-(2- phenylethynyl)-2-pyridyl]-5- (tetrahydrofuran-4- ylmethoxy)benzamide | 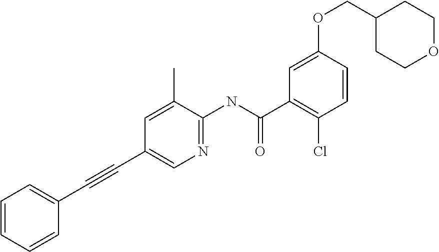 [SMILES: Cc1cc(C#Cc2ccccc2)cnc1NC(=O)c1cc(OCC2CCOCC2)ccc1Cl] | 461.2 | 0.09 |
| 21 | 2-chloro-5-[[(2R)-1,4-dioxan-2- yl]methoxy]-N-[3-methyl-5-(2- phenylethynyl)-2-pyridyl]benzamide | 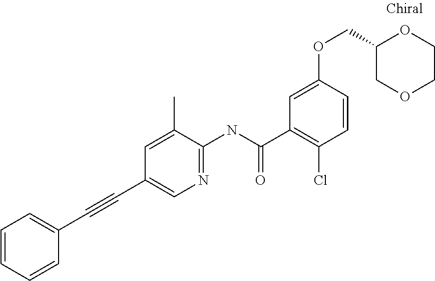 [SMILES: Cc1cc(C#Cc2ccccc2)cnc1NC(=O)c1cc(OC[C@H]2COCCO2)ccc1Cl] | 463.2 | 0.11 |
| 22 | 2-chloro-5-(1,4-dioxan-2- ylmethoxy)-N-[3-methyl-5-(2- phenylethynyl)-2-pyridyl]benzamide | 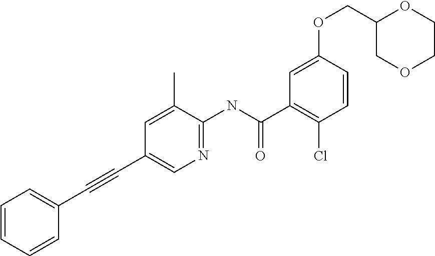 [SMILES: Cc1cc(C#Cc2ccccc2)cnc1NC(=O)c1cc(OCC2COCCO2)ccc1Cl] | 463 | 0.13 |
| 23 | 2-chloro-5-[[(2S)-1,4-dioxan-2- yl]methoxy]-N-(3-methyl-5-(2- phenylethynyl)-2-pyridyl]benzamide | 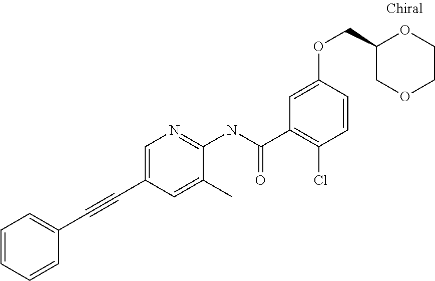 [SMILES: Cc1cc(C#Cc2ccccc2)cnc1NC(=O)c1cc(OC[C@@H]2COCCO2)ccc1Cl] | 463.3 | 0.21 |
| 24 | 2-chloro-5-(2-methoxyethoxy)-N-[3- methyl-5-(2-phenylethynyl)-2- pyridyl]benzamide | 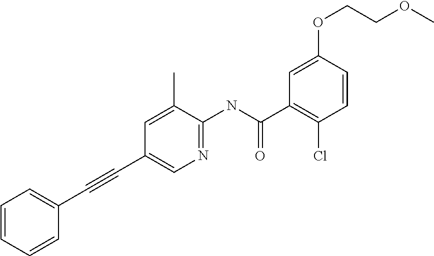 [SMILES: COCCOc1ccc(Cl)c(C(=O)Nc2ncc(C#Cc3ccccc3)cc2C)c1] | 421.3 | 0.23 |
| 25 | tert-butyl (3-((4-(benzyloxy)-2- methylphenyl)carbamoyl)-4- chlorophenyl)carbamate | 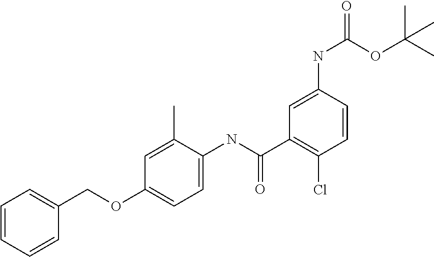 [SMILES: Cc1cc(OCc2ccccc2)ccc1NC(=O)c1cc(NC(=O)OC(C)(C)C)ccc1Cl] | 467.3 (meth- od-2) | 0.46 |
| 26 | tert-butyl (4-chloro-3-((2-methyl-4- (phenoxymethyl)phenyl)carbamoyl) phenyl)carbamate | 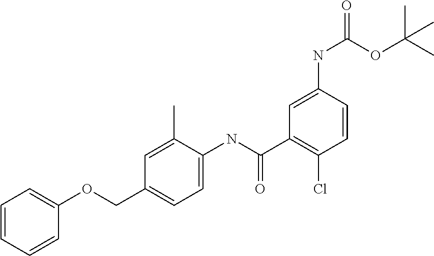 [SMILES: Cc1cc(COc2ccccc2)ccc1NC(=O)c1cc(NC(=O)OC(C)(C)C)ccc1Cl] | 467.2 (meth- od-3) | 0.67 |
| 27 | 2-chloro-5-[[(1S,2S)-2- methylcyclopropanecarbonyl]amino]- N-[3-methyl-5-(2-phenylethynyl)-2- pyridyl]benzamide | 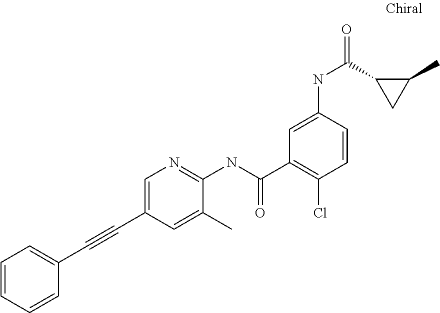 [SMILES: Cc1cc(C#Cc2ccccc2)cnc1NC(=O)c1cc(NC(=O)[C@H]2C[C@@H]2C)ccc1Cl] | 444 | 0.10 |
| 28 | tert-butyl N-[4-chloro-3-[[3-methyl- 5-(2-phenylethynyl)-2- pyridyl]carbamoyl]phenyl]carbamate | 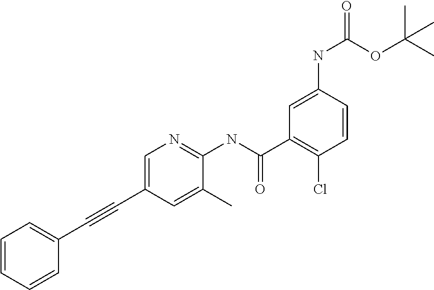 [SMILES: Cc1cc(C#Cc2ccccc2)cnc1NC(=O)c1cc(NC(=O)OC(C)(C)C)ccc1Cl] | 462.4 | 0.14 |
| 29 | tert-butyl N-[4-chloro-3-[[2-fluoro-6- methyl-4-(2- phenylethynyl)phenyl]carbamoyl] phenyl]carbamate | 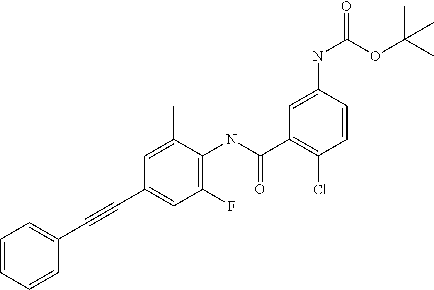 [SMILES: Cc1cc(C#Cc2ccccc2)cc(F)c1NC(=O)c1cc(NC(=O)OC(C)(C)C)ccc1Cl] | 423 | 0.21 |
| 30 | tert-butyl N-[4-chloro-3-[[2,6- difluoro-4-(2- phenylethynyl)phenyl]carbamoyl] phenyl]carbamate | 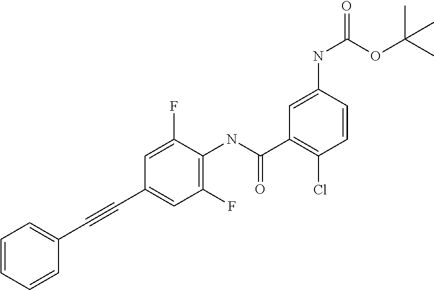 [SMILES: CC(C)(C)OC(=O)Nc1ccc(Cl)c(C(=O)Nc2c(F)cc(C#Cc3ccccc3)cc2F)c1] | 427 | 0.22 |
| 31 | N-[4-chloro-3-[[3-methyl-5-(2- phenylethynyl)-2- pyridyl]carbamoyl]phenyl]-1,4- dioxane-2-carboxamide | 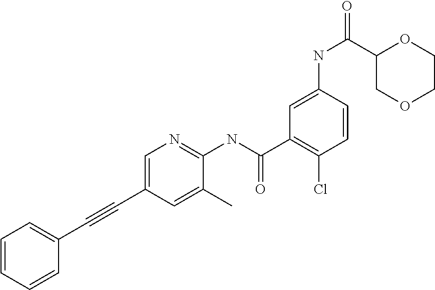 [SMILES: Cc1cc(C#Cc2ccccc2)cnc1NC(=O)c1cc(NC(=O)C2COCCO2)ccc1Cl] | 476 | 0.26 |
| 32 | 2-chloro-5-[(3-hydroxy-3-methyl- cyclobutanecarbonyl)amino]-N-[3- methyl-5-(2-phenylethynyl)-2- pyridyl]benzamide | 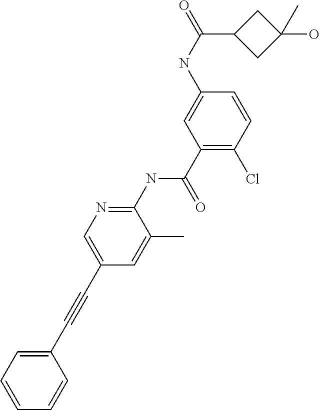 [SMILES: Cc1cc(C#Cc2ccccc2)cnc1NC(=O)c1cc(NC(=O)C2CC(C)(O)C2)ccc1Cl] | 474.4 | 0.28 |
| 33 | 3-chloro-6-[(2- methylcyclopropanecarbonyl)amino]- N-[3-methyl-5-(2-phenylethynyl)-2- pyridyl]pyridine-2-carboxamide | 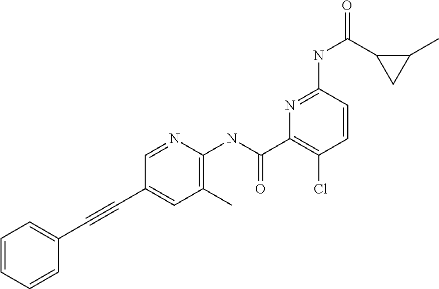 [SMILES: Cc1cc(C#Cc2ccccc2)cnc1NC(=O)c1nc(NC(=O)C2CC2C)ccc1Cl] | 445 | 0.29 |
| 34 | tert-butyl N-[4-chloro-2-fluoro-3-[[3- methyl-5-(2-phenylethynyl)-2- pyridyl]carbamoyl]phenyl]carbamate | 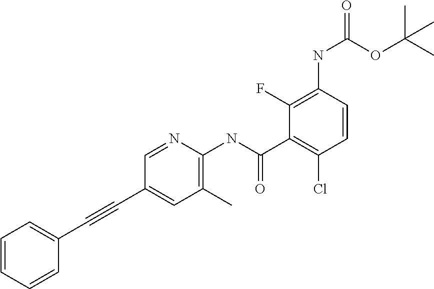 [SMILES: Cc1cc(C#Cc2ccccc2)cnc1NC(=O)c1c(Cl)ccc(NC(=O)OC(C)(C)C)c1F] | 480 | 0.36 |
| 35 | tert-butyl N-[4-chloro-3-fluoro-5-[[3- methyl-5-(2-phenylethynyl)-2- pyridyl]carbamoyl]phenyl]carbamate | 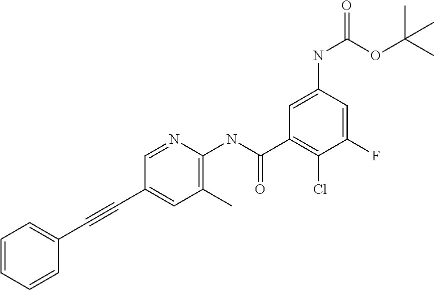 [SMILES: Cc1cc(C#Cc2ccccc2)cnc1NC(=O)c1cc(NC(=O)OC(C)(C)C)cc(F)c1Cl] | 480 | 0.40 |
| 36 | 2-chloro-5-[[(1R,2R)-2- methylcyclopropanecarbonyl]amino]- N-[3-methyl-5-(2-phenylethynyl)-2- pyridyl]benzamide | 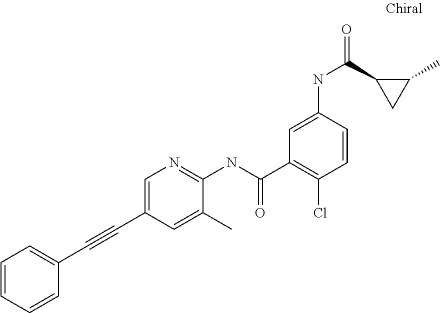 [SMILES: Cc1cc(C#Cc2ccccc2)cnc1NC(=O)c1cc(NC(=O)[C@@H]2C[C@H]2C)ccc1Cl] | 444.0 | 0.42 |
| 37 | tert-butyl N-[4-chloro-3-[[3-chloro-5- (2-phenylethynyl)-2- pyridyl]carbamoyl]phenyl]carbamate | 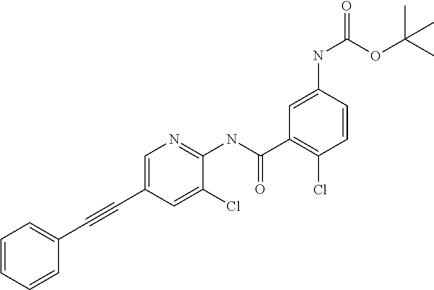 [SMILES: CC(C)(C)OC(=O)Nc1ccc(Cl)c(C(=O)Nc2ncc(C#Cc3ccccc3)cc2Cl)c1] | 482.2 | 0.43 |
| 38 | tert-butyl N-[5-chloro-6-[[3-fluoro-5- (2-phenylethynyl)-2- pyridyl]carbamoyl]-2- pyridyl]carbamate | 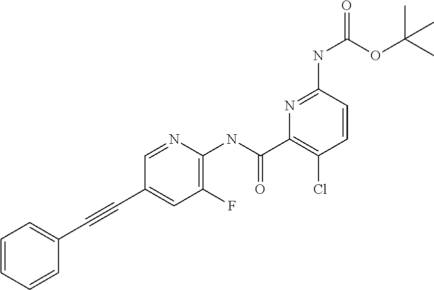 [SMILES: CC(C)(C)OC(=O)Nc1ccc(Cl)c(C(=O)Nc2ncc(C#Cc3ccccc3)cc2F)n1] | 467 | 0.44 |
| 39 | tert-butyl N-[4-chloro-3-[[3-methyl- 5-(2-pyrazin-2-ylethynyl)-2- pyridyl]carbamoyl]phenyl]carbamate | 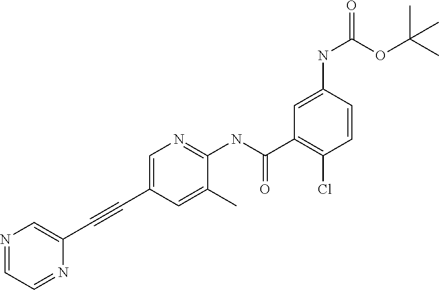 [SMILES: Cc1cc(C#Cc2cnccn2)cnc1NC(=O)c1cc(NC(=O)OC(C)(C)C)ccc1Cl] | 464.2 | 0.47 |
| 40 | tert-butyl N-[4-chloro-3-[[2,4- difluoro-6-(2-phenylethynyl)-3- pyridyl]carbamoyl]phenyl]carbamate | 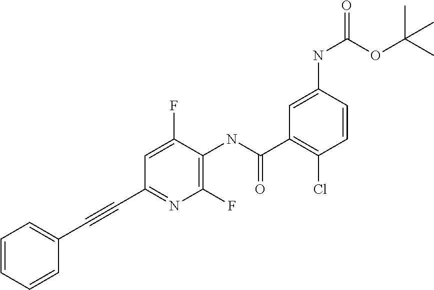 [SMILES: CC(C)(C)OC(=O)Nc1ccc(Cl)c(C(=O)Nc2c(F)cc(C#Cc3ccccc3)nc2F)c1] | 506 (M + Na⁺) | 0.47 |
| 41 | tert-butyl N-[4-chloro-3-[[3-methyl- 5-[2-(3-pyridyl)ethynyl]-2- pyridyl]carbamoyl]phenyl]carbamate | 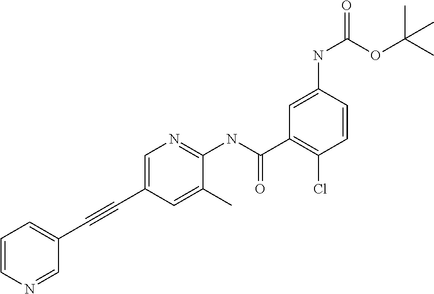 [SMILES: Cc1cc(C#Cc2cccnc2)cnc1NC(=O)c1cc(NC(=O)OC(C)(C)C)ccc1Cl] | 463 | 0.53 |
| 42 | tert-butyl N-[4-chloro-3-[[5-[2-(2- fluorophenyl)ethynyl]-3-methyl-2- pyridyl]carbamoyl]phenyl]carbamate | 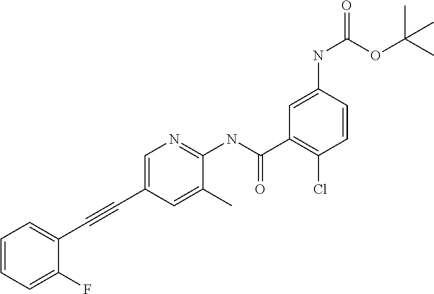 [SMILES: Cc1cc(C#Cc2ccccc2F)cnc1NC(=O)c1cc(NC(=O)OC(C)(C)C)ccc1Cl] | 480 | 0.54 |
| 43 | tert-butyl N-[5-chloro-6-[[3-methyl- 5-(2-phenylethynyl)-2- pyridyl]carbamoyl]-2- pyridyl]carbamate | 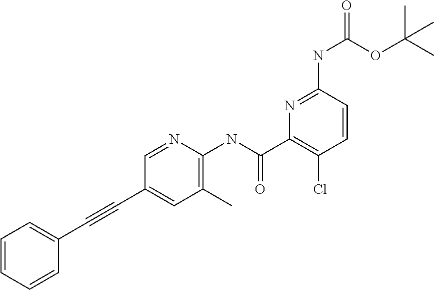 [SMILES: Cc1cc(C#Cc2ccccc2)cnc1NC(=O)c1nc(NC(=O)OC(C)(C)C)ccc1Cl] | 463 | 0.55 |
| 44 | tert-butyl N-[2-[3-methyl-5-(2- phenylethynyl)-2-pyridyl]-3-oxo- isoindolin-5-yl]carbamate | 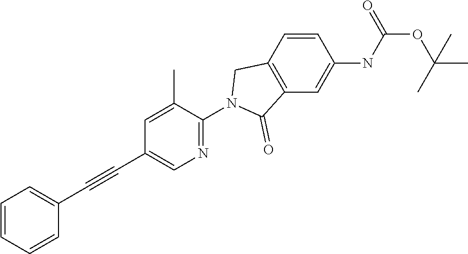 [SMILES: Cc1cc(C#Cc2ccccc2)cnc1N1Cc2ccc(NC(=O)OC(C)(C)C)cc2C1=O] | 440.4 | 0.58 |
| 45 | tert-butyl N-[4-chloro-3-[[3-methyl- 5-[2-(4-pyridyl)ethynyl]-2- pyridyl]carbamoyl]phenyl]carbamate | 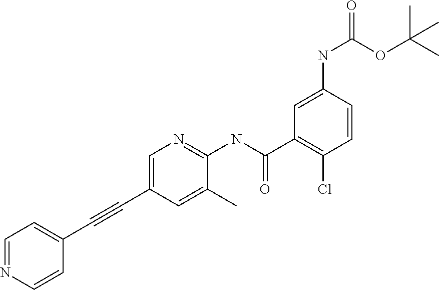 [SMILES: Cc1cc(C#Cc2ccncc2)cnc1NC(=O)c1cc(NC(=O)OC(C)(C)C)ccc1Cl] | 463.0 | 0.61 |
| 46 | tert-butyl N-[4-methyl-3-[[3-methyl- 5-(2-phenylethynyl)-2- pyridyl]carbamoyl]phenyl]carbamate | 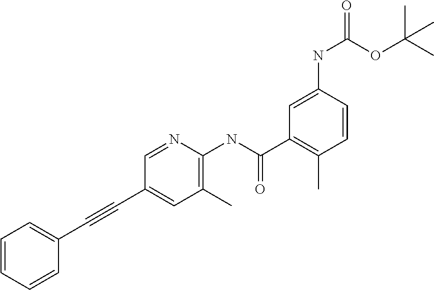 [SMILES: Cc1ccc(NC(=O)OC(C)(C)C)cc1C(=O)Nc1ncc(C#Cc2ccccc2)cc1C] | 442 | 0.87 |
| 47 | tert-butyl N-[4-chloro-3-[[3- methoxy-5-(2-phenylethynyl)-2- pyridyl]carbamoyl]phenyl]carbamate | 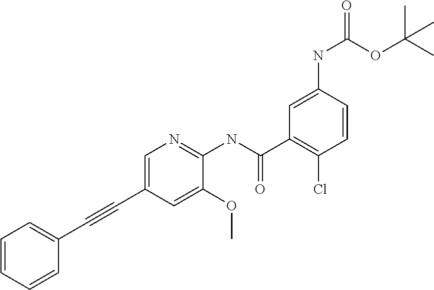 [SMILES: COc1cc(C#Cc2ccccc2)cnc1NC(=O)c1cc(NC(=O)OC(C)(C)C)ccc1Cl] | 478.2 | 0.91 |
| 48 | tert-butyl N-[4-chloro-3-[[2,4- dimethyl-6-(2-phenylethynyl)-3- pyridyl]carbamoyl]phenyl]carbamate | 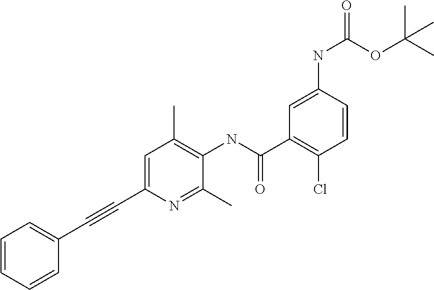 [SMILES: Cc1cc(C#Cc2ccccc2)nc(C)c1NC(=O)c1cc(NC(=O)OC(C)(C)C)ccc1Cl] | 476.2 | 0.95 |
| 49 | 2-chloro-N-[6-[2-(3- fluorophenyl)ethynyl]-2,4-dimethyl- 3-pyridyl]-5-[[(1S,2S)-2- methylcyclopropanecarbonyl]amino] benzamide | 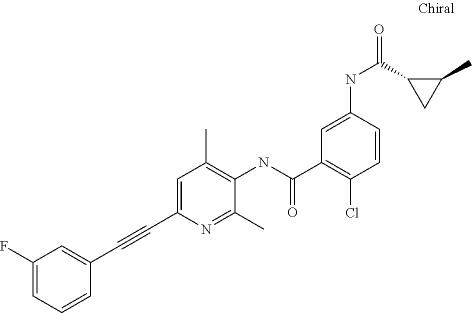 [SMILES: Cc1cc(C#Cc2cccc(F)c2)nc(C)c1NC(=O)c1cc(NC(=O)[C@H]2C[C@@H]2C)ccc1Cl] | 476.2 | 1.09 |
| 50 | tert-butyl N-[4-fluoro-3-[[3-methyl- 5-(2-phenylethynyl)-2- pyridyl]carbamoyl]phenyl]carbamate | 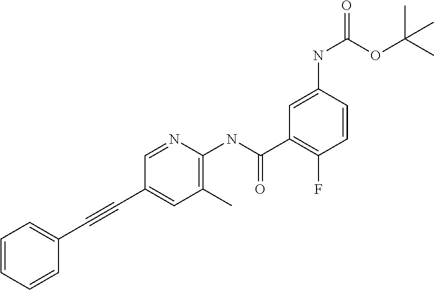 [SMILES: Cc1cc(C#Cc2ccccc2)cnc1NC(=O)c1cc(NC(=O)OC(C)(C)C)ccc1F] | 446 | 1.08 |
| 51 | tert-butyl N-[4-chloro-3-[[3-methyl- 5-(2-phenylethynyl)pyrazin-2- yl]carbamoyl]phenyl]carbamate | 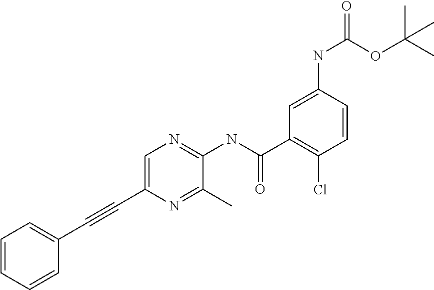 [SMILES: Cc1nc(C#Cc2ccccc2)cnc1NC(=O)c1cc(NC(=O)OC(C)(C)C)ccc1Cl] | 463.3 | 1.42 |
| 52 | tert-butyl N-[6-chloro-5-[[3-methyl- 5-(2-phenylethynyl)-2- pyridyl]carbamoyl]-3- pyridyl]carbamate | 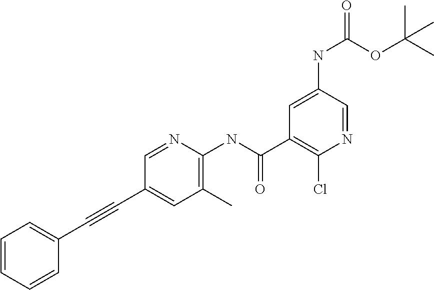 [SMILES: Cc1cc(C#Cc2ccccc2)cnc1NC(=O)c1cc(NC(=O)OC(C)(C)C)cnc1Cl] | 463 | 2.17 |
| 53 | 2-chloro-5-cyano-N-(3-methyl-5- (phenylethynyl)pyridin-2- yl)benzamide | 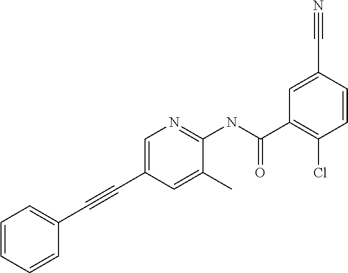 [SMILES: Cc1cc(C#Cc2ccccc2)cnc1NC(=O)c1cc(C#N)ccc1Cl] | 372.2 | 0.81 |
| 54 | 2-chloro-N-[3-fluoro-5-(2- phenylethynyl)-2-pyridyl]-5-(5- tetrahydrofuran-3-yl)-1,2,4-oxadiazol- 3-yl)benzamide | 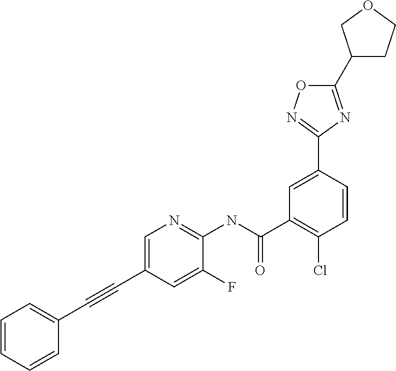 [SMILES: O=C(Nc1ncc(C#Cc2ccccc2)cc1F)c1cc(-c2noc(C3CCOC3)n2)ccc1Cl] | 489 | 0.08 |
| 55 | 2-chloro-N-[3-fluoro-5-(2- phenylethynyl)-2-pyridyl]-5-(1- tetrahydropyran-4ylpyrazol-4- yl)benzamide | 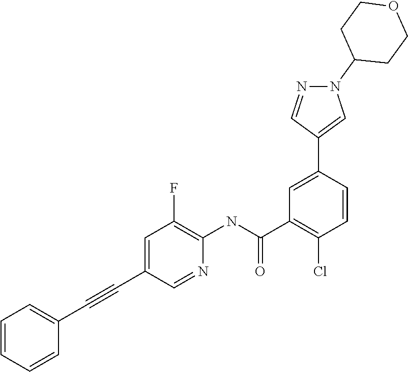 [SMILES: O=C(Nc1ncc(C#Cc2ccccc2)cc1F)c1cc(-c2cnn(C3CCOCC3)c2)ccc1Cl] | 501.3 | 0.10 |
| 56 | 2-chloro-N-[3-fluoro-5-(2- phenylethynyl)-2-pyridyl]-5-[1- (oxetan-3-yl)pyrazol-4-yl]benzamide | 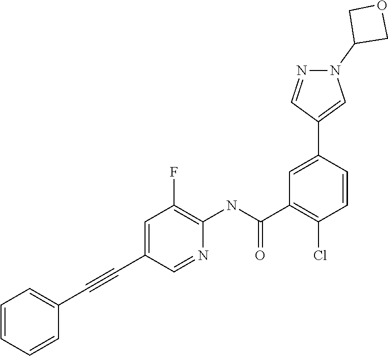 [SMILES: O=C(Nc1ncc(C#Cc2ccccc2)cc1F)c1cc(-c2cnn(C3COC3)c2)ccc1Cl] | 473.3 | 0.11 |
| 57 | 2-chloro-5-(3-cyclopropyl-1,2,4- oxadiazol-5-yl)-N-[3-methyl-5-(2- phenylethynyl)-2-pyridyl]benzamide | 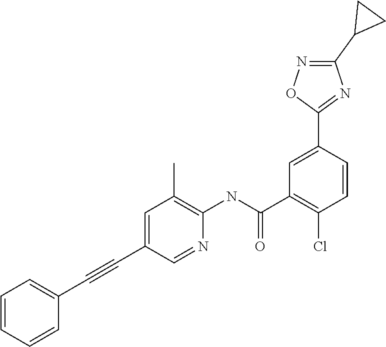 [SMILES: Cc1cc(C#Cc2ccccc2)cnc1NC(=O)c1cc(-c2nc(C3CC3)no2)ccc1Cl] | 455.3 | 0.13 |
| 58 | 2-chloro-N-[3-methyl-5-(2- phenylethynyl)-2-pyridyl]-5-[1- (oxetan-3-yl)pyrazol-4-yl]benzamide | 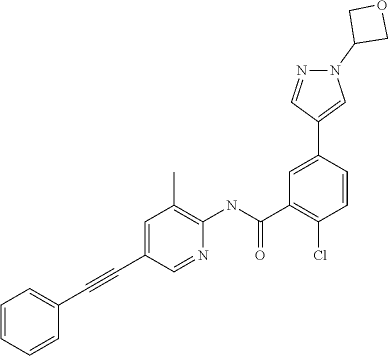 [SMILES: Cc1cc(C#Cc2ccccc2)cnc1NC(=O)c1cc(-c2cnn(C3COC3)c2)ccc1Cl] | 469.2 | 0.13 |
| 59 | 2-chloro-N-[3-fluoro-5-(2- phenylethynyl)-2-pyridyl]-5-(1- methylpyrazol-4-yl)benzamide | 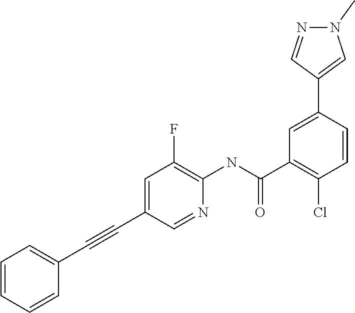 [SMILES: Cn1cc(-c2ccc(Cl)c(C(=O)Nc3ncc(C#Cc4ccccc4)cc3F)c2)cn1] | 431.3 | 0.13 |
| 60 | 2-chloro-5-(3,3-dimethylazetidine-1- carbonyl)-N-[3-methyl-5-(2- phenylethynyl)-2-pyridyl]benzamide | 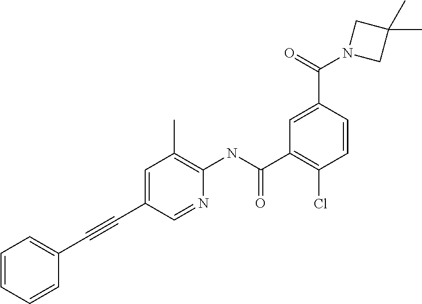 [SMILES: Cc1cc(C#Cc2ccccc2)cnc1NC(=O)c1cc(C(=O)N2CC(C)(C)C2)ccc1Cl] | 458.3 | 0.14 |
| 61 | 2-chloro-N-[3-methyl-5-(2- phenylethynyl)-2-pyridyl]-5-(2- methylpyrazol-3-yl)benzamide | 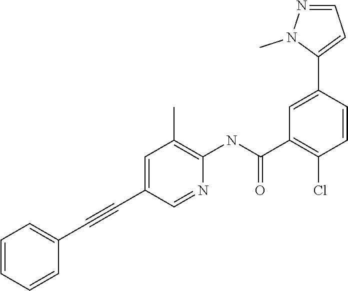 [SMILES: Cc1cc(C#Cc2ccccc2)cnc1NC(=O)c1cc(-c2ccnn2C)ccc1Cl] | 427.2 | 0.16 |
| 62 | 2-chloro-N-[3-methyl-5-(2- phenylethynyl)-2-pyridyl]-5-[1-(2- morpholinoethyl)pyrazol-4- yl]benzamide | 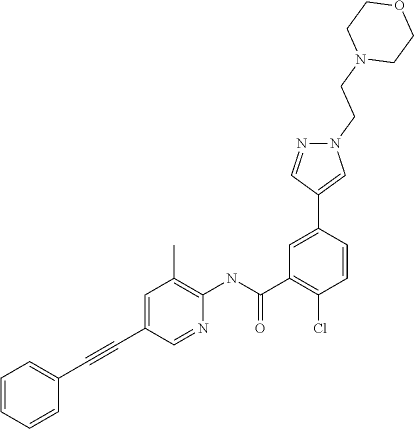 [SMILES: Cc1cc(C#Cc2ccccc2)cnc1NC(=O)c1cc(-c2cnn(CCN3CCOCC3)c2)ccc1Cl] | 526.2 | 0.17 |
| 63 | 2-chloro-5-(3-methyl-1,2,4- oxadiazol-5-yl)-N-[3-methyl-5-(2- phenylethynyl)-2-pyridyl]benzamide | 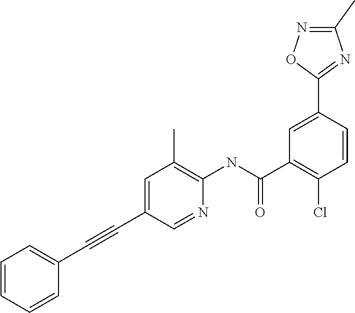 [SMILES: Cc1noc(-c2ccc(Cl)c(C(=O)Nc3ncc(C#Cc4ccccc4)cc3C)c2)n1] | 429.3 | 0.20 |
| 64 | 2-chloro-5-(1-ethylpyrazol-3-yl)-N- [3-methyl-5-(2-phenylethynyl)-2- pyridyl]benzamide | 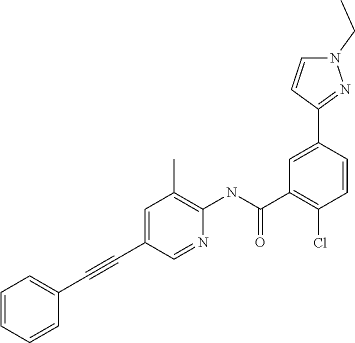 [SMILES: CCn1ccc(-c2ccc(Cl)c(C(=O)Nc3ncc(C#Cc4ccccc4)cc3C)c2)n1] | 441.2 | 0.20 |
| 65 | 2-chloro-N-[3-methyl-5-(2- phenylethynyl)-2-pyridyl]-5-(1- tetrahydropyran-4-ylpyrazol-4- yl)benzamide | 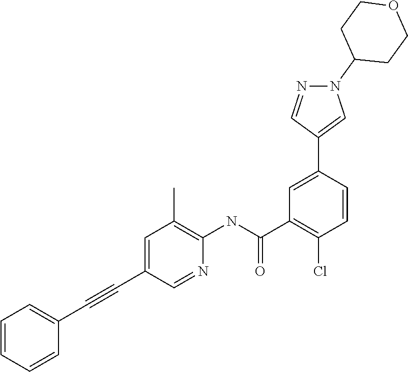 [SMILES: Cc1cc(C#Cc2ccccc2)cnc1NC(=O)c1cc(-c2cnn(C3CCOCC3)c2)ccc1Cl] | 497 | 0.20 |
| 66 | 2-chloro-N-(3-methyl-5- (phenylethynyl)pyridin-2-yl)-5-(1- (tetrahydrofuran-3-yl)-1H-pyrazol-4- yl)benzamide | 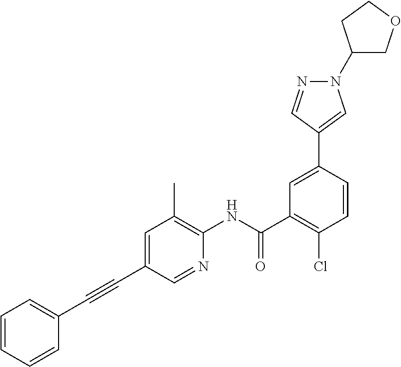 [SMILES: Cc1cc(C#Cc2ccccc2)cnc1NC(=O)c1cc(-c2cnn(C3CCOC3)c2)ccc1Cl] | 483.2 | 0.22 |
| 67 | 2-chloro-5-(2,4-dimethylthiazol-5- yl)-N-[3-fluoro-5-(2-phenylethynyl)- 2-pyridyl]benzamide | 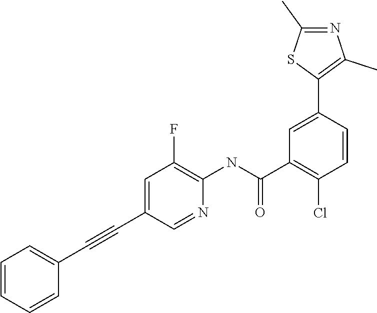 [SMILES: Cc1nc(C)c(-c2ccc(Cl)c(C(=O)Nc3ncc(C#Cc4ccccc4)cc3F)c2)s1] | 461.8 | 0.21 |
| 68 | 2-chloro-5-(1,3-dimethylpyrazol-4- yl)-N-[3-fluoro-5-(2-phenylethynyl)- 2-pyridyl]benzamide | 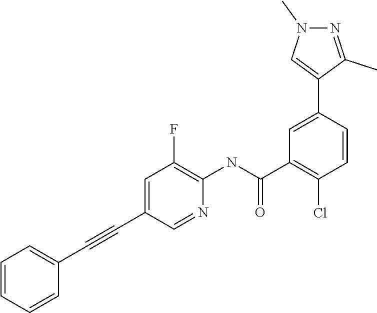 [SMILES: Cc1nn(C)cc1-c1ccc(Cl)c(C(=O)Nc2ncc(C#Cc3ccccc3)cc2F)c1] | 445.2 | 0.22 |
| 69 | 2-chloro-N-[3-methyl-5-(2- phenylethynyl)-2-pyridyl]-5-(2- methylpyrrolidine-1- carbonyl)benzamide | 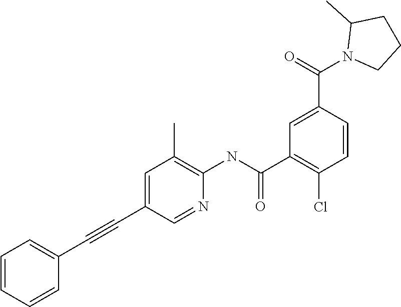 [SMILES: Cc1cc(C#Cc2ccccc2)cnc1NC(=O)c1cc(C(=O)N2CCCC2C)ccc1Cl] | 460.4 | 0.23 |
| 70 | 2-chloro-5-(2,4-dimethylthiazol-5- yl)-N-[3-methyl-5-(2-phenylethynyl)- 2-pyridyl]benzamide | 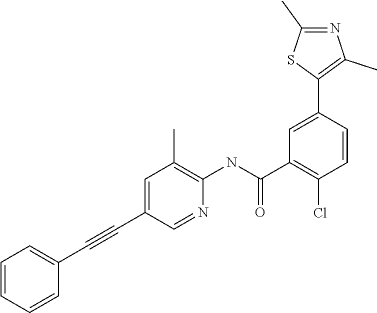 [SMILES: Cc1nc(C)c(-c2ccc(Cl)c(C(=O)Nc3ncc(C#Cc4ccccc4)cc3C)c2)s1] | 458 | 0.26 |
| 71 | 2-chloro-5-(3-hydroxyazetidine-1- carbonyl)-N-[3-methyl-5-(2- phenylethynyl)-2-pyridyl]benzamide | 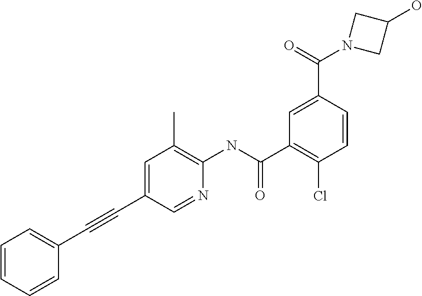 [SMILES: Cc1cc(C#Cc2ccccc2)cnc1NC(=O)c1cc(C(=O)N2CC(O)C2)ccc1Cl] | 446.3 | 0.30 |
| 72 | 2-chloro-5-(3,5-dimethylpyrazole-1- carbonyl)-N-[3-methyl-5-(2- phenylethynyl)-2-pyridyl]benzamide | 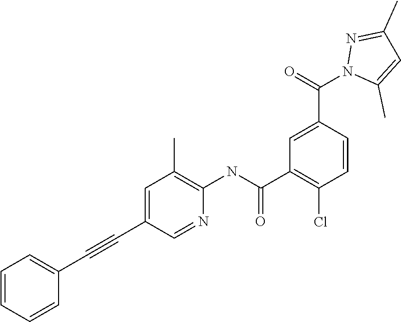 [SMILES: Cc1cc(C)n(C(=O)c2ccc(Cl)c(C(=O)Nc3ncc(C#Cc4ccccc4)cc3C)c2)n1] | 469.4 | 0.36 |
| 73 | 2-chloro-5-(3-fluoroazetidine-1- carbonyl)-N-[3-methyl-5-(2- phenylethynyl)-2-pyridyl]benzamide | 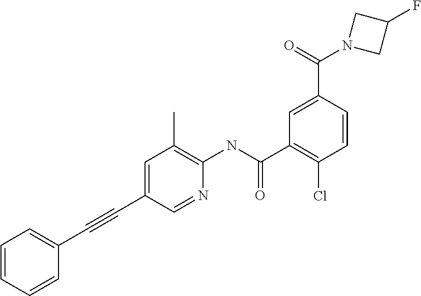 [SMILES: Cc1cc(C#Cc2ccccc2)cnc1NC(=O)c1cc(C(=O)N2CC(F)C2)ccc1Cl] | 448 | 0.39 |
| 74 | 2-chloro-5-(1,3-dimethylpyrazol-4- yl)-N-[3-methyl-5-(2-phenylethynyl)- 2-pyridyl]benzamide | 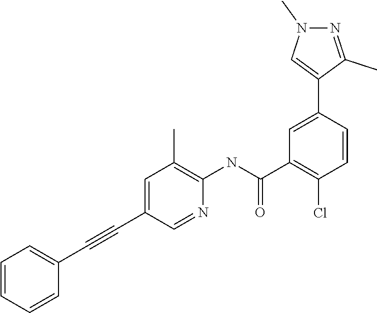 [SMILES: Cc1cc(C#Cc2ccccc2)cnc1NC(=O)c1cc(-c2cn(C)nc2C)ccc1Cl] | 441 | 0.41 |
| 75 | 2-chloro-N-[3-methyl-5-(2- phenylethynyl)-2-pyridyl]-5-(2- methylthiazol-5-yl)benzamide | 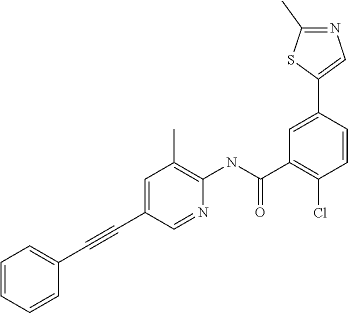 [SMILES: Cc1ncc(-c2ccc(Cl)c(C(=O)Nc3ncc(C#Cc4ccccc4)cc3C)c2)s1] | 444 | 0.45 |
| 76 | 2-chloro-5-(2,4-dimethylpyrazol-3- yl)-N-[3-methyl-5-(2-phenylethynyl)- 2-pyridyl]benzamide | 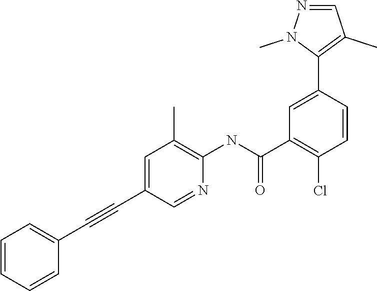 [SMILES: Cc1cc(C#Cc2ccccc2)cnc1NC(=O)c1cc(-c2c(C)cnn2C)ccc1Cl] | 441.2 | 0.45 |
| 77 | 2-chloro-N-[3-methyl-5-(2- phenylethynyl)-2-pyridyl]-5-(1,3,4- oxadiazol-2-yl)benzamide | 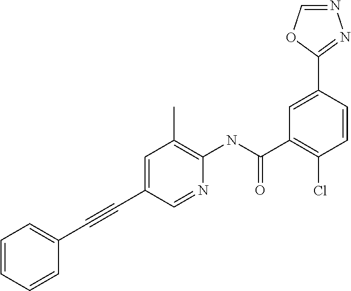 [SMILES: Cc1cc(C#Cc2ccccc2)cnc1NC(=O)c1cc(-c2nnco2)ccc1Cl] | 415.3 | 0.47 |
| 78 | 2-chloro-N-[3-methyl-5-(2- phenylethynyl)-2-pyridyl]-5-(1- methylpyrazol-4-yl)benzamide | 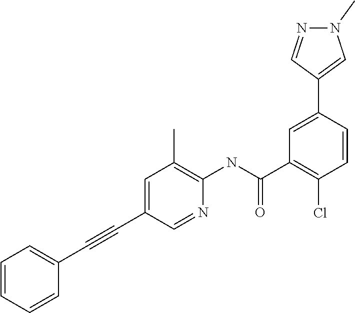 [SMILES: Cc1cc(C#Cc2ccccc2)cnc1NC(=O)c1cc(-c2cnn(C)c2)ccc1Cl] | 427 | 0.47 |
| 79 | 2-chloro-5-[3-hydroxy-3- (trifluoromethyl)azetidine-1- carbonyl]-N-[3-methyl-5-(2- phenylethynyl)-2-pyridyl]benzamide | 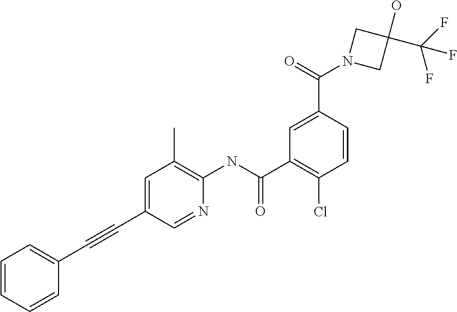 [SMILES: Cc1cc(C#Cc2ccccc2)cnc1NC(=O)c1cc(C(=O)N2CC(O)(C(F)(F)F)C2)ccc1Cl] | 514 | 0.87 |
| 80 | 2-chloro-5-[[(2R)-1,4-dioxan-2- yl]methoxy]-N-[3-fluoro-5-(2- phenylethynyl)-2-pyridyl]benzamide | 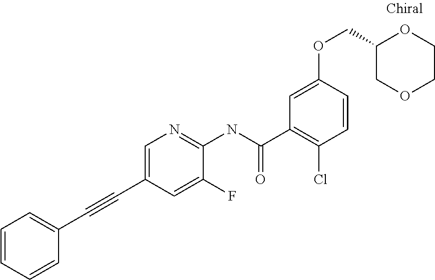 [SMILES: O=C(Nc1ncc(C#Cc2ccccc2)cc1F)c1cc(OC[C@H]2COCCO2)ccc1Cl] | 467.2 | 0.06 |
| 81 | 6-(1,4-dioxan-2-ylmethoxy)-2-[3- methyl-5-(2-phenylethynyl)-2- pyridyl]isoindolin-1-one | 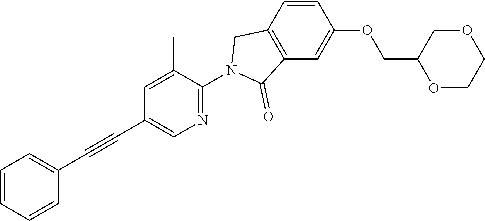 [SMILES: Cc1cc(C#Cc2ccccc2)cnc1N1Cc2ccc(OCC3COCCO3)cc2C1=O] | 441.4 | 0.36 |
| 82 | 2-(3-methyl-5- (phenylethynyl)pyridin-2-yl)-6- ((tetrahydro-2H-pyran-4- yl)methoxy)isoindolin-1-one | 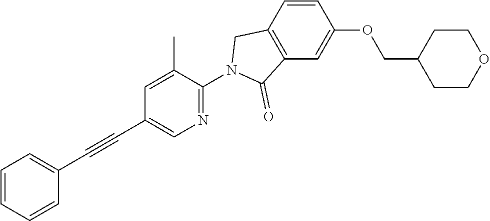 [SMILES: Cc1cc(C#Cc2ccccc2)cnc1N1Cc2ccc(OCC3CCOCC3)cc2C1=O] | 439.3 | 0.39 |
| 83 | 2-chloro-5-[(3-methylisoxazol-5- yl)methoxy]-N-[3-methyl-5-(2- phenylethynyl)-2-pyridyl]benzamide | 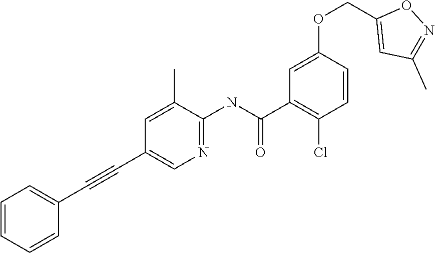 [SMILES: Cc1cc(COc2ccc(Cl)c(C(=O)Nc3ncc(C#Cc4ccccc4)cc3C)c2)on1] | 4582.2 | 0.39 |
| 84 | 2-chloro-N-[3-methyl-5-(2- phenylethynyl)-2-pyridyl]-5-(2- morpholinoethoxy)benzamide | 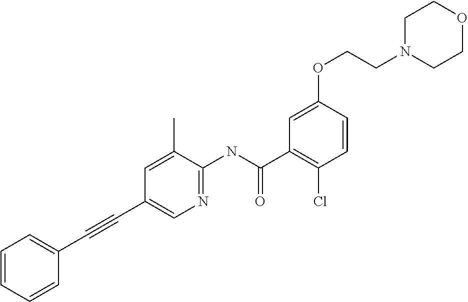 [SMILES: Cc1cc(C#Cc2ccccc2)cnc1NC(=O)c1cc(OCCN2CCOCC2)ccc1Cl] | 476.4 | 0.42 |
| 85 | 2-chloro-N-[3-methyl-5-(2- phenylethynyl)-2-pyridyl]-5- (tetrahydrofuran-3- ylmethoxy)benzamide | 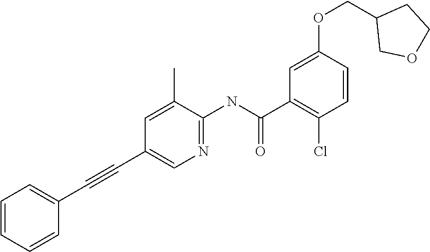 [SMILES: Cc1cc(C#Cc2ccccc2)cnc1NC(=O)c1cc(OCC2CCOC2)ccc1Cl] | 433 | 0.49 |
| 86 | tert-butyl N-[3-[(5-benzyloxy-3- methyl-pyrazin-2-yl)carbamoyl]-4- chloro-phenyl]carbamate | 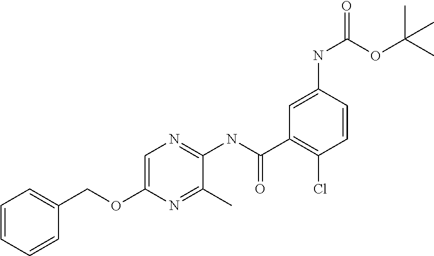 [SMILES: Cc1nc(OCc2ccccc2)cnc1NC(=O)c1cc(NC(=O)OC(C)(C)C)ccc1Cl] | 469.2 (meth- od-3) | 0.56 |
| 87 | tert-butyl N-[3-[(5-benzyloxy-3- methyl-2-pyridyl)carbamoyl]-4- chloro-phenyl]carbamate | 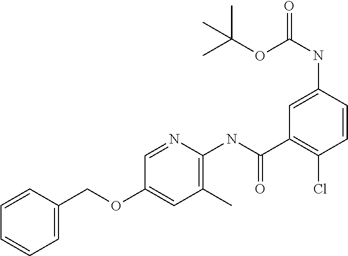 [SMILES: Cc1cc(OCc2ccccc2)cnc1NC(=O)c1cc(NC(=O)OC(C)(C)C)ccc1Cl] | 468.2 (meth- od-3) | 0.56 |
| 88 | tert-butyl (3-((4-(benzyloxy)-2,6- dimethylphenyl)carbamoyl)-4- chlorophenyl)carbamate | 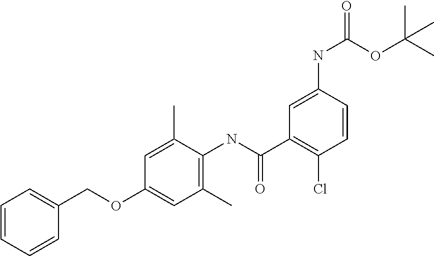 [SMILES: Cc1cc(OCc2ccccc2)cc(C)c1NC(=O)c1cc(NC(=O)OC(C)(C)C)ccc1Cl] | 481.2 (meth- od-3) | 0.36 |
| 89 | 4-amino-N-(4-(benzyloxy)-2- methylphenyl)-2-chlorobenzamide | 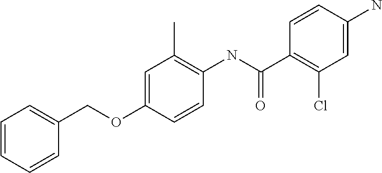 [SMILES: Cc1cc(OCc2ccccc2)ccc1NC(=O)c1ccc(N)cc1Cl] | 367.3 (meth- od-2) | 7.67 |
| 90 | tert-butyl (3-((6-(benzyloxy)-2- methylpyridin-3-yl)carbamoyl)-4- chlorophenyl)carbamate | 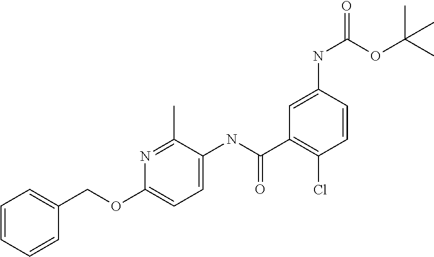 [SMILES: Cc1nc(OCc2ccccc2)ccc1NC(=O)c1cc(NC(=O)OC(C)(C)C)ccc1Cl] | 468.2 (meth- od-3) | 0.85 |
| 91 | 5-acetamido)-N-(4-(benzyloxy)-2- methylphenyl)-2-chlorobenzamide | 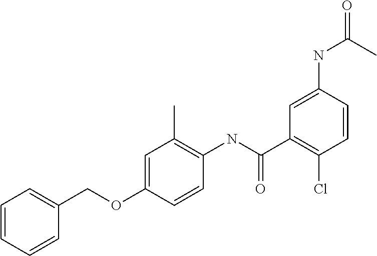 [SMILES: CC(=O)Nc1ccc(Cl)c(C(=O)Nc2ccc(OCc3ccccc3)cc2C)c1] | 409.4 (meth- od-2) | 1.03 |
| 92 | tert-butyl (4-chloro-3-((3-fluoro-5-(1- (4-fluorophenyl)-1H-pyrazol-4 yl)pyridin-2- yl)carbamoyl)phenyl)carbamate | 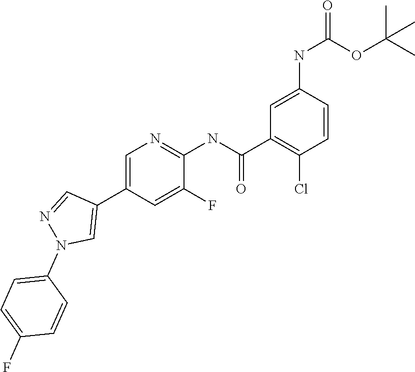 [SMILES: CC(C)(C)OC(=O)Nc1ccc(Cl)c(C(=O)Nc2ncc(-c3cnn(-c4ccc(F)cc4)c3)cc2F)c1] | 526.1 | 0.13 |
| 93 | tert-butyl N-[4-chloro-3-[[3-methyl- 5-(1-phenylpyrazol-4-yl)-2- pyridyl]carbamoyl]phenyl]carbamate | 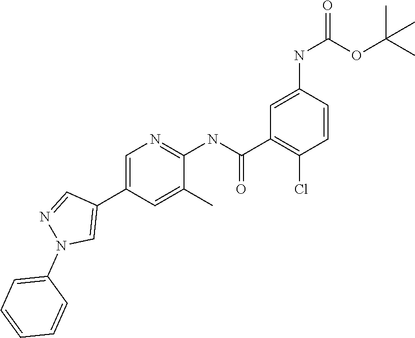 [SMILES: Cc1cc(-c2cnn(-c3ccccc3)c2)cnc1NC(=O)c1cc(NC(=O)OC(C)(C)C)ccc1Cl] | 504.2 | 0.19 |
| 94 | tert-butyl N-[4-chloro-3-[[5-[1-(4- fluorophenyl)pyrazol-4-yl]-3-methyl- 2- pyridyl]carbamoyl]phenyl]carbamate | 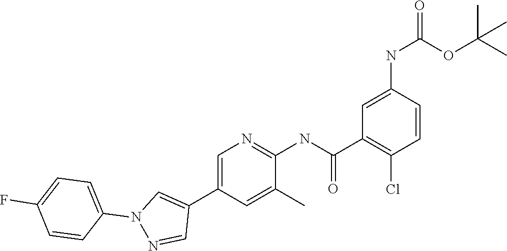 [SMILES: Cc1cc(-c2cnn(-c3ccc(F)cc3)c2)cnc1NC(=O)c1cc(NC(=O)OC(C)(C)C)ccc1Cl] | 522 | 0.20 |
| 95 | tert-butyl N-[4-chloro-3-[[5-[5-(4- fluorophenyl)-1,2,4-oxadiazol-3-yl]- 3-methyl-2- pyridyl]carbamoyl]phenyl]carbamate | 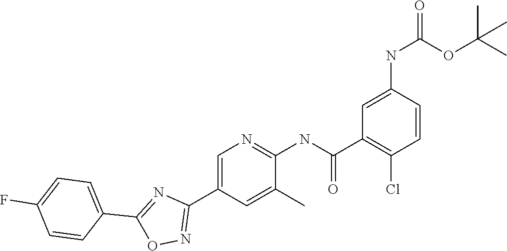 [SMILES: Cc1cc(-c2noc(-c3ccc(F)cc3)n2)cnc1NC(=O)c1cc(NC(=O)OC(C)(C)C)ccc1Cl] | 524.2 | 0.23 |
| 96 | tert-butyl N-[4-chloro-3-[[3-fluoro-5- (3-phenylisoxazol-5-yl)-2- pyridyl]carbamoyl]phenyl]carbamate | 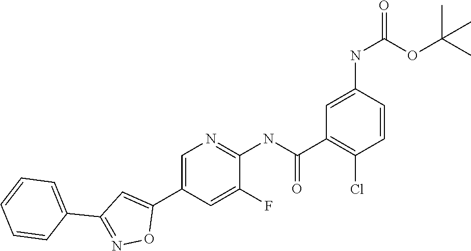 [SMILES: CC(C)(C)OC(=O)Nc1ccc(Cl)c(C(=O)Nc2ncc(-c3cc(-c4ccccc4)no3)cc2F)c1] | 509 | 0.23 |
| 97 | tert-butyl N-[4-chloro-3-[[3-fluoro-5- (1-phenylpyrazol-4-yl)-2- pyridyl]carbamoyl]phenyl]carbamate | 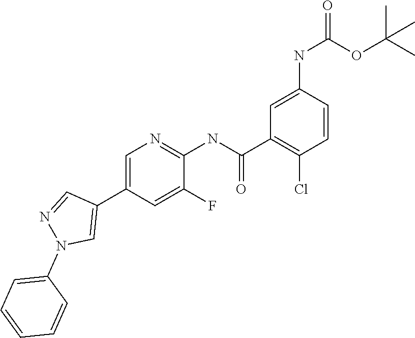 [SMILES: CC(C)(C)OC(=O)Nc1ccc(Cl)c(C(=O)Nc2ncc(-c3cnn(-c4ccccc4)c3)cc2F)c1] | 508 | 0.29 |
| 98 | N-[4-chloro-3-[[3-fluoro-5-[1-(4- fluorophenyl)pyrazol-3-yl]-2- pyridyl]carbamoyl]phenyl]1,4- dioxane-2-carboxamide | 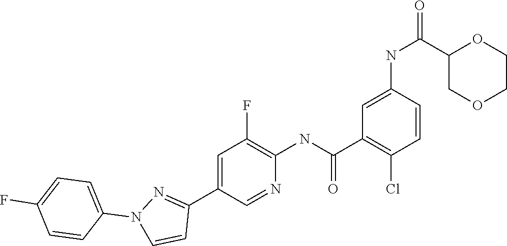 [SMILES: O=C(Nc1ncc(-c2ccn(-c3ccc(F)cc3)n2)cc1F)c1cc(NC(=O)C2COCCO2)ccc1Cl] | 540 | 0.56 |
| 99 | tert-butyl N-[4-chloro-3-[[3-methyl- 5-(5-phenyl-2-thienyl)-2- pyridyl]carbamoyl]phenyl]carbamate | 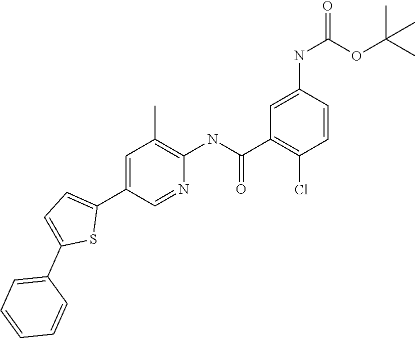 [SMILES: Cc1cc(-c2ccc(-c3ccccc3)s2)cnc1NC(=O)c1cc(NC(=O)OC(C)(C)C)ccc1Cl] | 520 | 1.83 |

ES-MS figures in Table 1 were detected by Reversed-phase LCMS method-1 unless specified.

The compounds shown in Table 2 were prepared in an analogous manner with the appropriate starting materials, and evaluated in our TREK-1 Manual Patch Clamp (hMPC) assay.

TABLE 2

| Cpd. | | | ES-MS | T1⁺ Assay |
|---|---|---|---|---|
| No. | Name | Structure | [M + 1]⁺ | IC₅₀ (μM) |
| 1 | tert-butyl N-[4- chloro-3-[[3-fluoro-5- (2-phenylethynyl)-2- pyridyl]carbamoyl] phenyl]carbamate | 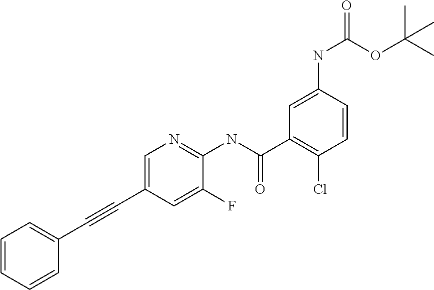 [SMILES: CC(C)(C)OC(=O)Nc1ccc(Cl)c(C(=O)Nc2ncc(C#Cc3ccccc3)cc2F)c1] | 466 | 0.005 |
| 2 | 2-chloro-N-[3-fluoro-5-(2- phenylethynyl)-2-pyridyl]-5- [[(1S,2S)-2- methylcyclopropane- carbonyl]amino] benzamide | 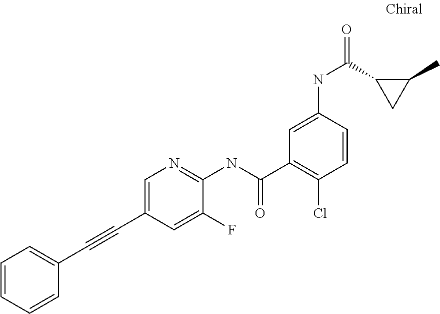 [SMILES: C[C@H]1C[C@@H]1C(=O)Nc1ccc(Cl)c(C(=O)Nc2ncc(C#Cc3ccccc3)cc2F)c1] | 448 | 0.005 |
| 3 | 2-chloro-N-[3-fluoro-5-(2- phenylethynyl)- 2-pyridyl]-5-[(2- methylcyclopropane- carbonyl)amino] benzamide | 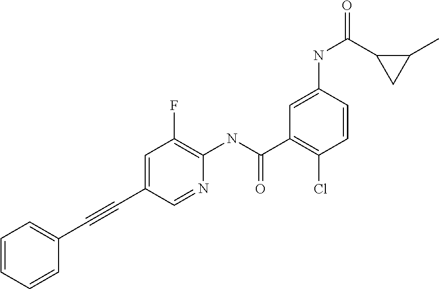 [SMILES: CC1CC1C(=O)Nc1ccc(Cl)c(C(=O)Nc2ncc(C#Cc3ccccc3)cc2F)c1] | 448.0 | 0.006 |
| 4 | 2-chloro-N-[3-fluoro-5-[2-(3- fluorophenyl) ethynyl]-2-pyridyl]-5- [[(1S,2S)-2- methylcyclopropane- carbonyl]amino] benzamide | 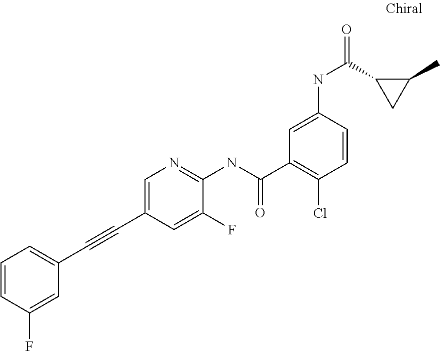 [SMILES: C[C@H]1C[C@@H]1C(=O)Nc1ccc(Cl)c(C(=O)Nc2ncc(C#Cc3cccc(F)c3)cc2F)c1] | 466 | 0.008 |
| 5 | tert-butyl N-[4- chloro-3-[[5-[2-(3- fluorophenyl) ethynyl]-3-methyl-2- pyridyl]carbamoyl] phenyl]carbamate | 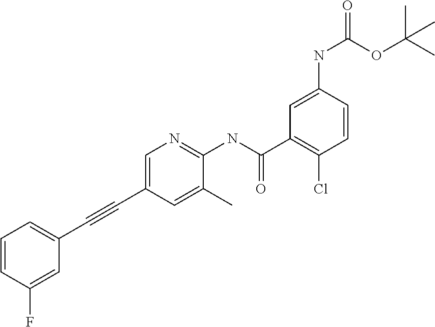 [SMILES: Cc1cc(C#Cc2cccc(F)c2)cnc1NC(=O)c1cc(NC(=O)OC(C)(C)C)ccc1Cl] | 480.3 | 0.013 |
| 6 | 2-chloro-5-[(1- fluorocyclopropane- carbonyl)amino]- N-[3-fluoro-5-(2- phenylethynyl)-2- pyridyl]benzamide | 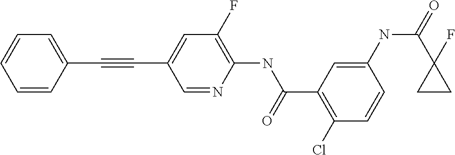 [SMILES: O=C(Nc1ncc(C#Cc2ccccc2)cc1F)c1cc(NC(=O)C2(F)CC2)ccc1Cl] | 452 | 0.013 |
| 7 | 2-chloro-5- (cyclopropane- carbonylamino)-N-[3- fluoro-5-[2-(4- fluorophenyl)ethynyl]-2- pyridyl]benzamide | 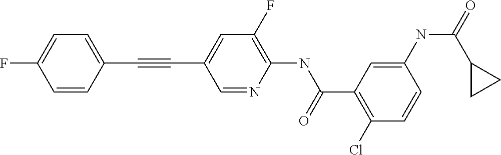 [SMILES: O=C(Nc1ncc(C#Cc2ccc(F)cc2)cc1F)c1cc(NC(=O)C2CC2)ccc1Cl] | 451.9 | 0.014 |
| 8 | tert-butyl N-[4- chloro-3-[[5-[2-(4- fluorophenyl) ethynyl]-3-methyl-2- pyridyl]carbamoyl] phenyl]carbamate | 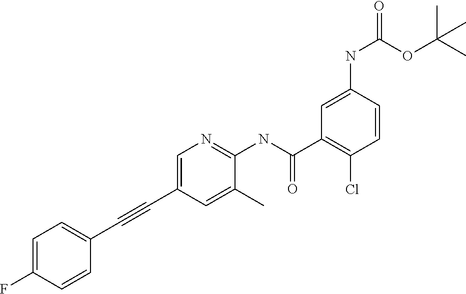 [SMILES: Cc1cc(C#Cc2ccc(F)cc2)cnc1NC(=O)c1cc(NC(=O)OC(C)(C)C)ccc1Cl] | 480.5 (method-2) | 0.015 |
| 9 | 2-chloro-5- (cyclopropane- carbonylamino)-N-[3- methyl-5-(2-phenylethynyl)-2- pyridyl]benzamide | 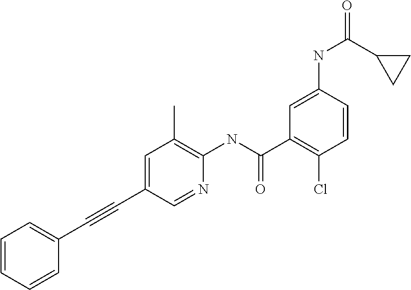 [SMILES: Cc1cc(C#Cc2ccccc2)cnc1NC(=O)c1cc(NC(=O)C2CC2)ccc1Cl] | 430.2 | 0.016 |
| 10 | N-[4-chloro-3- [[3-fluoro-5-(2- phenylethynyl)-2- pyridyl]carbamoyl] phenyl]-1,4- dioxane-2-carboxamide | 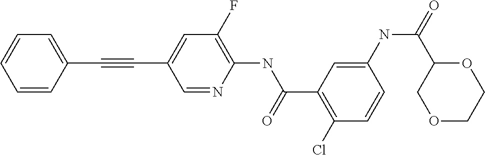 [SMILES: O=C(Nc1ncc(C#Cc2ccccc2)cc1F)c1cc(NC(=O)C2COCCO2)ccc1Cl] | 480 | 0.025 |
| 11 | tert-butyl N-[4- chloro-3-[[6-[2-(3- fluorophenyl) ethynyl]-2,4-dimethyl- 3- pyridyl]carbamoyl] phenyl\|carbamate | 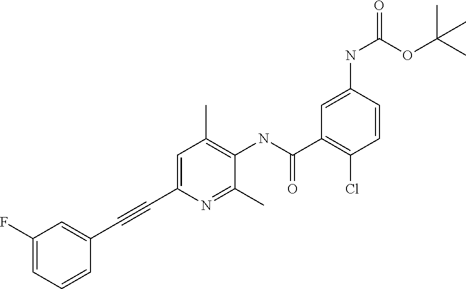 [SMILES: Cc1cc(C#Cc2cccc(F)c2)nc(C)c1NC(=O)c1cc(NC(=O)OC(C)(C)C)ccc1Cl] | 476.2 | 0.032 |
| 12 | N-[4-chloro-3-[[5-[2-(4- fluorophenyl) ethynyl]-3-methyl-2- pyridyl]carbamoyl] phenyl]oxetane- 2-carboxamide | 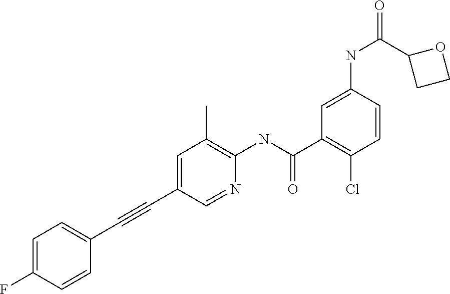 [SMILES: Cc1cc(C#Cc2ccc(F)cc2)cnc1NC(=O)c1cc(NC(=O)C2CCO2)ccc1Cl] | 464 | 0.064 |
| 13 | N-[4-chloro-3-[[5-[2-(3- fluorophenyl) ethynyl]-3-methyl-2- pyridyl]carbamoyl] phenyl]-1,4- dioxane-2-carboxamide | 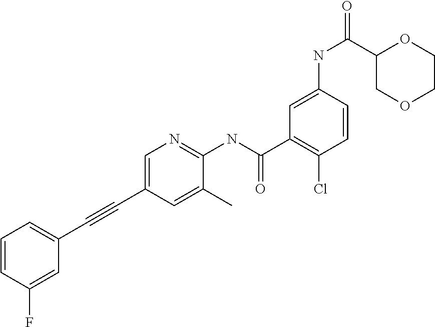 [SMILES: Cc1cc(C#Cc2cccc(F)c2)cnc1NC(=O)c1cc(NC(=O)C2COCCO2)ccc1Cl] | 494 | 0.068 |
| 14 | N-[4-chloro-3-[[5-[2-(4- fluorophenyl) ethynyl]-3-methyl-2- pyridyl]carbamoyl] phenyl]-1,4- dioxane-2-carboxamide | 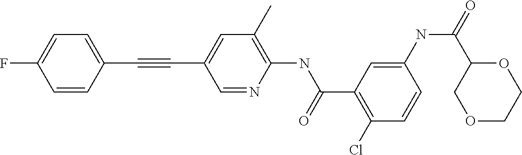 [SMILES: Cc1cc(C#Cc2ccc(F)cc2)cnc1NC(=O)c1cc(NC(=O)C2COCCO2)ccc1Cl] | 494 | 0.083 |
| 15 | tert-butyl N-[4- chloro-3-[[3-methyl- 5-[2-(2-pyridyl)ethynyl]-2- pyridyl]carbamoyl] phenyl]carbamate | 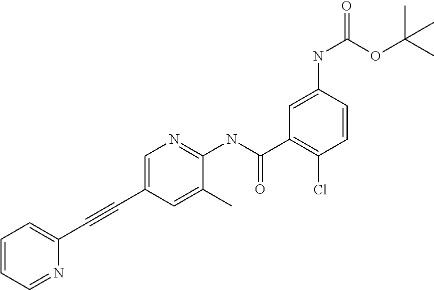 [SMILES: Cc1cc(C#Cc2ccccn2)cnc1NC(=O)c1cc(NC(=O)OC(C)(C)C)ccc1Cl] | 463.4 | 0.139 |
| 16 | 2-chloro-5-(3,3- difluoroazetidine-1- carbonyl)-N-[3-methyl-5-(2- phenylethynyl)-2- pyridyl]benzamide | 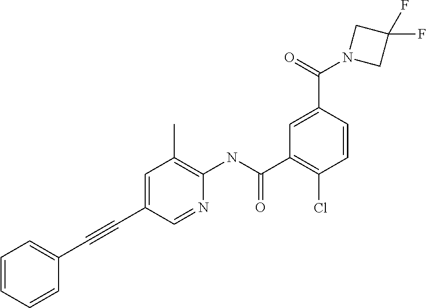 [SMILES: Cc1cc(C#Cc2ccccc2)cnc1NC(=O)c1cc(C(=O)N2CC(F)(F)C2)ccc1Cl] | 466 | 0.041 |
| 17 | 4-chloro-N1- (2-hydroxy-1,1- dimethyl-ethyl)- N3-[3-methyl-5-(2- phenylethynyl)- 2-pyridyl]benzene- 1,3-dicarboxamide | 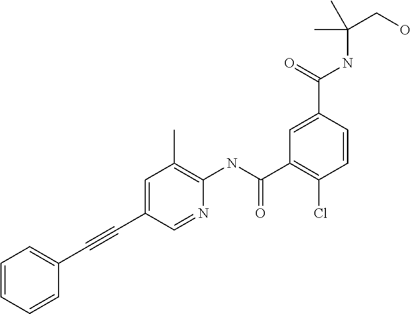 [SMILES: Cc1cc(C#Cc2ccccc2)cnc1NC(=O)c1cc(C(=O)NC(C)(C)CO)ccc1Cl] | 462.4 | 0.051 |
| 18 | 2-chloro-N-[5-[2-(3- fluorophenyl)ethynyl]- 3-methyl-2- pyridyl]-5-(3- hydroxy-3-methyl- azetidine-1- carbonyl)benzamide | 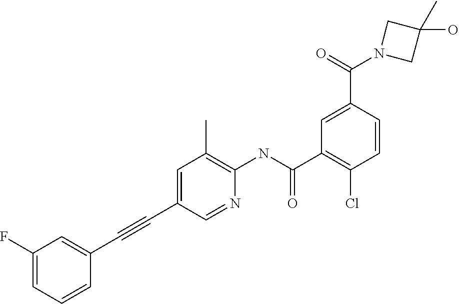 [SMILES: Cc1cc(C#Cc2cccc(F)c2)cnc1NC(=O)c1cc(C(=O)N2CC(C)(O)C2)ccc1Cl] | 478 | 0.086 |
| 19 | 2-chloro-N- [3-methyl-5-(2- phenylethynyl)- 2-pyridyl]-5-(5- tetrahydrofuran-3- yl-1,2,4-oxadiazol- 3-yl)benzamide | 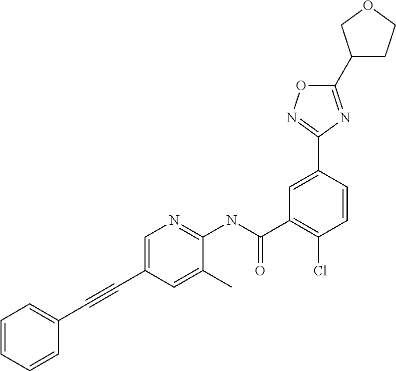 [SMILES: Cc1cc(C#Cc2ccccc2)cnc1NC(=O)c1cc(-c2noc(C3CCOC3)n2)ccc1Cl] | 485.2 | 0.191 |
| 20 | 2-chloro-N-[3-methyl-5-(2- phenylethynyl)-2-pyridyl]-5- (tetrahydropyran-4- ylmethoxy)benzamide | 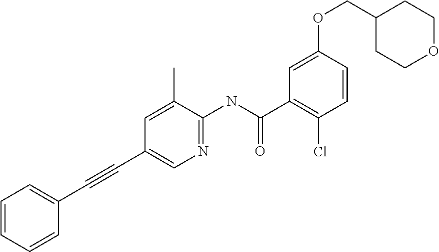 [SMILES: Cc1cc(C#Cc2ccccc2)cnc1NC(=O)c1cc(OCC2CCOCC2)ccc1Cl] | 461.2 | 0.009 |
| 21 | 2-chloro-5- [[(2R)-1,4-dioxan-2- yl]methoxy]-N- [3-methyl-5-(2- phenylethynyl)-2- pyridyl]benzamide | 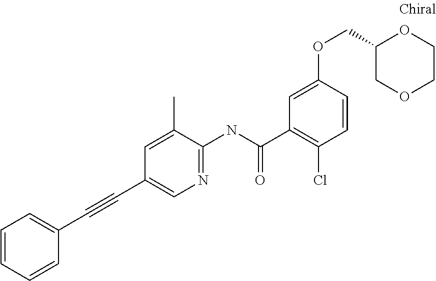 [SMILES: Cc1cc(C#Cc2ccccc2)cnc1NC(=O)c1cc(OC[C@H]2COCCO2)ccc1Cl] | 463.2 | 0.011 |
| 22 | 2-chloro-5-(1,4-dioxan-2- ylmethoxy)-N- [3-methyl-5-(2- phenylethynyl)-2- pyridyl]benzamide | 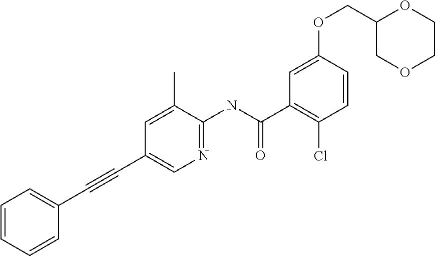 [SMILES: Cc1cc(C#Cc2ccccc2)cnc1NC(=O)c1cc(OCC2COCCO2)ccc1Cl] | 463 | 0.031 |
| 23 | 2-chloro-5-[(2S)- 1,4-dioxan-2- yl]methoxy]-N- [3-methyl-5-(2- phenylethynyl)-2- pyridyl]benzamide | 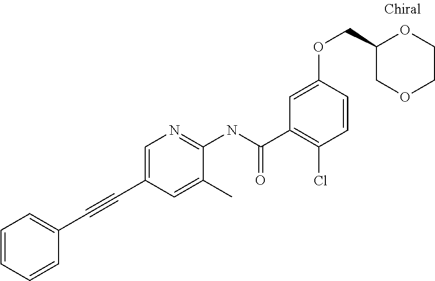 [SMILES: Cc1cc(C#Cc2ccccc2)cnc1NC(=O)c1cc(OC[C@@H]2COCCO2)ccc1Cl] | 463.3 | 0.079 |
| 24 | 2-chloro-5-(2- methoxyethoxy)-N-[3- methyl-5-(2- phenylethynyl)-2- pyridyl]benzamide | 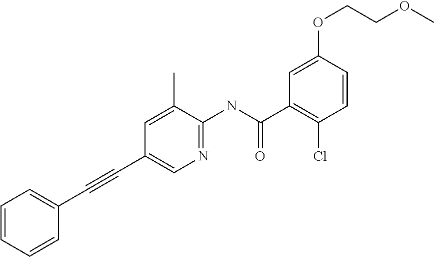 [SMILES: COCCOc1ccc(Cl)c(C(=O)Nc2ncc(C#Cc3ccccc3)cc2C)c1] | 421.3 | 0.084 |
| 25 | tert-butyl (3- ((4-(benzyloxy)-2- methylphenyl)carbamoyl)-4- chlorophenyl)carbamate | 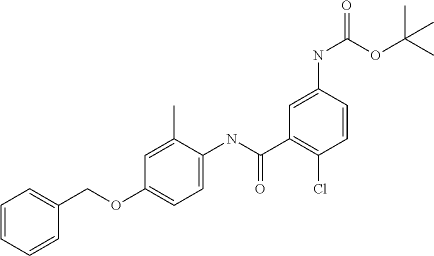 [SMILES: Cc1cc(OCc2ccccc2)ccc1NC(=O)c1cc(NC(=O)OC(C)(C)C)ccc1Cl] | 467.3 (method-2) | 0.066 |
| 26 | tert-butyl (4-chloro- 3-((2-methyl-4- (phenoxymethyl) phenyl)carbamoyl) phenyl)carbamate | 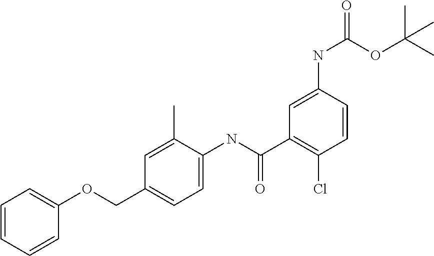 [SMILES: Cc1cc(COc2ccccc2)ccc1NC(=O)c1cc(NC(=O)OC(C)(C)C)ccc1Cl] | 467.2 (method-3) | 0.122 |

ES-MS figures in Table 2 were detected by Reversed-phase LCMS method-1 unless specified.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

The present compound has strong TREK inhibition, and thus is useful as a therapeutic and/or prophylactic agent for various the neurological and/or psychiatric disorders associated with TREK-1, TREK-2 or both TREK-1 and TREK-2 channel activity, particularly, depression, schizophrenia, anxiety disorders, bipolar disorder, Alzheimer's disease, Parkinson's disease, Huntington's disease, Amyotrophic lateral sclerosis, 22q11.2 deletion syndrome, neuropathic pain, cerebral infarction.

1. A compound of formula (I): 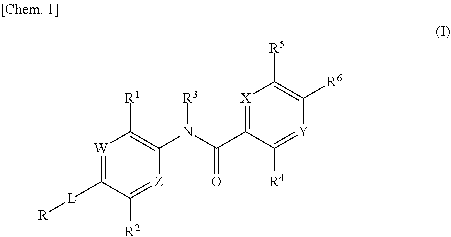
or a pharmaceutically acceptable salt thereof;
wherein:
L is selected from (1) bond, (2) C2-C4-alkynylene, (3) —(C1-C10-alkylene)-O—, (4) —O—(C1-C10-alkylene)-, (5)-(6 to 15 membered aryl)-, (6)-(5 to 15 membered heteroaryl)-, (7)-(3 to 15 membered heterocycle)-, and (8) —(C3-C10-cycloalkane)-;
W is selected from (1) CH, (2) CR⁷, and (3) N;
Z is selected from (1) CH, (2) CR⁸, and (3) N;
R⁷ and R⁸ are each independently selected from (1) cyano, (2) halogen, (3) pentahalosulfanyl, (4) C1-C10-thioalkyl, (5) C1-C10-alkoxy, (6) C1-C10-alkyl, (7) C2-C10-alkenyl, (8) C2-C10-alkynyl, (9) —OR⁹, (10) 6 to 15 membered aryl, (11) 5 to 15 membered heteroaryl, (12) C3-C10-cycloalkyl, (13) C2-C10-heteroalkyl, (14) 3 to 15 membered heterocycle, and (15) —(CR¹⁰R¹¹)ₙ-Q;
wherein each of (4)-(8) in R⁷ or R⁸ may be optionally substituted with 1 to 10 halogen, and each of (10)-(14) in R⁷ or R⁸ may be optionally substituted with 1 to 10 substituents selected from (1) halogen, (2) C1-C10-alkyl, and (3) C1-C10-haloalkyl;
R⁹ is selected from (1) hydrogen, (2) C1-C10-alkyl, and (3) C1-C10-haloalkyl;
R¹⁰ is selected from (1) hydrogen, (2) halogen, (3) C1-C10-alkyl, and (4) C1-C10-haloalkyl;
R¹¹ is selected from (1) hydrogen, (2) halogen, (3) C1-C10-alkyl, and (4) C1-C10-haloalkyl;
or when R¹⁰ and R¹¹ is C1-C10-alkyl, R¹⁰ and R¹¹ may optionally form a C3-C10-cycloalkyl together with the carbon atom to which they are attached;
n is 0, 1, 2, 3 or 4;
Q is selected from (1) halogen, (2) cyano, (3) —OR¹⁰¹, (4) —SR¹⁰², (5) —C(═O)R¹⁰³, (6) —C(═O)OR¹⁰⁴, (7) —S(═O)R¹⁰⁵, (8) —SO₂R¹⁰⁶, (9) —NR¹⁰⁷R¹⁰⁸, (10) —C(═O)NR¹⁰⁹R¹¹⁰, (11) —SO₂NR¹¹¹R¹¹², (12) 6 to 15 membered aryl, (13) 5 to 15 membered heteroaryl, (14) C3-C10-cycloalkyl, and (15) 3 to 15 membered heterocycle, wherein each of (12)-(15) in Q may be optionally substituted with 1 to 10 substituents selected from (1) halogen, (2) C1-C10-alkyl, and (3) C1-C10-haloalkyl;
R¹⁰¹, R¹⁰², R¹⁰³, R¹⁰⁴, R¹⁰⁵, R¹⁰⁶, R¹⁰⁷, R¹⁰⁸, R¹⁰⁹, R¹¹⁰, R¹¹¹, and R¹¹² are each independently selected from (1) hydrogen, (2) C1-C10-alkyl, and (3) C1-C10-haloalkyl;
R⁶ is selected from (1) hydrogen and (2) —NH₂;
X is selected from (1) CH, (2) CR¹², and (3) N;
R¹² is selected from (1) halogen, (2) C1-C10-alkyl and (3) C1-C10-haloalkyl;
Y is selected from (1) CH, (2) CR¹³, and (3) N;
R¹³ is selected from (1) halogen, (2) C1-C10-alkyl, and (3) C1-C10-haloalkyl;
R is selected from (1) 6 to 15 membered aryl and (2) 5 to 15 membered heteroaryl, each of which may be optionally substituted with 1 to 5 R¹⁴;
wherein multiple R¹⁴ may be the same as or different from each other;
R¹⁴ is selected from (1) halogen, (2) cyano, (3) pentahalosulfanyl, (4) C1-C10-alkyl, (5) C1-C10-thioalkyl, (6) C1-C10-alkoxy, (7) C2-C10-alkenyl, (8) C2-C10-alkynyl, (9) 6 to 15 membered aryl, and (10) —OR⁹, wherein each of (4)-(9) in R¹⁴ may be optionally substituted with 1 to 10 halogen;
R¹ is selected from (1) C1-C10-alkyl, (2) halogen, (3) C1-C10-alkoxy, (4) C1-C10-haloalkyl, (5) C1-C10-haloalkoxy, and (6) cyano;
R² is selected from (1) hydrogen, (2) C1-C10-alkyl, (3) halogen, (4) C1-C10-alkoxy, (5) C1-C10-haloalkyl, (6) C1-C10-haloalkoxy, and (7) cyano;
R³ is selected from (1) hydrogen and (2) C1-C10-alkyl;
R⁴ is selected from (1) hydrogen, (2) halogen, (3) C1-C10-alkyl, (4) C1-C10-haloalkyl, (5) cyano, and (6) C3-C10-cycloalkyl which may be optionally substituted with 1 to 10 substituents selected from (1) halogen (2) C1-C10-alkyl and (3) C1-C10-haloalkyl;
or R³ and R⁴ may be optionally taken together to form (1) —CR⁴¹R⁴²—, (2) —CR⁴³R⁴⁴—CR⁴⁵R⁴⁶—, and (3) —CR⁴⁷═CR⁴⁸—;
R⁴¹, R⁴², R⁴³, R⁴⁴, R⁴⁵, R⁴⁶, R⁴⁷, and R⁴⁸ are each independently selected from (1) hydrogen and (2) C1-C10-alkyl;
R⁵ is selected from (1) hydrogen, (2) cyano, (3) —NH₂, (4) C1-C10-alkyl, (5) C1-C10-alkoxy, (6) —NH—C(═O)—R¹⁵, (7) —NH—C(═O)—O—R¹⁶, (8) —O—R¹⁷, (9) —O—(C1-C10-alkylene)-R¹⁸, (10) —C(═O)—R¹⁹, (11) —C(═O)—NH—R²⁰, (12) —(C1-C10-alkylene)-(CR²¹R²²)ₚ—R²³, (13) 6 to 15 membered aryl, (14) 5 to 15 membered heteroaryl, (15) C3-C10-cycloalkyl, and (16) 3 to 15 membered heterocycle;
wherein each of (4), (5), (9), and (12) in R⁵ may be optionally substituted with 1 to 10 substituents selected from (1) halogen, (2) cyano and (3) —OH, and each of (13)-(16) in R⁵ may be optionally substituted with 1 to 5 R²⁴;
provided that when R⁵ is hydrogen, R⁶ is —NH₂;
R¹⁵, R¹⁶, R¹⁷, R¹⁸, R¹⁹, and R²⁰ are each independently selected from (1) C1-C10-alkyl, (2) C1-C10-alkoxy, (3) 6 to 15 membered aryl, (4) 5 to 15 membered heteroaryl, (5) C3-C10-cycloalkyl, and (6) 3 to 15 membered heterocycle, wherein each of (1) and (2) in R¹⁵, R¹⁶, R¹⁷, R¹⁸, R¹⁹, or R²⁰ may be optionally substituted with 1 to 10 substituents selected from (1) halogen, (2) cyano, and (3) —OH, and each of (3)-(6) in R¹⁵, R¹⁶, R¹⁷, R¹⁸, R¹⁹, or R²⁰ may be optionally substituted with 1 to 5 R²⁵;
R²¹ and R²² are each independently selected from (1) hydrogen, (2) halogen, (3) C1-C10-alkyl, and (4) C1-C10-haloalkyl;
or when R²¹ and R²² is C1-C10-alkyl, R²¹ and R²² may optionally form a C3-C10-cycloalkyl together with the carbon atom to which they are attached;
p is 0, 1, 2, 3, or 4;
R²³ is selected from (1) halogen, (2) cyano, (3) —OR²⁰¹, (4) —SR²⁰², (5) —C(═O)R²⁰³, (6) —C(═O)OR²⁰⁴, (7) —S(═O)R²⁰⁵, (8) —SO₂R²⁰⁶, (9) —NR²⁰⁷R²⁰⁸, (10) —C(═O)NR²⁰⁹R²¹⁰, (11) —SO₂NR²¹¹R²¹², (12) 6 to 15 membered aryl, (13) 5 to 15 membered heteroaryl, (14) C3-C10-cycloalkyl, and (15) 3 to 15 membered heterocycle, wherein each of (12)-(15) in R²³ may be optionally substituted with 1 to 10 substituents selected from (1) halogen, (2) C1-C10-alkyl and (3) C1-C10-haloalkyl;
R²⁰¹, R²⁰², R²⁰³, R²⁰⁴, R²⁰⁵, R²⁰⁶, R²⁰⁷, R²⁰⁸, R²⁰⁹, R²¹⁰, R²¹¹, and R²¹² are each independently selected from (1) hydrogen, (2) C1-C10-alkyl, and (3) C1-C10-haloalkyl;
R²⁴ and R²⁵ are each independently selected from (1) halogen, (2) cyano, (3) —OH, (4) oxo, (5) C1-C10-alkyl, (6) C1-C10-alkoxy, (7) —(C1-C10-alkylene)-NR²⁶R²⁷, (8) C3-C10-cycloalkyl, (9) 3 to 15 membered heterocycle, and (10) —(C1-C10-alkylene)-(3 to 15 membered heterocycle), wherein each of (5)-(7) in R²⁴ or R²⁵ may be optionally substituted with 1 to 5 substituents selected with (1) halogen, (2) —OH and (3) cyano, and each of (8)-(10) in R²⁴ or R²⁵ may be optionally substituted with 1 to 5 substituents selected with (1) halogen, (2) —OH, (3) cyano, (4) C1-C10-alkyl, and (5) C1-C10-haloalkyl;
R²⁶ and R²⁷ are each independently selected from (1) hydrogen and (2) C1-C10-alkyl;
multiple R²⁴ or R²⁵ may be the same as or different from each other.

2. The compound or a pharmaceutically acceptable salt thereof according to claim 1, which is a compound of formula (Ia):

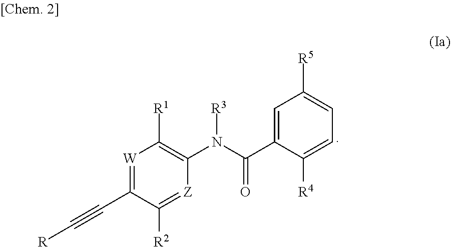

3. The compound or a pharmaceutically acceptable salt thereof according to claim 2, wherein
R⁵ is selected from (1) cyano, (2) —NH₂, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) 6 to 15 membered aryl, (10) 5 to 15 membered heteroaryl, (11) C3-C10-cycloalkyl, and (12) 3 to 15 membered heterocycle;
wherein (6) in R⁵ may be optionally substituted with 1 to 10 substituents selected from (1) halogen (2) cyano, and (3) —OH, and each of (9)-(12) in R⁵ may be optionally substituted with 1 to 5 R²⁴.

4. The compound or a pharmaceutically acceptable salt thereof according to claim 3, which is a compound of formula (Ia-1-1): 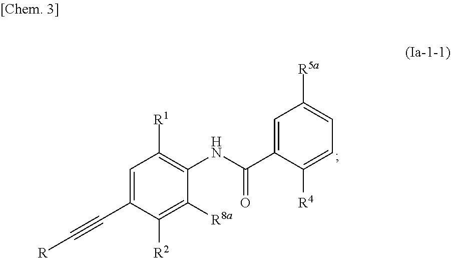
wherein
R^{1a} is selected from (1) halogen, (2) C1-C10-alkyl, and (3) C1-C10-haloalkyl;
R^{8a} is selected from (1) hydrogen, (2) halogen, (3) C1-C10-alkyl, and (4) C1-C10-haloalkyl;
R^{5a} is selected from (1) cyano, (2) —NH₂, (3) —NH—C(═O)—R¹⁵, (4) —NH—C(═O)—O—R¹⁶, (5) —O—R¹⁷, (6) —O—(C1-C10-alkylene)-R¹⁸, (7) —C(═O)—R¹⁹, (8) —C(═O)—NH—R²⁰, (9) 6 to 15 membered aryl, (10) 5 to 15 membered heteroaryl, (11) C3-C10-cycloalkyl, and (12) 3 to 15 membered heterocycle;
wherein (6) in R^{5a} may be optionally substituted with 1 to 10 substituents selected from (1) halogen (2) cyano and (3) —OH, and each of (9)-(12) in R⁵ may be optionally substituted with 1 to 5 R²⁴.

5. The compound or a pharmaceutically acceptable salt thereof according to claim 4, wherein R^{5a} is selected from (1) —NH—C(═O)—(C3-C10-cycloalkyl), (2) —NH—C(═O)-(3 to 15 membered heterocycle), (3) —NH—C(═O)—O—(C1-C10-alkyl) which may be optionally substituted with 1 to 5 substituents selected from halogen, cyano, and —OH, (4) —O(C1-C10-alkyl) which may be optionally substituted with 1 to 5 substituents selected from halogen, cyano, and —OH, (5) —O—(C1-C10-alkylene)-(3 to 15 membered heterocycle), (6) —C(═O)—(C3-C10-cycloalkyl), (7) —C(═O)-(3 to 15 membered heterocycle), (8) —C(═O)—NH—(C1-C10-alkyl) and (9) 5 to 15 membered heteroaryl;
wherein each of (1), (2) and (5)-(9) in R^{5a} may be optionally substituted with 1 to 5 R⁵¹;
multiple R⁵¹ may be the same as or different from each other;
R⁵¹ is independently selected from (1) halogen, (2) cyano, (3) —OH, (4) C1-C10-alkyl, (5) C1-C10-alkoxy, (6) —(C1-C10-alkylene)-(3 to 15 membered heterocycle), (7) C3-C10-cycloalkyl and (8)3 to 15 membered heterocycle,
wherein each of (4)-(8) in R⁵¹ may be optionally substituted with 1 to 10 substituents selected from (1) halogen, (2) —OH, (3) cyano, (4) C1-C10-alkyl and (5) C1-C10-haloalkyl.

6. The compound or a pharmaceutically acceptable salt thereof according to claim 3, which is a compound of formula (Ia-1-2):

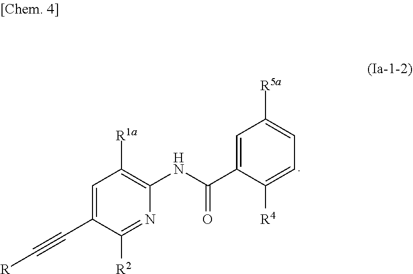

7. The compound or a pharmaceutically acceptable salt thereof according to claim 6, wherein R^{5a} is selected from (1) —NH—C(═O)—(C3-C10-cycloalkyl), (2) —NH—C(═O)-(3 to 15 membered heterocycle), (3) —NH—C(═O)—O—(C1-C10-alkyl) which may be optionally substituted with 1 to 5 substituents selected from halogen, cyano, and —OH, (4) —O(C1-C10-alkyl) which may be optionally substituted with 1 to 5 substituents selected from halogen, cyano, and —OH, (5) —O—(C1-C10-alkylene)-(3 to 15 membered heterocycle), (6) —C(═O)—(C3-C10-cycloalkyl), (7) —C(═O)-(3 to 15 membered heterocycle), (8) —C(═O)—NH—(C1-C10-alkyl), and (9) 5 to 15 membered heteroaryl;
wherein each of (1), (2) and (5)-(9) in R^{5a} may be optionally substituted with 1 to 5 R⁵¹;
multiple R⁵¹ may be the same as or different from each other;
R⁵¹ is independently selected from (1) halogen, (2) cyano, (3) —OH, (4) C1-C10-alkyl, (5) C1-C10-alkoxy, (6) —(C1-C10-alkylene)-(3 to 15 membered heterocycle), (7) C3-C10-cycloalkyl, and (8) 3 to 15 membered heterocycle,
wherein each of (4)-(8) in R⁵¹ may be optionally substituted with 1 to 10 substituents selected from (1) halogen, (2) —OH, (3) cyano, (4) C1-C10-alkyl and (5) C1-C10-haloalkyl.

8. The compound or a pharmaceutically acceptable salt thereof according to claim 1, which is a compound of formula (Ib):

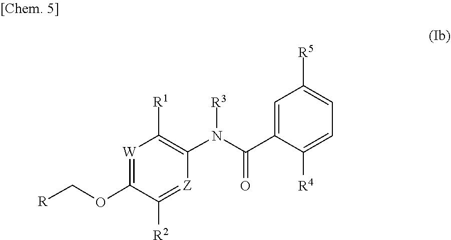

9. The compound or a pharmaceutically acceptable salt thereof according to claim 8, wherein
R is phenyl which may be optionally substituted with 1 to 5 R¹⁴;
R⁴ is halogen,
R⁵ is selected from (1) C1-C10-alkyl, (2) C1-C10-alkoxy, (3) —NH—C(═O)—R¹-5, (4) —NH—C(═O)—O—R¹⁶, (5) —O—R′ 7, (6) —O—(C1-C10-alkylene)-R¹-8, (7) —C(═O)—R¹⁹, (8) —C(═O)—NH—R²⁰, (9) —(C1-C10-alkylene)-(CR²¹R²²)ₚ—R²³, (10) 6 to 15 membered aryl, (11) 5 to 15 membered heteroaryl, (12) C3-C10-cycloalkyl, and (13) 3 to 15 membered heterocycle;
wherein each of (1), (2), (6) and (9) in R⁵ may be optionally substituted with 1 to 10 substituents selected from (1) halogen (2) cyano, and (3) —OH, and each of (10)-(13) in R⁵ may be optionally substituted with 1 to 5 R²⁴.

10. The compound or a pharmaceutically acceptable salt thereof according to claim 1, which is a compound of formula (Ic): 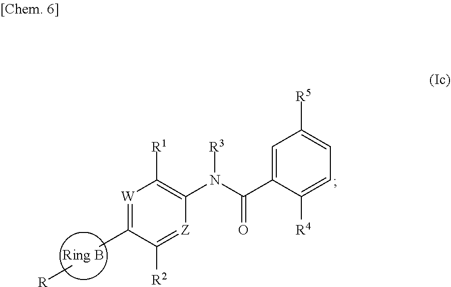
wherein Ring B is selected from (1) 6 to 15 membered aryl and (2) 5 to 15 membered heteroaryl.

11. The compound or a pharmaceutically acceptable salt thereof according to claim 10, wherein
Ring B is selected from 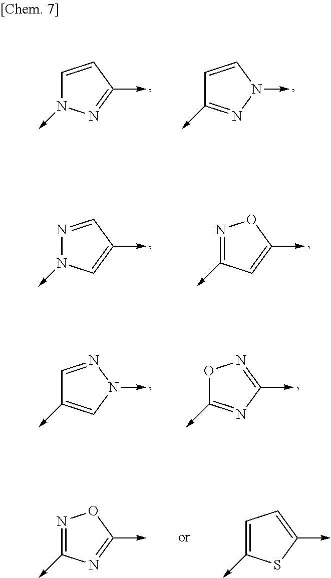
wherein right arrow is connecting position with 6 membered ring and left arrow is connecting position with R;
R is phenyl which may be optionally substituted with 1 to 5 R¹⁴;
R⁴ is halogen,
R⁵ is selected from (1) C1-C10-alkyl, (2) C1-C10-alkoxy, (3) —NH—C(═O)—R¹-5, (4) —NH—C(═O)—O—R¹⁶, (5) —O—R′ 7, (6) —O—(C1-C10-alkylene)-R¹-8, (7) —C(═O)—R¹⁹, (8) —C(═O)—NH—R²⁰, (9) —(C1-C10-alkylene)-(CR²¹R²²)ₚ—R²³, (10) 6 to 15 membered aryl-, (11) 5 to 15 membered heteroaryl, (12) C3-C10-cycloalkyl, and (13) 3 to 15 membered heterocycle;
wherein each of (1), (2), (6) and (9) in R⁵ may be optionally substituted with 1 to 10 substituents selected from (1) halogen (2) cyano, and (3) —OH, and each of (10)-(13) in R⁵ may be optionally substituted with 1 to 5 R²⁴.

12. The compound or a pharmaceutically acceptable salt thereof according to claim 1, wherein the compound is
(1) tert-butyl N-[4-chloro-3-[[3-fluoro-5-(2-phenylethynyl)-2-pyridyl]carbamoyl]phenyl]carbamate,
(2) 2-chloro-N-[3-fluoro-5-(2-phenylethynyl)-2-pyridyl]-5-[[(1S,2S)-2-methylcyclopropanecarbonyl]amino]benzamide,
(3) 2-chloro-N-[3-fluoro-5-(2-phenylethynyl)-2-pyridyl]-5-[(2-methylcyclopropanecarbonyl)amino]benzamide,
(4) 2-chloro-N-[3-fluoro-5-[2-(3-fluorophenyl)ethynyl]-2-pyridyl]-5-[[(1S,2S)-2-methylcyclopropanecarbonyl]amino]benzamide,
(5) tert-butyl N-[4-chloro-3-[[5-[2-(3-fluorophenyl)ethynyl]-3-methyl-2-pyridyl]carbamoyl]phenyl]carbamate,
(6) 2-chloro-5-[(1-fluorocyclopropanecarbonyl)amino]-N-[3-fluoro-5-(2-phenylethynyl)2-pyridyl]benzamide,
(7) 2-chloro-5-(cyclopropanecarbonylamino)-N-[3-fluoro-5-[2-(4-fluorophenyl)ethynyl]-2-pyridyl]benzamide,
(8) tert-butyl N-[4-chloro-3-[[5-[2-(4-fluorophenyl)ethynyl]-3-methyl-2-pyridyl]carbamoyl]phenyl]carbamate,
(9) 2-chloro-5-(cyclopropanecarbonylamino)-N-[3-methyl-5-(2-phenylethynyl)-2-pyridyl]benzamide,
(10) N-[4-chloro-3-[[3-fluoro-5-(2-phenylethynyl)-2-pyridyl]carbamoyl]phenyl]-1,4-dioxane-2-carboxamide,
(11) tert-butyl N-[4-chloro-3-[[6-[2-(3-fluorophenyl)ethynyl]-2,4-dimethyl-3-pyridyl]carbamoyl]phenyl]carbamate,
(12) N-[4-chloro-3-[[5-[2-(4-fluorophenyl)ethynyl]-3-methyl-2-pyridyl]carbamoyl]phenyl]oxetane-2-carboxamide,
(13) N-[4-chloro-3-[[5-[2-(3-fluorophenyl)ethynyl]-3-methyl-2-pyridyl]carbamoyl]phenyl]-1,4-dioxane-2-carboxamide,
(14) N-[4-chloro-3-[[5-[2-(4-fluorophenyl)ethynyl]-3-methyl-2-pyridyl]carbamoyl]phenyl]-1,4-dioxane-2-carboxamide,
(15) tert-butyl N-[4-chloro-3-[[3-methyl-5-[2-(2-pyridyl)ethynyl]-2-pyridyl]carbamoyl]phenyl]carbamate,
(16) 2-chloro-5-(3,3-difluoroazetidine-1-carbonyl)-N-[3-methyl-5-(2-phenylethynyl)-2-pyridyl]benzamide,
(17) 4-chloro-N1-(2-hydroxy-1,1-dimethyl-ethyl)-N3-[3-methyl-5-(2-phenylethynyl)-2-pyridyl]benzene-1,3-dicarboxamide,
(18) 2-chloro-N-[5-[2-(3-fluorophenyl)ethynyl]-3-methyl-2-pyridyl]-5-(3-hydroxy-3-methyl-azetidine-1-carbonyl)benzamide,
(19) 2-chloro-N-[3-methyl-5-(2-phenylethynyl)-2-pyridyl]-5-(5-tetrahydrofuran-3-yl-1,2,4-oxadiazol-3-yl)benzamide,
(20) 2-chloro-N-[3-methyl-5-(2-phenylethynyl)-2-pyridyl]-5-(tetrahydropyran-4-ylmethoxy)benzamide,
(21) 2-chloro-5-[[(2R)-1,4-dioxan-2-yl]methoxy]-N-[3-methyl-5-(2-phenylethynyl)-2-pyridyl]benzamide,
(22) 2-chloro-5-(1,4-dioxan-2-ylmethoxy)-N-[3-methyl-5-(2-phenylethynyl)-2-pyridyl]benzamide,
(23) 2-chloro-5-[[(2S)-1,4-dioxan-2-yl]methoxy]-N-[3-methyl-5-(2-phenylethynyl)-2-pyridyl]benzamide,
(24) 2-chloro-5-(2-methoxyethoxy)-N-[3-methyl-5-(2-phenylethynyl)-2-pyridyl]benzamide,
(25) tert-butyl (3-((4-(benzyloxy)-2-methylphenyl)carbamoyl)-4-chlorophenyl)carbamate, or
(26) tert-butyl (4-chloro-3-((2-methyl-4-(phenoxymethyl)phenyl)carbamoyl)phenyl)carbamate.

13. A pharmaceutical composition comprising a compound or a pharmaceutically acceptable salt thereof according to claim 1, and a pharmaceutically acceptable carrier.

14. The pharmaceutical composition according to claim 13, wherein the compound or pharmaceutically acceptable salt thereof is a TREK1, TREK2 or both TREK1/TREK2 inhibitor.

15. A method for treating a disorder associated with TREK1, TREK2, or dual TREK1/TREK2 dysfunction in which inhibitors of TREK1, or both TREK1/TREK2 would offer therapeutic benefit in a mammal, comprising administering to the mammal in need thereof a therapeutically effective amount a compound of or pharmaceutically acceptable salt thereof according to claim 1.

16. The method according to claim 15, wherein a disorder associated with TREK1, TREK2, or dual TREK1/TREK2 dysfunction in which inhibitors of TREK1, TREK2 or both TREK1/TREK2 would offer therapeutic benefit in a mammal is a neurological and/or psychiatric disorder.

17. The method according to claim 16, wherein the neurological and/or psychiatric disorder is selected from depression, schizophrenia, anxiety disorders, bipolar depression, Alzheimer's disease, Parkinson's disease, Huntington's disease, Amyotrophic lateral sclerosis, neuropathic pain or cerebral infarction.

18. A kit comprising a compound or pharmaceutically acceptable salt thereof of claim 1, and one or more of: (a) at least one agent known to decrease TREK1 channel activity; (b) at least one agent known to decrease TREK2 channel activity; (c) at least one agent known to prevent and/or treat a disorder associated with TREK channel dysfunction in which inhibitors of TREK1, TREK2 or both TREK1/TREK2 would offer therapeutic benefit in a mammal; (d) instructions for preventing and/or treating a disorder associated with TREK dysfunction in which inhibitors of TREK1, TREK2 or both TREK1/TREK2 would offer therapeutic benefit in a mammal; and (e) instructions for administering the compound in connection with cognitive behavioral therapy.
